(19) United States
(12) Patent Application Publication
(10) Pub. No.: US 2025/0144200 A1
(43) Pub. Date: May 8, 2025

(54) INTRANASAL IMMUNOGENIC COMPOSITIONS

(71) Applicants: Novavax, Inc., Gaithersburg, MD (US); Novavax AB, Uppsala (SE)

(72) Inventors: Gale SMITH, Germantown, MD (US); Nita PATEL, Gaithersburg, MD (US); George GRIFFITH, Gaithersburg, MD (US); Karin LÖVGREN BENGTSSON, Uppsala (SE); Linda STERTMAN, Uppsala (SE)

(21) Appl. No.: 18/939,463

(22) Filed: Nov. 6, 2024

Related U.S. Application Data
(60) Provisional application No. 63/596,421, filed on Nov. 6, 2023.

(51) Int. Cl. A61K 39/215; A61K 9/00; A61K 39/00; A61K 39/145; A61K 39/155; A61K 47/02; A61K 47/26; A61P 31/14; C12N 7/00

(52) U.S. Cl. CPC A61K 39/215; A61K 9/0043; A61K 39/145; A61K 39/155; A61K 47/02; A61K 47/26; A61P 31/14; C12N 7/00; A61K 2039/543; A61K 2039/55577; A61K 2039/575; A61K 2039/70; C12N 2760/16134; C12N 2760/16234; C12N 2760/18534; C12N 2770/20034

(57) ABSTRACT

Disclosed herein are intranasal immunogenic compositions comprising viral glycoprotein nanoparticles, which are suitable for use as vaccines. The nanoparticles present antigens from pathogens surrounded to and associated with a detergent core resulting in enhanced stability and good immunogenicity. Dosages, formulations, and methods for preparing the vaccines and nanoparticles are also disclosed. Methods for using the intranasal immunogenic compositions to stimulate an immune response in a subject against a virus are also disclosed.

### RELATED APPLICATIONS

The present application claims priority to U.S. Provisional Patent Application No. 63/596,421, filed on Nov. 6, 2023, the contents of which is incorporated herein in its entirety by reference.

### DESCRIPTION OF THE TEXT FILE SUBMITTED ELECTRONICALLY

This application contains a Sequence Listing which has been submitted electronically in XML format and is hereby incorporated by reference in its entirety. Said XML copy, created on Nov. 6, 2024, is named 1450_107US1_Sequence_Listing_11_06_2024 and is 848,477 bytes in size.

### FIELD OF THE INVENTION

The present disclosure is generally related to immunogenic compositions for intranasal delivery. The immunogenic compositions contain nanoparticles comprising viral glycoproteins and optionally an adjuvant. Exemplary viral glycoproteins include SARS-COV-2 Spike(S) glycoproteins, hemagglutinin, and respiratory syncytial virus (RSV) fusion (F) glycoproteins. The nanoparticles have improved stability and enhanced epitope presentation. The disclosure also provides methods for producing the immunogenic compositions and methods of stimulating immune responses with the compositions.

### BACKGROUND OF THE INVENTION

Intranasal immunogenic compositions are highly desirable because they avoid needle-based injections and are therefore more acceptable to the consumer.

However, the development of intranasal immunogenic compositions is challenging because proteases within the nasal cavity (e.g., aminopeptidase, exopeptidases, and endopeptidases) can degrade intranasal immunogenic compositions and prevent the development of an immune response. This is particular true of subunit vaccines. Indeed, there is only one FDA approved intranasal vaccine, which is a live attenuated influenza vaccine known as FLUMIST®. FLUMIST® was taken off the market after a study showed that its efficacy was only three percent in children, providing almost no protective benefit.

There is a need in the art for the development of effective intranasal immunogenic compositions.

### SUMMARY OF THE INVENTION

Provided herein are intranasal immunogenic compositions which induce both mucosal immunity and systemic immunity against viruses. The COVID-19 experience shows that efforts to increase vaccination are essential. Needle-free vaccinations like intranasal vaccination will make individuals more willing to undergo vaccination. In developing countries, needle-free vaccinations are particularly desirable to prevent the transmission of blood-borne pathogens like HIV and hepatitis that can occur through vaccine administration with a needle.

Although intranasal administration has been long sought after, intranasal vaccines have not been successful. For example, a live attenuated influenza vaccine was approved by the FDA, but removed after a study showed that the vaccine had almost no benefit. Protein subunit vaccines are not believed to be suitable for intranasal administration because the protein subunits get degraded by proteases in the nasal cavity. Remarkably, Applicant have demonstrated that nanoparticles comprising a non-ionic detergent and a viral glycoprotein comprising a transmembrane domain, wherein the viral glycoprotein is anchored to the detergent core via the transmembrane domain of the viral glycoprotein are particularly good at inducing an immune response, even in the presence of proteases in the nasal cavity.

Provided herein are intranasal immunogenic compositions comprising: (i) a glycoprotein nanoparticle comprising a non-ionic detergent and a viral glycoprotein comprising a transmembrane domain, wherein the viral glycoprotein is anchored to the detergent core via the transmembrane domain of the viral glycoprotein; and (ii) a pharmaceutically acceptable buffer. In embodiments, the viral glycoprotein is selected from one or more of a hemagglutinin (HA) glycoprotein, a SARS-COV-2 Spike(S) glycoprotein, and a respiratory syncytial virus (RSV) fusion (F) glycoprotein. Also provided herein are prefilled syringes comprising the intranasal immunogenic compositions. Also provided herein are dry powder compositions comprising the intranasal immunogenic compositions. Also provided herein are intranasal delivery devices comprising the intranasal immunogenic compositions. Also provided herein are intranasal delivery devices comprising an aerosol comprising an intranasal immunogenic composition described herein. Also provided herein are intranasal delivery devices comprising an intranasal immunogenic composition described herein, wherein the composition is atomized. Also provided herein are methods of stimulating an immune response against SARS-COV-2, influenza, RSV, or a combination thereof in a patient comprising intranasally administering an intranasal immunogenic composition described herein. Also provided herein are methods of inducing mucosal immunity against SARS-COV-2, influenza, RSV, or a combination thereof in a patient comprising intranasally administering an intranasal immunogenic composition described herein. Also provided herein are methods of inducing production of neutralizing antibodies against SARS-COV-2, influenza, RSV, or a combination thereof in a patient comprising administering to the patient an intranasal immunogenic composition described herein. Also provided herein are methods of stimulating an immune response against SARS-COV-2, influenza, RSV, or a combination thereof in the nasal cavity of a patient, comprising administering an intranasal immunogenic composition described herein.

### BRIEF DESCRIPTION OF THE DRAWINGS

FIG. 1 shows an image of the study design of Example 3. The study compares the immune response resulting from intranasal and intramuscular boost with immunogenic compositions comprising SARS-COV-2 S glycoproteins in the presence or absence of a saponin adjuvant.

FIGS. 2A-2B shows the titer of anti-SARS-COV-2 Spike(S) glycoprotein antibodies against a SARS-COV-2 Spike glycoprotein of SEQ ID NO: 87 induced by intranasal or intramuscular boost with an immunogenic composition comprising a SARS-COV-2 Spike (“CoV S”) glycoprotein of SEQ ID NO: 274 in the presence (FIG. 2A) or absence (FIG. 2B) of a saponin adjuvant. The saponin adjuvant contained Fraction A of Quillaja Saponaria Molina and not Fraction C of Quillaja Saponaria Molina, and (ii) a second iscom particle containing Fraction C of Quillaja Saponaria Molina and not Fraction A of Quillaja Saponaria Molina. Fraction A and Fraction C accounted for 85% and 15% by weight, respectively, of the sum of the weights of fraction A of Quillaja Saponaria Molina and fraction C of Quillaja Saponaria Molina in the adjuvant.

FIGS. 3A-3B shows the titer of anti-SARS-COV-2 Spike(S) glycoprotein antibodies against the SARS-COV-2 Spike glycoprotein of the SARS-COV-2 XBB.1.5 strain induced by intranasal or intramuscular boost with an immunogenic composition comprising a SARS-COV-2 Spike (“CoV S”) glycoprotein of SEQ ID NO: 274 in the presence (FIG. 3A) or absence (FIG. 3B) of a saponin adjuvant. The saponin adjuvant contained Fraction A of Quillaja Saponaria Molina and not Fraction C of Quillaja Saponaria Molina, and (ii) a second iscom particle containing Fraction C of Quillaja Saponaria Molina and not Fraction A of Quillaja Saponaria Molina. Fraction A and Fraction C accounted for 85% and 15% by weight, respectively, of the sum of the weights of fraction A of Quillaja Saponaria Molina and fraction C of Quillaja Saponaria Molina in the adjuvant.

FIGS. 4A-4B shows the titer of anti-SARS-COV-2 Spike(S) glycoprotein antibodies against the SARS-COV-2 Spike glycoprotein of the SARS-COV-2 XBB.1.16.6 strain (SEQ ID NO: 312) induced by intranasal or intramuscular boost with an immunogenic composition comprising a SARS-COV-2 Spike (“CoV S”) glycoprotein of SEQ ID NO: 274 in the presence (FIG. 4A) or absence (FIG. 4B) of a saponin adjuvant. The saponin adjuvant contained Fraction A of Quillaja Saponaria Molina and not Fraction C of Quillaja Saponaria Molina, and (ii) a second iscom particle containing Fraction C of Quillaja Saponaria Molina and not Fraction A of Quillaja Saponaria Molina. Fraction A and Fraction C accounted for 85% and 15% by weight, respectively, of the sum of the weights of fraction A of Quillaja Saponaria Molina and fraction C of Quillaja Saponaria Molina in the adjuvant.

FIGS. 5A-5B shows the titer of anti-SARS-COV-2 Spike(S) glycoprotein antibodies against the SARS-COV-2 Spike glycoprotein of the SARS-COV-2 EG.5.1 (SEQ ID NO: 295) strain induced by intranasal or intramuscular boost with an immunogenic composition comprising a SARS-COV-2 Spike (“CoV S”) glycoprotein of SEQ ID NO: 274 in the presence (FIG. 5A) or absence (FIG. 5B) of a saponin adjuvant. The saponin adjuvant contained Fraction A of Quillaja Saponaria Molina and not Fraction C of Quillaja Saponaria Molina, and (ii) a second iscom particle containing Fraction C of Quillaja Saponaria Molina and not Fraction A of Quillaja Saponaria Molina. Fraction A and Fraction C accounted for 85% and 15% by weight, respectively, of the sum of the weights of fraction A of Quillaja Saponaria Molina and fraction C of Quillaja Saponaria Molina in the adjuvant.

FIG. 6 shows that serum collected from mice immunized with an immunogenic composition comprising a SARS-COV-2 S glycoprotein of SEQ ID NO: 222 and a saponin adjuvant neutralized a pseudovirus encoding an XBB.1.5 SARS-COV-2 S glycoprotein. The saponin adjuvant contained Fraction A of Quillaja Saponaria Molina and not Fraction C of Quillaja Saponaria Molina, and (ii) a second iscom particle containing Fraction C of Quillaja Saponaria Molina and not Fraction A of Quillaja Saponaria Molina. Fraction A and Fraction C accounted for 85% and 15% by weight, respectively, of the sum of the weights of fraction A of Quillaja Saponaria Molina and fraction C of Quillaja Saponaria Molina in the adjuvant.

FIG. 7 shows that serum collected from mice immunized with an immunogenic composition comprising a SARS-COV-2 S glycoprotein of SEQ ID NO: 222 and a saponin adjuvant neutralized a pseudovirus encoding an XBB.1.16.6 SARS-COV-2 S glycoprotein. The saponin adjuvant contained Fraction A of Quillaja Saponaria Molina and not Fraction C of Quillaja Saponaria Molina, and (ii) a second iscom particle containing Fraction C of Quillaja Saponaria Molina and not Fraction A of Quillaja Saponaria Molina. Fraction A and Fraction C accounted for 85% and 15% by weight, respectively, of the sum of the weights of fraction A of Quillaja Saponaria Molina and fraction C of Quillaja Saponaria Molina in the adjuvant.

FIG. 8 shows that serum collected from mice immunized with an immunogenic composition comprising a SARS-COV-2 S glycoprotein of SEQ ID NO: 222 and a saponin adjuvant neutralized a pseudovirus encoding an EG.5.1 SARS-COV-2 S glycoprotein. The saponin adjuvant contained Fraction A of Quillaja Saponaria Molina and not Fraction C of Quillaja Saponaria Molina, and (ii) a second iscom particle containing Fraction C of Quillaja Saponaria Molina and not Fraction A of Quillaja Saponaria Molina. Fraction A and Fraction C accounted for 85% and 15% by weight, respectively, of the sum of the weights of fraction A of Quillaja Saponaria Molina and fraction C of Quillaja Saponaria Molina in the adjuvant.

FIGS. 9A-9B shows anti-SARS-COV-2 IgA antibodies against a SARS-COV-2 Spike glycoprotein of SEQ ID NO: 87 present in the bronchoalveolar lavage (BAL) of mice immunized intranasally or intramuscularly with an immunogenic composition comprising a SARS-COV-2 S glycoprotein of SEQ ID NO: 222 in the presence (FIG. 9A) and absence of saponin adjuvant (FIG. 9B). The saponin adjuvant contained Fraction A of Quillaja Saponaria Molina and not Fraction C of Quillaja Saponaria Molina, and (ii) a second iscom particle containing Fraction C of Quillaja Saponaria Molina and not Fraction A of Quillaja Saponaria Molina. Fraction A and Fraction C accounted for 85% and 15% by weight, respectively, of the sum of the weights of fraction A of Quillaja Saponaria Molina and fraction C of Quillaja Saponaria Molina in the adjuvant.

FIGS. 9C-9D shows anti-SARS-COV-2 IgG antibodies against a SARS-COV-2 Spike glycoprotein of SEQ ID NO: 87 present in the BAL of mice immunized intranasally or intramuscularly with an immunogenic composition comprising a SARS-COV-2 S glycoprotein of SEQ ID NO: 222 in the presence (FIG. 9C) and absence of saponin adjuvant (FIG. 9D). The saponin adjuvant contained Fraction A of Quillaja Saponaria Molina and not Fraction C of Quillaja Saponaria Molina, and (ii) a second iscom particle containing Fraction C of Quillaja Saponaria Molina and not Fraction A of Quillaja Saponaria Molina. Fraction A and Fraction C accounted for 85% and 15% by weight, respectively, of the sum of the weights of fraction A of Quillaja Saponaria Molina and fraction C of Quillaja Saponaria Molina in the adjuvant.

FIGS. 10A-10B shows anti-SARS-COV-2 IgA antibodies against a SARS-COV-2 XBB.1.5 S glycoprotein present in the bronchoalveolar lavage (BAL) of mice immunized intranasally or intramuscularly with an immunogenic composition comprising a SARS-COV-2 S glycoprotein of SEQ ID NO: 222 in the presence (FIG. 10A) and absence of saponin adjuvant (FIG. 10B). The saponin adjuvant contained Fraction A of Quillaja Saponaria Molina and not Fraction C of Quillaja Saponaria Molina, and (ii) a second iscom particle containing Fraction C of Quillaja Saponaria Molina and not Fraction A of Quillaja Saponaria Molina. Fraction A and Fraction C accounted for 85% and 15% by weight, respectively, of the sum of the weights of fraction A of Quillaja Saponaria Molina and fraction C of Quillaja Saponaria Molina in the adjuvant.

FIGS. 10C-10D shows anti-SARS-COV-2 IgG antibodies against a SARS-COV-2 XBB.1.5 S glycoprotein present in the BAL of mice immunized intranasally or intramuscularly with an immunogenic composition comprising a SARS-COV-2 S glycoprotein of SEQ ID NO: 222 in the presence (FIG. 10C) and absence of saponin adjuvant (FIG. 10D). The saponin adjuvant contained Fraction A of Quillaja Saponaria Molina and not Fraction C of Quillaja Saponaria Molina, and (ii) a second iscom particle containing Fraction C of Quillaja Saponaria Molina and not Fraction A of Quillaja Saponaria Molina. Fraction A and Fraction C accounted for 85% and 15% by weight, respectively, of the sum of the weights of fraction A of Quillaja Saponaria Molina and fraction C of Quillaja Saponaria Molina in the adjuvant.

FIGS. 11A-11B shows anti-SARS-COV-2 IgA antibodies against a SARS-COV-2 EG.5.1 S glycoprotein present in the bronchoalveolar lavage (BAL) of mice immunized intranasally or intramuscularly with an immunogenic composition comprising a SARS-COV-2 S glycoprotein of SEQ ID NO: 222 in the presence (FIG. 11A) and absence of saponin adjuvant (FIG. 11B). The saponin adjuvant contained Fraction A of Quillaja Saponaria Molina and not Fraction C of Quillaja Saponaria Molina, and (ii) a second iscom particle containing Fraction C of Quillaja Saponaria Molina and not Fraction A of Quillaja Saponaria Molina. Fraction A and Fraction C accounted for 85% and 15% by weight, respectively, of the sum of the weights of fraction A of Quillaja Saponaria Molina and fraction C of Quillaja Saponaria Molina in the adjuvant.

FIGS. 11C-11D shows anti-SARS-COV-2 IgG antibodies against a SARS-COV-2 EG.5.1S glycoprotein present in the BAL of mice immunized intranasally or intramuscularly with an immunogenic composition comprising a SARS-COV-2 S glycoprotein of SEQ ID NO: 222 in the presence (FIG. 11C) and absence of saponin adjuvant (FIG. 11D). The saponin adjuvant contained Fraction A of Quillaja Saponaria Molina and not Fraction C of Quillaja Saponaria Molina, and (ii) a second iscom particle containing Fraction C of Quillaja Saponaria Molina and not Fraction A of Quillaja Saponaria Molina. Fraction A and Fraction C accounted for 85% and 15% by weight, respectively, of the sum of the weights of fraction A of Quillaja Saponaria Molina and fraction C of Quillaja Saponaria Molina in the adjuvant.

FIG. 12 shows neutralizing antibodies against a pseudovirus expressing the SARS-COV-2 S glycoprotein of XBB.1.5 that are induced by an immunogenic composition comprising a SARS-CoV-2 S glycoprotein of SEQ ID NO: 222 in the presence of saponin adjuvant. Intranasal administration induces a 19.8 fold increase in neutralizing antibodies against a pseudovirus expressing the SARS-COV-2 S glycoprotein of XBB.1.5, whereas intramuscular administration only induces a 2.12 fold increase in neutralizing antibodies against the same pseudovirus. The saponin adjuvant contained Fraction A of Quillaja Saponaria Molina and not Fraction C of Quillaja Saponaria Molina, and (ii) a second iscom particle containing Fraction C of Quillaja Saponaria Molina and not Fraction A of Quillaja Saponaria Molina. Fraction A and Fraction C accounted for 85% and 15% by weight, respectively, of the sum of the weights of fraction A of Quillaja Saponaria Molina and fraction C of Quillaja Saponaria Molina in the adjuvant.

FIG. 13 shows IFN-γ secretion from Th1 CD4+ T cells after immunization with an immunogenic composition comprising a SARS-COV-2 S glycoprotein of SEQ ID NO: 222 in the presence of saponin adjuvant. The saponin adjuvant contained Fraction A of Quillaja Saponaria Molina and not Fraction C of Quillaja Saponaria Molina, and (ii) a second iscom particle containing Fraction C of Quillaja Saponaria Molina and not Fraction A of Quillaja Saponaria Molina. Fraction A and Fraction C accounted for 85% and 15% by weight, respectively, of the sum of the weights of fraction A of Quillaja Saponaria Molina and fraction C of Quillaja Saponaria Molina in the adjuvant.

FIG. 14 shows triple positive Th1 CD4+ T cells after immunization with an immunogenic composition comprising a SARS-COV-2 S glycoprotein of SEQ ID NO: 222 in the presence of saponin adjuvant. The saponin adjuvant contained Fraction A of Quillaja Saponaria Molina and not Fraction C of Quillaja Saponaria Molina, and (ii) a second iscom particle containing Fraction C of Quillaja Saponaria Molina and not Fraction A of Quillaja Saponaria Molina. Fraction A and Fraction C accounted for 85% and 15% by weight, respectively, of the sum of the weights of fraction A of Quillaja Saponaria Molina and fraction C of Quillaja Saponaria Molina in the adjuvant.

FIG. 15 shows the percentage of germinal center B cells out of total B cells after immunization with an immunogenic composition comprising a SARS-COV-2 S glycoprotein of SEQ ID NO: 222 in the presence of saponin adjuvant. The saponin adjuvant contained Fraction A of Quillaja Saponaria Molina and not Fraction C of Quillaja Saponaria Molina, and (ii) a second iscom particle containing Fraction C of Quillaja Saponaria Molina and not Fraction A of Quillaja Saponaria Molina. Fraction A and Fraction C accounted for 85% and 15% by weight, respectively, of the sum of the weights of fraction A of Quillaja Saponaria Molina and fraction C of Quillaja Saponaria Molina in the adjuvant.

FIG. 16 shows triple positive Th1 C8+ T cells after immunization with an immunogenic composition comprising a SARS-COV-2 S glycoprotein of SEQ ID NO: 222 in the presence of saponin adjuvant. The saponin adjuvant contained Fraction A of Quillaja Saponaria Molina and not Fraction C of Quillaja Saponaria Molina, and (ii) a second iscom particle containing Fraction C of Quillaja Saponaria Molina and not Fraction A of Quillaja Saponaria Molina. Fraction A and Fraction C accounted for 85% and 15% by weight, respectively, of the sum of the weights of fraction A of Quillaja Saponaria Molina and fraction C of Quillaja Saponaria Molina in the adjuvant.

FIG. 17 provides a timeline of the experiment of Example 5. Rhesus macaques are boosted on day 447 intranasally or intramuscularly with an immunogenic composition containing a RSV

F glycoprotein and a saponin adjuvant or an immunogenic composition containing a HA glycoprotein and a SARS-COV-2 S glycoprotein.

FIG. 18 is a schematic of the experiment of Example 5. Rhesus macaques are boosted intramuscularly or intranasally with an immunogenic containing a SARS-COV-2 S glycoprotein and a saponin adjuvant.

FIG. 19A shows the primary structure of a wild-type SARS-COV-2 S polypeptide, containing a signal peptide, numbered with respect to SEQ ID NO: 1. FIG. 19B shows the primary structure of a wild-type SARS-COV-2 S polypeptide, without a signal peptide, numbered with respect to SEQ ID NO: 2.

FIGS. 20A-20B show results from of Example 4 showing bronchiole alveolar lavage anti-XBB.1.5 IgG and IgA responses to XBB.1.5 Primary and XBB.1.5 Boost intranasal vaccination.

FIGS. 21A-21B show results from of Example 4 showing bronchiole alveolar lavage anti-JN.1 IgG and IgA responses to XBB.1.5 Primary and XBB.1.5 Boost intranasal vaccination.

FIGS. 22A-22B show results from of Example 4 showing bronchiole alveolar lavage anti-XBB.1.5 IgG and IgA responses to Bivalent BA.5 Primary and XBB.1.5 Boost intranasal vaccination.

FIGS. 23A-23B show results from of Example 4 showing bronchiole alveolar lavage anti-JN.1 IgG and IgA responses to Bivalent BA.5 Primary and XBB.1.5 Boost intranasal vaccination.

FIG. 24 shows bronchiole results from of Example 4 showing alveolar lavage pseudovirus neutralization responses to Primary Two Doses of Bivalent BA.5 and XBB.1.5 intranasal vaccination.

FIG. 25 shows results from of Example 4 showing nasal wash-anti-XBB.1.5 IgA responses to XBB.1.5 Primary and XBB.1.5 Boost.

FIG. 26 shows results from of Example 4 showing nasal wash-anti-XBB.1.5 IgA responses to Bivalent BA.5 Primary and XBB.1.5 Boost.

FIGS. 27A-27B show results from of Example 4 showing Effector CD4+ T cells Th1/Th2 responses using Intracellular Cytokine Staining (ICCS) assay 21 days post primary two doses of Prototype+BA.5 (Intranasal) (5 μg rS+5 μg Matrix-M) versus XBB.1.5 (Intranasal) (5 μg rS+5 μg Matrix-M).

FIG. 28 shows results from of Example 4 showing CD4+ T cells Th1/Th2 responses in BAL using Intracellular Cytokine Staining (ICCS) assay 21 days post primary dose Prototype+BA.5 (Intranasal) (5 μg rS+5 μg Matrix-M) versus XBB.1.5 (Intranasal) (5 μg rS+5 μg Matrix-M).

FIG. 29 shows results from of Example 4 showing CD8+ T cells Th1 responses in BAL using Intracellular Cytokine Staining (ICCS) assay 21 days post primary dose Prototype+BA.5 (Intranasal) (5 μg rS+5 μg Matrix-M) versus XBB.1.5 (Intranasal) (5 μg rS+5 μg Matrix-M).

FIGS. 30A-30B show results from of Example 4 showing CD4+ T cells Th1/Th2 responses to two doses of Bivalent BA.5 Primary followed by XBB.1.5 Intramuscular Boost (1 μg rS+5 μg Matrix-M) versus Bivalent BA.5 Primary followed by XBB.1.5 Intranasal Boost (1 μg rS+5 μg Matrix-M) using Intracellular Cytokine Staining (ICCS) assay.

FIGS. 31A-31B show results from of Example 4 showing CD4+ T cells Th1/Th2 responses to two doses of Bivalent BA.5 Primary followed by XBB.1.5 Intranasal Boost (1 μg rS+5 μg Matrix-M) versus two doses of Bivalent BA.5 Primary followed by XBB.1.5 Intranasal Boost (5 μg rS+5 μg Matrix-M) using Intracellular Cytokine Staining (ICCS) assay.

FIGS. 32A-32C show results from of Example 4 showing CD4+ T cells Th1/Th2 responses to two doses of XBB.1.5 Primary followed by XBB.1.5 Intramuscular Boost (1 μg rS+5 μg Matrix-M) versus two doses of XBB.1.5 Primary followed by XBB.1.5 Intranasal Boost (1 μg rS+5 μg Matrix-M) versus two doses of XBB.1.5 Primary followed by XBB.1.5 Intranasal Boost (5 μg rS+5 μg Matrix-M) using Intracellular Cytokine Staining (ICCS) assay.

FIG. 33 shows results from of Example 4 showing Triple Th1 CD4+ T cells response two doses of Bivalent BA.5 Primary followed by XBB.1.5 Intramuscular Boost (1 μg rS) versus two doses of Bivalent BA.5 Primary followed by XBB.1.5 Intranasal Boost (1 μg rS) versus two doses of Bivalent BA.5 Primary followed by XBB.1.5 Intranasal Boost (5 μg rS) using Intracellular Cytokine Staining (ICCS) assay.

FIG. 34 shows results from of Example 4 showing Triple Th1 CD4+ T cells response two doses of XBB.1.5 Primary followed by XBB.1.5 Intramuscular Boost (1 μg rS) versus two doses of XBB.1.5 Primary followed by XBB.1.5 Intranasal Boost (1 μg rS) versus two doses of XBB.1.5 Primary followed by XBB.1.5 Intranasal Boost (5 μg rS) using Intracellular Cytokine Staining (ICCS) assay.

FIGS. 35A-35B show results from of Example 4 showing CD4+ T cells Th1/Th2 responses to intramuscular versus intranasal XBB.1.5 Boost following primary Prototype+BA.5 or XBB.1.5 using Peptide Pool Stimulation.

FIGS. 36A-36B show results from of Example 4 showing CD8+ T cells Th1 responses to intramuscular versus intranasal XBB.1.5 Boost following primary Prototype+BA.5 or XBB.1.5 using Peptide Pool Stimulation.

FIGS. 37A-37B show results from of Example 4 showing T follicular helper cells (Tfh) and germinal center B cells in response to XBB.1.5 Boost following primary Bivalent BA.5 or XBB.1.5.

FIGS. 38A-38B show results from of Example 4 showing bronchiole alveolar lavage CD4+ T cells Th1/Th2 responses to post XBB.1.5 intranasal boost following primary Bivalent BA.5 or XBB.1.5.

FIG. 39 shows results from of Example 4 showing Triple Th1 CD4+ T cells in spleen cells in response to intranasal vaccination of XBB.1.5 rS (5 μg rS+5 μg Matrix-M) at day 21, day 41 and day 56 using Intracellular Cytokine Staining (ICCS) assay.

FIG. 40 shows results from of Example 4 showing CD8+ T cells Th1 responses to Primary and Boost with XBB.1.5 (5 μg rS) by intranasal vaccination.

### DETAILED DESCRIPTION OF THE INVENTION

### A. Definitions

As used herein, and in the appended claims, the singular forms “a”, “an”, and “the” include plural referents unless the context clearly dictates otherwise. Thus, for example, reference to “a protein” can refer to one protein or to mixtures of such protein, and reference to “the method” includes reference to equivalent steps and/or methods known to those skilled in the art, and so forth.

As used herein, the term “adjuvant” refers to a compound that, when used in combination with an immunogen, augments or otherwise alters or modifies the immune response induced against the immunogen. Modification of the immune response may include intensification or broadening the specificity of either or both antibody and cellular immune responses.

As used herein, the term “about” or “approximately” when preceding a numerical value indicates the value plus or minus a range of 10%. For example, “about 100” encompasses 90 and 110.

As used herein, the terms “immunogen,” “antigen,” and “epitope” refer to substances such as proteins, including glycoproteins, and peptides that are capable of eliciting an immune response.

As used herein, an “immunogenic composition” is a composition that comprises an antigen where administration of the composition to a subject results in the development in the subject of a humoral and/or a cellular immune response to the antigen.

As used herein, a “subunit” composition, for example a vaccine, that includes one or more selected antigens but not all antigens from a pathogen. Such a composition is substantially free of intact virus or the lysate of such cells or particles and is typically prepared from at least partially purified, often substantially purified immunogenic polypeptides from the pathogen. The antigens in the subunit composition disclosed herein are typically prepared recombinantly, often using a baculovirus system.

As used herein, “substantially” refers to isolation of a substance (e.g. a compound, polynucleotide, or polypeptide) such that the substance forms the majority percent of the sample in which it is contained. For example, in a sample, a substantially purified component comprises 85%, preferably 85%-90%, more preferably at least 95%-99.5%, and most preferably at least 99% of the sample. If a component is substantially replaced the amount remaining in a sample is less than or equal to about 0.5% to about 10%, preferably less than about 0.5% to about 1.0%.

The terms “treat,” “treatment,” and “treating,” as used herein, refer to an approach for obtaining beneficial or desired results, for example, clinical results. For the purposes of this disclosure, beneficial or desired results may include inhibiting or suppressing the initiation or progression of an infection or a disease; ameliorating, or reducing the development of, symptoms of an infection or disease; or a combination thereof.

“Prevention,” as used herein, is used interchangeably with “prophylaxis” and can mean complete prevention of an infection or disease, or prevention of the development of symptoms of that infection or disease; a delay in the onset of an infection or disease or its symptoms; or a decrease in the severity of a subsequently developed infection or disease or its symptoms.

As used herein an “effective dose” or “effective amount” refers to an amount of an immunogen sufficient to induce an immune response that reduces at least one symptom of pathogen infection. An effective dose or effective amount may be determined e.g., by measuring amounts of neutralizing secretory and/or serum antibodies, e.g., by plaque neutralization, complement fixation, enzyme-linked immunosorbent (ELISA), or microneutralization assay.

As used herein, the term “vaccine” refers to an immunogenic composition, such as an immunogen derived from a pathogen, which is used to induce an immune response against the pathogen that provides protective immunity (e.g., immunity that protects a subject against infection with the pathogen and/or reduces the severity of the disease or condition caused by infection with the pathogen). The protective immune response may include formation of antibodies and/or a cell-mediated response. Depending on context, the term “vaccine” may also refer to a suspension or solution of an immunogen that is administered to a subject to produce protective immunity.

As used herein, the term “subject” includes humans and other animals. Typically, the subject is a human. For example, the subject may be an adult, a teenager, a child (2 years to 14 years of age), an infant (birth to 2 year), or a neonate (up to 2 months). In particular aspects, the subject is up to 4 months old, or up to 6 months old. In aspects, the adults are seniors about 65 years or older, or about 60 years or older. In aspects, the subject is a pregnant woman or a woman intending to become pregnant. In other aspects, subject is not a human; for example a non-human primate; for example, a baboon, a chimpanzee, a gorilla, or a macaque. In certain aspects, the subject may be a pet, such as a dog or cat. In embodiments, the subject is immunocompromised. In embodiments, the immunocompromised subject is administered a medication that causes immunosuppression. Non-limiting examples of medications that cause immunosuppression include corticosteroids (e.g., prednisone), alkylating agents (e.g., cyclophosphamide), antimetabolites (e.g., azathioprine or 6-mercaptopurine), transplant-related immunosuppressive drugs (e.g., cyclosporine, tacrolimus, sirolimus, or mycophenolate mofetil), mitoxantrone, chemotherapeutic agents, methotrexate, tumor necrosis factor (TNF)-blocking agents (e.g., etanercept, adalimumab, infliximab). In embodiments, the immunocompromised subject is infected with a virus (e.g., human immunodeficiency virus or Epstein-Barr virus). In embodiments, the virus is a respiratory virus, such as respiratory syncytial virus, influenza, parainfluenza, adenovirus, or a picornavirus. In embodiments, the immunocompromised subject has acquired immunodeficiency syndrome (AIDS). In embodiments, the immunocompromised subject is a person living with human immunodeficiency virus (HIV). In embodiments, the immunocompromised subject is immunocompromised due to a treatment regiment designed to prevent inflammation or prevent rejection of a transplant. In embodiments, the immunocompromised subject is a subject who has received a transplant. In embodiments, the immunocompromised subject has undergone radiation therapy or a splenectomy. In embodiments, the immunocompromised subject has been diagnosed with cancer, an autoimmune disease, tuberculosis, a substance use disorder (e.g., an alcohol, opioid, or cocaine use disorder), stroke or cerebrovascular disease, a solid organ or blood stem cell transplant, sickle cell disease, thalassemia, autoimmune lymphoproliferative syndrome (ALPS), autoimmune polyglandular syndrome type 1 (APS-1), B-cell expansion with NF-κB and T-cell anergy (BENTA) disease, capsase eight deficiency state (CEDS), chronic granulomatous disease (CGD), common variable immunodeficiency (CVID), congenital neutropenia syndromes, a deficiency in the cytotoxic T-lymphocyte-associated antigen 4 (CTLA-4), a DOCK8 deficiency, a GATA2 deficiency, a glycosylation disorder with immunodeficiency, a hyper-immunoglobulin E syndrome (HIES), hyper-immunoglobulin M syndrome, diabetes, type 1 diabetes, type 2 diabetes, interferon gamma deficiency, interleukin 12 deficiency, interleukin 23 deficiency, leukocyte adhesion deficiency, lipopolysaccharide-responsive beige-like anchor (LRBA) deficiency, PI3 kinase disease, PLCG2-associated antibody deficiency and immune dysregulation (PLAID), severe combined immunodeficiency (SCID), STAT3 dominant-negative disease, STAT3 gain-of-function disease, warts, hypogammaglobulinemia, infections, and myelokathexis (WHIM) syndrome, Wisckott-Aldrich syndrome (WAS), X-linked agammaglobulinemia (XLA), X-linked lymphoproliferative disease (XLP), uremia, malnutrition, or XMEN disease. In embodiments, the immunocompromised subject is a current or former cigarette smoker. In embodiments, the immunocompromised subject has a B-cell defect, T-cell defect, macrophage defect, cytokine defect, phagocyte deficiency, phagocyte dysfunction, complement deficiency or a combination thereof. In embodiments, the subject is overweight or obese. In embodiments, an overweight subject has a body mass index (BMI)≥25 kg/m2 and <30 kg/m2. In embodiments, an obese subject has a BMI that is ≥30 kg/m2. In embodiments, the subject has a mental health condition. In embodiments, the mental health condition is depression, schizophrenia, or anxiety.

As used herein, the term “pharmaceutically acceptable” means being approved by a regulatory agency of a U.S. Federal or a state government or listed in the U.S. Pharmacopeia, European Pharmacopeia or other generally recognized pharmacopeia for use in mammals, and more particularly in humans. These compositions can be useful as a vaccine and/or antigenic compositions for inducing a protective immune response in a vertebrate.

As used herein, the term “NVX-CoV2373” refers to a vaccine composition comprising the BV2373 Spike glycoprotein (SEQ ID NO: 87) and a saponin adjuvant, wherein the saponin adjuvant contains Fraction A and Fraction C iscom matrix, wherein the Fraction A ISCOM matrix is present at about 85% by weight of the saponin adjuvant, and wherein Fraction C ISCOM matrix is present at about 15% by weight of the saponin adjuvant.

As used herein, the term “modification” as it refers to a viral glycoprotein refers to mutation, deletion, or addition of one amino acid of the viral glycoprotein. The location of a modification within a SARS-COV-2 Spike(S) glycoprotein (alternatively referred to herein as a “CoV S glycoprotein”) can be determined by aligning the sequence of the polypeptide to SEQ ID NO: 1 (CoV S polypeptide containing signal peptide) or SEQ ID NO: 2 (mature CoV S polypeptide lacking a signal peptide). The location of a modification within a respiratory syncytial virus (RSV) fusion (F) glycoprotein can be determined by aligning the sequence of the polypeptide to SEQ ID NO: 330 (mature RSV F glycoprotein lacking signal peptide) or SEQ ID NO: 329 (RSV F glycoprotein containing a signal peptide).

The term SARS-COV-2 “variant”, used interchangeably herein with a “heterogeneous SARS-COV-2 strain,” refers to a SARS-COV-2 virus comprising a CoV S polypeptide having one or more modifications as compared to a SARS-COV S polypeptide having the amino acid sequence of SEQ ID NO: 2. For example, a SARS-COV-2 variant may have at least about 2, at least about 3, at least about 4, at least about 5, at least about 6, at least about 7, at least about 8, at least about 9, at least about 10, at least about 11, at least about 12, at least about 13, at least about 14, at least about 15, at least about 16, at least about 17, at least about 18, at least about 19, at least about 20, at least about 21, at least about 22, at least about 23, at least about 24, at least about 25, at least about 26, at least about 27, at least about 28, at least about 29, at least about 30, at least about 31, at least about 32, at least about 33, at least about 34, or at least about 35 modifications, as compared to a CoV S polypeptide having the amino acid sequence of SEQ ID NO: 2. For example, a SARS-CoV-2 variant may have at least one and up to 2, up to 3, up to 4, up to 5, up to 6, up to 7, up to 8, up to 9, up to 10, up to 11, up to 12, up to 13, up to 14, up to 15, up to 16, up to 17, up to 18, up to 19, up to 20, up to 21, up to 22, up to 23, up to 24, up to 25, up to 26, up to 27, up to 28, up to 29, up to 30, up to 31, up to 32, up to 33, up to 34, up to 35 modifications, up to 40 modifications, up to 45 modifications, up to 50 modifications, up to 55 modifications, up to 60 modifications, up to 65 modifications, up to 70 modifications, up to 75 modifications, up to 80 modifications, up to 85 modifications, up to 90 modifications, up to 95 modifications, or up to 100 modifications as compared to a CoV S polypeptide having the amino acid sequence of SEQ ID NO: 2. In aspects, a SARS-COV-2 variant may have between about 2 and about 35 modifications, between about 5 and about 10 modifications, between about 5 and about 20 modifications, between about 10 and about 20 modifications, between about 15 and about 25 modifications, between about 20 and 30 modifications, between about 20 and about 40 modifications, between about 25 and about 45 modifications, between about 25 and about 100 modifications, between about 25 and about 45 modifications, or between about 35 and about 100 modifications, as compared to a CoV S polypeptide having the amino acid sequence of SEQ ID NO: 2.

The term “efficacy” of an immunogenic composition or vaccine composition described herein refers to the percentage reduction of disease (e.g., COVID-19) in a group administered an immunogenic composition as compared to a group that is not administered the immunogenic composition. In embodiments, efficacy (E) is calculated using the following equation: E (%)=(1-RR)×100, where RR=relative risk of incidence rates between the group administered the immunogenic composition and the group that is not administered the immunogenic composition. In embodiments, immunogenic compositions described herein have an efficacy against a SARS-CoV-2 virus or heterogeneous SARS-COV-2 strain that is at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, between about 50% and about 99%, between about 50% and about 98%, between about 60% and about 99%, between about 60% and about 98%, between about 70% and about 98%, between about 70% and about 95%, between about 70% and about 99%, between about 80% and about 99%, between about 80% and about 98%, between about 80% and about 95%, between about 85% and about 99%, between about 85% and about 98%, between about 85% and about 95%, between about 90% and about 95%, between about 90% and 98%, or between about 90% and about 99%.

The term “percent identity” in the context of two or more nucleic acid or polypeptide sequences, refers to two or more sequences or subsequences that have a specified percentage of nucleotides or amino acid residues that are the same, when compared. Unless otherwise indicated, percent identity is calculated over the entire length of the compared sequences using the tool EMBOSS Needle, which is available online at ebi.ac.uk/Tools/psa/emboss_needle/. When the percent identity of two polypeptides is compared, the following default parameters are used: Output Format=pair; Matrix=BLOSUM62; Gap Open=10; Gap Extend 0.5, End Gap Penalty=false, End Gap Open=10, End Gap Extend=0.5. When the percent identity of two nucleic acids is compared, the following default parameters are used: Output Format=pair; Matrix=DNAfull; Gap Open=10; Gap Extend 0.5, End Gap Penalty=false, End Gap Open=10, End Gap Extend=0.5.

As used herein, the terms “intranasal administration” and “nasal administration” refer to administration of an immunogenic composition described herein to the nasal cavity of a subject. In embodiments, intranasal administration is achieved using a liquid preparation (e.g., an aqueous preparation), an aerosolized preparation, or a dry powder preparation. In embodiments, the liquid preparation, aerosolized preparation, or dry powder preparation is administered via an externally propelled nasal delivery device. In embodiments, the liquid preparation, aerosolized preparation, or dry powder preparation is administered via a self-propelled (i.e., via inhalation) nasal delivery device. In embodiments, the liquid preparation, aerosolized preparation, or dry powder preparation is administered via nasal insufflation (when an immunogenic composition is blown into the nose) or nasal instillation (when an immunogenic composition is dropped into the nose). In embodiments, the liquid preparation, aerosolized preparation, or dry powder preparation is administered via a gel, cream, ointment, lotion, or paste applied to one or more nasal epithelium (e.g., olfactory epithelium or nasal respiratory epithelium). In embodiments, the immunogenic composition is applied to mucus in the nasal cavity of a subject.

As used herein, the term “non-invasive nasal delivery device” refers an instrument that is capable of delivering an immunogenic composition to the nasal cavity without piercing the epithelium of the subject. Non-limiting examples of non-invasive nasal delivery devices include propellant (e.g., a pressurized inhaler) and non-propellant (e.g., a pump-type inhaler) types of aerosol or atomizer devices, particle dispersion devices, nebulizers, and pressurized olfactory delivery devices for delivery of liquid or powder formulations.

As used herein, the term “dry powder composition” refers to a lyophilized or spray dried form of an immunogenic composition described herein. In embodiments, a dry powder composition contains less than 10%, 9%, 8%, 7%, 6%, 5%, 4%, 3%, 2%, 1%, or less residual water content.

As used herein, the term “permeability enhancer” refers to a component of an immunogenic composition formulated for intranasal administration which promotes the passage of a viral glycoprotein through the nasal epithelium.

As used herein, the terms “bedside mix,” “bedside formulations,” “bedside vaccine compositions,” “bedside vials,” “bedside vial formulations” refer to vaccine formulations that are prepared immediately prior to administration. Such vaccine formulations contain viral antigens and adjuvants that are separately stored in different containers and are administered to a subject (e.g., either administering two consecutive injections, or combining the antigens and the adjuvants into one injection prior to administration).

As used herein, the terms “co-formulation mix,” “co-formulation,” “co-formulation immunogenic compositions,” “prefilled syringes,” “pre-mix,” refer to vaccine formulations that are prepared for short to long-term storage prior to the time of administration to a subject. Such vaccine formulations contain a combination of antigens and adjuvant in the same container and prepared in advance of administration. In embodiments, formulations contain hemagglutinin and adjuvant (e.g., saponin adjuvant) and form HaSMaNs (Hemagglutinin Saponin Matrix Nanoparticles).

As used herein, the term “split-virion” refers to a virus (e.g., an influenza virus or a SARS-CoV-2 virus), which has a viral membrane that has been disrupted with a surfactant. Examples of surfactants are described throughout this disclosure. Split-virions do not undergo further purification, so they typically contain multiple viral proteins.

As used herein, the term “recombinant” as it refers to a protein (e.g. hemagglutinin) that is produced in a cell by transcription and translation of a nucleic acid that is introduced into a cell. The nucleic acid may be introduced via a vector or a virus encoding the nucleic acid.

As used herein, the term “whole influenza virus” refers to a virus that comprises all of its envelope, viral membrane, nucleocapsid, and genetic material. In embodiments, the whole influenza virus is inactivated.

As used herein, the term “inactivated virus” refers to a virus that has undergone treatment to substantially reduce or eliminate its virulence compared to the wild-type virus.

The term “bronchoalveolar lavage,” also referred to as “BAL” refers to a fluid sample retrieved from a patient's lungs. In embodiments, the BAL is retrieved during a bronchoscopy. During a bronchoscopy, a bronchoscope containing a solution (e.g., saline) is passed through the mouth or nose into the lungs. The solution is subsequently collected from the lungs.

The term “mucosal immunity” refers to the cellular and humoral immune response that occurs in mucosal membranes. In embodiments, the methods of the disclosure provided herein result in mucosal immunity in the respiratory system.

The term “atomization” refers to the generation of fine, inhalable droplets of a liquid. The typical dimensions of atomized droplets are in the range of several microns.

The term “aerosol” refers to a dispersion of solid or liquid particles in a gas phase. In embodiments, the gas phase is air.

### B. Intranasal Immunogenic Compositions

Provided herein are intranasal immunogenic compositions comprising nanoparticles comprising a viral glycoprotein. In embodiments, the immunogenic compositions are for intranasal administration. Advantageously, intranasal administration of the intranasal immunogenic compositions described herein induces mucosal immunity. The induction of mucosal immunity may result in reduction of viral transmission.

In embodiments, the viral glycoproteins are associated with a detergent core. In embodiments, the detergent core is a non-ionic detergent. In embodiments, the nanoparticles comprise viral glycoproteins assembled into multi-oligomeric glycoprotein-detergent nanoparticles with head regions projecting outward and the detergent forming a central core surrounded by the glycoprotein. In embodiments, the viral glycoproteins comprise a transmembrane domain. In embodiments, the transmembrane domain associates with the detergent.

In embodiments, the immunogenic compositions comprise multiple nanoparticles that contain different viral glycoproteins. In embodiments, the immunogenic compositions comprise from 1 to 50, from 1 to 45, from 1 to 40, from 1 to 35, from 1 to 30, from 1 to 25, from 1 to 20, from 1 to 14, from 1 to 10, from 1 to 9, from 1 to 8, from 1 to 7, from 1 to 6, from 1 to 4, from 1 to 3, or from 1 to 2 different nanoparticles. In embodiments, the viral glycoproteins are selected from one or more of an influenza hemagglutinin (HA) glycoprotein, a SARS-COV-2 S glycoprotein, and an RSV F glycoprotein.

Non-limiting examples of viral glycoproteins are described below.

SARS-COV-2 S glycoproteins (“CoV S glycoproteins”)

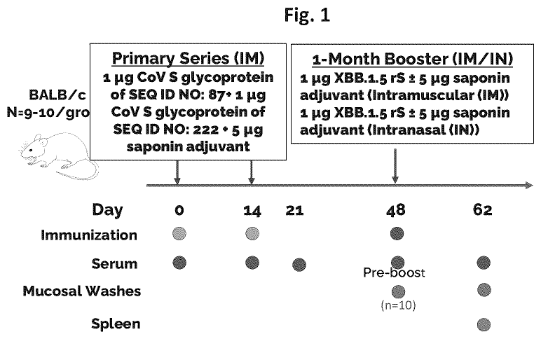

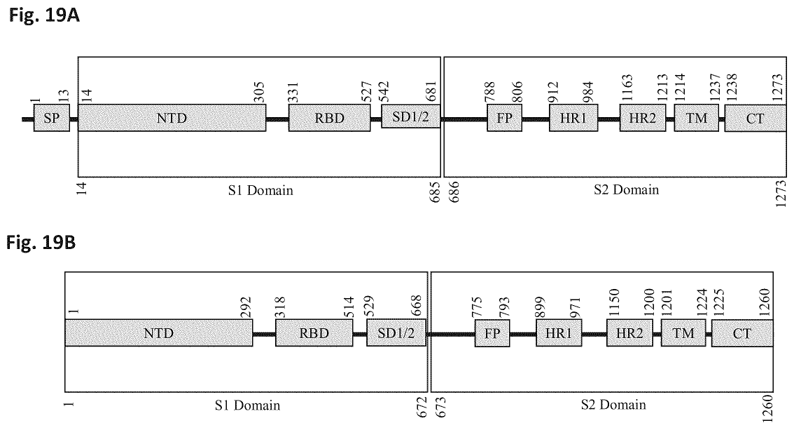

In embodiments, the intranasal immunogenic compositions comprise from 1 to about 50 CoV S glycoproteins. The CoV S glycoprotein is synthesized as an inactive precursor (SO) that is proteolytically cleaved at the furin cleavage site into S1 and S2 subunits which remain non-covalently linked to form prefusion trimers. The S2 domain of the CoV S glycoprotein comprises a fusion peptide (FP), two heptad repeats (HR1 and HR2), a transmembrane (TM) domain, and a cytoplasmic tail (CT). The S1 domain of the SARS-COV-2 S protein folds into four distinct domains: the N-terminal domain (NTD) and the C-terminal domain, which contains the receptor binding domain (RBD) and two subdomains SD1 and SD2. The prefusion SARS-COV-2 S protein trimers undergo a structural rearrangement from a prefusion to a postfusion conformation upon S-protein receptor binding and cleavage. In embodiments, the CoV S polypeptides are glycoproteins, due to post-translational glycosylation. The glycoproteins comprise one or more domains, including a signal peptide, an S1 subunit, an S2 subunit, a NTD, a, RBD, two subdomains (SD1 and SD2, labeled SD1/2 in FIGS. 1A-B and referred to as “SD1/2” herein), an intact or modified fusion peptide, an HR1 domain, an HR2 domain, a TM, and a CD. In embodiments, the amino acids for each domain are given in FIG. 19A (shown according to SEQ ID NO: 1), FIG. 19B (shown according to SEQ ID NO: 2). SARS-COV-2 S glycoproteins also contain a furin cleavage site.

In embodiments, each of the CoV S glycoproteins described herein contains proline at amino acid positions 973 and 974 and comprises an inactive furin cleavage site, wherein the CoV S glycoprotein is numbered according to the CoV S glycoprotein of SEQ ID NO: 2.

In embodiments, the CoV S glycoproteins described herein are expressed with an N-terminal signal peptide. In embodiments, the N-terminal signal peptide has an amino acid sequence of SEQ ID NO: 5 (MFVFLVLLPLVSS). In embodiments, the N-terminal signal peptide has an amino acid sequence of SEQ ID NO: 117 (MFVFLVLLPLVSI). In embodiments, the N-terminal signal peptide has an amino acid sequence of SEQ ID NO: 154 (MFVFFVLLPLVSS). In embodiments, the N-terminal signal peptide has an amino acid sequence of SEQ ID NO: 193 (MFGFLVLLPLVSS). In embodiments, the signal peptide may be replaced with any signal peptide that enables expression of the CoV S glycoprotein. In embodiments, one or more of the CoV S glycoprotein signal peptide amino acids may be deleted or mutated. An initiating methionine residue is maintained to initiate expression. In embodiments, the CoV S glycoproteins are encoded by a nucleic acid sequence selected from the group consisting of SEQ ID NO: 35, SEQ ID NO: 37, SEQ ID NO: 95, SEQ ID NO: 43, SEQ ID NO: 47, SEQ ID NO: 50, SEQ ID NO: 53, SEQ ID NO: 55, SEQ ID NO: 57, SEQ ID NO: 96, SEQ ID NO: 60, SEQ ID NO: 131, SEQ ID NO: 135, SEQ ID NO: 142, SEQ ID NO: 145, SEQ ID NO: 148, SEQ ID NO: 150, SEQ ID NO: 196, SEQ ID NO: 197; SEQ ID NO: 198; SEQ ID NO: 199; SEQ ID NO: 201; SEQ ID NO: 202; SEQ ID NO: 204; SEQ ID NO: 206; SEQ ID NO:N 208; SEQ ID NO: 210; SEQ ID NO: 212; SEQ ID NO: 214; and SEQ ID NO: 216. In embodiments, the N-terminal signal peptide of the CoV S glycoprotein contains a mutation at Ser-13 relative to the native CoV Spike(S) signal polypeptide (SEQ ID NO: 5). In embodiments, Ser-13 is mutated to any natural amino acid. In embodiments, Ser-13 is mutated to alanine, methionine, isoleucine, leucine, threonine, or valine. In embodiments, Ser-13 is mutated to isoleucine.

Following expression of the CoV S glycoprotein in a host cell, the N-terminal signal peptide is cleaved to provide the mature CoV glycoprotein sequence (SEQ ID NO: 87, SEQ ID NO: 89, SEQ ID NO: 106, SEQ ID NO: 112, SEQ ID NO: 113, SEQ ID NO: 114, SEQ ID NO: 115, SEQ ID NO: 132, SEQ ID NO: 144, SEQ ID NO: 151, SEQ ID NO: 153, SEQ ID NO: 156, SEQ ID NO: 158, SEQ ID NO: 174, SEQ ID NO: 175, SEQ ID NO: 176, SEQ ID NO: 181, SEQ ID NO: 182, SEQ ID NO: 183, SEQ ID NO: 184, SEQ ID NO: 186, SEQ ID NO: 188, SEQ ID NO: 190, SEQ ID NO: 192, SEQ ID NO: 195, SEQ ID NO: 217, SEQ ID NO: 218, SEQ ID NO: 219, SEQ ID NO: 220, SEQ ID NO: 221, SEQ ID NO: 222, SEQ ID NO: 223, SEQ ID NO: 224, SEQ ID NO: 225, SEQ ID NO: 226, SEQ ID NO: 227, SEQ ID NO: 228, SEQ ID NO: 233, SEQ ID NO: 234, SEQ ID NO: 235, SEQ ID NO: 236, SEQ ID NO: 260, SEQ ID NO: 261, SEQ ID NO: 262, SEQ ID NO: 263, SEQ ID NO: 264, SEQ ID NO: 274, SEQ ID NO: 276, SEQ ID NO: 278, SEQ ID NO: 280, SEQ ID NO: 284, SEQ ID NO: 288, SEQ ID NO: 292, SEQ ID NO: 296, SEQ ID NO: 300, SEQ ID NO: 304, SEQ ID NO: 308, SEQ ID NO: 312, SEQ ID NO: 316, SEQ ID NO: 320, SEQ ID NO: 324, SEQ ID NO: 328). In embodiments, the signal peptide is cleaved by host cell proteases. In aspects, the full-length protein may be isolated from the host cell and the signal peptide cleaved subsequently.

Following cleavage of the signal peptide from a CoV Spike(S) polypeptide with an amino acid sequence corresponding to any one of SEQ ID NO: 88, SEQ ID NO: 105, SEQ ID NO: 130, SEQ ID NO: 136, SEQ ID NO: 143, SEQ ID NO: 149, SEQ ID NO: 152, SEQ ID NO: 155, SEQ ID NO: 157, SEQ ID NO: 159, SEQ ID NO: 160, SEQ ID NO: 167, SEQ ID NO: 170, SEQ ID NO: 173, SEQ ID NO: 177, SEQ ID NO: 178, SEQ ID NO: 179, SEQ ID NO: 180, SEQ ID NO: 185, SEQ ID NO: 187, SEQ ID NO: 189, SEQ ID NO: 191, SEQ ID NO: 194, SEQ ID NO: 200, SEQ ID NO: 203, SEQ ID NO: 205, SEQ ID NO: 207, SEQ ID NO: 209, SEQ ID NO: 211, SEQ ID NO: 213, SEQ ID NO: 215, SEQ ID NO: 229, SEQ ID NO: 230, SEQ ID NO: 231, SEQ ID NO: 232, SEQ ID NO: 242, SEQ ID NO: 243, SEQ ID NO: 244, SEQ ID NO: 250, SEQ ID NO: 251, SEQ ID NO: 252, SEQ ID NO: 253, SEQ ID NO: 254, SEQ ID NO: 266, SEQ ID NO: 268, SEQ ID NO: 270, SEQ ID NO: 272, SEQ ID NO: 282, SEQ ID NO: 286, SEQ ID NO: 290, SEQ ID NO: 295, SEQ ID NO: 299, SEQ ID NO: 303, SEQ ID NO: 307, SEQ ID NO: 311, SEQ ID NO: 315, SEQ ID NO: 319, SEQ ID NO: 323, and SEQ ID NO: 327 during expression and purification, a mature polypeptide having an amino acid sequence selected from the group consisting of SEQ SEQ ID NO: 87, SEQ ID NO: 89, SEQ ID NO: 106, SEQ ID NO: 112, SEQ ID NO: 113, SEQ ID NO: 114, SEQ ID NO: 115, SEQ ID NO: 132, SEQ ID NO: 144, SEQ ID NO: 151, SEQ ID NO: 153, SEQ ID NO: 156, SEQ ID NO: 158, SEQ ID NO: 174, SEQ ID NO: 175, SEQ ID NO: 176, SEQ ID NO: 181, SEQ ID NO: 182, SEQ ID NO: 183, SEQ ID NO: 184, SEQ ID NO: 186, SEQ ID NO: 188, SEQ ID NO: 190, SEQ ID NO: 192, SEQ ID NO: 195, SEQ ID NO: 217, SEQ ID NO: 218, SEQ ID NO: 219, SEQ ID NO: 220, SEQ ID NO: 221, SEQ ID NO: 222, SEQ ID NO: 223, SEQ ID NO: 224, SEQ ID NO: 225, SEQ ID NO: 226, SEQ ID NO: 227, SEQ ID NO: 228, SEQ ID NO: 233, SEQ ID NO: 234, SEQ ID NO: 235, SEQ ID NO: 236, SEQ ID NO: 260, SEQ ID NO: 261, SEQ ID NO: 262, SEQ ID NO: 263, SEQ ID NO: 264, SEQ ID NO: 274, SEQ ID NO: 276, SEQ ID NO: 278, SEQ ID NO: 280, SEQ ID NO: 284, SEQ ID NO: 288, SEQ ID NO: 292, SEQ ID NO: 296, SEQ ID NO: 300, SEQ ID NO: 304, SEQ ID NO: 308, SEQ ID NO: 312, SEQ ID NO: 316, SEQ ID NO: 320, SEQ ID NO: 324, and SEQ ID NO: 328 is obtained and used to produce a CoV S nanoparticle vaccine or CoV S nanoparticles.

Advantageously, the disclosed CoV S polypeptides may have enhanced protein expression and stability relative to a native CoV Spike(S) protein.

In embodiments, the CoV S polypeptides described herein contain further modifications from a native coronavirus S protein (e.g., SEQ ID NO: 2). In embodiments, the coronavirus S proteins described herein exhibit at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% identity to a native CoV S glycoprotein. A person of skill in the art would use known techniques to calculate the percent identity of the recombinant coronavirus S protein to the native protein or to any of the CoV S glycoproteins described herein.

In embodiments, the CoV S polypeptides described herein are at least 90%, at least 91%, at least 92%, at least 93%, at least 94%, at least 95%, at least 96%, at least 97%, at least 98%, at least 99%, or at least 99.5% identical to the CoV S polypeptide having an amino acid sequence of any one of SEQ ID NO: 87, SEQ ID NO: 174, SEQ ID NO: 175, SEQ ID NO: 176, SEQ ID NOS: 181-184, SEQ ID NO: 186, SEQ ID NO: 188, SEQ ID NO: 190, SEQ ID NO: 195; SEQ ID NOS: 217-228, SEQ ID NOS: 233-236, and SEQ ID NO: 243, SEQ ID NOS 255-328. A CoV S polypeptide may have a deletion, an insertion, or mutation of up to about 1, up to about 2, up to about 3, up to about 4, up to about 5, up to about 10, up to about 15, up to about 20, up to about 25, up to about 30, up to about 35, up to about 40, up to about 45, or up to about 50 amino acids compared to the amino acid sequence of the CoV S polypeptide having an amino acid sequence of any one of SEQ ID NO: 87, SEQ ID NO: 174, SEQ ID NO: 175, SEQ ID NO: 176, SEQ ID NOS: 181-184, SEQ ID NO: 186, SEQ ID NO: 188, SEQ ID NO: 190, SEQ ID NO: 195; SEQ ID NOS: 217-228, SEQ ID NOS: 233-236, SEQ ID NO: 243, and SEQ ID NOS: 255-328. A CoV S polypeptide may have may have a deletion, an insertion, or mutation of from 1 to about 70 amino acids, from 1 to about 5 amino acids, from about 3 to about 10 amino acids, from about 5 to about 10 amino acids, from about 8 to about 12 amino acids, from about 10 to about 15 amino acids, from about 12 to about 17 amino acids, from about 15 to about 20 amino acids, from about 18 to about 23 amino acids, from about 20 to about 25 amino acids, from about 22 to about 27 amino acids, from about 25 to about 30 amino acids, from about 30 to about 35 amino acids, from about 35 to about 40 amino acids, from about 40 to about 45 amino acids, or from about 45 to about 50 amino acids, as compared to the CoV S polypeptide having an amino acid sequence of any one of SEQ ID NO: 87, SEQ ID NO: 174, SEQ ID NO: 175, SEQ ID NO: 176, SEQ ID NOS: 181-184, SEQ ID NO: 186, SEQ ID NO: 188, SEQ ID NO: 190, SEQ ID NO: 195; SEQ ID NOS: 217-228, SEQ ID NOS: 233-236, SEQ ID NO: 243, and SEQ ID NOS: 255-328. In embodiments, the CoV S polypeptides described herein comprise about 1, about 2, about 3, about 4, about 5, about 6, about 7, about 8, about 9, about 10, about 11, about 12, about 13, about 14, about 15, about 16, about 17, about 18, about 19, about 20, about 21, about 22, about 23, about 24, about 25, about 26, about 27, about 28, about 29, about 30, about 31, about 32, about 33, about 34, about 35, about 36, about 37, about 38, about 39, about 40, about 41, about 42, about 43, about 44, about 45, about 46, about 47, about 48, about 49, about 50, about 51, about 52, about 53, about 54, about 55, about 56, about 57, about 58, about 59, about 60, about 61, about 62, about 63, about 64, about 65, about 66, about 67, about 68, about 69, about 70, about 71, about 72, about 73, about 74, or about 75 deletions, insertions, or mutations compared to the coronavirus S protein having an amino acid sequence of any one of SEQ ID NO: 87, SEQ ID NO: 174, SEQ ID NO: 175, SEQ ID NO: 176, SEQ ID NOS: 181-184, SEQ ID NO: 186, SEQ ID NO: 188, SEQ ID NO: 190, SEQ ID NO: 195; SEQ ID NOS: 217-228, SEQ ID NOS: 233-236, SEQ ID NO: 243, and SEQ ID NOS: 255-328.

In embodiments, the coronavirus S polypeptide is extended at the N-terminus, the C-terminus, or both the N-terminus and the C-terminus. In aspects, the extension is a tag useful for a function, such as purification or detection. In aspects the tag contains an epitope. For example, the tag may be a polyglutamate tag, a FLAG-tag, a HA-tag, a polyHis-tag (having about 5-10 histidines) (SEQ ID NO: 101), a hexahistidine tag (SEQ ID NO: 100), an 8X-His-tag (having eight histidines) (SEQ ID NO: 102), a Myc-tag, a Glutathione-S-transferase-tag, a Green fluorescent protein-tag, Maltose binding protein-tag, a Thioredoxin-tag, or an Fc-tag. In other aspects, the extension may be an N-terminal signal peptide fused to the protein to enhance expression. While such signal peptides are often cleaved during expression in the cell, some nanoparticles may contain the antigen with an intact signal peptide. Thus, when a nanoparticle comprises an antigen, the antigen may contain an extension and thus may be a fusion protein when incorporated into nanoparticles. For the purposes of calculating identity to the sequence, extensions are not included. In embodiments, the tag is a protease cleavage site. Non-limiting examples of protease cleavage sites include the HRV3C protease cleavage site, chymotrypsin, trypsin, elastase, endopeptidase, caspase-1, caspase-2, caspase-3, caspase-4, caspase-5, caspase-6, caspase-7, caspase-8, caspase-9, caspase-10, enterokinase, factor Xa, Granzyme B, TEV protease, and thrombin. In embodiments, the protease cleavage site is an HRV3C protease cleavage site. In embodiments, the protease cleavage site comprises an amino acid sequence of SEQ ID NO: 98.

In embodiments, the CoV S glycoprotein comprises a fusion protein. In embodiments, the CoV S glycoprotein comprises an N-terminal fusion protein. In embodiments, the Cov S glycoprotein comprises a C-terminal fusion protein. In embodiments, the fusion protein encompasses a tag useful for protein expression, purification, or detection. In embodiments, the tag is a polyHis-tag (having about 5-10 histidines), a Myc-tag, a Glutathione-S-transferase-tag, a Green fluorescent protein-tag, Maltose binding protein-tag, a Thioredoxin-tag, a Strep-tag, a Twin-Strep-tag, or an Fc-tag. In embodiments, the tag is an Fc-tag. In embodiments, the Fc-tag is monomeric, dimeric, or trimeric. In embodiments, the tag is a hexahistidine tag, e.g. a polyHis-tag which contains six histidines (SEQ ID NO: 100). In embodiments, the tag is a Twin-Strep-tag with an amino acid sequence of SEQ ID NO: 99.

In embodiments, the CoV S polypeptide is a fusion protein comprising another coronavirus protein. In embodiments, the other coronavirus protein is from the same coronavirus. In embodiments, the other coronavirus protein is from a different coronavirus.

In aspects, the CoV S glycoprotein may be truncated. For example, the N-terminus may be truncated by about 10 amino acids, about 30 amino acids, about 50 amino acids, about 75 amino acids, about 100 amino acids, or about 200 amino acids. The C-terminus may be truncated instead of or in addition to the N-terminus. For example, the C-terminus may be truncated by about 10 amino acids, about 30 amino acids, about 50 amino acids, about 75 amino acids, about 100 amino acids, or about 200 amino acids. For purposes of calculating identity to the protein having truncations, identity is measured over the remaining portion of the protein.

In embodiments, the CoV S glycoproteins contain one or more modifications to the S1 subunit having an amino acid sequence of SEQ ID NO: 121.

The amino acid sequence of the S1 subunit (SEQ ID NO: 121) is shown below.

QCVNLTTRTQLPPAYTNSFTRGVYYPDKVFRSSVLHSTQDLFLPFFSNV
TWFHAIHVSGTNGTKRFDNPVLPFNDGVYFASTEKSNIIRGWIFGTTLD
SKTQSLLIVNNATNVVIKVCEFQFCNDPFLGVYYHKNNKSWMESEFRVY
SSANNCTFEYVSQPFLMDLEGKQGNFKNLREFVFKNIDGYFKIYSKHTP
INLVRDLPQGFSALEPLVDLPIGINITRFQTLLALHRSYLTPGDSSSGW
TAGAAAYYVGYLQPRTFLLKYNENGTITDAVDCALDPLSETKCTLKSFT
VEKGIYQTSNFRVQPTESIVRFPNITNLCPFGEVENATRFASVYAWNRK
RISNCVADYSVLYNSASFSTFKCYGVSPTKLNDLCFTNVYADSFVIRGD
EVRQIAPGQTGKIADYNYKLPDDFTGCVIAWNSNNLDSKVGGNYNYLYR
LFRKSNLKPFERDISTEIYQAGSTPCNGVEGFNCYFPLQSYGFQPTNGV
GYQPYRVVVLSFELLHAPATVCGPKKSTNLVKNKCVNFNFNGLTGTGVL
TESNKKFLPFQQFGRDIADTTDAVRDPQTLEILDITPCSFGGVSVITPG
TNTSNQVAVLYQDVNCTEVPVAIHADQLTPTWRVYSTGSNVFQTRAGCL
IGAEHVNNSYECDIPIGAGICASYQTQTNSPRRAR

In embodiments, the CoV S polypeptides described herein comprise an S1 subunit with at least 90%, at least 91%, at least 92%, at least 93%, at least 94%, at least 95%, at least 96%, at least 97%, at least 98%, at least 99%, at least 99.5%, or 100% identity to the S1 subunit of SEQ ID NO: 1 or SEQ ID NO: 2. The S1 subunit may have a deletion, an insertion, or mutation of up to about 1, up to about 2, up to about 3, up to about 4, up to about 5, up to about 10, up to about 15, up to about 20, up to about 25, up to about 30 amino acids, up to about 35 amino acids, up to about 40 amino acids, up to about 45 amino acids, or up to about 50 amino acids compared to the amino acid sequence of the S1 subunit of SEQ ID NO: 1 or SEQ ID NO: 2. The S1 subunit may have a deletion, an insertion, or mutation of between about 1 and about 5 amino acids, between about 3 and about 10 amino acids, between about 5 and 10 amino acids, between about 8 and 12 amino acids, between about 10 and 15 amino acids, between about 12 and 17 amino acids, between about 15 and 20 amino acids, between about 18 and 23 amino acids, between about 20 and 25 amino acids, between about 22 and about 27 amino acids, or between about 25 and 30 amino acids as compared to the S1 subunit of SEQ ID NO: 1 or SEQ ID NO: 2.

In embodiments, the S1 subunit may contain any combination of modifications shown in Table 1A.

TABLE 1A
Modifications to S1 (SEQ ID NO: 121)

| Position | Position | Position | |
|---|---|---|---|
| within | within | within | |
| SEQ ID | SEQ ID | SEQ ID | |
| NO: 1 | NO: 2 | NO: 121 | Potential Modifications |
| 14-305 | 1-292 | 1-292 | deletion of up to about 1, 2, 3, 4, 5, 10, 20, |
| | | | 30, 40, 50, 60, 70, 80, 90, 100, 110, 120, |
| | | | 130, 140, 150, 160, 170, 180, 190, 200, |
| | | | 210, 220, 230, 240, 250, 260, 270, 280, |
| | | | 290, or 292 amino acids |
| 18 | 5 | 5 | mutation to phenylalanine |
| | | | mutation to tyrosine |
| | | | mutation to tryptophan |
| 19 | 6 | 6 | mutation to arginine |
| | | | mutation to lysine |
| | | | mutation to histidine |
| | | | mutation to isoleucine |
| 20 | 7 | 7 | mutation to asparagine |
| | | | mutation to glutamine |
| | | | mutation to isoleucine |
| | | | mutation to valine |
| 24 | 11 | 11 | mutation to serine |
| | | | mutation to threonine |
| | | | deletion |
| 25 | 12 | 12 | insertion of amino acids proline-proline- |
| | | | alanine (PPA) after amino acid 25 |
| | | | deletion |
| 26 | 13 | 13 | mutation to serine |
| | | | mutation to threonine |
| | | | deletion |
| 27 | 14 | 14 | mutation to serine |
| | | | mutation to threonine |
| 52 | 39 | 39 | mutation to arginine |
| | | | mutation to lysine |
| | | | mutation to histidine |
| 64 | 51 | 51 | mutation to histidine |
| | | | mutation to lysine |
| | | | mutation to arginine |
| 66 | 53 | 53 | mutation to tryptophan |
| | | | mutation to tyrosine |
| | | | mutation to phenylalanine |
| 67 | 54 | 54 | mutation to valine |
| | | | mutation to isoleucine |
| | | | mutation to leucine |
| 69 | 56 | 56 | Deletion of amino acid |
| 70 | 57 | 57 | Deletion of amino acid |
| | | | Mutation to phenylalanine |
| | | | Mutation to tyrosine |
| | | | Mutation to tryptophan |
| 75 | 62 | 62 | Mutation to valine |
| | | | Mutation to leucine |
| | | | Mutation to isoleucine |
| 76 | 63 | 63 | Mutation to isoleucine |
| | | | Mutation to valine |
| | | | Mutation to leucine |
| 80 | 67 | 67 | mutation to alanine |
| | | | mutation to glycine |
| 83 | 70 | 70 | mutation to alanine |
| 95 | 82 | 82 | mutation to beta branched amino acid |
| | | | mutation to isoleucine |
| | | | mutation to valine |
| 138 | 125 | 125 | mutation to tyrosine |
| | | | mutation to phenylalanine |
| | | | mutation to tryptophan |
| 142 | 129 | 129 | mutation to aspartic acid |
| | | | mutation to glutamic acid |
| 143 | 130 | 130 | deletion of amino acid |
| 144 | 131 | 131 | Deletion of amino acid |
| | | | Mutation to serine |
| 145 | 132 | 132 | Deletion of amino acid |
| | | | Mutation to histidine |
| | | | Mutation to asparagine |
| | | | Mutation to glutamine |
| 146 | 133 | 133 | mutation to aromatic amino acid |
| | | | mutation to tyrosine |
| | | | mutation to phenylalanine |
| | | | mutation to tryptophan |
| | | | mutation to glutamine |
| 147 | 134 | 134 | mutation to glutamic acid |
| | | | mutation to aspartic acid |
| 152 | 139 | 139 | mutation to cysteine |
| | | | mutation to methionine |
| | | | mutation to serine |
| | | | mutation to threonine |
| | | | mutation to arginine |
| | | | mutation to lysine |
| 156 | 143 | 143 | mutation to glycine |
| | | | mutation to alanine |
| 157 | 144 | 144 | deletion of amino acid |
| | | | mutation to leucine |
| | | | mutation to isoleucine |
| | | | mutation to valine |
| | | | mutation to beta-branched amino acid |
| 158 | 145 | 145 | deletion of amino acid |
| 180 | 167 | 167 | mutation to beta-branched amino acid |
| | | | mutation to valine |
| | | | mutation to isoleucine |
| | | | mutation to leucine |
| 183 | 170 | 170 | mutation to glutamic acid |
| | | | mutation to aspartic acid |
| 190 | 177 | 177 | mutation to serine |
| | | | mutation to threonine |
| | | | mutation to cysteine |
| 210 | 197 | 197 | mutation to valine |
| | | | mutation to isoleucine |
| | | | mutation to leucine |
| | | | mutation to beta branched amino acid |
| 211 | 198 | 198 | mutation to isoleucine |
| | | | mutation to valine |
| | | | mutation to leucine |
| | | | mutation to beta branched amino acid |
| | | | deletion of amino acid |
| 212 | 199 | 199 | mutation to isoleucine |
| | | | mutation to valine |
| | | | mutation to leucine |
| | | | mutation to beta branched amino acid |
| | | | deletion of amino acid |
| 213 | 200 | 200 | mutation to valine |
| | | | mutation to leucine |
| | | | mutation to isoleucine |
| | | | mutation to beta branched amino acid |
| | | | mutation to proline |
| | | | mutation to glycine |
| | | | mutation to glutamic acid |
| | | | mutation to aspartic acid |
| 214 | 201 | 201 | mutation to arginine |
| | | | mutation to lysine |
| | | | mutation to histidine |
| | | | mutation to aspartic acid |
| | | | mutation to glutamic acid |
| | | | insertion of amino acids glutamic acid- |
| | | | proline-glutamic acid (EPE) after 214 |
| 215 | 202 | 202 | mutation to glycine |
| | | | mutation to alanine |
| | | | insertion of amino acids glutamic acid- |
| | | | proline-glutamic acid (EPE) after 215 |
| 222 | 209 | 209 | mutation to valine |
| | | | mutation to leucine |
| | | | mutation to isoleucine |
| 241-244 | 228-231 | 228-231 | deletion of 1, 2, 3, or 4 amino acids |
| 242 | 229 | 229 | mutation to histidine |
| | | | mutation to lysine |
| | | | mutation to arginine |
| 246 | 233 | 233 | mutation to beta-branched amino acid |
| | | | mutation to isoleucine |
| | | | mutation to valine |
| | | | mutation to threonine |
| | | | mutation to asparagine |
| 247 | 234 | 234 | deletion of amino acid |
| 248 | 235 | 235 | deletion of amino acid |
| 249 | 236 | 236 | deletion of amino acid |
| 250 | 237 | 237 | deletion of amino acid |
| 251 | 238 | 238 | deletion of amino acid |
| 252 | 239 | 239 | deletion of amino acid |
| | | | mutation to valine |
| | | | mutation to leucine |
| | | | mutation to isoleucine |
| | | | mutation to beta branched amino acid |
| | | | mutation to glycine |
| 253 | 240 | 240 | mutation to glycine |
| | | | deletion of amino acid |
| 257 | 244 | 244 | mutation to serine |
| | | | mutation to threonine |
| | | | mutation to asparagine |
| | | | mutation to glutamine |
| 258 | 245 | 245 | mutation to isoleucine |
| | | | mutation to valine |
| | | | mutation to leucine |
| | | | mutation to beta branched amino acid |
| 339 | 326 | 326 | mutation to aspartic acid |
| | | | mutation to glutamic acid |
| | | | mutation to histidine |
| 346 | 333 | 333 | mutation to lysine |
| | | | mutation to arginine |
| | | | mutation to histidine |
| | | | mutation to threonine |
| | | | mutation to serine |
| 368 | 355 | 355 | mutation to isoleucine |
| | | | mutation to leucine |
| | | | mutation to valine |
| | | | mutation to beta-branched amino acid |
| 371 | 358 | 358 | mutation to leucine |
| | | | mutation to isoleucine |
| | | | mutation to valine |
| | | | mutation to phenylalanine |
| 373 | 360 | 360 | mutation to proline |
| 375 | 362 | 362 | mutation to phenylalanine |
| | | | mutation to tyrosine |
| | | | mutation to tryptophan |
| 376 | 363 | 363 | mutation to alanine |
| | | | mutation to glycine |
| 405 | 392 | 392 | mutation to asparagine |
| | | | mutation to glutamine |
| 408 | 395 | 395 | mutation to serine |
| | | | mutation to threonine |
| 417 | 404 | 404 | mutation to asparagine |
| | | | mutation to threonine |
| | | | mutation to isoleucine |
| | | | mutation to valine |
| | | | mutation to serine |
| | | | mutation to glutamine |
| | | | mutation to beta-branched amino acid |
| 432 | 419 | 419 | mutation to lysine |
| | | | mutation to arginine |
| | | | mutation to histidine |
| 439 | 426 | 426 | mutation to lysine |
| | | | mutation to arginine |
| | | | mutation to histidine |
| 440 | 427 | 427 | Mutation to lysine |
| | | | Mutation to arginine |
| | | | Mutation to histidine |
| 444 | 431 | 431 | Mutation to threonine |
| | | | Mutation to serine |
| 445 | 432 | 432 | Mutation to proline |
| 446 | 433 | 433 | Mutation to serine |
| | | | Mutation to threonine |
| | | | Mutation to asparagine |
| | | | Mutation to glutamine |
| 452 | 439 | 439 | mutation to arginine |
| | | | mutation to lysine |
| | | | mutation to histidine |
| | | | mutation to glutamine |
| | | | mutation to asparagine |
| 453 | 440 | 440 | mutation to phenylalanine |
| | | | mutation to tryptophan |
| 456 | 443 | 443 | mutation to leucine |
| | | | mutation to valine |
| | | | mutation to isoleucine |
| 460 | 447 | 447 | mutation to lysine |
| | | | mutation to arginine |
| 477 | 464 | 464 | mutation to asparagine |
| | | | mutation to glutamine |
| 478 | 465 | 465 | mutation to lysine |
| | | | mutation to arginine |
| | | | mutation to histidine |
| 484 | 471 | 471 | mutation to alanine |
| | | | mutation to lysine |
| | | | mutation to arginine |
| | | | mutation to histidine |
| | | | mutation to glutamine |
| | | | mutation to asparagine |
| 486 | 473 | 473 | mutation to valine |
| | | | mutation to leucine |
| | | | mutation to isoleucine |
| | | | mutation to serine |
| | | | mutation to threonine |
| | | | mutation to proline |
| 490 | 477 | 477 | mutation to serine |
| | | | mutation to threonine |
| 493 | 480 | 480 | mutation to lysine |
| | | | mutation to arginine |
| | | | mutation to histidine |
| 494 | 481 | 481 | mutation to proline |
| 496 | 483 | 483 | mutation to serine |
| | | | mutation to threonine |
| 498 | 485 | 485 | mutation to lysine |
| | | | mutation to arginine |
| | | | mutation to histidine |
| 501 | 488 | 488 | mutation to tyrosine |
| | | | mutation to phenylalanine |
| | | | mutation to tryptophan |
| 505 | 492 | 492 | mutation to histidine |
| 521 | 508 | 508 | mutation to serine |
| | | | mutation to threonine |
| 547 | 534 | 534 | mutation to lysine |
| | | | mutation to arginine |
| | | | mutation to histidine |
| 570 | 557 | 557 | Mutation to aspartic acid |
| | | | Mutation to glutamic acid |
| 604 | 591 | 591 | Mutation to isoleucine |
| | | | Mutation to leucine |
| | | | Mutation to valine |
| | | | Mutation to beta branched amino acid |
| 613 | 600 | 600 | Mutation to histidine |
| | | | Mutation to lysine |
| | | | Mutation to arginine |
| 614 | 601 | 601 | Mutation to glycine |
| | | | Mutation to alanine |
| 639 | 626 | 626 | Mutation to valine |
| | | | Mutation to leucine |
| | | | Mutation to isoleucine |
| 655 | 642 | 642 | Mutation to tyrosine |
| | | | Mutation to phenylalanine |
| | | | Mutation to tryptophan |
| 658 | 645 | 645 | Mutation to serine |
| | | | Mutation to threonine |
| 677 | 664 | 664 | Mutation to histidine |
| 679 | 666 | 666 | Mutation to lysine |
| | | | Mutation to arginine |
| | | | Mutation to histidine |
| 681 | 668 | 668 | Mutation to histidine |
| | | | Mutation to lysine |
| | | | Mutation to arginine |
| 682-685 | 669-672 | 669-672 | inactive furin cleavage site (See |
| | | | Table 1E) |

* amino acids 14-685 of SEQ ID NO: 1 and amino acids 1-672 of SEQ ID NO: 2

In embodiments, the CoV S polypeptides contain one or more modifications to the NTD. In embodiments, the NTD has an amino acid sequence of SEQ ID NO: 118, which corresponds to amino acids 14-305 of SEQ ID NO: 1 or amino acids 1-292 of SEQ ID NO: 2.

The amino acid sequence of an NTD (SEQ ID NO: 118) is shown below.

QCVNLTTRTQLPPAYTNSFTRGVYYPDKVFRSSVLHSTQDLFLPFFSNV
TWFHAIHVSGTNGTKRFDNPVLPFNDGVYFASTEKSNIIRGWIFGTTLD
SKTQSLLIVNNATNVVIKVCEFQFCNDPFLGVYYHKNNKSWMESEFRVY
SSANNCTFEYVSQPFLMDLEGKQGNFKNLREFVFKNIDGYFKIYSKHTP
INLVRDLPQGFSALEPLVDLPIGINITRFQTLLALHRSYLTPGDSSSGW
TAGAAAYYVGYLQPRTFLLKYNENGTITDAVDCALDPLSETKCTLKS

In embodiments, the NTD has an amino acid sequence of SEQ ID NO: 45, which corresponds to amino acids 14 to 331 of SEQ ID NO: 1 or amino acids 1-318 of SEQ ID NO: 2.

The amino acid sequence of an NTD (SEQ ID NO: 45) is shown below.

QCVNLTTRTQLPPAYTNSFTRGVYYPDKVFRSSVLHSTQDLFLPFFSNV
TWFHAIHVSGTNGTKRFDNPVLPFNDGVYFASTEKSNIIRGWIFGTTLD
SKTQSLLIVNNATNVVIKVCEFQFCNDPFLGVYYHKNNKSWMESEFRVY
SSANNCTFEYVSQPFLMDLEGKQGNFKNLREFVFKNIDGYFKIYSKHTP
INLVRDLPQGFSALEPLVDLPIGINITRFQTLLALHRSYLTPGDSSSGW
TAGAAAYYVGYLQPRTFLLKYNENGTITDAVDCALDPLSETKCTLKSFT
VEKGIYQTSNFRVQPTESIVRFPN

In embodiments, the NTD and RBD overlap by up to about 1 amino acid, up to about 5 amino acids, up to about 10 amino acids, or up to about 20 amino acids.

In embodiments, an NTD as provided herein may be extended at the C-terminus by up to 5, up to 10, up to 15, up to 20, up to 25, or up to 30 amino acids.

In embodiments, the CoV S polypeptides described herein comprise a NTD with at least 95%, at least 96%, at least 97%, at least 98%, at least 99%, at least 99.5%, or 100% identity to the NTD of SEQ ID NO: 1 or SEQ ID NO: 2. The NTD may have a deletion, an insertion, or mutation of up to about 1, up to about 2, up to about 3, up to about 4, up to about 5, up to about 10, up to about 15, up to about 20, up to about 25, or up to about 30 amino acids compared to the amino acid sequence of the NTD of SEQ ID NO: 1 or SEQ ID NO: 2. The NTD may have a deletion, an insertion, or mutation of between about 1 and about 5 amino acids, between about 3 and about 10 amino acids, between about 5 and 10 amino acids, between about 8 and 12 amino acids, between about 10 and 15 amino acids, between about 12 and 17 amino acids, between about 15 and 20 amino acids, between about 18 and 23 amino acids, between about 20 and 25 amino acids, between about 22 and about 27 amino acids, or between about 25 and 30 amino acids as compared to the NTD of SEQ ID NO: 1 or SEQ ID NO: 2.

In embodiments, the CoV S polypeptides contain a deletion of one or more amino acids from the N-terminal domain (NTD) (corresponding to amino acids 1-292 of SEQ ID NO: 2. In embodiments, the CoV S polypeptides contain a deletion of up to about 10, 20, 30, 40, 50, 60, 70, 80, 90, 100, 110, 120, 130, 140, 150, 160, 170, 180, 190, 200, 210, 220, 230, 240, 250, 260, 270, 280, 290, or 292 amino acids of the NTD.

In embodiments, the CoV S polypeptides contain a deletion of one or more amino acids from the NTD (corresponding to amino acids 1-318 of SEQ ID NO: 2). In embodiments, the CoV S polypeptides contain a deletion of amino acids 1-318 of the NTD of SEQ ID NO: 2. In embodiments, deletion of the NTD enhances protein expression of the CoV Spike(S) polypeptide. In embodiments, the CoV S polypeptides which have an NTD deletion have amino acid sequences represented by SEQ ID NOS: 46, 48, 49, 51, 52, and 54. In embodiments, the CoV S polypeptides which have an NTD deletion are encoded by an isolated nucleic acid sequence selected from the group consisting of SEQ ID NO: 47, SEQ ID NO: 50, and SEQ ID NO: 53.

In embodiments, the NTD may contain any combination of modifications shown in Table 1B. The modifications are shown with respect to SEQ ID NO: 2, the mature S polypeptide sequence for reference.

TABLE 1B
Modifications to NTD (SEQ ID NO: 118)

| | | SEQ ID | |
|---|---|---|---|
| Position | SEQ | NO: 118 | |
| within | ID | or SEQ | |
| SEQ | NO: | ID | |
| ID | 2 | NO: 45 | |
| NO: 1 | residue | residue | Modifications |
| 14-305 | 1-292 | 1-292 | deletion of up to about 1, 2, 3, 4, 5, 10, |
| | | | 20, 30, 40, 50, 60, 70, 80, 90, 100, 110, |
| | | | 120, 130, 140, 150, 160, 170, 180, |
| | | | 190, 200, 210, 220, 230, 240, 250, |
| | | | 260, 270, 280, 290, or 292 amino acids |
| 18 | 5 | 5 | mutation to phenylalanine |
| | | | mutation to tyrosine |
| | | | mutation to tryptophan |
| 19 | 6 | 6 | mutation to arginine |
| | | | mutation to lysine |
| | | | mutation to histidine |
| | | | mutation to isoleucine |
| 20 | 7 | 7 | mutation to asparagine |
| | | | mutation to glutamine |
| | | | mutation to isoleucine |
| | | | mutation to valine |
| 24 | 11 | 11 | mutation to serine |
| | | | mutation to threonine |
| | | | deletion |
| 25 | 12 | 12 | insertion of amino acids proline-proline- |
| | | | alanine (PPA) after amino acid 25 |
| | | | deletion |
| 26 | 13 | 13 | mutation to serine |
| | | | mutation to threonine |
| | | | deletion |
| 27 | 14 | 14 | mutation to serine |
| | | | mutation to threonine |
| 52 | 39 | 39 | mutation to arginine |
| | | | mutation to lysine |
| | | | mutation to histidine |
| 64 | 51 | 51 | mutation to histidine |
| | | | mutation to lysine |
| | | | mutation to arginine |
| 66 | 53 | 53 | mutation to tryptophan |
| | | | mutation to tyrosine |
| | | | mutation to phenylalanine |
| 67 | 54 | 54 | mutation to valine |
| | | | mutation to isoleucine |
| | | | mutation to leucine |
| 69 | 56 | 56 | Deletion of amino acid |
| 70 | 57 | 57 | Deletion of amino acid |
| | | | Mutation to phenylalanine |
| | | | Mutation to tyrosine |
| | | | Mutation to tryptophan |
| 75 | 62 | 62 | Mutation to valine |
| | | | Mutation to leucine |
| | | | Mutation to isoleucine |
| 76 | 63 | 63 | Mutation to isoleucine |
| | | | Mutation to valine |
| | | | Mutation to leucine |
| 80 | 67 | 67 | mutation to alanine |
| | | | mutation to glycine |
| 83 | 70 | 70 | mutation to alanine |
| 95 | 82 | 82 | mutation to beta branched amino acid |
| | | | mutation to isoleucine |
| | | | mutation to valine |
| 138 | 125 | 125 | mutation to tyrosine |
| | | | mutation to phenylalanine |
| | | | mutation to tryptophan |
| 142 | 129 | 129 | mutation to aspartic acid |
| | | | mutation to glutamic acid |
| 143 | 130 | 130 | deletion of amino acid |
| 144 | 131 | 131 | Deletion of amino acid |
| | | | Mutation to serine |
| 145 | 132 | 132 | Deletion of amino acid |
| | | | Mutation to histidine |
| | | | Mutation to asparagine |
| | | | Mutation to glutamine |
| 146 | 133 | 133 | mutation to aromatic amino acid |
| | | | mutation to tyrosine |
| | | | mutation to phenylalanine |
| | | | mutation to tryptophan |
| | | | mutation to glutamine |
| | | | mutation to asparagine |
| 147 | 134 | 134 | mutation to glutamic acid |
| | | | mutation to aspartic acid |
| 152 | 139 | 139 | mutation to cysteine |
| | | | mutation to methionine |
| | | | mutation to serine |
| | | | mutation to threonine |
| | | | mutation to arginine |
| | | | mutation to lysine |
| 156 | 143 | 143 | mutation to glycine |
| | | | mutation to alanine |
| 157 | 144 | 144 | deletion of amino acid |
| | | | mutation to leucine |
| 158 | 145 | 145 | deletion of amino acid |
| 180 | 167 | 167 | mutation to isoleucine |
| | | | mutation to valine |
| | | | mutation to leucine |
| | | | mutation to beta branched amino acid |
| 183 | 170 | 170 | mutation to glutamic acid |
| | | | mutation to aspartic acid |
| 190 | 177 | 177 | mutation to serine |
| | | | mutation to threonine |
| | | | mutation to cysteine |
| 210 | 197 | 197 | mutation to valine |
| | | | mutation to isoleucine |
| | | | mutation to leucine |
| | | | mutation to beta branched amino acid |
| 211 | 198 | 198 | mutation to isoleucine |
| | | | mutation to valine |
| | | | mutation to leucine |
| | | | mutation to beta branched amino acid |
| | | | deletion of amino acid |
| 212 | 199 | 199 | mutation to isoleucine |
| | | | mutation to valine |
| | | | mutation to leucine |
| | | | mutation to beta branched amino acid |
| | | | deletion of amino acid |
| 213 | 200 | 200 | mutation to valine |
| | | | mutation to leucine |
| | | | mutation to isoleucine |
| | | | mutation to beta branched amino acid |
| | | | mutation to proline |
| | | | mutation to glycine |
| | | | mutation to glutamic acid |
| | | | mutation to aspartic acid |
| 214 | 201 | 201 | mutation to arginine |
| | | | mutation to lysine |
| | | | mutation to histidine |
| | | | mutation to aspartic acid |
| | | | mutation to glutamic acid |
| | | | insertion of amino acids glutamic acid- |
| | | | proline-glutamic acid (EPE) after 214 |
| 215 | 202 | 202 | mutation to glycine |
| | | | mutation to alanine |
| | | | insertion of amino acids glutamic acid- |
| | | | proline-glutamic acid (EPE) after 215 |
| 222 | 209 | 209 | mutation to valine |
| | | | mutation to leucine |
| | | | mutation to isoleucine |
| 241-244 | 228-231 | 228-231 | deletion of 1, 2, 3, or 4 amino acids |
| 242 | 229 | 229 | mutation to histidine |
| | | | mutation to lysine |
| | | | mutation to arginine |
| 246 | 233 | 233 | mutation to beta-branched amino acid |
| | | | mutation to isoleucine |
| | | | mutation to valine |
| | | | mutation to threonine |
| | | | mutation to asparagine |
| 247 | 234 | 234 | deletion of amino acid |
| 248 | 235 | 235 | deletion of amino acid |
| 249 | 236 | 236 | deletion of amino acid |
| 250 | 237 | 237 | deletion of amino acid |
| 251 | 238 | 238 | deletion of amino acid |
| 252 | 239 | 239 | deletion of amino acid |
| | | | mutation to valine |
| | | | mutation to isoleucine |
| | | | mutation to leucine |
| | | | mutation to beta branched amino acid |
| | | | mutation to glycine |
| 253 | 240 | 240 | mutation to glycine |
| | | | deletion of amino acid |
| 257 | 244 | 244 | mutation to serine |
| | | | mutation to threonine |
| 258 | 245 | 245 | mutation to isoleucine |
| | | | mutation to valine |
| | | | mutation to leucine |
| | | | mutation to beta branched amino acid |

* amino acids 14-305 of SEQ ID NO: 1 and amino acids 1-292 of SEQ ID NO: 2

In embodiments, the CoV S polypeptides contain one or more modifications to the RBD.

In embodiments, the RBD has an amino acid sequence of SEQ ID NO: 126, which corresponds to amino acids 331-527 of SEQ ID NO: 1 or amino acids 318-514 of SEQ ID NO: 2.

The amino acid sequence of the RBD (SEQ ID NO: 126) is shown below:

NITNLCPFGEVENATRFASVYAWNRKRISNCVADYSVLYNSASFSTFKC
YGVSPTKLNDLCFTNVYADSFVIRGDEVRQIAPGQTGKIADYNYKLPDD
FTGCVIAWNSNNLDSKVGGNYNYLYRLFRKSNLKPFERDISTEIYQAGS
TPCNGVEGENCYFPLQSYGFQPTNGVGYQPYRVVVLSFELLHAPATVCG
P

In embodiments, the RBD has an amino acid sequence of SEQ ID NO: 116, which corresponds to amino acids 335-530 of SEQ ID NO: 1 or amino acids 322-517 of SEQ ID NO: 2.

The amino acid sequence of the RBD (SEQ ID NO: 116) is shown below.

LCPFGEVENATRFASVYAWNRKRISNCVADYSVLYNSASFSTFKCYGVS
PTKLNDLCFTNVYADSFVIRGDEVRQIAPGQTGKIADYNYKLPDDFTGC
VIAWNSNNLDSKVGGNYNYLYRLFRKSNLKPFERDISTEIYQAGSTPCN
GVEGENCYFPLQSYGFQPTNGVGYQPYRVVVLSFELLHAPATVCGPKKS

In embodiments, an RBD as provided herein may be extended at the N-terminus or C-terminus by up to 1 amino acid, up to 5 amino acids, up to 10 amino acids, up to 15 amino acids, up to 20 amino acids, up to 25 amino acids, or up to 30 amino acids.

In embodiments, the CoV S polypeptides described herein comprise a RBD with at least 95%, at least 96%, at least 97%, at least 98%, at least 99%, at least 99.5%, or 100% identity to the RBD of SEQ ID NO: 1 or SEQ ID NO: 2. The RBD may have a deletion, an insertion, or mutation of up to about 1, up to about 2, up to about 3, up to about 4, up to about 5, up to about 10, up to about 15, up to about 20, up to about 25, or up to about 30 amino acids compared to the amino acid sequence of the RBD of SEQ ID NO: 1 or SEQ ID NO: 2. The RBD may have a deletion, an insertion, or mutation of from about 1 to about 50 amino acids, from about 1 to about 5 amino acids, from about 3 to about 10 amino acids, from about 5 to about 10 amino acids, from about 8 to about 12 amino acids, from about 10 to about 15 amino acids, from about 12 to about 17 amino acids, from about 15 to about 20 amino acids, from about 18 to about 23 amino acids, from about 20 to about 25 amino acids, from about 22 to about 27 amino acids, or from about 25 to about 30 amino acids as compared to the RBD of SEQ ID NO: 1 or SEQ ID NO: 2.

In embodiments, the CoV S polypeptide has at least one, at least two, at least three, at least four, at least four, at least 5, at least 6, at least 7, at least 8, at least 9, at least 10, at least 11, at least 12, at least 13, at least 14, at least 15, at least 16, at least 17, at least 18, at least 19, or at least 20 modifications in the RBD. In aspects, there are up to about 20, up to about 25, up to about 30, up to about 35, up to about 40, up to about 45, or up to about 50 modifications in the RBD. In embodiments, the RBD may contain any combination of modifications as shown in Table 1C.

TABLE 1C
Modifications to RBD (SEQ ID NO: 126)

| Position | Position | Position | |
|---|---|---|---|
| within | within | within | |
| SEQ ID | SEQ ID | SEQ ID | |
| NO: 1 | NO: 2 | NO: 126 | Potential Modifications |
| 339 | 326 | 9 | mutation to aspartic acid |
| | | | mutation to glutamic acid |
| | | | mutation to histidine |
| 346 | 333 | 16 | mutation to lysine |
| | | | mutation to arginine |
| | | | mutation to histidine |
| | | | mutation to threonine |
| | | | mutation to serine |
| 368 | 355 | 38 | mutation to isoleucine |
| | | | mutation to leucine |
| | | | mutation to valine |
| | | | mutation to beta-branched amino acid |
| 371 | 358 | 41 | mutation to leucine |
| | | | mutation to isoleucine |
| | | | mutation to valine |
| | | | mutation to phenylalanine |
| | | | mutation to tyrosine |
| | | | mutation to tryptophan |
| 373 | 360 | 43 | mutation to proline |
| 375 | 362 | 45 | mutation to phenylalanine |
| | | | mutation to tyrosine |
| | | | mutation to tryptophan |
| 376 | 363 | 46 | mutation to alanine |
| | | | mutation to glycine |
| 405 | 392 | 75 | mutation to asparagine |
| | | | mutation to glutamine |
| 408 | 395 | 78 | mutation to serine |
| | | | mutation to threonine |
| 417 | 404 | 87 | mutation to asparagine |
| | | | mutation to threonine |
| | | | mutation to isoleucine |
| | | | mutation to valine |
| | | | mutation to serine |
| | | | mutation to glutamine |
| | | | mutation to beta-branched amino acid |
| 432 | 419 | 102 | mutation to lysine |
| | | | mutation to arginine |
| | | | mutation to histidine |
| 439 | 426 | 109 | mutation to lysine |
| | | | mutation to arginine |
| | | | mutation to histidine |
| 440 | 427 | 110 | Mutation to lysine |
| | | | Mutation to arginine |
| | | | Mutation to histidine |
| 444 | 431 | 114 | Mutation to threonine |
| | | | Mutation to serine |
| 445 | 432 | 115 | Mutation to proline |
| 446 | 433 | 116 | Mutation to serine |
| | | | Mutation to threonine |
| 452 | 439 | 122 | mutation to arginine |
| | | | mutation to lysine |
| | | | mutation to histidine |
| | | | mutation to glutamine |
| | | | mutation to asparagine |
| 453 | 440 | 123 | mutation to phenylalanine |
| | | | mutation to tryptophan |
| 456 | 443 | 126 | mutation to leucine |
| | | | mutation to isoleucine |
| | | | mutation to valine |
| 460 | 447 | 130 | mutation to lysine |
| | | | mutation to arginine |
| 477 | 464 | 147 | mutation to asparagine |
| | | | mutation to glutamine |
| 478 | 465 | 148 | mutation to lysine |
| | | | mutation to arginine |
| | | | mutation to histidine |
| 484 | 471 | 154 | mutation to alanine |
| | | | mutation to lysine |
| | | | mutation to arginine |
| | | | mutation to histidine |
| | | | mutation to glutamine |
| | | | mutation to asparagine |
| 486 | 473 | 156 | mutation to valine |
| | | | mutation to leucine |
| | | | mutation to isoleucine |
| | | | mutation to serine |
| | | | mutation to threonine |
| 490 | 477 | 160 | mutation to serine |
| | | | mutation to threonine |
| 493 | 480 | 163 | mutation to lysine |
| | | | mutation to arginine |
| | | | mutation to histidine |
| 494 | 481 | 164 | mutation to proline |
| 496 | 483 | 166 | mutation to serine |
| | | | mutation to threonine |
| 498 | 485 | 168 | mutation to lysine |
| | | | mutation to arginine |
| | | | mutation to histidine |
| 501 | 488 | 171 | mutation to tyrosine |
| | | | mutation to phenylalanine |
| | | | mutation to tryptophan |
| 505 | 492 | 175 | mutation to histidine |
| 521 | 508 | 191 | mutation to serine |
| | | | mutation to threonine |

* amino acids 331-527 of SEQ ID NO: 1 and amino acids 318-514 of SEQ ID NO: 2

In embodiments, the CoV S polypeptides contain one or more modifications to the SD1/2 having an amino acid sequence of SEQ ID NO: 122, which corresponds to amino acids 542-681 of SEQ ID NO: 1 or amino acids 529-668 of SEQ ID NO: 2.

The amino acid sequence of the SD1/2 (SEQ ID NO: 122) is shown below.

NFNGLTGTGVLTESNKKFLPFQQFGRDIADTTDAVRDPQTLEILDITPC
SFGGVSVITPGTNTSNQVAVLYQDVNCTEVPVAIHADQLTPTWRVYSTG
SNVFQTRAGCLIGAEHVNNSYECDIPIGAGICASYQTQTNSP

In embodiments, the CoV S polypeptides described herein comprise a SD1/2 with at least 95%, at least 96%, at least 97%, at least 98%, at least 99%, at least 99.5%, or 100% identity to the SD1/2 of SEQ ID NO: 1 or SEQ ID NO: 2. The SD1/2 may have a deletion, an insertion, or mutation of up to about 1, up to about 2, up to about 3, up to about 4, up to about 5, up to about 10, up to about 15, up to about 20, up to about 25, or up to about 30 amino acids compared to the amino acid sequence of the SD1/2 of SEQ ID NO: 1 or SEQ ID NO: 2. The SD1/2 may have a deletion, an insertion, or mutation of between about 1 and about 5 amino acids, between about 3 and about 10 amino acids, between about 5 and 10 amino acids, between about 8 and 12 amino acids, between about 10 and 15 amino acids, between about 12 and 17 amino acids, between about 15 and 20 amino acids, between about 18 and 23 amino acids, between about 20 and 25 amino acids, between about 22 and about 27 amino acids, or between about 25 and 30 amino acids as compared to the SD1/2 of SEQ ID NO: 1 or SEQ ID NO: 2.

In embodiments, the CoV S polypeptide has at least one, at least two, at least three, at least four, at least four, at least 5, at least 6, at least 7, at least 8, at least 9, at least 10, at least 11, at least 12, at least 13, at least 14, at least 15, at least 16, at least 17, at least 18, at least 19, or at least 20 modifications in the SD1/2. In aspects, there are up to about 20, up to about 25, up to about 30, up to about 35, up to about 40, up to about 45, or up to about 50 modifications in the SD1/2. In embodiments, the SD1/2 may contain any combination of modifications as shown in Table 1D.

TABLE 1D
Modifications to SD1/2 (SEQ ID NO: 122)

| Position | Position | Position | |
|---|---|---|---|
| within | within | within | |
| SEQ | SEQ | SEQ | |
| ID | ID | ID | |
| NO: | NO: | NO: | |
| 1 | 2 | 122 | Potential Modifications |
| 547 | 534 | 6 | mutation to lysine |
| | | | mutation to arginine |
| | | | mutation to histidine |
| 570 | 557 | 29 | Mutation to aspartic acid |
| | | | Mutation to glutamic acid |
| 604 | 591 | 63 | Mutation to isoleucine |
| | | | Mutation to leucine |
| | | | Mutation to valine |
| | | | Mutation to beta-branched amino acid |
| 613 | 600 | 72 | Mutation to histidine |
| | | | Mutation to lysine |
| | | | Mutation to arginine |
| 614 | 601 | 73 | Mutation to glycine |
| | | | Mutation to alanine |
| 639 | 626 | 98 | Mutation to valine |
| | | | Mutation to leucine |
| | | | Mutation to isoleucine |
| 655 | 642 | 114 | Mutation to tyrosine |
| | | | Mutation to phenylalanine |
| | | | Mutation to tryptophan |
| 658 | 645 | 117 | Mutation to serine |
| 677 | 664 | 136 | Mutation to histidine |
| 679 | 666 | 138 | Mutation to lysine |
| | | | Mutation to arginine |
| | | | Mutation to histidine |
| 681 | 668 | 140 | Mutation to histidine |
| | | | Mutation to lysine |
| | | | Mutation to arginine |
| 547 | 534 | 6 | mutation to lysine |
| | | | mutation to arginine |
| | | | mutation to histidine |
| 570 | 557 | 29 | Mutation to aspartic acid |
| | | | Mutation to glutamic acid |
| 613 | 600 | 72 | Mutation to histidine |
| | | | Mutation to lysine |
| | | | Mutation to arginine |
| 614 | 601 | 73 | Mutation to glycine |
| | | | Mutation to alanine |
| 655 | 642 | 114 | Mutation to tyrosine |
| | | | Mutation to phenylalanine |
| | | | Mutation to tryptophan |
| 677 | 664 | 136 | Mutation to histidine |
| 679 | 666 | 138 | Mutation to lysine |
| | | | Mutation to arginine |
| | | | Mutation to histidine |

* amino acids 542-681 of SEQ ID NO: 1 or amino acids 529-668 of SEQ ID NO: 2

In embodiments, the CoV S polypeptides contain a furin site (RRAR), which corresponds to amino acids 682-685 of SEQ ID NO: 1 or amino acids 669-672 of SEQ ID NO: 2, that is inactivated by one or more mutations. Inactivation of the furin cleavage site prevents furin from cleaving the CoV S polypeptide. In embodiments, the CoV S polypeptides described herein which contain an inactivated furin cleavage site are expressed as a single chain.

In embodiments, one or more of the amino acids comprising the native furin cleavage site is mutated to any natural amino acid. In embodiments, the amino acids are L-amino acids. Non-limiting examples of amino acids include alanine, arginine, glycine, asparagine, aspartic acid, cysteine, glutamine, glutamic acid, serine, threonine, histidine, lysine, methionine, proline, valine, isoleucine, leucine, tyrosine, tryptophan, and phenylalanine.

In embodiments, one or more of the amino acids comprising the native furin cleavage site is mutated to glutamine. In embodiments, 1, 2, 3, or 4 amino acids may be mutated to glutamine. In embodiments, one of the arginines comprising the native furin cleavage site is mutated to glutamine. In embodiments, two of the arginines comprising the native furin cleavage site are mutated to glutamine. In embodiments, three of the arginines comprising the native furin cleavage site are mutated to glutamine.

In embodiments, one or more of the amino acids comprising the native furin cleavage site, is mutated to alanine. In embodiments, 1, 2, 3, or 4 amino acids may be mutated to alanine. embodiments, one of the arginines comprising the native furin cleavage site is mutated to alanine. In embodiments, two of the arginines comprising the native furin cleavage site are mutated to alanine. In embodiments, three of the arginines comprising the native furin cleavage site are mutated to alanine.

In embodiments, one or more of the amino acids comprising the native furin cleavage site is mutated to glycine. In embodiments, 1, 2, 3, or 4 amino acids may be mutated to glycine. In embodiments, one of the arginines of the native furin cleavage site is mutated to glycine. In embodiments, two of the arginines comprising the native furin cleavage site are mutated to glycine. In embodiments, three of the arginines comprising the native furin cleavage site are mutated to glycine.

In embodiments, one or more of the amino acids comprising the native furin cleavage site, is mutated to asparagine. For example 1, 2, 3, or 4 amino acids may be mutated to asparagine. In embodiments, one of the arginines comprising the native furin cleavage site is mutated to asparagine. In embodiments, two of the arginines comprising the native furin cleavage site are mutated to asparagine. In embodiments, three of the arginines comprising the native furin cleavage site are mutated to asparagine.

In embodiments, in lieu of an active furin cleavage site (SEQ ID NO: 6) the CoV S polypeptides described herein contain an inactivated furin cleavage site. In embodiments, the amino acid sequence of the inactivated furin cleavage site is represented by any one of SEQ ID NO: 7-34 or SEQ ID NO: 97. In embodiments, the amino acid sequence of the inactivated furin cleavage site is QQAQ (SEQ ID NO: 7). In embodiments, the amino acid sequence of the inactivated furin cleavage site is GSAS (SEQ ID NO: 97). In embodiments, the amino acid sequence of the inactivated furin cleavage site is GSGA (SEQ ID NO: 111). In embodiments, the amino acid sequence of the inactivated furin cleavage site is GG, GGG (SEQ ID NO: 127), GGGG (SEQ ID NO: 128), or GGGGG (SEQ ID NO: 129).

Non-limiting examples of the amino acid sequences of the inactivated furin sites contained within the CoV S polypeptides are found in Table 1E.

TABLE 1E

| Amino Acid Sequence of | Active or Inactive Furin |
|---|---|
| Furin Cleavage Site | Cleavage Site |
| RRAR (SEQ ID NO: 6) | Active |
| QQAQ (SEQ ID NO: 7) | Inactive |
| QRAR (SEQ ID NO: 8) | Inactive |
| RQAR (SEQ ID NO: 9) | Inactive |
| RRAQ (SEQ ID NO: 10) | Inactive |
| QQAR (SEQ ID NO: 11) | Inactive |
| RQAQ (SEQ ID NO: 12) | Inactive |
| QRAQ (SEQ ID NO: 13) | Inactive |
| NNAN (SEQ ID NO: 14) | Inactive |
| NRAR (SEQ ID NO: 15) | Inactive |
| RNAR (SEQ ID NO: 16) | Inactive |
| RRAN (SEQ ID NO: 17) | Inactive |
| NNAR (SEQ ID NO: 18) | Inactive |
| RNAN (SEQ ID NO: 19) | Inactive |
| NRAN (SEQ ID NO: 20) | Inactive |
| AAAA (SEQ ID NO: 21) | Inactive |
| ARAR (SEQ ID NO: 22) | Inactive |
| RAAR (SEQ ID NO: 23) | Inactive |
| RRAA (SEQ ID NO: 24) | Inactive |
| AAAR (SEQ ID NO: 25) | Inactive |
| RAAA (SEQ ID NO: 26) | Inactive |
| ARAA (SEQ ID NO: 27) | Inactive |
| GGAG (SEQ ID NO: 28) | Inactive |
| GRAR (SEQ ID NO: 29) | Inactive |
| RGAR (SEQ ID NO: 30) | Inactive |
| RRAG (SEQ ID NO: 31) | Inactive |
| GGAR (SEQ ID NO: 32) | Inactive |
| RGAG (SEQ ID NO: 33) | Inactive |
| GRAG (SEQ ID NO: 34) | Inactive |
| GSAS (SEQ ID NO: 97) | Inactive |
| GSGA (SEQ ID NO: 111) | Inactive |

In embodiments, the CoV S polypeptides contain one or more modifications to the S2 subunit having an amino acid sequence of SEQ ID NO: 120, which corresponds to amino acids 686-1273 of SEQ ID NO: 1 or amino acids 673-1260 of SEQ ID NO: 2.

The amino acid sequence of the S2 subunit (SEQ ID NO: 120) is shown below.

SVASQSIIAYTMSLGAENSVAYSNNSIAIPTNFTISVTTEILPVSMTKT
SVDCTMYICGDSTECSNLLLQYGSFCTQLNRALTGIAVEQDKNTQEVFA
QVKQIYKTPPIKDFGGFNFSQILPDPSKPSKRSFIEDLLFNKVTLADAG
FIKQYGDCLGDIAARDLICAQKFNGLTVLPPLLTDEMIAQYTSALLAGT
ITSGWTFGAGAALQIPFAMQMAYRENGIGVTQNVLYENQKLIANQFNSA
IGKIQDSLSSTASALGKLQDVVNQNAQALNTLVKQLSSNFGAISSVLND
ILSRLDKVEAEVQIDRLITGRLQSLQTYVTQQLIRAAEIRASANLAATK
MSECVLGQSKRVDFCGKGYHLMSFPQSAPHGVVFLHVTYVPAQEKNFTT
APAICHDGKAHFPREGVFVSNGTHWFVTQRNFYEPQIITTDNTFVSGNC
DVVIGIVNNTVYDPLQPELDSFKEELDKYFKNHTSPDVDLGDISGINAS
VVNIQKEIDRLNEVAKNLNESLIDLQELGKYEQYIKWPWYIWLGFIAGL
IAIVMVTIMLCCMTSCCSCLKGCCSCGSCCKFDEDDSEPVLKGVKLHYT

In embodiments, the CoV S polypeptides described herein comprise an S2 subunit with at least 95%, at least 96%, at least 97%, at least 98%, at least 99%, at least 99.5%, at least 99.7%, at least 99.9%, or 100% identity to the S2 subunit of SEQ ID NO: 1 or SEQ ID NO: 2. The S2 subunit may have a deletion, an insertion, or mutation of up to about 1, up to about 2, up to about 3, up to about 4, up to about 5, up to about 10, up to about 15, up to about 20, up to about 25, or up to about 30 amino acids compared to the amino acid sequence of the S2 subunit of SEQ ID NO: 1 or SEQ ID NO: 2. The S2 subunit may have a deletion, an insertion, or mutation of from about 1 to about 50 amino acids, from about 1 to about 5 amino acids, from about 3 to about 10 amino acids, from about 5 to 10 amino acids, from about 8 to 12 amino acids, from about 10 to 15 amino acids, from about 12 to 17 amino acids, from about 15 to 20 amino acids, from about 18 to 23 amino acids, from about 20 to 25 amino acids, from about 22 to about 27 amino acids, or from about 25 to 30 amino acids as compared to the S2 subunit of SEQ ID NO: 1 or SEQ ID NO: 2.

In embodiments, the CoV S polypeptides contain a mutation at Lys-973 of the native CoV Spike(S) polypeptide (SEQ ID NO: 2). In embodiments, Lys-973 is mutated to any natural amino acid. In embodiments, Lys-973 is mutated to proline. In embodiments, Lys-973 is mutated to glycine. In embodiments, the CoV S polypeptides containing a mutation at amino acid 973 are selected from the group consisting of SEQ ID NO: 84-89, 105-106, and 109-110.

In embodiments, the CoV S polypeptides contain a mutation at Val-974 of the native CoV Spike(S) polypeptide (SEQ ID NO: 2). In embodiments, Val-974 is mutated to any natural amino acid. In embodiments, Val-974 is mutated to proline. In embodiments, Val-974 is mutated to glycine. In embodiments, the CoV S polypeptides containing a mutation at amino acid 974 are selected from the group consisting of SEQ ID NO: 84-89, 105-106, and 109-110.

In embodiments, the CoV S polypeptides contain a mutation at Lys-973 and Val-974 of the native CoV Spike(S) polypeptide (SEQ ID NO: 2). In embodiments, Lys-973 and Val-974 are mutated to any natural amino acid. In embodiments, Lys-973 and Val-974 are mutated to proline. In embodiments, the CoV S polypeptides containing a mutation at amino acids 973 and 974 are selected from SEQ ID NOS: 84-89, 105-106, 109-110, 175, 220, and 217-228.

In embodiments, the S2 subunit may contain any combination of modifications as shown in Table 1F.

TABLE 1F
Modifications to S2 (SEQ ID NO: 120)

| Position | Position | Position | |
|---|---|---|---|
| within | within | within | |
| SEQ | SEQ | SEQ | |
| ID | ID | ID NO: | |
| NO: 1 | NO: 2 | 120 | Possible Modifications |
| 689-698 | 676-685 | 4-13 | Deletion of up to about 1, up to about 2, |
| | | | up to about 3, up to about 4, up to about |
| | | | 5, up to about 6, up to about 7, up to |
| | | | about 8, up to about 9, or up to |
| | | | about 10 amino acids |
| 701 | 688 | 16 | Mutation to beta-branched amino acid |
| | | | Mutation to valine |
| | | | Mutation to isoleucine |
| | | | Mutation to threonine |
| 704 | 691 | 19 | Mutation to leucine |
| | | | Mutation to isoleucine |
| | | | Mutation to valine |
| 715-724 | 702-711 | 30-39 | Deletion of up to about 1, up to about 2, |
| | | | up to about 3, up to about 4, up to about |
| | | | 5, up to about 6, up to about 7, up to |
| | | | about 8, up to about 9, or up to about |
| | | | 10 amino acids |
| 716 | 703 | 31 | Mutation to beta-branched amino acid |
| | | | Mutation to valine |
| | | | Mutation to isoleucine |
| 764 | 751 | 79 | Mutation to lysine |
| | | | Mutation to arginine |
| | | | Mutation to histidine |
| 788-806 | 775-793 | 103-121 | Deletion of up to about 1, up to about 2, |
| | | | up to about 3, up to about 4, up to about |
| | | | 5, up to about 6, up to about 7, up to |
| | | | about 8, up to about 9, up to about 10, |
| | | | up to about 11, up to about 12, up to |
| | | | about 13, up to about 14, up to about |
| | | | 15, up to about 16, up to about 17, up to |
| | | | about 18, or up to about 19 amino acids |
| 796 | 783 | 111 | Mutation to tyrosine |
| | | | Mutation to phenylalanine |
| | | | Mutation to tryptophan |
| 819-828 | 806-815 | 134-143 | Deletion of up to about 1, up to about 2, |
| | | | up to about 3, up to about 4, up to about |
| | | | 5, up to about 6, up to about 7, up to |
| | | | about 8, up to about 9, or up to about |
| | | | 10 amino acids |
| 856 | 843 | 171 | Mutation to lysine |
| | | | Mutation to arginine |
| | | | Mutation to histidine |
| 859 | 846 | 174 | Mutation to asparagine |
| | | | Mutation to glutamine |
| 888 | 875 | 203 | Mutation to leucine |
| | | | Mutation to isoleucine |
| | | | Mutation to valine |
| 950 | 937 | 265 | Mutation to asparagine |
| | | | Mutation to glutamine |
| 954 | 941 | 269 | Mutation to histidine |
| | | | Mutation to lysine |
| | | | Mutation to arginine |
| 969 | 956 | 284 | Mutation to lysine |
| | | | Mutation to arginine |
| | | | Mutation to histidine |
| 981 | 968 | 296 | Mutation to phenylalanine |
| | | | Mutation to tyrosine |
| | | | Mutation to tryptophan |
| 982 | 969 | 297 | Mutation to alanine |
| | | | Mutation to glycine |
| | | | Mutation to threonine |
| 986 | 973 | 301 | Mutation to proline |
| | | | Mutation to glycine |
| 987 | 974 | 302 | Mutation to proline |
| | | | Mutation to glycine |
| 1027 | 1014 | 342 | Mutation to isoleucine |
| | | | Mutation to valine |
| | | | Mutation to serine |
| 1071 | 1058 | 386 | Mutation to histidine |
| | | | Mutation to arginine |
| | | | Mutation to lysine |
| 1118 | 1105 | 433 | Mutation to histidine |
| | | | Mutation to lysine |
| | | | Mutation to arginine |
| | | | Mutation to asparagine |
| | | | Mutation to glutamine |
| 1176 | 1163 | 491 | Mutation to phenylalanine |
| | | | Mutation to tyrosine |
| | | | Mutation to tryptophan |
| 1199 | 1186 | 514 | Mutation to asparagine |
| | | | Mutation to glutamine |
| 1214- | 1201- | 1-24 | Deletion of one or more amino acids |
| 1237 | 1224 | | of TM |
| 1238- | 1225- | 1-36 | Deletion of one or more amino acids |
| 1273 | 1260 | | of CD |

* amino acids 686-1273 of SEQ ID NO: 1 and amino acids 673-1260 of SEQ ID NO: 2

In embodiments, the intranasal immunogenic compositions comprise 1, 2, 3, 4, about 5, about 6, about 7, about 8, about 9, about 10, about 11, about 12, about 13, about 14, about 15, about 16, about 17, about 18, about 19, about 20, about 21, about 22, about 23, about 24, about 25, about 26, about 27, about 28, about 29, about 30, about 31, about 32, about 33, about 34, about 35, about 36, about 37, about 38, about 39, about 40, about 41, about 42, about 43, about 44, about 45, about 46, about 47, about 48, about 49, or about 50 SARS-COV-2 S glycoproteins, including all values and ranges therebetween.

Multiple examples of SARS-COV-2 S glycoproteins are described in the following patent documents, which are each incorporated by reference herein in their entirety for all purposes: International Publication No. 2021/015220, International Publication No. 2022/020974, International Publication No. 2022/235662, International Publication No. 2023/102448.

### Influenza Glycoproteins

In embodiments, the intranasal immunogenic compositions comprise from 1 to about 50 influenza glycoproteins (e.g., hemagglutinin and/or neuraminidase). In embodiments, the intranasal immunogenic compositions comprise 1, 2, 3, 4, about 5, about 6, about 7, about 8, about 9, about 10, about 11, about 12, about 13, about 14, about 15, about 16, about 17, about 18, about 19, about 20, about 21, about 22, about 23, about 24, about 25, about 26, about 27, about 28, about 29, about 30, about 31, about 32, about 33, about 34, about 35, about 36, about 37, about 38, about 39, about 40, about 41, about 42, about 43, about 44, about 45, about 46, about 47, about 48, about 49, or about 50 influenza glycoproteins, including all values and ranges therebetween.

Multiple examples of influenza HA glycoproteins are described in the following patent documents, which are each incorporated by reference herein in their entirety for all purposes: International Publication No. 2017/041100 and International Publication No. 2019/022930.

In embodiments, an immunogenic composition of the disclosure comprises at least three HA glycoproteins or at least four HA glycoproteins. In embodiments, an immunogenic composition of the disclosure comprises three or four HA glycoproteins. In embodiments, each of the HA glycoproteins are from a different influenza strain. In embodiments, three HA glycoproteins are from a Type A influenza strain, and one HA glycoprotein is from a Type B influenza strain. In embodiments, two HA glycoproteins are from a Type A influenza strain, and two HA glycoproteins are from a Type B influenza strain. In embodiments, two HA glycoproteins are from a Type A influenza strain, and one HA glycoprotein is from a Type B influenza strain.

In embodiments, each of the at least three HA glycoproteins is isolated separately using an egg-based manufacturing process. In embodiments, an egg-based manufacturing process comprises (a) propagating an influenza virus in an egg and (b) harvesting the influenza virus.

In embodiments, the influenza virus is a live virus. In embodiments, the influenza virus is a weakened or “attenuated” virus. In embodiments, the influenza virus is optimized to grow in an egg. In embodiments, the optimized influenza virus lacks the polybasic cleavage site of hemagglutinin. The following article describes the development of an optimized influenza virus (referred to as a “candidate vaccine virus”) in detail and is incorporated by reference herein in its entirety: Belser et al. Virology. 2017 November; 511:135-141.

In embodiments, the egg is a chicken egg. In embodiments, the chicken egg is an embryonated chicken egg. In embodiments, the egg is a pathogen-free egg. In embodiments, an influenza virus is propagated by inoculating the virus in the allantoic cavity of an embryonated chicken egg. The following article describes an exemplary inoculation method and is incorporated by reference herein in its entirety: Brauer et al. J Vis Exp. 2015; (97): 52421.

In embodiments, the egg-based manufacturing process comprises purifying the influenza virus. In embodiments, an influenza virus is purified using any of the following techniques: centrifugation, chromatography, precipitation, or nanofiltration. In embodiments, the centrifugation technique is ultracentrifugation. In embodiments, the virus is purified using zonal centrifugation. In embodiments, the zonal centrifugation is continuous flow zonal centrifugation.

In embodiments, the egg-based manufacturing process comprises inactivating (also referred to herein as “killing”) an influenza virus. In embodiments, an influenza virus is inactivated using low pH (e.g., a pH from about 3.5-5.5), heat, ethanol, ultraviolet light, exposure to a detergent (e.g, octylphenol), or exposure to a chemical (e.g., 2-propanol, ethanol, iodopovidone). In embodiments, the purified virus is inactivated with ultraviolet light, betapropiolactone, sodium deoxycholate, formaldehyde, or any combination thereof.

In embodiments, the egg-based manufacturing process comprises exposing the influenza virus to a surfactant. The surfactant may be sodium taurodeoxycholate, octylphenol ethoxylate (Triton®-X 100), or cetyl trimethyl ammonium bromide. Exposing an influenza virus to a surfactant results in the formation of an influenza split-virion. In embodiments, the at least three HA glycoproteins are in the form of an influenza split-virion.

In embodiments, the egg-based manufacturing process comprises purifying an influenza antigen (e.g., hemagglutinin) from the virus.

The following Food and Drug Administration (FDA) approved influenza vaccines are produced using an egg-based manufacturing process: AFLURIA® QUADRIVALENT, FLUARIX® QUADRIVALENT, FLULAVAL QUADRIVALENT, FLUZONE® QUADRIVALENT, FLUZONE® HIGH-DOSE QUADRIVALENT, FLUMIST® QUADRIVALENT, FLUAD®, and FLUAD® QUADRIVALENT.

In embodiments, each of the at least three HA glycoproteins is isolated using a cell-culture based process. In embodiments, a cell-culture based process comprises (i) growing an influenza virus in a cell and (ii) harvesting the virus from the cell. In embodiments, a cell-culture based process comprises (i) transfecting a cell with a vector comprising a hemagglutinin and (ii) harvesting the hemagglutinin from the cell. In embodiments, a cell-culture based process comprises (i) transducing a cell with a virus encoding a hemagglutinin and (ii) harvesting the hemagglutinin from the cell. In embodiments, the harvested hemagglutinin is recombinant hemagglutinin.

In embodiments, the cell is an animal cell, a bacterial cell, an insect cell, or a fungal cell. In embodiments, the animal is a human, a bird (e.g., a chicken), a dog, a reptile, a goat, a pig, a mouse, a rabbit, or a rat.

In embodiments, the virus encoding a hemagglutinin is a baculovirus, a lentivirus, or an adeno-associated virus.

In embodiments, an influenza virus produced using a cell-culture based process is purified. Any of the purification techniques described to purify influenza virus produced using an egg-based manufacturing process can be used to purify influenza virus produced using a cell-cultured based process.

In embodiments, a hemagglutinin produced using a cell-culture based process is purified. Purification techniques include chromatography, centrifugation, precipitation, and nanofiltration.

The Food and Drug Administration (FDA) approved influenza vaccine FLUCELVAX® QUADRIVALENT is produced using a cell-culture based process.

In embodiments, each of the at least three HA glycoproteins is a recombinant hemagglutinin. The Food and Drug Administration (FDA) approved influenza vaccine FLUBLOK® QUADRIVALENT is a recombinant hemagglutinin.

In embodiments, the at least three hemagglutinins are in the form of recombinant hemagglutinin. Recombinant hemagglutinin is isolated from a cell that produces hemagglutinin. In embodiments, a cell that produces hemagglutinin has been transfected with a vector encoding hemagglutinin. In embodiments, a cell that produces hemagglutinin has been transduced with a virus encoding hemagglutinin.

In embodiments, the compositions disclosed herein comprise detergent-core nanoparticles comprising hemagglutinin from an influenza virus. The aforementioned detergent-core nanoparticles are described in detail in U.S. Pat. No. 10,426,829, which is incorporated herein by reference in its entirety for all purposes.

Detergent-core nanoparticles comprise a hemagglutinin from an influenza virus, which is associated with a detergent core. In embodiments, the hemagglutinin is a trimer. Each nanoparticle may contain 1, 2, 3, 4, 5, 6, 7, 8, 9, 10, 11, 12, 13, 14, or 15 trimers. In embodiments, the nanoparticle contains between about 2 to about 9, about 2 to about 6, or about 5 hemagglutinin trimers. The hemagglutinin is associated with the non-ionic detergent containing core of the nanoparticle. In embodiments, the detergent is selected from polysorbate-20 (PS20), polysorbate-40 (PS40), polysorbate-60 (PS60), polysorbate-65 (PS65) and polysorbate-80 (PS80). The presence of the detergent facilitates formation of the nanoparticles by forming a core that organizes and presents the antigens. In embodiments, the nanoparticles may contain the antigens assembled into multi-oligomeric glycoprotein-PS80 protein-detergent nanoparticles with the head regions projecting outward and hydrophobic regions and PS80 detergent forming a central core surrounded by the antigens.

The nanoparticles disclosed herein range in Z-ave size from about 20 nm to about 60 nm, about 20 nm to about 50 nm, about 20 nm to about 45 nm, or about 25 nm to about 45 nm.

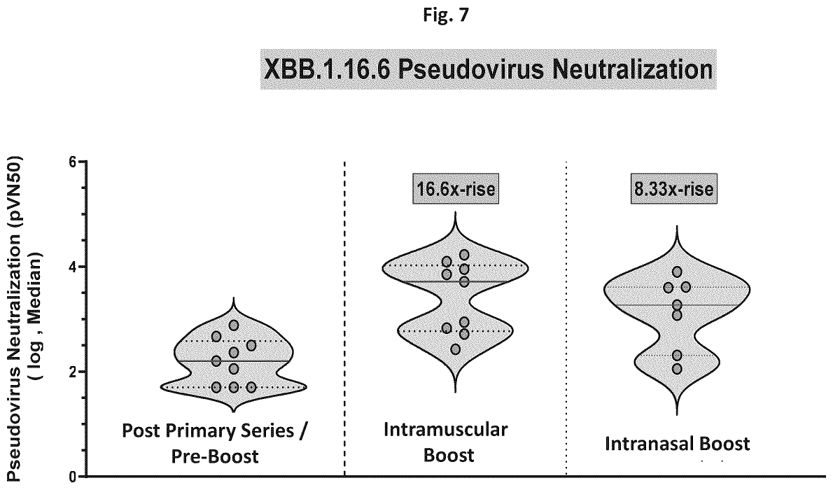

In embodiments, a detergent-core nanoparticle is produced in insect cells by expressing HA proteins using a baculovirus expression system and extracting the HA protein with a detergent. During purification, the first detergent is exchanged for a second detergent, typically a non-ionic detergent resulting in nanoparticles having a non-ionic detergent core in which the transmembrane domain of the HA protein, in trimer form, is embedded into. FIG. 7 (left panel) illustrates these structures as observed under an electron microscope.

The hemagglutinin contained in the detergent-core nanoparticles or in the HaSMaNs described herein may be from any influenza virus strain. Human influenza Type A and Type B viruses cause seasonal epidemics of disease almost every winter in the United States.

The HA protein may be selected from the sub-types H1, H2, H3, H4, H5, H6, H7, H8, H9, H10, H11, H12, H13, H14, H15, H16, H17, and H18. Phylogenetically, the influenza is split into groups. For HA, Group 1 contains H1, H2, H5, H6, H8, H9, H11, H12, H13, H16, H17, and H18 and group 2 contains H3, H4, H7, H10, H14, and H15.

In embodiments, the detergent-core nanoparticles or HaSMaNs are trypsin-resistant nanoparticles produced using neutral pH purification. Trypsin resistance is achieved by neutral pH range of above 6.9 to 8.5 during purification and formulation of the HA nanoparticles. Trypsin resistant influenza glycoproteins and trypsin resistant influenza nanoparticles; and methods of making thereof are described in detail in U.S. Pat. No. 10,426,829.

In embodiments, the hemagglutinin of the detergent-core nanoparticles or HaSMaNs described herein comprises the full-length wild type hemagglutinin amino acid sequence. In embodiments, the hemagglutinin is a hemagglutinin variant. In embodiments, the hemagglutinin exhibits at least 80%, at least 85%, at least 90%, at least 95%, at least 97%, at least 98%, or at least 99% identity to the wild-type hemagglutinin protein.

HA is a homotrimer with each monomer consisting of ˜550 amino acid residues. Each monomer of HA has been conceptually divided into three domains: the ectodomain of ˜515 residues constitutes the extraviral part of the molecule; a single stretch of 27 residues defines the transmembrane (TM) domain; and ˜10 residues constitute the cytoplasmic tail (CT). While some changes may be made to hemagglutinin, formation of both detergent core nanoparticles and HaSMaNs requires an intact transmembrane domain™. Thus, in particular examples, a modified HA protein sequence comprises 100% identity to the wild-type™ and CT domains with some flexibility in the remaining ectodomain portion, where identity may be at least 90% or at least 95%. The domains may be identified by homology to the amino acid sequences of the TM domains and CT of Japan/305/57 HA shown in FIG. 1 of Melikyan et al. (Mol Biol Cell. 1999 June; 10 (6): 1821-1836) though it should be noted that the boundaries between ectodomain, TM, and CT domains may vary from HA protein to HA protein by up to three amino acids.

(iib) influenza vaccine-HaSMaNs (Hemagglutinin Saponin Matrix Nanoparticles)

In embodiments, the immunogenic compositions and vaccine compositions described herein comprise a HaSMaN (Hemagglutinin Saponin Matrix Nanoparticle.) FIG. 7 (right panel) illustrates HaSMaN structures as observed under an electron microscope. The HA glycoproteins decorate the Matrix cage-like structures. The HaSMaN structures are formed by preparing detergent-corenanoparticles comprising hemagglutinin from an influenza virus and then incubating them with ISCOM matrix adjuvant particles for a period of time. ISCOM matrix particles are shown in the center panel of FIG. 7. Notably, the HaSMaNs form readily with Type A influenza HA proteins, but not Type B influenza HA proteins.

HaSMaNs disclosed herein are produced by incubating the detergent-core nanoparticles with an ISCOM Matrix adjuvant comprising a saponin fraction, cholesterol and a phospholipid. In embodiments, a HaSMaN is formed by incubating a detergent-core nanoparticle with an ISCOM matrix adjuvant for between about 24 hours and about 48 hours. For example, the detergent-core nanoparticle may be incubated with an ISCOM matrix adjuvant for about 24 hours, about 25 hours, about 26 hours, about 27 hours, about 28 hours, about 29 hours, about 30 hours, about 31 hours, about 32 hours, about 33 hours, about 34 hours, about 35 hours, about 36 hours, about 37 hours, about 38 hours, about 39 hours, about 40 hours, about 41 hours, about 42 hours, about 43 hours, about 44 hours, about 45 hours, about 46 hours, about 47 hours, about 48 hours, or more. In some embodiments, a HaSMaN is formed by incubating a detergent-core nanoparticle with an ISCOM matrix adjuvant for at least about 24 hours, at least about 25 hours, at least about 26 hours, at least about 27 hours, at least about 28 hours, at least about 29 hours, at least about 30 hours, at least about 31 hours, at least about 32 hours, at least about 33 hours, at least about 34 hours, at least about 35 hours, at least about 36 hours, at least about 37 hours, at least about 38 hours, at least about 39 hours, at least about 40 hours, at least about 41 hours, at least about 42 hours, at least about 43 hours, at least about 44 hours, at least about 45 hours, at least about 46 hours, at least about 47 hours, or at least about 48 hours. In embodiments, a HaSMaN is formed by incubating a detergent-core nanoparticle with an ISCOM matrix adjuvant at a temperature from about 4° C. to about 25° C. For example, the HaSMaN may be formed by incubating a detergent-core nanoparticle with an ISCOM matrix adjuvant at a temperature of about 4° C., about 5° C., about 6° C., about 7° C., about 8° C., about 9° C., about 10° C., about 11° C., about 12° C., about 13° C., about 14° C., about 15° C., about 16° C., about 17° C., about 18° C., about 19° C., about 20° C., about 21° C., about 22° C., about 23° C., about 24° C., about 25° C., or higher. In embodiments, a HaSMaN is formed by incubating a detergent-core nanoparticle with an ISCOM matrix adjuvant at a temperature of at least about 4° C., at least about 5° C., at least about 6° C., at least about 7° C., at least about 8° C., at least about 9° C., at least about 10° C., at least about 11° C., at least about 12° C., at least about 13° C., at least about 14° C., at least about 15° C., at least about 16° C., at least about 17° C., at least about 18° C., at least about 19° C., at least about 20° C., at least about 21° C., at least about 22° C., at least about 23° C., at least about 24° C., at least about 25° C. Typically, about 24 to 48 hours at 4° C. or 25° C. incubation is required for formation. Formation of HaSMaNs is promoted by higher temperatures. In embodiments, formation of HaSMaN occurs by incubation of detergent-core nanoparticles with an ISCOM Matrix adjuvant for at least 24 hours at about 25° C. Mixing detergent core nanoparticles with ISCOM Matrix adjuvant shortly prior to administering to a subject—i.e. bedside mix, does not produce HaSMaNs. Longer incubation periods do not negatively impact HaSMaNs formation.

### RSV Glycoproteins

In embodiments, the intranasal immunogenic compositions comprise from 1 to about 50 respiratory syncytial virus (RSV) fusion (F) glycoproteins. The wild-type RSV F glycoprotein is synthesized as a single-chain inactive precursor called F0 that contains three subunits: F1, F2, and a 27-amino acid glycopeptide called pep27 (“p27”). The F1 subunit is located at amino acids 137-574 of the RSV F glycoprotein of SEQ ID NO: 330. The F2 subunit is located at amino acids 26-110 of the RSV F glycoprotein of SEQ ID NO: 2. p27 is located at amino acids 111-136 of the RSV F glycoprotein of SEQ ID NO: 330. The C-terminal F1 subunit contains a transmembrane domain, two heptad repeats, and an N-terminal fusion peptide. The F0 precursor is cleaved by a furin-like protease to form the mature, fusion competent protein. The mature RSV F protein contains an F1 subunit and an F2 subunit connected by a disulfide bond. Additional sources that describe the RSV F protein structure are found at Swanson et al. A Monomeric Uncleaved Respiratory Syncytial Virus F Antigen Retains Prefusion-Specific Neutralizing Epitopes. Journal of Virology, 2014, 88, 11802-11810. Jason S. Mclellan et al. Structure of RSV Fusion Glycoprotein Trimer Bound to a Prefusion-Specific Neutralizing Antibody. Science, 2013, 340, 1113-1117.

In embodiments, the intranasal immunogenic compositions comprise 1, 2, 3, 4, about 5, about 6, about 7, about 8, about 9, about 10, about 11, about 12, about 13, about 14, about 15, about 16, about 17, about 18, about 19, about 20, about 21, about 22, about 23, about 24, about 25, about 26, about 27, about 28, about 29, about 30, about 31, about 32, about 33, about 34, about 35, about 36, about 37, about 38, about 39, about 40, about 41, about 42, about 43, about 44, about 45, about 46, about 47, about 48, about 49, or about 50 RSV F glycoproteins, including all values and ranges therebetween.

Multiple examples of RSV F glycoproteins are described in the following patent documents, which are each incorporated by reference herein in their entirety for all purposes: International Publication No. 2017/041100 and International Publication No. 2010/077717.

In embodiments, provided herein are RSV F glycoproteins that have one or more modifications compared to a native (also referred to as “wild-type”) RSV F glycoprotein. In embodiments, an RSV F glycoprotein from the wild type RSV F A2 strain is the native RSV F glycoprotein. The RSV F glycoprotein from the wild type RSV F A2 strain comprises the amino acid sequence of SEQ ID NO: 330. The wild type RSV F A2 strain has sequencing errors (A102P, V379I, V447M). These sequencing errors are corrected in the non-naturally occurring RSV F glycoproteins described herein, i.e., amino acid 102 is alanine, amino acid 379 is valine, and amino acid 447 is valine.

In embodiments, the RSV F glycoproteins have one or more modifications selected from the group consisting of: (a) deletion of one or more of amino acids 137-146, which are within the fusion domain; (b) inactivation of the primary furin cleavage site (also referred to as Site II) through mutation of one or more of amino acids 131-136; (c) inactivation of the secondary furin cleavage site (also referred to as Site I) through mutation of one or more of amino acids 106-109; (d) S155C; (e) S290C; (f) S190F; (g) V207L; and (h) N116Q; wherein the modifications are numbered according to the RSV F glycoprotein of SEQ ID NO: 330.

In embodiments, the RSV F glycoproteins have one or more modifications selected from the group consisting of: (a) deletion of one or more of amino acids 137-146, which are within the fusion domain; (b) inactivation of the primary furin cleavage site (also referred to as Site II) through mutation of one or more of amino acids 131-136; (c) inactivation of the secondary furin cleavage site (also referred to as Site I) through mutation of one or more of amino acids 106-109; (d) S155C; (e) S290C; (f) S190F; and (g) V207L; wherein the modifications are numbered according to the RSV F glycoprotein of SEQ ID NO: 330.

In aspects, RSV F glycoproteins disclosed herein comprise deletions in the fusion peptide compared to the wild-type RSV F glycoprotein of SEQ ID NO: 330. In embodiments, the RSV F glycoproteins contain an inactivated primary furin cleavage site. In aspects, the cysteine residues are mutated. Typically, the N-linked glycosylation sites are not mutated compared to the corresponding wild-type sequence.

In embodiments, the RSV F glycoproteins described herein exhibit one or more of the antigenic sites Ø, II, IV, and V. In embodiments, the RSV F proteins described herein exhibit mutations, which result in the presence of one or more of the RSV F antigenic sites Ø, II, IV, and V. The RSV F antigenic site Ø, which is located at the membrane-distal apex of the prefusion RSV F trimer, is targeted by potent RSV F neutralizing antibodies, including 5C4, AM22, and D25. The RSV F antigenic site II, also referred to herein as the palivizumab site, is the target of the antibodies pavilizumab and motavizumab. Antigenic site II is composed of a helix-turn-helix motif that spans residues 253-278. The RSV F antigenic site IV is located on the F1 subunit of the RSV F protein and encompasses residues 422-471 of SEQ ID NO: 330. The RSV F antigenic site V is composed of a2-a3 and B3-B4.

In embodiments, a particular antigenic site contains epitopes associated with pre-fusion or post-fusion structure. For example, the RSV F antigenic sites Ø and V are exhibited in pre-fusion structures of the RSV F glycoprotein. In contrast, the RSV F antigenic sites II and IV are exhibited in both pre-fusion and post-fusion structures of the RSV F glycoprotein. In embodiments, RSV F proteins contain antigenic sites that induce the formation of neutralizing antibodies against the pre-fusion conformation of the RSV F glycoprotein. In embodiments, the RSV F glycoproteins contain antigenic sites that induce the formation of neutralizing antibodies against the post-fusion conformation of the RSV F glycoprotein. In embodiments, the RSV F glycoproteins described herein contain antigenic sites that induce the production of neutralizing antibodies against both pre-fusion and post-fusion conformations of the RSV F glycoprotein.

In embodiments, the RSV F glycoproteins comprise a deletion of amino acids 137-146; an inactivated primary furin cleavage site; a S155C mutation; and a S290C mutation, wherein the modifications are numbered according to the RSV F glycoprotein of SEQ ID NO: 330. In embodiments, the RSV F glycoproteins comprise a deletion of amino acids 137-146; an inactivated primary furin cleavage site; a S155C mutation; a S290C mutation; a S190F mutation; and a V207L mutation, wherein the modifications are numbered according to the RSV F glycoprotein of SEQ ID NO: 330. In embodiments, the RSV F glycoproteins comprise a deletion of amino acids 137-146; an inactivated primary furin cleavage site; a S19OF mutation; and a V207L mutation, wherein the modifications are numbered according to the RSV F glycoprotein of SEQ ID NO: 330.

In embodiments, the RSV F glycoproteins comprise a deletion of amino acids 137-146; an inactivated primary furin cleavage site; and a N116Q mutation, wherein the modifications are numbered according to the RSV F glycoprotein of SEQ ID NO: 330. In embodiments, the RSV F glycoproteins comprise a deletion of amino acids 137-146; an inactivated primary furin cleavage site; a S155C mutation; a S290C mutation; and a N116Q mutation, wherein the modifications are numbered according to the RSV F glycoprotein of SEQ ID NO: 330. In embodiments, the RSV F glycoproteins comprise a deletion of amino acids 137-146; an inactivated primary furin cleavage site; a S190F mutation; a V207L mutation; and a N116Q mutation, wherein the modifications are numbered according to the RSV F glycoprotein of SEQ ID NO: 330. In embodiments, the RSV F glycoproteins comprise a deletion of amino acids 137-146; an inactivated primary furin cleavage site; a S155C mutation; a S290C mutation; a S190F mutation; a V207L mutation; and a N116Q mutation, wherein the modifications are numbered according to the RSV F glycoprotein of SEQ ID NO: 330.

In embodiments, particular mutations cause the mature RSV F proteins of the disclosure to retain p27 at the N-terminus of the F1 subunit. RSV F proteins which retain p27 contain a p27-F1 subunit and an F2 subunit that are connected by a disulfide bond. In embodiments, a mutation in the primary furin cleavage site leads to retention of p27.

The primary furin cleavage site, also referred to as Site II herein, is located at residues 131 to 136 of SEQ ID NO: 330. The wild-type primary furin cleavage site has the amino acid sequence of KKRKRR (SEQ ID NO: 341). Inactivation of the primary fusion cleavage site may be achieved by mutating residues in the site, with the result that furin can no longer recognize the consensus site. For example, inactivation of the primary furin cleavage site may be accomplished by introducing at least one, at least two, or three amino acid substitutions at positions corresponding to arginine 133, arginine 135, and arginine 136 of the wild-type RSV F protein (SEQ ID NO:330). In particular aspects, one, two, or all three of the arginines are mutated to glutamine. In other aspects, inactivation is accomplished by mutating the wild-type primary furin cleavage site to one of the following sequences: KKQKQQ (SEQ ID NO: 342), QKQKQQ (SEQ ID NO:343), KKQKRQ (SEQ ID NO: 344), and GRRQQR (SEQ ID NO: 345).

In embodiments, RSV F glycoproteins described herein comprise a wild-type secondary furin cleavage site. The secondary furin cleavage site, also referred to as Site I herein, is located at residues 106 to 109 of SEQ ID NO: 330. The wild-type secondary furin cleavage site has the amino acid sequence RARR (SEQ ID NO: 6). In embodiments, RSV F glycoproteins described herein comprise an inactive secondary furin-cleavage site. In embodiments, inactivation of the secondary furin cleavage site is accomplished by mutating the wild-type site to one of the following sequences: QQAQ (SEQ ID NO: 7), RAQQ (SEQ ID NO: 348), or RANN (SEQ ID NO: 349).

In embodiments, the RSV F glycoproteins of the disclosure lack one or more amino acids of the RSV F fusion peptide (amino acids 137-154 of SEQ ID NO: 330). In particular aspects, from 1 to 10 amino acids of the corresponding amino acids 137 to 146 of SEQ ID NO: 330 may be deleted. For example, up to 1, up to 2, up to 3, up to 4, up to 5, up to 6, up to 7, up to 8, up to 9, or up to 10 amino acids within the fusion peptide may be deleted. Alternatively, at least 1, at least 2, at least 3, at least 4, at least 5, at least 6, at least 7, at least 8, at least 9, or at least 10 amino acids within the fusion peptide may be deleted. In embodiments, the deleted amino acids are contiguous. In embodiments, the deleted amino acids are non-contiguous. In embodiments, the RSV F glycoproteins described herein lack the entire fusion peptide.

In embodiments, the non-naturally occurring RSV F glycoproteins described herein may have a deletion, an insertion, or mutation of up to about 1, up to about 2, up to about 3, up to about 4, up to about 5, up to about 10, up to about 15, up to about 20, up to about 25, or up to about 30 amino acids compared to the amino acid sequence of the F1 domain of SEQ ID NO: 330. The wild-type F1 domain has the amino acid sequence of SEQ ID NO: 350. In embodiments, the RSV F glycoproteins described herein comprise an F1 domain with at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, at least 99.5%, identity, or 100% identity to the F1 domain of SEQ ID NO: 350. The F1 domain of an RSV F glycoprotein herein may have a deletion, an insertion, or mutation of between about 1 and about 5 amino acids, between about 3 and about 10 amino acids, between about 5 and 10 amino acids, between about 8 and 12 amino acids, between about 10 and 15 amino acids, between about 12 and 17 amino acids, between about 15 and 20 amino acids, between about 18 and 23 amino acids, between about 20 and 25 amino acids, between about 22 and about 27 amino acids, or between about 25 and 30 amino acids as compared to the F1 domain of SEQ ID NO: 350.

In embodiments, the F1 domain of the RSV F glycoproteins comprise one or more mutations selected from the group consisting of S190F, V207L, S155C, S290C, 1379A, and M447V, wherein the modifications are numbered according to the RSV F glycoprotein of SEQ ID NO: 330. In embodiments, the F1 domain of the RSV F glycoproteins comprise S190F and V207 L mutations, wherein the modifications are numbered according to the RSV F glycoprotein of SEQ ID NO: 330. In embodiments, the F1 domain of the RSV F glycoproteins comprise S155C and S290C mutations, wherein the mutations are numbered according to the RSV F glycoprotein of SEQ ID NO: 330. In embodiments, the F1 domain of the RSV F glycoproteins comprise S190F, V207L, S155C, and S290C mutations, wherein the mutations are numbered according to the RSV F glycoprotein of SEQ ID NO: 330.

In embodiments, the F1 domain of the RSV F glycoproteins comprises an amino acid sequence with at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, at least 99.5% identity, or 100% identity any one of SEQ ID NOS: 350-355.

In embodiments, the non-naturally occurring RSV F glycoproteins described herein may have a deletion, an insertion, or mutation of up to about 1, up to about 2, up to about 3, up to about 4, up to about 5, up to about 10, up to about 15, up to about 20, up to about 25, or up to about 30 amino acids compared to the amino acid sequence of the F2 domain of SEQ ID NO: 330. The wild-type F2 domain has the amino acid sequence of SEQ ID NO: 356. In embodiments, the RSV F glycoproteins described herein comprise an F2 domain with at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, at least 99.5%, identity, or 100% identity to the F2 domain of SEQ ID NO: 356. The F2 domain of an RSV F glycoprotein herein may have a deletion, an insertion, or mutation of between about 1 and about 5 amino acids, between about 3 and about 10 amino acids, between about 5 and 10 amino acids, between about 8 and 12 amino acids, between about 10 and 15 amino acids, between about 12 and 17 amino acids, between about 15 and 20 amino acids, between about 18 and 23 amino acids, between about 20 and 25 amino acids, between about 22 and about 27 amino acids, or between about 25 and 30 amino acids as compared to the F2 domain of SEQ ID NO: 356.

In embodiments, the F2 domain of the RSV F glycoproteins may comprise a mutation of P102A, wherein the mutation is numbered according to the RSV F glycoprotein of SEQ ID NO: 330.

In embodiments, the F2 domain of the RSV F glycoproteins comprises an amino acid sequence with at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, at least 99.5% identity, or 100% identity any one of SEQ ID NOS: 356-357.

In embodiments, the non-naturally occurring RSV F glycoproteins described herein may have a deletion, an insertion, or mutation of up to about 1, up to about 2, up to about 3, up to about 4, up to about 5, up to about 10, up to about 15, up to about 20, up to about 25, or up to about 30 amino acids compared to the amino acid sequence of p27 of SEQ ID NO: 330. The wild-type p27 has the amino acid sequence of SEQ ID NO: 359. In embodiments, the RSV F glycoproteins described herein comprise a p27 with at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, at least 99.5%, identity, or 100% identity to the p27 of SEQ ID NO: 359. The p27 of an RSV F glycoprotein herein may have a deletion, an insertion, or mutation of between about 1 and about 5 amino acids, between about 3 and about 10 amino acids, between about 5 and 10 amino acids, between about 8 and 12 amino acids, between about 10 and 15 amino acids, or between about 12 and 17 amino acids compared to the p27 of SEQ ID NO: 359.

In embodiments, the p27 domain of the RSV F glycoproteins may comprise a mutation of N116Q, wherein the mutation is numbered according to the RSV F glycoprotein of SEQ ID NO: 330.

In embodiments, p27 of the RSV F glycoproteins comprises an amino acid sequence with at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, at least 99.5% identity, or 100% identity any one of SEQ ID NOS: 358-359.

In embodiments, the RSV F glycoproteins described herein comprise a transmembrane domain. In embodiments, the transmembrane domain has the sequence of ITTIIIVIIVILLSLIAVGLLLYCKARSTPVTLSKDQLSGINNIAFSN (SEQ ID NO: 362). In embodiments, the RSV F glycoproteins described herein may have a deletion, an insertion, or mutation of up to about 1, up to about 2, up to about 3, up to about 4, up to about 5, up to about 10, up to about 15, up to about 20, up to about 25, or up to about 30 amino acids compared to the amino acid sequence of the transmembrane domain. In embodiments, the RSV F glycoprotein lacks the transmembrane domain.

In embodiments, the RSV F glycoproteins described herein are expressed with an N-terminal signal peptide. In embodiments, the N-terminal signal peptide has an amino acid sequence of SEQ ID NO: 360 (MELLILKANAITTILTAVTFCFASG). In embodiments, the signal peptide may be replaced with any signal peptide that enables expression of the RSV F glycoprotein. In embodiments, one or more of the RSV F glycoprotein signal peptide amino acids may be deleted or mutated. An initiating methionine residue is maintained to initiate expression. In embodiments, the RSV F glycoprotein is encoded by a nucleic acid sequence selected from the group consisting of SEQ ID NO: 361.

Following expression of the RSV F glycoprotein in a host cell, the N-terminal signal peptide is cleaved to provide the mature RSV F glycoprotein sequence (SEQ ID NOS: 331 and 337-340). In embodiments, the signal peptide is cleaved by host cell proteases. In aspects, the full-length protein may be isolated from the host cell and the signal peptide cleaved subsequently.

Following cleavage of the signal peptide from the RSV F glycoprotein with an amino acid sequence corresponding to SEQ ID NOS: 332-336 during expression and purification, a mature polypeptide having an amino acid sequence selected from the group consisting of SEQ ID NOS: 331 and 337-340 is obtained and used to produce an RSV F nanoparticle vaccine or RSV F nanoparticles.

Advantageously, the disclosed RSV F glycoproteins may have enhanced protein expression, stability, and immunogenicity relative to the native RSV F glycoproteins.

In embodiments, the RSV F glycoproteins described herein contain further modifications from the native RSV F glycoprotein (SEQ ID NO: 330). In embodiments, the RSV F glycoproteins described herein exhibit at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to the native RSV F glycoprotein.

In embodiments, the RSV F glycoproteins described herein are at least 90%, at least 91%, at least 92%, at least 93%, at least 94%, at least 95%, at least 96%, at least 97%, at least 98%, at least 99%, or at least 99.5% identical to an RSV F glycoprotein having an amino acid selected from the group consisting of SEQ ID NOS: 329-340. A RSV F glycoprotein may have a deletion, an insertion, or mutation of up to about 1, up to about 2, up to about 3, up to about 4, up to about 5, up to about 10, up to about 15, up to about 20, up to about 25, up to about 30, up to about 35, up to about 40, up to about 45, or up to about 50 amino acids compared to the amino acid sequence of the RSV F glycoprotein having an amino acid sequence of any one of SEQ ID NO: 329-340. A RSV F glycoprotein may have may have a deletion, an insertion, or mutation of between about 1 and about 5 amino acids, between about 3 and about 10 amino acids, between about 5 and 10 amino acids, between about 8 and 12 amino acids, between about 10 and 15 amino acids, between about 12 and 17 amino acids, between about 15 and 20 amino acids, between about 18 and 23 amino acids, between about 20 and 25 amino acids, between about 22 and about 27 amino acids, between about 25 and 30 amino acids, between about 30 and 35 amino acids, between about 35 and 40 amino acids, between about 40 and 45 amino acids, or between about 45 and 50 amino acids, as compared to the RSV F glycoprotein having an amino acid sequence of any one of SEQ ID NOS: 329-340. In embodiments, RSV F glycoproteins described herein comprise about 1, about 2, about 3, about 4, about 5, about 6, about 7, about 8, about 9, about 10, about 11, about 12, about 13, about 14, about 15, about 16, about 17, about 18, about 19, about 20, about 21, about 22, about 23, about 24, or about 25 modifications compared to the RSV F glycoprotein having an amino acid sequence of any one of SEQ ID NOS: 329-340.

In embodiments, the RSV F glycoprotein is extended at the N-terminus, the C-terminus, or both the N-terminus and the C-terminus. In embodiments, the extension is a tag useful for a function, such as purification or detection. In embodiments, the extension may be an N-terminal signal peptide fused to the protein to enhance expression. While such signal peptides are often cleaved during expression in the cell, some nanoparticles may contain the antigen with an intact signal peptide. Thus, when a nanoparticle comprises an antigen, the antigen may contain an extension and thus may be a fusion protein when incorporated into nanoparticles.

In embodiments, the RSV F glycoprotein comprises a fusion protein. In embodiments, the RSV F glycoprotein comprises an N-terminal fusion protein. In embodiments, the RSV F glycoprotein comprises a C-terminal fusion protein. In embodiments, the fusion protein encompasses any tag described herein. In embodiments, the tag is useful for protein expression, purification, or detection. In embodiments, the tag is a protease cleavage site. Non-limiting examples of protease cleavage sites include the HRV3C protease cleavage site, chymotrypsin, trypsin, elastase, endopeptidase, caspase-1, caspase-2, caspase-3, caspase-4, caspase-5, caspase-6, caspase-7, caspase-8, caspase-9, caspase-10, enterokinase, factor Xa, Granzyme B, TEV protease, and thrombin. In embodiments, the protease cleavage site is an HRV3C protease cleavage site.

### Adjuvants

In embodiments, the compositions disclosed herein may be combined with one or more adjuvants to enhance an immune response. In embodiments, the compositions are prepared without adjuvants, and are thus available to be administered as adjuvant-free compositions.

Advantageously, adjuvant-free compositions disclosed herein may provide protective immune responses when administered as a single dose. Alum-free compositions that induce robust immune responses are especially useful in adults about 60 and older.

### Aluminum-Based Adjuvants

In embodiments, the adjuvant may be alum (e.g. AlPO4 or Al(OH)3). In embodiments, the nanoparticle is substantially bound to the alum. In embodiments, the nanoparticle may be at least 80% bound, at least 85% bound, at least 90% bound or at least 95% bound to the alum. In embodiments, the nanoparticle is 92% to 97% bound to the alum in a composition. The amount of alum is present per dose is typically in a range between about 400 μg to about 1250 μg. For example, the alum may be present in a per dose amount of about 300 μg to about 900 μg, about 400 μg to about 800 μg, about 500 μg to about 700 μg, about 400 μg to about 600 μg, or about 400 μg to about 500 μg. Typically, the alum is present at about 400 μg for a dose of 120 μg of the protein nanoparticle.

### Saponin Adjuvants

Adjuvants containing saponin may also be combined with the immunogens disclosed herein. Saponins are glycosides derived from the bark of the Quillaja Saponaria Molina tree. Typically, saponin is prepared using a multi-step purification process resulting in multiple fractions. As used, herein, the term “a saponin fraction from Quillaja Saponaria Molina” is used generically to describe a semi-purified or defined saponin fraction of Quillaja saponaria or a substantially pure fraction thereof.

Several approaches for producing saponin fractions are suitable. Fractions A, B, and C are described in U.S. Pat. No. 6,352,697 and may be prepared as follows. A lipophilic fraction from Quil A, a crude aqueous Quillaja Saponaria Molina extract, is separated by chromatography and eluted with 70% acetonitrile in water to recover the lipophilic fraction. This lipophilic fraction is then separated by semi-preparative HPLC with elution using a gradient of from 25% to 60% acetonitrile in acidic water. The fraction referred to herein as “Fraction A” or “QH-A” is, or corresponds to, the fraction, which is eluted at approximately 39% acetonitrile. The fraction referred to herein as “Fraction B” or “QH-B” is, or corresponds to, the fraction, which is eluted at approximately 47% acetonitrile. The fraction referred to herein as “Fraction C” or “QH-C” is, or corresponds to, the fraction, which is eluted at approximately 49% acetonitrile. Additional information regarding purification of Fractions is found in U.S. Pat. No. 5,057,540. When prepared as described herein, Fractions A, B and C of Quillaja Saponaria Molina each represent groups or families of chemically closely related molecules with definable properties. The chromatographic conditions under which they are obtained are such that the batch-to-batch reproducibility in terms of elution profile and biological activity is highly consistent.

Other saponin fractions have been described. Fractions B3, B4 and B4b are described in EP 0436620. Fractions QA1-QA22 are described EP03632279 B2, Q-VAC (Nor-Feed, AS Denmark), Quillaja Saponaria Molina Spikoside (Isconova AB, Ultunaallén 2B, 756 51 Uppsala, Sweden). Fractions QA-1, QA-2, QA-3, QA-4, QA-5, QA-6, QA-7, QA-8, QA-9, QA-10, QA-11, QA-12, QA-13, QA-14, QA-15, QA-16, QA-17, QA-18, QA-19, QA-20, QA-21, and QA-22 of EP 0 3632 279 B2, especially QA-7, QA-17, QA-18, and QA-21 may be used. They are obtained as described in EP 0 3632 279 B2, especially at page 6 and in Example 1 on page 8 and 9.

The saponin fractions described herein and used for forming adjuvants are often substantially pure fractions; that is, the fractions are substantially free of the presence of contamination from other materials. In particular aspects, a substantially pure saponin fraction may contain up to 40% by weight, up to 30% by weight, up to 25% by weight, up to 20% by weight, up to 15% by weight, up to 10% by weight, up to 7% by weight, up to 5% by weight, up to 2% by weight, up to 1% by weight, up to 0.5% by weight, or up to 0.1% by weight of other compounds such as other saponins or other adjuvant materials.

Saponin fractions may be administered in the form of a cage-like particle referred to as an ISCOM (Immune Stimulating COMplex). ISCOMs may be prepared as described in EP0109942B1, EP0242380B1 and EP0180546 B1. In embodiments, a transport and/or a passenger antigen may be used, as described in EP 9600647-3 (PCT/SE97/00289).

In embodiments, the ISCOM is an ISCOM matrix complex. An ISCOM matrix complex comprises at least one saponin fraction and a lipid. The lipid is at least a sterol, such as cholesterol. In embodiments, the ISCOM matrix complex also contains a phospholipid. The ISCOM matrix complexes may also contain one or more other immunomodulatory (adjuvant-active) substances, not necessarily a glycoside, and may be produced as described in EP0436620B1, which is incorporated by reference in its entirety herein.

In other aspects, the ISCOM is an ISCOM complex. An ISCOM complex contains at least one saponin, at least one lipid, and at least one kind of antigen or epitope. The ISCOM complex contains antigen associated by detergent treatment such that that a portion of the antigen integrates into the particle. In contrast, ISCOM matrix is formulated as an admixture with antigen and the association between ISCOM matrix particles and antigen is mediated by electrostatic and/or hydrophobic interactions.

In embodiments, the saponin fraction is integrated into an ISCOM matrix complex or an ISCOM complex, or at least one additional adjuvant, which also is integrated into the ISCOM or ISCOM matrix complex or mixed therewith, is selected from fraction A, fraction B, or fraction C of Quillaja saponaria, a semipurified preparation of Quillaja saponaria, a purified preparation of Quillaja saponaria, or any purified sub-fraction e.g., QA 1-21.

In particular aspects, each ISCOM particle may contain at least two saponin fractions. Any combinations of weight % of different saponin fractions may be used. Any combination of weight % of any two fractions may be used. For example, the particle may contain any weight % of fraction A and any weight % of another saponin fraction, such as a crude saponin fraction or fraction C, respectively. Accordingly, in particular aspects, each ISCOM matrix particle or each ISCOM complex particle may contain from 0.1 to 99.9 by weight, 5 to 95% by weight, 10 to 90% by weight 15 to 85% by weight, 20 to 80% by weight, 25 to 75% by weight, 30 to 70% by weight, 35 to 65% by weight, 40 to 60% by weight, 45 to 55% by weight, 40 to 60% by weight, or 50% by weight of one saponin fraction, e.g. fraction A and the rest up to 100% in each case of another saponin e.g. any crude fraction or any other faction e.g. fraction C. The weight is calculated as the total weight of the saponin fractions. Examples of ISCOM matrix complex and ISCOM complex adjuvants are disclosed in U.S Published Application No. 2013/0129770, which is incorporated by reference in its entirety herein.

In embodiments, the ISCOM matrix or ISCOM complex comprises from 5-99% by weight of one fraction, e.g. fraction A and the rest up to 100% of weight of another fraction e.g. a crude saponin fraction or fraction C. The weight is calculated as the total weight of the saponin fractions.

In embodiment, the ISCOM matrix or ISCOM complex comprises from 40% to 99% by weight of one fraction, e.g. fraction A and from 1% to 60% by weight of another fraction, e.g. a crude saponin fraction or fraction C. The weight is calculated as the total weight of the saponin fractions.

In embodiments, the ISCOM matrix or ISCOM complex comprises from 70% to 95% by weight of one fraction e.g., fraction A, and from 30% to 5% by weight of another fraction, e.g., a crude saponin fraction, or fraction C. The weight is calculated as the total weight of the saponin fractions. In other embodiments, the saponin fraction from Quillaja Saponaria Molina is selected from any one of QA 1-21.

In addition to particles containing mixtures of saponin fractions, ISCOM matrix particles and ISCOM complex particles may each be formed using only one saponin fraction. Compositions disclosed herein may contain multiple particles wherein each particle contains only one saponin fraction. That is, certain compositions may contain one or more different types of ISCOM-matrix complexes particles and/or one or more different types of ISCOM complexes particles, where each individual particle contains one saponin fraction from Quillaja Saponaria Molina, wherein the saponin fraction in one complex is different from the saponin fraction in the other complex particles.

In embodiments, one type of saponin fraction or a crude saponin fraction may be integrated into one ISCOM matrix complex or particle and another type of substantially pure saponin fraction, or a crude saponin fraction, may be integrated into another ISCOM matrix complex or particle. A composition or vaccine may comprise at least two types of complexes or particles each type having one type of saponins integrated into physically different particles.

In the compositions, mixtures of ISCOM matrix complex particles and/or ISCOM complex particles may be used in which one saponin fraction Quillaja Saponaria Molina and another saponin fraction Quillaja Saponaria Molina are separately incorporated into different ISCOM matrix complex particles and/or ISCOM complex particles.

The ISCOM matrix or ISCOM complex particles, which each have one saponin fraction, may be present in composition at any combination of weight %. In particular aspects, a composition may contain 0.1% to 99.9% by weight, 5% to 95% by weight, 10% to 90% by weight, 15% to 85% by weight, 20% to 80% by weight, 25% to 75% by weight, 30% to 70% by weight, 35% to 65% by weight, 40% to 60% by weight, 45% to 55% by weight, 40 to 60% by weight, or 50% by weight, of an ISCOM matrix or complex containing a first saponin fraction with the remaining portion made up by an ISCOM matrix or complex containing a different saponin fraction. In aspects, the remaining portion is one or more ISCOM matrix or complexes where each matrix or complex particle contains only one saponin fraction. In other aspects, the ISCOM matrix or complex particles may contain more than one saponin fraction.

In particular compositions, the only saponin fraction in a first ISCOM matrix or ISCOM complex particle is Fraction A and the only saponin fraction in a second ISCOM matrix or ISCOM complex particle is Fraction C.

In embodiments, the Fraction A of Quillaja Saponaria Molina accounts for at least about 80%, 81%, 82%, 83%, 84%, 85%, 86%, 87%, 88%, 89%, 90%, 91%, 92%, 93%, 94%, 95%, 96%, 97%, 98%, or 99% by weight, and fraction C of Quillaja Saponaria Molina accounts for the remainder, respectively, of the sum of the weights of fraction A of Quillaja Saponaria Molina and fraction C of Quillaja Saponaria Molina in the adjuvant.

Preferred compositions comprise a first ISCOM matrix containing Fraction A and a second ISCOM matrix containing Fraction C, wherein the Fraction A ISCOM matrix constitutes about 70% per weight of the total saponin adjuvant, and the Fraction C ISCOM matrix constitutes about 30% per weight of the total saponin adjuvant. In another preferred composition, the Fraction A ISCOM matrix constitutes about 85% per weight of the total saponin adjuvant, and the Fraction C ISCOM matrix constitutes about 15% per weight of the total saponin adjuvant. In another preferred composition, the Fraction A ISCOM matrix constitutes about 92% per weight of the total saponin adjuvant, and the Fraction C ISCOM matrix constitutes about 8% per weight of the total saponin adjuvant. Thus, in certain compositions, the Fraction A ISCOM matrix is present in a range of about 70% to about 85%, and Fraction C ISCOM matrix is present in a range of about 15% to about 30%, of the total weight amount of saponin adjuvant in the composition. In certain compositions, the Fraction A ISCOM matrix is present in a range of about 70% to about 92%, and Fraction C ISCOM matrix is present in a range of about 8% to about 30%, of the total weight amount of saponin adjuvant in the composition. In embodiments, the Fraction A ISCOM matrix accounts for 50-96% by weight and Fraction C ISCOM matrix accounts for the remainder, respectively, of the sums of the weights of Fraction A ISCOM matrix and Fraction C ISCOM in the adjuvant. In a particularly preferred composition, referred to herein as MATRIX-M®, the Fraction A ISCOM matrix is present at about 85% and Fraction C ISCOM matrix is present at about 15% of the total weight amount of saponin adjuvant in the composition. MATRIX-M® may be referred to interchangeably as Matrix-M1.

Exemplary QS-7 and QS-21 fractions, their production and their use is described in U.S. Pat. Nos. 5,057,540; 6,231,859; 6,352,697; 6,524,584; 6,846,489; 7,776,343, and 8,173,141, which are incorporated by reference herein.

In embodiments, other adjuvants may be used in addition or as an alternative. The inclusion of any adjuvant described in Vogel et al., “A Compendium of Vaccine Adjuvants and Excipients (2nd Edition),” herein incorporated by reference in its entirety for all purposes, is envisioned within the scope of this disclosure. Other adjuvants include complete Freund's adjuvant (a non-specific stimulator of the immune response containing killed Mycobacterium tuberculosis), incomplete Freund's adjuvants and aluminum hydroxide adjuvant. Other adjuvants comprise GMCSP, BCG, MDP compounds, such as thur-MDP and nor-MDP, CGP (MTP-PE), lipid A, and monophosphoryl lipid A (MPL), MF-59, RIBI, which contains three components extracted from bacteria, MPL, trehalose dimycolate (TDM) and cell wall skeleton (CWS) in a 2% squalene/TWEEN® polysorbate 80 emulsion. In embodiments, the adjuvant may be a paucilamellar lipid vesicle; for example, NOVASOMES®. NOVASOMES® are paucilamellar nonphospholipid vesicles ranging from about 100 nm to about 500 nm. They comprise BRIJ® alcohol ethoxylate 72, cholesterol, oleic acid and squalene. NOVASOMES® have been shown to be an effective adjuvant (see, U.S. Pat. Nos. 5,629,021, 6,387,373, and 4,911,928).

### Production of Nanoparticles Comprising Viral Glycoproteins

The nanoparticles of the present disclosure are non-naturally occurring products, the components of which do not occur together in nature. Generally, the methods disclosed herein use a detergent exchange approach wherein a first detergent is used to isolate a viral glycoprotein and then that first detergent is exchanged for a second detergent to form the nanoparticles.

The viral glycoproteins contained in the nanoparticles are typically produced by recombinant expression in host cells. Standard recombinant techniques may be used. In embodiments, the viral glycoproteins are expressed in insect host cells using a baculovirus system. In embodiments, from 1-50 viral glycoproteins are co-expressed in a host cell. In embodiments, the baculovirus is a cathepsin-L knock-out baculovirus, a chitinase knock-out baculovirus. Optionally, the baculovirus is a double knock-out for both cathepsin-L and chitinase. High level expression may be obtained in insect cell expression systems. Non limiting examples of insect cells are, Spodoptera frugiperda (Sf) cells, e.g. Sf9, Sf21, Trichoplusiani cells, e.g. High Five cells, and Drosophila S2 cells. In embodiments, the viral glycoproteins described herein are produced in any suitable host cell. In embodiments, the host cell is an insect cell. In embodiments, the insect cell is an Sf9 cell.

Typical transfection and cell growth methods can be used to culture the cells. Vectors, e.g., vectors comprising polynucleotides that encode fusion proteins, can be transfected into host cells according to methods well known in the art. For example, introducing nucleic acids into eukaryotic cells can be achieved by calcium phosphate co-precipitation, electroporation, microinjection, lipofection, and transfection employing polyamine transfection reagents. In one embodiment, the vector is a recombinant baculovirus.

Methods to grow host cells include, but are not limited to, batch, batch-fed, continuous and perfusion cell culture techniques. Cell culture means the growth and propagation of cells in a bioreactor (a fermentation chamber) where cells propagate and express protein (e.g. recombinant proteins) for purification and isolation. Typically, cell culture is performed under sterile, controlled temperature and atmospheric conditions in a bioreactor. A bioreactor is a chamber used to culture cells in which environmental conditions such as temperature, atmosphere, agitation and/or pH can be monitored. In one embodiment, the bioreactor is a stainless steel chamber. In another embodiment, the bioreactor is a pre-sterilized plastic bag (e.g. Cellbag®, Wave Biotech, Bridgewater, N.J.). In other embodiment, the pre-sterilized plastic bags are about 50 L to 3500 L bags.

After growth of the host cells, the protein may be harvested from the host cells using detergents and purification protocols. In embodiments, multiple viral glycoproteins are purified simultaneously. In embodiments, host cells expressing multiple viral glycoproteins are pooled together. Once the host cells have grown for 48 to 96 hours, the cells are isolated from the media and a detergent-containing solution is added to solubilize the cell membrane, releasing the protein in a detergent extract. Triton X-100 and TERGITOL® nonylphenol ethoxylate, also known as NP-9, are each preferred detergents for extraction. The detergent may be added to a final concentration of about 0.1% to about 1.0%. For example, the concentration may be about 0.1%, about 0.2%, about 0.3%, about 0.5%, about 0.7%, about 0.8%, or about 1.0%. The range may be about 0.1% to about 0.3%. In aspects, the concentration is about 0.5%.

In other aspects, different first detergents may be used to isolate the protein from the host cell. For example, the first detergent may be Bis(polyethylene glycol bis [imidazoylcarbonyl]), nonoxynol-9, Bis(polyethylene glycol bis [imidazoyl carbonyl]), BRIJ® Polyethylene glycol dodecyl ether 35, BRIJ® Polyethylene glycol (3) cetyl ether 56, BRIJ® alcohol ethoxylate 72, BRIJ® Polyoxyl 2 stearyl ether 76, BRIJ® polyethylene glycol monoolelyl ether 92V, BRIJ® Polyoxyethylene (10) oleyl ether 97, BRIJ® Polyethylene glycol hexadecyl ether 58P, CREMOPHOR® EL Macrogolglycerol ricinoleate, Decaethyleneglycol monododecyl ether, N-Decanoyl-N-methylglucamine, n-Decyl alpha-Dglucopyranoside,Decyl beta-D-maltopyranoside, n-Dodecanoyl-N-methylglucamide, nDodecyl alpha-D-maltoside, n-Dodecyl beta-D-maltoside, n-Dodecyl beta-D-maltoside,Heptaethylene glycol monodecyl ether, Heptaethylene glycol monododecyl ether, Heptaethylene glycol monotetradecyl ether, n-Hexadecyl beta-D-maltoside, Hexaethylene glycol monododecyl ether, Hexaethylene glycol monohexadecyl ether, Hexaethylene glycol monooctadecyl ether, Hexaethylene glycol monotetradecyl ether, Igepal CA-630,Igepal CA-630, Methyl-6-O-(N-heptylcarbamoyl)-alpha-D-glucopyranoside,Nonaethylene glycol monododecyl ether, N-Nonanoyl-N-methylglucamine, N-NonanoyIN-methylglucamine, Octaethylene glycol monodecyl ether, Octaethylene glycolmonododecyl ether, Octaethylene glycol monohexadecyl ether, Octaethylene glycol monooctadecyl ether, Octaethylene glycol monotetradecyl ether, Octyl-beta-D glucopyranoside, Pentaethylene glycol monodecyl ether, Pentaethylene glycol monododecyl ether, Pentaethylene glycol monohexadecyl ether, Pentaethylene glycol monohexyl ether, Pentaethylene glycol monooctadecyl ether, Pentaethylene glycol monooctyl ether, Polyethylene glycol diglycidyl ether, Polyethylene glycol ether W-1, Polyoxyethylene 10 tridecyl ether, Polyoxyethylene 100 stearate, Polyoxyethylene 20 isohexadecyl ether, Polyoxyethylene 20 oleyl ether, Polyoxyethylene 40 stearate, Polyoxyethylene 50 stearate, Polyoxyethylene 8 stearate, Polyoxyethylene bis(imidazolyl carbonyl), Polyoxyethylene 25 propylene glycol stearate, Saponin from Quillaja bark, SPAN® 20 sorbitan laurate, SPAN® 40 sorbitan monopalmitate, SPAN® 60 sorbitan stearate, SPAN® 65 sorbitan tristearate, SPAN® 80 sorbitane monooleate, SPAN® 85 sorbitane trioleate, TERGITOL® secondary alcohol ethoxylate Type 15-S-12, TERGITOL® secondary alcohol ethoxylate Type 15-S-30, TERGITOL® secondary alcohol ethoxylate Type 15-S-5, TERGITOL® secondary alcohol ethoxylate Type 15-S-7, TERGITOL® secondary alcohol ethoxylate Type 15-S-9, TERGITOL® nonylphenol ethoxylate Type NP-10, TERGITOL® nonylphenol ethoxylate Type NP-4, TERGITOL® nonylphenol ethoxylate Type NP-40, TERGITOL® nonylphenol ethoxylate Type NP-7, TERGITOL® nonylphenol ethoxylate Type NP-9, TERGITOL® branched secondary alcohol ethoxylate Type TMN-10, TERGITOL® branched secondary alcohol ethoxylate Type TMN-6, TRITON™ X-100 Polyethylene glycol tert-octylphenyl ether or combinations thereof.

The nanoparticles may then be isolated from cellular debris using centrifugation. In embodiments, gradient centrifugation, such as using cesium chloride, sucrose and iodixanol, may be used. Other techniques may be used as alternatives or in addition, such as standard purification techniques including, e.g., ion exchange, affinity, and gel filtration chromatography.

For example, the first column may be an ion exchange chromatography resin, such as FRACTOGEL® EMD methacrylate based polymeric beads TMAE (EMD Millipore), the second column may be a lentil (Lens culinaris) lectin affinity resin, and the third column may be a cation exchange column such as a FRACTOGEL® EMD methacrylate based polymeric beads SO3 (EMD Millipore) resin. In other aspects, the cation exchange column may be an MMC column or a Nuvia C Prime column (Bio-Rad Laboratories, Inc). Preferably, the methods disclosed herein do not use a detergent extraction column; for example a hydrophobic interaction column. Such a column is often used to remove detergents during purification but may negatively impact the methods disclosed here.

To form nanoparticles, the first detergent, used to extract the protein from the host cell is substantially replaced with a second detergent to arrive at the nanoparticle structure. NP-9 is a preferred extraction detergent. Typically, the nanoparticles do not contain detectable NP-9 when measured by HPLC. The second detergent is typically selected from the group consisting of PS20, PS40, PS60, PS65, and PS80. Preferably, the second detergent is PS80.

In particular aspects, detergent exchange is performed using affinity chromatography to bind glycoproteins via their carbohydrate moiety. For example, the affinity chromatography may use a legume lectin column. Legume lectins are proteins originally identified in plants and found to interact specifically and reversibly with carbohydrate residues. See, for example, Sharon and Lis, “Legume lectins—a large family of homologous proteins,” FASEB J. 1990 November;4 (14): 3198-208; Liener, “The Lectins: Properties, Functions, and Applications in Biology and Medicine,” Elsevier, 2012. Suitable lectins include concanavalin A (con A), pea lectin, sainfoin lect, and lentil lectin. Lentil lectin is a preferred column for detergent exchange due to its binding properties. Lectin columns are commercially available; for example, Capto Lentil Lectin, is available from GE Healthcare. In certain aspects, the lentil lectin column may use a recombinant lectin. At the molecular level, it is thought that the carbohydrate moieties bind to the lentil lectin, freeing the amino acids of the protein to coalesce around the detergent resulting in the formation of a detergent core providing nanoparticles having multiple copies of the antigen, e.g., glycoprotein oligomers which can be dimers, trimers, or tetramers anchored in the detergent. In embodiments, the viral glycoproteins form trimers. In embodiments, the viral glycoprotein trimers are anchored in detergent. In embodiments, each viral glycoprotein nanoparticle contains at least one trimer associated with a non-ionic core.

The detergent, when incubated with the protein to form the nanoparticles during detergent exchange, may be present at up to about 0.1% (w/v) during early purifications steps and this amount is lowered to achieve the final nanoparticles having optimum stability. For example, the non-ionic detergent (e.g., PS80) may be about 0.005% (v/v) to about 0.1% (v/v), for example, about 0.005% (v/v), about 0.006% (v/v), about 0.007% (v/v), about 0.008% (v/v), about 0.009% (v/v), about 0.01% (v/v), about 0.015% (v/v), about 0.02% (v/v), about 0.025% (v/v), about 0.03% (v/v), about 0.035% (v/v), about 0.04% (v/v), about 0.045% (v/v), about 0.05% (v/v), about 0.055% (v/v), about 0.06% (v/v), about 0.065% (v/v), about 0.07% (v/v), about 0.075% (v/v), about 0.08% (v/v), about 0.085% (v/v), about 0.09% (v/v), about 0.095% (v/v), or about 0.1% (v/v) PS80. In embodiments, the nanoparticle contains about 0.03% to about 0.05% PS80. In embodiments, the nanoparticle contains about 0.01% (v/v) PS80.

In embodiments, purified viral glycoproteins are dialyzed. In embodiments, dialysis occurs after purification. In embodiments, the viral glycoproteins are dialyzed in a solution comprising sodium phosphate, NaCl, and PS80. In embodiments, the dialysis solution comprising sodium phosphate contains between about 5 mM and about 100 mM of sodium phosphate, for example, about 5 mM, about 10 mM, about 15 mM, about 20 mM, about 25 mM, about 30 mM, about 35 mM, about 40 mM, about 45 mM, about 50 mM, about 55 mM, about 60 mM, about 65 mM, about 70 mM, about 75 mM, about 80 mM, about 85 mM, about 90 mM, about 95 mM, or about 100 mM sodium phosphate. In embodiments, the pH of the solution comprising sodium phosphate is about 6.5, about 6.6, about 6.7, about 6.8, about 6.9, about 7.0, about 7.1, about 7.2, about 7.3, about 7.4, or about 7.5. In embodiments, the dialysis solution comprising sodium chloride comprises about 50 mM NaCl to about 750 mM NaCl, for example, about 50 mM, about 60 mM, about 70 mM, about 80 mM, about 90 mM, about 100 mM, about 110 mM, about 120 mM, about 130 mM, about 140 mM, about 150 mM, about 160 mM, about 170 mM, about 180 mM, about 190 mM, about 200 mM, about 210 mM, about 220 mM, about 230 mM, about 240 mM, about 250 mM, about 260 mM, about 270 mM, about 280 mM, about 290 mM, about 300 mM, about 310 mM, about 320 mM, about 330 mM, about 340 mM, about 350 mM, about 360 mM, about 370 mM, about 380 mM, about 390 mM, about 400 mM, about 410 mM, about 420 mM, about 430 mM, about 440 mM, about 450 mM, about 460 mM, about 470 mM, about 480 mM, about 490 mM, about 500 mM, about 510 mM, about 520 mM, about 530 mM, about 540 mM, about 550 mM, about 560 mM, about 570 mM, about 580 mM, about 590 mM, about 600 mM, about 610 mM, about 620 mM, about 630 mM, about 640 mM, about 650 mM, about 660 mM, about 670 mM, about 680 mM, about 690, about 700 mM, about 710 mM, about 720 mM, about 730 mM, about 740 mM, or about 750 mM NaCl. In embodiments, the dialysis solution comprising PS80 comprises about 0.005% (v/v), about 0.006% (v/v), about 0.007% (v/v), about 0.008% (v/v), about 0.009% (v/v), about 0.01% (v/v), about 0.015% (v/v), about 0.02% (v/v), about 0.025% (v/v), about 0.03% (v/v), about 0.035% (v/v), about 0.04% (v/v), about 0.045% (v/v), about 0.05% (v/v), about 0.055% (v/v), about 0.06% (v/v), about 0.065% (v/v), about 0.07% (v/v), about 0.075% (v/v), about 0.08% (v/v), about 0.085% (v/v), about 0.09% (v/v), about 0.095% (v/v), or about 0.1% (v/v) PS80. In embodiments, the dialysis solution comprises about 25 mM sodium phosphate (pH 7.2), about 300 mM NaCl, and about 0.01% (v/v) PS80.

In embodiments, the pharmaceutically acceptable buffer comprises 10 mM sodium phosphate, 150 mM NaCl, 100 mM arginine, 5% trehalose, and 0.03% PS80 at a pH of 7.5. In embodiments, the pharmaceutically acceptable buffer comprises 25 mM sodium phosphate, 300 mM NaCl, and 0.03% PS80 at a pH of 7.2. In embodiments, the pharmaceutically acceptable buffer comprises 25 mM sodium phosphate, 600 mM NaCl, and 0.01% PS80 at a pH of 6.8.

Detergent exchange may be performed with proteins purified as discussed above and purified, frozen for storage, and then thawed for detergent exchange.

Stability of compositions disclosed herein may be measured in a variety of ways. In one approach, a peptide map may be prepared to determine the integrity of the antigen protein after various treatments designed to stress the nanoparticles by mimicking harsh storage conditions. Thus, a measure of stability is the relative abundance of antigen peptides in a stressed sample compared to a control sample. For example, the stability of nanoparticles containing the viral glycoproteins may be evaluated by exposing the nanoparticles to various pHs, proteases, salt, oxidizing agents, including but not limited to hydrogen peroxide, various temperatures, freeze/thaw cycles, and agitation. It is thought that the position of the glycoprotein anchored into the detergent core provides enhanced stability by reducing undesirable interactions. For example, the improved protection against protease-based degradation may be achieved through a shielding effect whereby anchoring the glycoproteins into the core at the molar ratios disclosed herein results in steric hindrance blocking protease access. Stability may also be measured by monitoring intact proteins.

In embodiments, provided herein are immunogenic compositions containing nanoparticles comprising SARS-COV-2 S glycoproteins.

### Additional Excipients in Intranasal Immunogenic Compositions

In embodiments, the intranasal immunogenic compositions comprise one or more additional ingredient selected from the group consisting of a viscosity agent, a bulking agent, a dispersant, a filler, a carrier agent, a moisturizing agent, and a flavoring agent. Examples of each of these ingredients are described herein.

Amounts of Viral Glycoproteins and/or Adjuvants in Immunogenic Compositions

In embodiments, the immunogenic compositions described herein comprise from about 1 μg to about 400 μg, from 1 μg to about 350 μg, from 1 μg to about 300 μg, from 1 μg to about 250 μg, from 1 μg to about 200 μg, from 1 μg to about 150 μg, from 1 μg to about 100 μg, from 1 μg to about 50 μg, or from 1 μg to about 25 μg of each viral glycoprotein. In embodiments, the immunogenic compositions comprise about 1 μg, about 2 μg, about 3 μg, about 4 μg, about 5 μg, about 6 μg, about 7 μg, about 8 μg, about 9 μg, about 10 μg, about 11 μg, about 12 μg, about 13 μg, about 14 μg, about 15 μg, about 16 μg, about 17 μg, about 18 μg, about 19 μg, about 20 μg, about 21 μg, about 22 μg, about 23 μg, about 24 μg, about 25 μg, about 26 μg, about 27 μg, about 28 μg, about 29 μg, about 30 μg, about 31 μg, about 32 μg, about 33 μg, about 34 μg, about 35 μg, about 36 μg, about 37 μg, about 38 μg, about 39 μg, about 40 μg, about 41 μg, about 42 μg, about 43 μg, about 44 μg, about 45 μg, about 46 μg, about 47 μg, about 48 μg, about 49 μg, about 50 μg, about 51 μg, about 52 μg, about 53 μg, about 54 μg, about 55 μg, about 56 μg, about 57 μg, about 58 μg, about 59 μg, about 60 μg, about 61 μg, about 62 μg, about 63 μg, about 64 μg, about 65 μg, about 66 μg, about 67 μg, about 68 μg, about 69 μg, about 70 μg, about 71 μg, about 72 μg, about 73 μg, about 74 μg, about 75 μg, about 76 μg, about 77 μg, about 78 μg, about 79 μg, about 80 μg, about 81 μg, about 82 μg, about 83 μg, about 84 μg, about 85 μg, about 86 μg, about 87 μg, about 88 μg, about 89 μg, about 90 μg, about 91 μg, about 92 μg, about 93 μg, about 94 μg, about 95 μg, about 96 μg, about 97 μg, about 98 μg, about 99 μg, about 100 μg, about 101 μg, about 102 μg, about 103 μg, about 104 μg, about 105 μg, about 106 μg, about 107 μg, about 108 μg, about 109 μg, about 110 μg, about 111 μg, about 112 μg, about 113 μg, about 114 μg, about 115 μg, about 116 μg, about 117 μg, about 118 μg, about 119 μg, about 120 μg, about 121 μg, about 122 μg, about 123 μg, about 124 μg, about 125 μg, about 126 μg, about 127 μg, about 128 μg, about 129 μg, about 130 μg, about 131 μg, about 132 μg, about 133 μg, about 134 μg, about 135 μg, about 136 μg, about 137 μg, about 138 μg, about 139 μg, about 140 μg, about 141 μg, about 142 μg, about 143 μg, about 144 μg, about 145 μg, about 146 μg, about 147 μg, about 148 μg, about 149 μg, about 150 μg, about 151 μg, about 152 μg, about 153 μg, about 154 μg, about 155 μg, about 156 μg, about 157 μg, about 158 μg, about 159 μg, about 160 μg, about 161 μg, about 162 μg, about 163 μg, about 164 μg, about 165 μg, about 166 μg, about 167 μg, about 168 μg, about 169 μg, about 170 μg, about 171 μg, about 172 μg, about 173 μg, about 174 μg, about 175 μg, about 176 μg, about 177 μg, about 178 μg, about 179 μg, about 180 μg, about 181 μg, about 182 μg, about 183 μg, about 184 μg, about 185 μg, about 186 μg, about 187 μg, about 188 μg, about 189 μg, about 190 μg, about 191 μg, about 192 μg, about 193 μg, about 194 μg, about 195 μg, about 196 μg, about 197 μg, about 198 μg, about 199 μg, about 200 μg, about 201 μg, about 202 μg, about 203 μg, about 204 μg, about 205 μg, about 206 μg, about 207 μg, about 208 μg, about 209 μg, about 210 μg, about 211 μg, about 212 μg, about 213 μg, about 214 μg, about 215 μg, about 216 μg, about 217 μg, about 218 μg, about 219 μg, about 220 μg, about 221 μg, about 222 μg, about 223 μg, about 224 μg, about 225 μg, about 226 μg, about 227 μg, about 228 μg, about 229 μg, about 230 μg, about 231 μg, about 232 μg, about 233 μg, about 234 μg, about 235 μg, about 236 μg, about 237 μg, about 238 μg, about 239 μg, about 240 μg, about 241 μg, about 242 μg, about 243 μg, about 244 μg, about 245 μg, about 246 μg, about 247 μg, about 248 μg, about 249 μg, about 250 μg, about 251 μg, about 252 μg, about 253 μg, about 254 μg, about 255 μg, about 256 μg, about 257 μg, about 258 μg, about 259 μg, about 260 μg, about 261 μg, about 262 μg, about 263 μg, about 264 μg, about 265 μg, about 266 μg, about 267 μg, about 268 μg, about 269 μg, about 270 μg, about 271 μg, about 272 μg, about 273 μg, about 274 μg, about 275 μg, about 276 μg, about 277 μg, about 278 μg, about 279 μg, about 280 μg, about 281 μg, about 282 μg, about 283 μg, about 284 μg, about 285 μg, about 286 μg, about 287 μg, about 288 μg, about 289 μg, about 290 μg, about 291 μg, about 292 μg, about 293 μg, about 294 μg, about 295 μg, about 296 μg, about 297 μg, about 298 μg, about 299 μg, about 300 μg, about 301 μg, about 302 μg, about 303 μg, about 304 μg, about 305 μg, about 306 μg, about 307 μg, about 308 μg, about 309 μg, about 310 μg, about 311 μg, about 312 μg, about 313 μg, about 314 μg, about 315 μg, about 316 μg, about 317 μg, about 318 μg, about 319 μg, about 320 μg, about 321 μg, about 322 μg, about 323 μg, about 324 μg, about 325 μg, about 326 μg, about 327 μg, about 328 μg, about 329 μg, about 330 μg, about 331 μg, about 332 μg, about 333 μg, about 334 μg, about 335 μg, about 336 μg, about 337 μg, about 338 μg, about 339 μg, about 340 μg, about 341 μg, about 342 μg, about 343 μg, about 344 μg, about 345 μg, about 346 μg, about 347 μg, about 348 μg, about 349 μg, about 350 μg, about 351 μg, about 352 μg, about 353 μg, about 354 μg, about 355 μg, about 356 μg, about 357 μg, about 358 μg, about 359 μg, about 360 μg, about 361 μg, about 362 μg, about 363 μg, about 364 μg, about 365 μg, about 366 μg, about 367 μg, about 368 μg, about 369 μg, about 370 μg, about 371 μg, about 372 μg, about 373 μg, about 374 μg, about 375 μg, about 376 μg, about 377 μg, about 378 μg, about 379 μg, about 380 μg, about 381 μg, about 382 μg, about 383 μg, about 384 μg, about 385 μg, about 386 μg, about 387 μg, about 388 μg, about 389 μg, about 390 μg, about 391 μg, about 392 μg, about 393 μg, about 394 μg, about 395 μg, about 396 μg, about 397 μg, about 398 μg, about 399 μg, or about 400 μg of each viral glycoprotein.

In embodiments, the immunogenic compositions comprise about 1 μg of each viral glycoprotein. In embodiments, the immunogenic compositions comprise about 5 μg of each viral glycoprotein. In embodiments, the immunogenic compositions comprise about 25 μg of each viral glycoprotein. In embodiments, the immunogenic compositions comprise about 60 μg of each viral glycoprotein. In embodiments, the immunogenic compositions comprise about 240 μg of each viral glycoprotein.

In embodiments, the immunogenic compositions described herein comprise from about 1 μg to about 100 μg of each CoV S glycoprotein. In embodiments, the immunogenic compositions described herein comprise from about 2.5 μg to about 35 μg of each CoV S glycoprotein. In embodiments, the immunogenic compositions described herein comprise from about 5 μg to about 25 μg of each CoV S glycoprotein. In embodiments, the immunogenic compositions described herein comprise from about 14 μg to about 15 μg of each CoV S glycoprotein. In embodiments, the immunogenic compositions described herein comprise from about 15 μg to about 35 μg of each CoV S glycoprotein. In embodiments, the immunogenic compositions described herein comprise from about 1 μg to about 100 μg of total CoV S glycoproteins. In embodiments, the immunogenic compositions comprise about 1 μg, about 2 μg, about 3 μg, about 4 μg, about 5 μg, about 6 μg, about 7 μg, about 8 μg, about 9 μg, about 10 μg, about 11 μg, about 12 μg, about 13 μg, about 14 μg, about 15 μg, about 16 μg, about 17 μg, about 18 μg, about 19 μg, about 20 μg, about 21 μg, about 22 μg, about 23 μg, about 24 μg, about 25 μg, about 26 μg, about 27 μg, about 28 μg, about 29 μg, about 30 μg, about 31 μg, about 32 μg, about 33 μg, about 34 μg, about 35 μg, about 36 μg, about 37 μg, about 38 μg, about 39 μg, about 40 μg, about 41 μg, about 42 μg, about 43 μg, about 44 μg, about 45 μg, about 46 μg, about 47 μg, about 48 μg, about 49 μg, about 50 μg, about 51 μg, about 52 μg, about 53 μg, about 54 μg, about 55 μg, about 56 μg, about 57 μg, about 58 μg, about 59 μg, about 60 μg, about 61 μg, about 62 μg, about 63 μg, about 64 μg, about 65 μg, about 66 μg, about 67 μg, about 68 μg, about 69 μg, about 70 μg, about 71 μg, about 72 μg, about 73 μg, about 74 μg, about 75 μg, about 76 μg, about 77 μg, about 78 μg, about 79 μg, about 80 μg, about 81 μg, about 82 μg, about 83 μg, about 84 μg, about 85 μg, about 86 μg, about 87 μg, about 88 μg, about 89 μg, about 90 μg, about 91 μg, about 92 μg, about 93 μg, about 94 μg, about 95 μg, about 96 μg, about 97 μg, about 98 μg, about 99 μg, about 100 μg, including all values and ranges therebetween, of each CoV S glycoprotein. In embodiments, the immunogenic compositions comprise about 1 μg, about 2 μg, about 3 μg, about 4 μg, about 5 μg, about 6 μg, about 7 μg, about 8 μg, about 9 μg, about 10 μg, about 11 μg, about 12 μg, about 13 μg, about 14 μg, about 15 μg, about 16 μg, about 17 μg, about 18 μg, about 19 μg, about 20 μg, about 21 μg, about 22 μg, about 23 μg, about 24 μg, about 25 μg, about 26 μg, about 27 μg, about 28 μg, about 29 μg, about 30 μg, about 31 μg, about 32 μg, about 33 μg, about 34 μg, about 35 μg, about 36 μg, about 37 μg, about 38 μg, about 39 μg, about 40 μg, about 41 μg, about 42 μg, about 43 μg, about 44 μg, about 45 μg, about 46 μg, about 47 μg, about 48 μg, about 49 μg, about 50 μg, about 51 μg, about 52 μg, about 53 μg, about 54 μg, about 55 μg, about 56 μg, about 57 μg, about 58 μg, about 59 μg, about 60 μg, about 61 μg, about 62 μg, about 63 μg, about 64 μg, about 65 μg, about 66 μg, about 67 μg, about 68 μg, about 69 μg, about 70 μg, about 71 μg, about 72 μg, about 73 μg, about 74 μg, about 75 μg, about 76 μg, about 77 μg, about 78 μg, about 79 μg, about 80 μg, about 81 μg, about 82 μg, about 83 μg, about 84 μg, about 85 μg, about 86 μg, about 87 μg, about 88 μg, about 89 μg, about 90 μg, about 91 μg, about 92 μg, about 93 μg, about 94 μg, about 95 μg, about 96 μg, about 97 μg, about 98 μg, about 99 μg, about 100 μg of total CoV S glycoproteins, including all values and ranges therebetween. In embodiments, the immunogenic compositions comprise about 1 μg of a CoV S glycoprotein. In embodiments, the immunogenic compositions comprise about 13 μg of a CoV S glycoprotein. In embodiments, the immunogenic compositions comprise about 5 μg of a CoV S glycoprotein. In embodiments, the immunogenic compositions comprise about 25 μg of a CoV S glycoprotein.

In embodiments, the immunogenic compositions described herein comprise from about 1 μg to about 300 μg of each HA glycoprotein. In embodiments, the immunogenic compositions described herein comprise from about 5 μg to about 60 μg of each HA glycoprotein. In embodiments, the immunogenic compositions described herein comprise from about 24 μg to about 40 μg of each HA glycoprotein. In embodiments, the immunogenic compositions described herein comprise from about 30 μg to about 40 μg of each HA glycoprotein. In embodiments, the immunogenic compositions described herein comprise from about 30 μg to about 60 μg of each HA glycoprotein. In embodiments, the immunogenic compositions described herein comprise from about 33 μg to about 39 μg of each HA glycoprotein. In embodiments, the immunogenic compositions described herein comprise from about 20 μg to about 240 μg of each HA glycoprotein. In embodiments, the immunogenic compositions described herein comprise from about 30 μg to about 60 μg of each HA glycoprotein. In embodiments, the immunogenic compositions described herein comprise from about 24 μg to about 40 μg of each HA glycoprotein. In embodiments, the immunogenic compositions comprise about 1 μg, about 2 μg, about 3 μg, about 4 μg, about 5 μg, about 6 μg, about 7 μg, about 8 μg, about 9 μg, about 10 μg, about 11 μg, about 12 μg, about 13 μg, about 14 μg, about 15 μg, about 16 μg, about 17 μg, about 18 μg, about 19 μg, about 20 μg, about 21 μg, about 22 μg, about 23 μg, about 24 μg, about 25 μg, about 26 μg, about 27 μg, about 28 μg, about 29 μg, about 30 μg, about 31 μg, about 32 μg, about 33 μg, about 34 μg, about 35 μg, about 36 μg, about 37 μg, about 38 μg, about 39 μg, about 40 μg, about 41 μg, about 42 μg, about 43 μg, about 44 μg, about 45 μg, about 46 μg, about 47 μg, about 48 μg, about 49 μg, about 50 μg, about 51 μg, about 52 μg, about 53 μg, about 54 μg, about 55 μg, about 56 μg, about 57 μg, about 58 μg, about 59 μg, about 60 μg, about 61 μg, about 62 μg, about 63 μg, about 64 μg, about 65 μg, about 66 μg, about 67 μg, about 68 μg, about 69 μg, about 70 μg, about 71 μg, about 72 μg, about 73 μg, about 74 μg, about 75 μg, about 76 μg, about 77 μg, about 78 μg, about 79 μg, about 80 μg, about 81 μg, about 82 μg, about 83 μg, about 84 μg, about 85 μg, about 86 μg, about 87 μg, about 88 μg, about 89 μg, about 90 μg, about 91 μg, about 92 μg, about 93 μg, about 94 μg, about 95 μg, about 96 μg, about 97 μg, about 98 μg, about 99 μg, about 100 μg, about 101 μg, about 102 μg, about 103 μg, about 104 μg, about 105 μg, about 106 μg, about 107 μg, about 108 μg, about 109 μg, about 110 μg, about 111 μg, about 112 μg, about 113 μg, about 114 μg, about 115 μg, about 116 μg, about 117 μg, about 118 μg, about 119 μg, about 120 μg, about 121 μg, about 122 μg, about 123 μg, about 124 μg, about 125 μg, about 126 μg, about 127 μg, about 128 μg, about 129 μg, about 130 μg, about 131 μg, about 132 μg, about 133 μg, about 134 μg, about 135 μg, about 136 μg, about 137 μg, about 138 μg, about 139 μg, about 140 μg, about 141 μg, about 142 μg, about 143 μg, about 144 μg, about 145 μg, about 146 μg, about 147 μg, about 148 μg, about 149 μg, about 150 μg, about 151 μg, about 152 μg, about 153 μg, about 154 μg, about 155 μg, about 156 μg, about 157 μg, about 158 μg, about 159 μg, about 160 μg, about 161 μg, about 162 μg, about 163 μg, about 164 μg, about 165 μg, about 166 μg, about 167 μg, about 168 μg, about 169 μg, about 170 μg, about 171 μg, about 172 μg, about 173 μg, about 174 μg, about 175 μg, about 176 μg, about 177 μg, about 178 μg, about 179 μg, about 180 μg, about 181 μg, about 182 μg, about 183 μg, about 184 μg, about 185 μg, about 186 μg, about 187 μg, about 188 μg, about 189 μg, about 190 μg, about 191 μg, about 192 μg, about 193 μg, about 194 μg, about 195 μg, about 196 μg, about 197 μg, about 198 μg, about 199 μg, about 200 μg, about 201 μg, about 202 μg, about 203 μg, about 204 μg, about 205 μg, about 206 μg, about 207 μg, about 208 μg, about 209 μg, about 210 μg, about 211 μg, about 212 μg, about 213 μg, about 214 μg, about 215 μg, about 216 μg, about 217 μg, about 218 μg, about 219 μg, about 220 μg, about 221 μg, about 222 μg, about 223 μg, about 224 μg, about 225 μg, about 226 μg, about 227 μg, about 228 μg, about 229 μg, about 230 μg, about 231 μg, about 232 μg, about 233 μg, about 234 μg, about 235 μg, about 236 μg, about 237 μg, about 238 μg, about 239 μg, about 240 μg, about 241 μg, about 242 μg, about 243 μg, about 244 μg, about 245 μg, about 246 μg, about 247 μg, about 248 μg, about 249 μg, about 250 μg, about 251 μg, about 252 μg, about 253 μg, about 254 μg, about 255 μg, about 256 μg, about 257 μg, about 258 μg, about 259 μg, about 260 μg, about 261 μg, about 262 μg, about 263 μg, about 264 μg, about 265 μg, about 266 μg, about 267 μg, about 268 μg, about 269 μg, about 270 μg, about 271 μg, about 272 μg, about 273 μg, about 274 μg, about 275 μg, about 276 μg, about 277 μg, about 278 μg, about 279 μg, about 280 μg, about 281 μg, about 282 μg, about 283 μg, about 284 μg, about 285 μg, about 286 μg, about 287 μg, about 288 μg, about 289 μg, about 290 μg, about 291 μg, about 292 μg, about 293 μg, about 294 μg, about 295 μg, about 296 μg, about 297 μg, about 298 μg, about 299 μg, or about 300 μg of each HA glycoprotein, including all values and ranges therebetween. In embodiments, the immunogenic compositions described herein comprise about 30 μg of each HA glycoprotein. In embodiments, the immunogenic compositions described herein comprise about 33 μg of each HA glycoprotein. In embodiments, the immunogenic compositions described herein comprise about 39 μg of each HA glycoprotein. In embodiments, the immunogenic compositions described herein comprise about 54 μg of each HA glycoprotein. In embodiments, the immunogenic compositions described herein comprise about 60 μg of each HA glycoprotein. In embodiments, the immunogenic compositions described herein comprise about 240 μg of each HA glycoprotein.

In embodiments, the immunogenic compositions described herein comprise from about 1 μg to about 1000 μg of total HA glycoproteins. In embodiments, the immunogenic compositions comprise about 1 μg, about 2 μg, about 3 μg, about 4 μg, about 5 μg, about 6 μg, about 7 μg, about 8 μg, about 9 μg, about 10 μg, about 11 μg, about 12 μg, about 13 μg, about 14 μg, about 15 μg, about 16 μg, about 17 μg, about 18 μg, about 19 μg, about 20 μg, about 21 μg, about 22 μg, about 23 μg, about 24 μg, about 25 μg, about 26 μg, about 27 μg, about 28 μg, about 29 μg, about 30 μg, about 31 μg, about 32 μg, about 33 μg, about 34 μg, about 35 μg, about 36 μg, about 37 μg, about 38 μg, about 39 μg, about 40 μg, about 41 μg, about 42 μg, about 43 μg, about 44 μg, about 45 μg, about 46 μg, about 47 μg, about 48 μg, about 49 μg, about 50 μg, about 51 μg, about 52 μg, about 53 μg, about 54 μg, about 55 μg, about 56 μg, about 57 μg, about 58 μg, about 59 μg, about 60 μg, about 61 μg, about 62 μg, about 63 μg, about 64 μg, about 65 μg, about 66 μg, about 67 μg, about 68 μg, about 69 μg, about 70 μg, about 71 μg, about 72 μg, about 73 μg, about 74 μg, about 75 μg, about 76 μg, about 77 μg, about 78 μg, about 79 μg, about 80 μg, about 81 μg, about 82 μg, about 83 μg, about 84 μg, about 85 μg, about 86 μg, about 87 μg, about 88 μg, about 89 μg, about 90 μg, about 91 μg, about 92 μg, about 93 μg, about 94 μg, about 95 μg, about 96 μg, about 97 μg, about 98 μg, about 99 μg, about 100 μg, about 101 μg, about 102 μg, about 103 μg, about 104 μg, about 105 μg, about 106 μg, about 107 μg, about 108 μg, about 109 μg, about 110 μg, about 111 μg, about 112 μg, about 113 μg, about 114 μg, about 115 μg, about 116 μg, about 117 μg, about 118 μg, about 119 μg, about 120 μg, about 121 μg, about 122 μg, about 123 μg, about 124 μg, about 125 μg, about 126 μg, about 127 μg, about 128 μg, about 129 μg, about 130 μg, about 131 μg, about 132 μg, about 133 μg, about 134 μg, about 135 μg, about 136 μg, about 137 μg, about 138 μg, about 139 μg, about 140 μg, about 141 μg, about 142 μg, about 143 μg, about 144 μg, about 145 μg, about 146 μg, about 147 μg, about 148 μg, about 149 μg, about 150 μg, about 151 μg, about 152 μg, about 153 μg, about 154 μg, about 155 μg, about 156 μg, about 157 μg, about 158 μg, about 159 μg, about 160 μg, about 161 μg, about 162 μg, about 163 μg, about 164 μg, about 165 μg, about 166 μg, about 167 μg, about 168 μg, about 169 μg, about 170 μg, about 171 μg, about 172 μg, about 173 μg, about 174 μg, about 175 μg, about 176 μg, about 177 μg, about 178 μg, about 179 μg, about 180 μg, about 181 μg, about 182 μg, about 183 μg, about 184 μg, about 185 μg, about 186 μg, about 187 μg, about 188 μg, about 189 μg, about 190 μg, about 191 μg, about 192 μg, about 193 μg, about 194 μg, about 195 μg, about 196 μg, about 197 μg, about 198 μg, about 199 μg, about 200 μg, about 201 μg, about 202 μg, about 203 μg, about 204 μg, about 205 μg, about 206 μg, about 207 μg, about 208 μg, about 209 μg, about 210 μg, about 211 μg, about 212 μg, about 213 μg, about 214 μg, about 215 μg, about 216 μg, about 217 μg, about 218 μg, about 219 μg, about 220 μg, about 221 μg, about 222 μg, about 223 μg, about 224 μg, about 225 μg, about 226 μg, about 227 μg, about 228 μg, about 229 μg, about 230 μg, about 231 μg, about 232 μg, about 233 μg, about 234 μg, about 235 μg, about 236 μg, about 237 μg, about 238 μg, about 239 μg, about 240 μg, about 241 μg, about 242 μg, about 243 μg, about 244 μg, about 245 μg, about 246 μg, about 247 μg, about 248 μg, about 249 μg, about 250 μg, about 251 μg, about 252 μg, about 253 μg, about 254 μg, about 255 μg, about 256 μg, about 257 μg, about 258 μg, about 259 μg, about 260 μg, about 261 μg, about 262 μg, about 263 μg, about 264 μg, about 265 μg, about 266 μg, about 267 μg, about 268 μg, about 269 μg, about 270 μg, about 271 μg, about 272 μg, about 273 μg, about 274 μg, about 275 μg, about 276 μg, about 277 μg, about 278 μg, about 279 μg, about 280 μg, about 281 μg, about 282 μg, about 283 μg, about 284 μg, about 285 μg, about 286 μg, about 287 μg, about 288 μg, about 289 μg, about 290 μg, about 291 μg, about 292 μg, about 293 μg, about 294 μg, about 295 μg, about 296 μg, about 297 μg, about 298 μg, about 299 μg, about 300 μg, about 301 μg, about 302 μg, about 303 μg, about 304 μg, about 305 μg, about 306 μg, about 307 μg, about 308 μg, about 309 μg, about 310 μg, about 311 μg, about 312 μg, about 313 μg, about 314 μg, about 315 μg, about 316 μg, about 317 μg, about 318 μg, about 319 μg, about 320 μg, about 321 μg, about 322 μg, about 323 μg, about 324 μg, about 325 μg, about 326 μg, about 327 μg, about 328 μg, about 329 μg, about 330 μg, about 331 μg, about 332 μg, about 333 μg, about 334 μg, about 335 μg, about 336 μg, about 337 μg, about 338 μg, about 339 μg, about 340 μg, about 341 μg, about 342 μg, about 343 μg, about 344 μg, about 345 μg, about 346 μg, about 347 μg, about 348 μg, about 349 μg, about 350 μg, about 351 μg, about 352 μg, about 353 μg, about 354 μg, about 355 μg, about 356 μg, about 357 μg, about 358 μg, about 359 μg, about 360 μg, about 361 μg, about 362 μg, about 363 μg, about 364 μg, about 365 μg, about 366 μg, about 367 μg, about 368 μg, about 369 μg, about 370 μg, about 371 μg, about 372 μg, about 373 μg, about 374 μg, about 375 μg, about 376 μg, about 377 μg, about 378 μg, about 379 μg, about 380 μg, about 381 μg, about 382 μg, about 383 μg, about 384 μg, about 385 μg, about 386 μg, about 387 μg, about 388 μg, about 389 μg, about 390 μg, about 391 μg, about 392 μg, about 393 μg, about 394 μg, about 395 μg, about 396 μg, about 397 μg, about 398 μg, about 399 μg, about 400 μg, about 401 μg, about 402 μg, about 403 μg, about 404 μg, about 405 μg, about 406 μg, about 407 μg, about 408 μg, about 409 μg, about 410 μg, about 411 μg, about 412 μg, about 413 μg, about 414 μg, about 415 μg, about 416 μg, about 417 μg, about 418 μg, about 419 μg, about 420 μg, about 421 μg, about 422 μg, about 423 μg, about 424 μg, about 425 μg, about 426 μg, about 427 μg, about 428 μg, about 429 μg, about 430 μg, about 431 μg, about 432 μg, about 433 μg, about 434 μg, about 435 μg, about 436 μg, about 437 μg, about 438 μg, about 439 μg, about 440 μg, about 441 μg, about 442 μg, about 443 μg, about 444 μg, about 445 μg, about 446 μg, about 447 μg, about 448 μg, about 449 μg, about 450 μg, about 451 μg, about 452 μg, about 453 μg, about 454 μg, about 455 μg, about 456 μg, about 457 μg, about 458 μg, about 459 μg, about 460 μg, about 461 μg, about 462 μg, about 463 μg, about 464 μg, about 465 μg, about 466 μg, about 467 μg, about 468 μg, about 469 μg, about 470 μg, about 471 μg, about 472 μg, about 473 μg, about 474 μg, about 475 μg, about 476 μg, about 477 μg, about 478 μg, about 479 μg, about 480 μg, about 481 μg, about 482 μg, about 483 μg, about 484 μg, about 485 μg, about 486 μg, about 487 μg, about 488 μg, about 489 μg, about 490 μg, about 491 μg, about 492 μg, about 493 μg, about 494 μg, about 495 μg, about 496 μg, about 497 μg, about 498 μg, about 499 μg, about 500 μg, about 501 μg, about 502 μg, about 503 μg, about 504 μg, about 505 μg, about 506 μg, about 507 μg, about 508 μg, about 509 μg, about 510 μg, about 511 μg, about 512 μg, about 513 μg, about 514 μg, about 515 μg, about 516 μg, about 517 μg, about 518 μg, about 519 μg, about 520 μg, about 521 μg, about 522 μg, about 523 μg, about 524 μg, about 525 μg, about 526 μg, about 527 μg, about 528 μg, about 529 μg, about 530 μg, about 531 μg, about 532 μg, about 533 μg, about 534 μg, about 535 μg, about 536 μg, about 537 μg, about 538 μg, about 539 μg, about 540 μg, about 541 μg, about 542 μg, about 543 μg, about 544 μg, about 545 μg, about 546 μg, about 547 μg, about 548 μg, about 549 μg, about 550 μg, about 551 μg, about 552 μg, about 553 μg, about 554 μg, about 555 μg, about 556 μg, about 557 μg, about 558 μg, about 559 μg, about 560 μg, about 561 μg, about 562 μg, about 563 μg, about 564 μg, about 565 μg, about 566 μg, about 567 μg, about 568 μg, about 569 μg, about 570 μg, about 571 μg, about 572 μg, about 573 μg, about 574 μg, about 575 μg, about 576 μg, about 577 μg, about 578 μg, about 579 μg, about 580 μg, about 581 μg, about 582 μg, about 583 μg, about 584 μg, about 585 μg, about 586 μg, about 587 μg, about 588 μg, about 589 μg, about 590 μg, about 591 μg, about 592 μg, about 593 μg, about 594 μg, about 595 μg, about 596 μg, about 597 μg, about 598 μg, about 599 μg, about 600 μg, about 601 μg, about 602 μg, about 603 μg, about 604 μg, about 605 μg, about 606 μg, about 607 μg, about 608 μg, about 609 μg, about 610 μg, about 611 μg, about 612 μg, about 613 μg, about 614 μg, about 615 μg, about 616 μg, about 617 μg, about 618 μg, about 619 μg, about 620 μg, about 621 μg, about 622 μg, about 623 μg, about 624 μg, about 625 μg, about 626 μg, about 627 μg, about 628 μg, about 629 μg, about 630 μg, about 631 μg, about 632 μg, about 633 μg, about 634 μg, about 635 μg, about 636 μg, about 637 μg, about 638 μg, about 639 μg, about 640 μg, about 641 μg, about 642 μg, about 643 μg, about 644 μg, about 645 μg, about 646 μg, about 647 μg, about 648 μg, about 649 μg, about 650 μg, about 651 μg, about 652 μg, about 653 μg, about 654 μg, about 655 μg, about 656 μg, about 657 μg, about 658 μg, about 659 μg, about 660 μg, about 661 μg, about 662 μg, about 663 μg, about 664 μg, about 665 μg, about 666 μg, about 667 μg, about 668 μg, about 669 μg, about 670 μg, about 671 μg, about 672 μg, about 673 μg, about 674 μg, about 675 μg, about 676 μg, about 677 μg, about 678 μg, about 679 μg, about 680 μg, about 681 μg, about 682 μg, about 683 μg, about 684 μg, about 685 μg, about 686 μg, about 687 μg, about 688 μg, about 689 μg, about 690 μg, about 691 μg, about 692 μg, about 693 μg, about 694 μg, about 695 μg, about 696 μg, about 697 μg, about 698 μg, about 699 μg, about 700 μg, about 701 μg, about 702 μg, about 703 μg, about 704 μg, about 705 μg, about 706 μg, about 707 μg, about 708 μg, about 709 μg, about 710 μg, about 711 μg, about 712 μg, about 713 μg, about 714 μg, about 715 μg, about 716 μg, about 717 μg, about 718 μg, about 719 μg, about 720 μg, about 721 μg, about 722 μg, about 723 μg, about 724 μg, about 725 μg, about 726 μg, about 727 μg, about 728 μg, about 729 μg, about 730 μg, about 731 μg, about 732 μg, about 733 μg, about 734 μg, about 735 μg, about 736 μg, about 737 μg, about 738 μg, about 739 μg, about 740 μg, about 741 μg, about 742 μg, about 743 μg, about 744 μg, about 745 μg, about 746 μg, about 747 μg, about 748 μg, about 749 μg, about 750 μg, about 751 μg, about 752 μg, about 753 μg, about 754 μg, about 755 μg, about 756 μg, about 757 μg, about 758 μg, about 759 μg, about 760 μg, about 761 μg, about 762 μg, about 763 μg, about 764 μg, about 765 μg, about 766 μg, about 767 μg, about 768 μg, about 769 μg, about 770 μg, about 771 μg, about 772 μg, about 773 μg, about 774 μg, about 775 μg, about 776 μg, about 777 μg, about 778 μg, about 779 μg, about 780 μg, about 781 μg, about 782 μg, about 783 μg, about 784 μg, about 785 μg, about 786 μg, about 787 μg, about 788 μg, about 789 μg, about 790 μg, about 791 μg, about 792 μg, about 793 μg, about 794 μg, about 795 μg, about 796 μg, about 797 μg, about 798 μg, about 799 μg, about 800 μg, about 801 μg, about 802 μg, about 803 μg, about 804 μg, about 805 μg, about 806 μg, about 807 μg, about 808 μg, about 809 μg, about 810 μg, about 811 μg, about 812 μg, about 813 μg, about 814 μg, about 815 μg, about 816 μg, about 817 μg, about 818 μg, about 819 μg, about 820 μg, about 821 μg, about 822 μg, about 823 μg, about 824 μg, about 825 μg, about 826 μg, about 827 μg, about 828 μg, about 829 μg, about 830 μg, about 831 μg, about 832 μg, about 833 μg, about 834 μg, about 835 μg, about 836 μg, about 837 μg, about 838 μg, about 839 μg, about 840 μg, about 841 μg, about 842 μg, about 843 μg, about 844 μg, about 845 μg, about 846 μg, about 847 μg, about 848 μg, about 849 μg, about 850 μg, about 851 μg, about 852 μg, about 853 μg, about 854 μg, about 855 μg, about 856 μg, about 857 μg, about 858 μg, about 859 μg, about 860 μg, about 861 μg, about 862 μg, about 863 μg, about 864 μg, about 865 μg, about 866 μg, about 867 μg, about 868 μg, about 869 μg, about 870 μg, about 871 μg, about 872 μg, about 873 μg, about 874 μg, about 875 μg, about 876 μg, about 877 μg, about 878 μg, about 879 μg, about 880 μg, about 881 μg, about 882 μg, about 883 μg, about 884 μg, about 885 μg, about 886 μg, about 887 μg, about 888 μg, about 889 μg, about 890 μg, about 891 μg, about 892 μg, about 893 μg, about 894 μg, about 895 μg, about 896 μg, about 897 μg, about 898 μg, about 899 μg, about 900 μg, about 901 μg, about 902 μg, about 903 μg, about 904 μg, about 905 μg, about 906 μg, about 907 μg, about 908 μg, about 909 μg, about 910 μg, about 911 μg, about 912 μg, about 913 μg, about 914 μg, about 915 μg, about 916 μg, about 917 μg, about 918 μg, about 919 μg, about 920 μg, about 921 μg, about 922 μg, about 923 μg, about 924 μg, about 925 μg, about 926 μg, about 927 μg, about 928 μg, about 929 μg, about 930 μg, about 931 μg, about 932 μg, about 933 μg, about 934 μg, about 935 μg, about 936 μg, about 937 μg, about 938 μg, about 939 μg, about 940 μg, about 941 μg, about 942 μg, about 943 μg, about 944 μg, about 945 μg, about 946 μg, about 947 μg, about 948 μg, about 949 μg, about 950 μg, about 951 μg, about 952 μg, about 953 μg, about 954 μg, about 955 μg, about 956 μg, about 957 μg, about 958 μg, about 959 μg, about 960 μg, about 961 μg, about 962 μg, about 963 μg, about 964 μg, about 965 μg, about 966 μg, about 967 μg, about 968 μg, about 969 μg, about 970 μg, about 971 μg, about 972 μg, about 973 μg, about 974 μg, about 975 μg, about 976 μg, about 977 μg, about 978 μg, about 979 μg, about 980 μg, about 981 μg, about 982 μg, about 983 μg, about 984 μg, about 985 μg, about 986 μg, about 987 μg, about 988 μg, about 989 μg, about 990 μg, about 991 μg, about 992 μg, about 993 μg, about 994 μg, about 995 μg, about 996 μg, about 997 μg, about 998 μg, about 999 μg, about 1000 μg, of total HA glycoproteins, including all values and ranges therebetween.

In embodiments, the immunogenic compositions described herein comprise from about 1 μg to about 300 μg of each RSV F glycoprotein. In embodiments, the immunogenic compositions described herein comprise from about 5 μg to about 60 μg of each RSV F glycoprotein. In embodiments, the immunogenic compositions described herein comprise from about 24 μg to about 40 μg of each RSV F glycoprotein. In embodiments, the immunogenic compositions described herein comprise from about 30 μg to about 40 μg of each RSV F glycoprotein. In embodiments, the immunogenic compositions described herein comprise from about 30 μg to about 60 μg of each RSV F glycoprotein. In embodiments, the immunogenic compositions described herein comprise from about 33 μg to about 39 μg of each RSV F glycoprotein. In embodiments, the immunogenic compositions described herein comprise from about 20 μg to about 240 μg of each RSV F glycoprotein. In embodiments, the immunogenic compositions described herein comprise from about 30 μg to about 60 μg of each RSV F glycoprotein. In embodiments, the immunogenic compositions described herein comprise from about 24 μg to about 40 μg of each RSV F glycoprotein. In embodiments, the immunogenic compositions comprise about 1 μg, about 2 μg, about 3 μg, about 4 μg, about 5 μg, about 6 μg, about 7 μg, about 8 μg, about 9 μg, about 10 μg, about 11 μg, about 12 μg, about 13 μg, about 14 μg, about 15 μg, about 16 μg, about 17 μg, about 18 μg, about 19 μg, about 20 μg, about 21 μg, about 22 μg, about 23 μg, about 24 μg, about 25 μg, about 26 μg, about 27 μg, about 28 μg, about 29 μg, about 30 μg, about 31 μg, about 32 μg, about 33 μg, about 34 μg, about 35 μg, about 36 μg, about 37 μg, about 38 μg, about 39 μg, about 40 μg, about 41 μg, about 42 μg, about 43 μg, about 44 μg, about 45 μg, about 46 μg, about 47 μg, about 48 μg, about 49 μg, about 50 μg, about 51 μg, about 52 μg, about 53 μg, about 54 μg, about 55 μg, about 56 μg, about 57 μg, about 58 μg, about 59 μg, about 60 μg, about 61 μg, about 62 μg, about 63 μg, about 64 μg, about 65 μg, about 66 μg, about 67 μg, about 68 μg, about 69 μg, about 70 μg, about 71 μg, about 72 μg, about 73 μg, about 74 μg, about 75 μg, about 76 μg, about 77 μg, about 78 μg, about 79 μg, about 80 μg, about 81 μg, about 82 μg, about 83 μg, about 84 μg, about 85 μg, about 86 μg, about 87 μg, about 88 μg, about 89 μg, about 90 μg, about 91 μg, about 92 μg, about 93 μg, about 94 μg, about 95 μg, about 96 μg, about 97 μg, about 98 μg, about 99 μg, about 100 μg, about 101 μg, about 102 μg, about 103 μg, about 104 μg, about 105 μg, about 106 μg, about 107 μg, about 108 μg, about 109 μg, about 110 μg, about 111 μg, about 112 μg, about 113 μg, about 114 μg, about 115 μg, about 116 μg, about 117 μg, about 118 μg, about 119 μg, about 120 μg, about 121 μg, about 122 μg, about 123 μg, about 124 μg, about 125 μg, about 126 μg, about 127 μg, about 128 μg, about 129 μg, about 130 μg, about 131 μg, about 132 μg, about 133 μg, about 134 μg, about 135 μg, about 136 μg, about 137 μg, about 138 μg, about 139 μg, about 140 μg, about 141 μg, about 142 μg, about 143 μg, about 144 μg, about 145 μg, about 146 μg, about 147 μg, about 148 μg, about 149 μg, about 150 μg, about 151 μg, about 152 μg, about 153 μg, about 154 μg, about 155 μg, about 156 μg, about 157 μg, about 158 μg, about 159 μg, about 160 μg, about 161 μg, about 162 μg, about 163 μg, about 164 μg, about 165 μg, about 166 μg, about 167 μg, about 168 μg, about 169 μg, about 170 μg, about 171 μg, about 172 μg, about 173 μg, about 174 μg, about 175 μg, about 176 μg, about 177 μg, about 178 μg, about 179 μg, about 180 μg, about 181 μg, about 182 μg, about 183 μg, about 184 μg, about 185 μg, about 186 μg, about 187 μg, about 188 μg, about 189 μg, about 190 μg, about 191 μg, about 192 μg, about 193 μg, about 194 μg, about 195 μg, about 196 μg, about 197 μg, about 198 μg, about 199 μg, about 200 μg, about 201 μg, about 202 μg, about 203 μg, about 204 μg, about 205 μg, about 206 μg, about 207 μg, about 208 μg, about 209 μg, about 210 μg, about 211 μg, about 212 μg, about 213 μg, about 214 μg, about 215 μg, about 216 μg, about 217 μg, about 218 μg, about 219 μg, about 220 μg, about 221 μg, about 222 μg, about 223 μg, about 224 μg, about 225 μg, about 226 μg, about 227 μg, about 228 μg, about 229 μg, about 230 μg, about 231 μg, about 232 μg, about 233 μg, about 234 μg, about 235 μg, about 236 μg, about 237 μg, about 238 μg, about 239 μg, about 240 μg, about 241 μg, about 242 μg, about 243 μg, about 244 μg, about 245 μg, about 246 μg, about 247 μg, about 248 μg, about 249 μg, about 250 μg, about 251 μg, about 252 μg, about 253 μg, about 254 μg, about 255 μg, about 256 μg, about 257 μg, about 258 μg, about 259 μg, about 260 μg, about 261 μg, about 262 μg, about 263 μg, about 264 μg, about 265 μg, about 266 μg, about 267 μg, about 268 μg, about 269 μg, about 270 μg, about 271 μg, about 272 μg, about 273 μg, about 274 μg, about 275 μg, about 276 μg, about 277 μg, about 278 μg, about 279 μg, about 280 μg, about 281 μg, about 282 μg, about 283 μg, about 284 μg, about 285 μg, about 286 μg, about 287 μg, about 288 μg, about 289 μg, about 290 μg, about 291 μg, about 292 μg, about 293 μg, about 294 μg, about 295 μg, about 296 μg, about 297 μg, about 298 μg, about 299 μg, or about 300 μg of each RSV F glycoprotein, including all values and ranges therebetween. In embodiments, the immunogenic compositions described herein comprise about 60 μg of each RSV F glycoprotein. In embodiments, the immunogenic compositions described herein comprise about 240 μg of each RSV F glycoprotein.

In embodiments, the immunogenic compositions described herein comprise from about 1 μg to about 1000 μg of total RSV F glycoproteins. In embodiments, the immunogenic compositions comprise about 1 μg, about 2 μg, about 3 μg, about 4 μg, about 5 μg, about 6 μg, about 7 μg, about 8 μg, about 9 μg, about 10 μg, about 11 μg, about 12 μg, about 13 μg, about 14 μg, about 15 μg, about 16 μg, about 17 μg, about 18 μg, about 19 μg, about 20 μg, about 21 μg, about 22 μg, about 23 μg, about 24 μg, about 25 μg, about 26 μg, about 27 μg, about 28 μg, about 29 μg, about 30 μg, about 31 μg, about 32 μg, about 33 μg, about 34 μg, about 35 μg, about 36 μg, about 37 μg, about 38 μg, about 39 μg, about 40 μg, about 41 μg, about 42 μg, about 43 μg, about 44 μg, about 45 μg, about 46 μg, about 47 μg, about 48 μg, about 49 μg, about 50 μg, about 51 μg, about 52 μg, about 53 μg, about 54 μg, about 55 μg, about 56 μg, about 57 μg, about 58 μg, about 59 μg, about 60 μg, about 61 μg, about 62 μg, about 63 μg, about 64 μg, about 65 μg, about 66 μg, about 67 μg, about 68 μg, about 69 μg, about 70 μg, about 71 μg, about 72 μg, about 73 μg, about 74 μg, about 75 μg, about 76 μg, about 77 μg, about 78 μg, about 79 μg, about 80 μg, about 81 μg, about 82 μg, about 83 μg, about 84 μg, about 85 μg, about 86 μg, about 87 μg, about 88 μg, about 89 μg, about 90 μg, about 91 μg, about 92 μg, about 93 μg, about 94 μg, about 95 μg, about 96 μg, about 97 μg, about 98 μg, about 99 μg, about 100 μg, about 101 μg, about 102 μg, about 103 μg, about 104 μg, about 105 μg, about 106 μg, about 107 μg, about 108 μg, about 109 μg, about 110 μg, about 111 μg, about 112 μg, about 113 μg, about 114 μg, about 115 μg, about 116 μg, about 117 μg, about 118 μg, about 119 μg, about 120 μg, about 121 μg, about 122 μg, about 123 μg, about 124 μg, about 125 μg, about 126 μg, about 127 μg, about 128 μg, about 129 μg, about 130 μg, about 131 μg, about 132 μg, about 133 μg, about 134 μg, about 135 μg, about 136 μg, about 137 μg, about 138 μg, about 139 μg, about 140 μg, about 141 μg, about 142 μg, about 143 μg, about 144 μg, about 145 μg, about 146 μg, about 147 μg, about 148 μg, about 149 μg, about 150 μg, about 151 μg, about 152 μg, about 153 μg, about 154 μg, about 155 μg, about 156 μg, about 157 μg, about 158 μg, about 159 μg, about 160 μg, about 161 μg, about 162 μg, about 163 μg, about 164 μg, about 165 μg, about 166 μg, about 167 μg, about 168 μg, about 169 μg, about 170 μg, about 171 μg, about 172 μg, about 173 μg, about 174 μg, about 175 μg, about 176 μg, about 177 μg, about 178 μg, about 179 μg, about 180 μg, about 181 μg, about 182 μg, about 183 μg, about 184 μg, about 185 μg, about 186 μg, about 187 μg, about 188 μg, about 189 μg, about 190 μg, about 191 μg, about 192 μg, about 193 μg, about 194 μg, about 195 μg, about 196 μg, about 197 μg, about 198 μg, about 199 μg, about 200 μg, about 201 μg, about 202 μg, about 203 μg, about 204 μg, about 205 μg, about 206 μg, about 207 μg, about 208 μg, about 209 μg, about 210 μg, about 211 μg, about 212 μg, about 213 μg, about 214 μg, about 215 μg, about 216 μg, about 217 μg, about 218 μg, about 219 μg, about 220 μg, about 221 μg, about 222 μg, about 223 μg, about 224 μg, about 225 μg, about 226 μg, about 227 μg, about 228 μg, about 229 μg, about 230 μg, about 231 μg, about 232 μg, about 233 μg, about 234 μg, about 235 μg, about 236 μg, about 237 μg, about 238 μg, about 239 μg, about 240 μg, about 241 μg, about 242 μg, about 243 μg, about 244 μg, about 245 μg, about 246 μg, about 247 μg, about 248 μg, about 249 μg, about 250 μg, about 251 μg, about 252 μg, about 253 μg, about 254 μg, about 255 μg, about 256 μg, about 257 μg, about 258 μg, about 259 μg, about 260 μg, about 261 μg, about 262 μg, about 263 μg, about 264 μg, about 265 μg, about 266 μg, about 267 μg, about 268 μg, about 269 μg, about 270 μg, about 271 μg, about 272 μg, about 273 μg, about 274 μg, about 275 μg, about 276 μg, about 277 μg, about 278 μg, about 279 μg, about 280 μg, about 281 μg, about 282 μg, about 283 μg, about 284 μg, about 285 μg, about 286 μg, about 287 μg, about 288 μg, about 289 μg, about 290 μg, about 291 μg, about 292 μg, about 293 μg, about 294 μg, about 295 μg, about 296 μg, about 297 μg, about 298 μg, about 299 μg, about 300 μg, about 301 μg, about 302 μg, about 303 μg, about 304 μg, about 305 μg, about 306 μg, about 307 μg, about 308 μg, about 309 μg, about 310 μg, about 311 μg, about 312 μg, about 313 μg, about 314 μg, about 315 μg, about 316 μg, about 317 μg, about 318 μg, about 319 μg, about 320 μg, about 321 μg, about 322 μg, about 323 μg, about 324 μg, about 325 μg, about 326 μg, about 327 μg, about 328 μg, about 329 μg, about 330 μg, about 331 μg, about 332 μg, about 333 μg, about 334 μg, about 335 μg, about 336 μg, about 337 μg, about 338 μg, about 339 μg, about 340 μg, about 341 μg, about 342 μg, about 343 μg, about 344 μg, about 345 μg, about 346 μg, about 347 μg, about 348 μg, about 349 μg, about 350 μg, about 351 μg, about 352 μg, about 353 μg, about 354 μg, about 355 μg, about 356 μg, about 357 μg, about 358 μg, about 359 μg, about 360 μg, about 361 μg, about 362 μg, about 363 μg, about 364 μg, about 365 μg, about 366 μg, about 367 μg, about 368 μg, about 369 μg, about 370 μg, about 371 μg, about 372 μg, about 373 μg, about 374 μg, about 375 μg, about 376 μg, about 377 μg, about 378 μg, about 379 μg, about 380 μg, about 381 μg, about 382 μg, about 383 μg, about 384 μg, about 385 μg, about 386 μg, about 387 μg, about 388 μg, about 389 μg, about 390 μg, about 391 μg, about 392 μg, about 393 μg, about 394 μg, about 395 μg, about 396 μg, about 397 μg, about 398 μg, about 399 μg, about 400 μg, about 401 μg, about 402 μg, about 403 μg, about 404 μg, about 405 μg, about 406 μg, about 407 μg, about 408 μg, about 409 μg, about 410 μg, about 411 μg, about 412 μg, about 413 μg, about 414 μg, about 415 μg, about 416 μg, about 417 μg, about 418 μg, about 419 μg, about 420 μg, about 421 μg, about 422 μg, about 423 μg, about 424 μg, about 425 μg, about 426 μg, about 427 μg, about 428 μg, about 429 μg, about 430 μg, about 431 μg, about 432 μg, about 433 μg, about 434 μg, about 435 μg, about 436 μg, about 437 μg, about 438 μg, about 439 μg, about 440 μg, about 441 μg, about 442 μg, about 443 μg, about 444 μg, about 445 μg, about 446 μg, about 447 μg, about 448 μg, about 449 μg, about 450 μg, about 451 μg, about 452 μg, about 453 μg, about 454 μg, about 455 μg, about 456 μg, about 457 μg, about 458 μg, about 459 μg, about 460 μg, about 461 μg, about 462 μg, about 463 μg, about 464 μg, about 465 μg, about 466 μg, about 467 μg, about 468 μg, about 469 μg, about 470 μg, about 471 μg, about 472 μg, about 473 μg, about 474 μg, about 475 μg, about 476 μg, about 477 μg, about 478 μg, about 479 μg, about 480 μg, about 481 μg, about 482 μg, about 483 μg, about 484 μg, about 485 μg, about 486 μg, about 487 μg, about 488 μg, about 489 μg, about 490 μg, about 491 μg, about 492 μg, about 493 μg, about 494 μg, about 495 μg, about 496 μg, about 497 μg, about 498 μg, about 499 μg, about 500 μg, about 501 μg, about 502 μg, about 503 μg, about 504 μg, about 505 μg, about 506 μg, about 507 μg, about 508 μg, about 509 μg, about 510 μg, about 511 μg, about 512 μg, about 513 μg, about 514 μg, about 515 μg, about 516 μg, about 517 μg, about 518 μg, about 519 μg, about 520 μg, about 521 μg, about 522 μg, about 523 μg, about 524 μg, about 525 μg, about 526 μg, about 527 μg, about 528 μg, about 529 μg, about 530 μg, about 531 μg, about 532 μg, about 533 μg, about 534 μg, about 535 μg, about 536 μg, about 537 μg, about 538 μg, about 539 μg, about 540 μg, about 541 μg, about 542 μg, about 543 μg, about 544 μg, about 545 μg, about 546 μg, about 547 μg, about 548 μg, about 549 μg, about 550 μg, about 551 μg, about 552 μg, about 553 μg, about 554 μg, about 555 μg, about 556 μg, about 557 μg, about 558 μg, about 559 μg, about 560 μg, about 561 μg, about 562 μg, about 563 μg, about 564 μg, about 565 μg, about 566 μg, about 567 μg, about 568 μg, about 569 μg, about 570 μg, about 571 μg, about 572 μg, about 573 μg, about 574 μg, about 575 μg, about 576 μg, about 577 μg, about 578 μg, about 579 μg, about 580 μg, about 581 μg, about 582 μg, about 583 μg, about 584 μg, about 585 μg, about 586 μg, about 587 μg, about 588 μg, about 589 μg, about 590 μg, about 591 μg, about 592 μg, about 593 μg, about 594 μg, about 595 μg, about 596 μg, about 597 μg, about 598 μg, about 599 μg, about 600 μg, about 601 μg, about 602 μg, about 603 μg, about 604 μg, about 605 μg, about 606 μg, about 607 μg, about 608 μg, about 609 μg, about 610 μg, about 611 μg, about 612 μg, about 613 μg, about 614 μg, about 615 μg, about 616 μg, about 617 μg, about 618 μg, about 619 μg, about 620 μg, about 621 μg, about 622 μg, about 623 μg, about 624 μg, about 625 μg, about 626 μg, about 627 μg, about 628 μg, about 629 μg, about 630 μg, about 631 μg, about 632 μg, about 633 μg, about 634 μg, about 635 μg, about 636 μg, about 637 μg, about 638 μg, about 639 μg, about 640 μg, about 641 μg, about 642 μg, about 643 μg, about 644 μg, about 645 μg, about 646 μg, about 647 μg, about 648 μg, about 649 μg, about 650 μg, about 651 μg, about 652 μg, about 653 μg, about 654 μg, about 655 μg, about 656 μg, about 657 μg, about 658 μg, about 659 μg, about 660 μg, about 661 μg, about 662 μg, about 663 μg, about 664 μg, about 665 μg, about 666 μg, about 667 μg, about 668 μg, about 669 μg, about 670 μg, about 671 μg, about 672 μg, about 673 μg, about 674 μg, about 675 μg, about 676 μg, about 677 μg, about 678 μg, about 679 μg, about 680 μg, about 681 μg, about 682 μg, about 683 μg, about 684 μg, about 685 μg, about 686 μg, about 687 μg, about 688 μg, about 689 μg, about 690 μg, about 691 μg, about 692 μg, about 693 μg, about 694 μg, about 695 μg, about 696 μg, about 697 μg, about 698 μg, about 699 μg, about 700 μg, about 701 μg, about 702 μg, about 703 μg, about 704 μg, about 705 μg, about 706 μg, about 707 μg, about 708 μg, about 709 μg, about 710 μg, about 711 μg, about 712 μg, about 713 μg, about 714 μg, about 715 μg, about 716 μg, about 717 μg, about 718 μg, about 719 μg, about 720 μg, about 721 μg, about 722 μg, about 723 μg, about 724 μg, about 725 μg, about 726 μg, about 727 μg, about 728 μg, about 729 μg, about 730 μg, about 731 μg, about 732 μg, about 733 μg, about 734 μg, about 735 μg, about 736 μg, about 737 μg, about 738 μg, about 739 μg, about 740 μg, about 741 μg, about 742 μg, about 743 μg, about 744 μg, about 745 μg, about 746 μg, about 747 μg, about 748 μg, about 749 μg, about 750 μg, about 751 μg, about 752 μg, about 753 μg, about 754 μg, about 755 μg, about 756 μg, about 757 μg, about 758 μg, about 759 μg, about 760 μg, about 761 μg, about 762 μg, about 763 μg, about 764 μg, about 765 μg, about 766 μg, about 767 μg, about 768 μg, about 769 μg, about 770 μg, about 771 μg, about 772 μg, about 773 μg, about 774 μg, about 775 μg, about 776 μg, about 777 μg, about 778 μg, about 779 μg, about 780 μg, about 781 μg, about 782 μg, about 783 μg, about 784 μg, about 785 μg, about 786 μg, about 787 μg, about 788 μg, about 789 μg, about 790 μg, about 791 μg, about 792 μg, about 793 μg, about 794 μg, about 795 μg, about 796 μg, about 797 μg, about 798 μg, about 799 μg, about 800 μg, about 801 μg, about 802 μg, about 803 μg, about 804 μg, about 805 μg, about 806 μg, about 807 μg, about 808 μg, about 809 μg, about 810 μg, about 811 μg, about 812 μg, about 813 μg, about 814 μg, about 815 μg, about 816 μg, about 817 μg, about 818 μg, about 819 μg, about 820 μg, about 821 μg, about 822 μg, about 823 μg, about 824 μg, about 825 μg, about 826 μg, about 827 μg, about 828 μg, about 829 μg, about 830 μg, about 831 μg, about 832 μg, about 833 μg, about 834 μg, about 835 μg, about 836 μg, about 837 μg, about 838 μg, about 839 μg, about 840 μg, about 841 μg, about 842 μg, about 843 μg, about 844 μg, about 845 μg, about 846 μg, about 847 μg, about 848 μg, about 849 μg, about 850 μg, about 851 μg, about 852 μg, about 853 μg, about 854 μg, about 855 μg, about 856 μg, about 857 μg, about 858 μg, about 859 μg, about 860 μg, about 861 μg, about 862 μg, about 863 μg, about 864 μg, about 865 μg, about 866 μg, about 867 μg, about 868 μg, about 869 μg, about 870 μg, about 871 μg, about 872 μg, about 873 μg, about 874 μg, about 875 μg, about 876 μg, about 877 μg, about 878 μg, about 879 μg, about 880 μg, about 881 μg, about 882 μg, about 883 μg, about 884 μg, about 885 μg, about 886 μg, about 887 μg, about 888 μg, about 889 μg, about 890 μg, about 891 μg, about 892 μg, about 893 μg, about 894 μg, about 895 μg, about 896 μg, about 897 μg, about 898 μg, about 899 μg, about 900 μg, about 901 μg, about 902 μg, about 903 μg, about 904 μg, about 905 μg, about 906 μg, about 907 μg, about 908 μg, about 909 μg, about 910 μg, about 911 μg, about 912 μg, about 913 μg, about 914 μg, about 915 μg, about 916 μg, about 917 μg, about 918 μg, about 919 μg, about 920 μg, about 921 μg, about 922 μg, about 923 μg, about 924 μg, about 925 μg, about 926 μg, about 927 μg, about 928 μg, about 929 μg, about 930 μg, about 931 μg, about 932 μg, about 933 μg, about 934 μg, about 935 μg, about 936 μg, about 937 μg, about 938 μg, about 939 μg, about 940 μg, about 941 μg, about 942 μg, about 943 μg, about 944 μg, about 945 μg, about 946 μg, about 947 μg, about 948 μg, about 949 μg, about 950 μg, about 951 μg, about 952 μg, about 953 μg, about 954 μg, about 955 μg, about 956 μg, about 957 μg, about 958 μg, about 959 μg, about 960 μg, about 961 μg, about 962 μg, about 963 μg, about 964 μg, about 965 μg, about 966 μg, about 967 μg, about 968 μg, about 969 μg, about 970 μg, about 971 μg, about 972 μg, about 973 μg, about 974 μg, about 975 μg, about 976 μg, about 977 μg, about 978 μg, about 979 μg, about 980 μg, about 981 μg, about 982 μg, about 983 μg, about 984 μg, about 985 μg, about 986 μg, about 987 μg, about 988 μg, about 989 μg, about 990 μg, about 991 μg, about 992 μg, about 993 μg, about 994 μg, about 995 μg, about 996 μg, about 997 μg, about 998 μg, about 999 μg, about 1000 μg, of total RSV F glycoproteins, including all values and ranges therebetween.

In embodiments, the immunogenic compositions described herein comprise from about 1 μg to about 400 μg, from 1 μg to about 350 μg, from 1 μg to about 300 μg, from 1 μg to about 250 μg, from 1 μg to about 200 μg, from 1 μg to about 150 μg, from 1 μg to about 100 μg, from 1 μg to about 50 μg, or from 1 μg to about 25 μg of adjuvant. In embodiments, the immunogenic compositions comprise about 1 μg, about 2 μg, about 3 μg, about 4 μg, about 5 μg, about 6 μg, about 7 μg, about 8 μg, about 9 μg, about 10 μg, about 11 μg, about 12 μg, about 13 μg, about 14 μg, about 15 μg, about 16 μg, about 17 μg, about 18 μg, about 19 μg, about 20 μg, about 21 μg, about 22 μg, about 23 μg, about 24 μg, about 25 μg, about 26 μg, about 27 μg, about 28 μg, about 29 μg, about 30 μg, about 31 μg, about 32 μg, about 33 μg, about 34 μg, about 35 μg, about 36 μg, about 37 μg, about 38 μg, about 39 μg, about 40 μg, about 41 μg, about 42 μg, about 43 μg, about 44 μg, about 45 μg, about 46 μg, about 47 μg, about 48 μg, about 49 μg, about 50 μg, about 51 μg, about 52 μg, about 53 μg, about 54 μg, about 55 μg, about 56 μg, about 57 μg, about 58 μg, about 59 μg, about 60 μg, about 61 μg, about 62 μg, about 63 μg, about 64 μg, about 65 μg, about 66 μg, about 67 μg, about 68 μg, about 69 μg, about 70 μg, about 71 μg, about 72 μg, about 73 μg, about 74 μg, about 75 μg, about 76 μg, about 77 μg, about 78 μg, about 79 μg, about 80 μg, about 81 μg, about 82 μg, about 83 μg, about 84 μg, about 85 μg, about 86 μg, about 87 μg, about 88 μg, about 89 μg, about 90 μg, about 91 μg, about 92 μg, about 93 μg, about 94 μg, about 95 μg, about 96 μg, about 97 μg, about 98 μg, about 99 μg, about 100 μg, about 101 μg, about 102 μg, about 103 μg, about 104 μg, about 105 μg, about 106 μg, about 107 μg, about 108 μg, about 109 μg, about 110 μg, about 111 μg, about 112 μg, about 113 μg, about 114 μg, about 115 μg, about 116 μg, about 117 μg, about 118 μg, about 119 μg, about 120 μg, about 121 μg, about 122 μg, about 123 μg, about 124 μg, about 125 μg, about 126 μg, about 127 μg, about 128 μg, about 129 μg, about 130 μg, about 131 μg, about 132 μg, about 133 μg, about 134 μg, about 135 μg, about 136 μg, about 137 μg, about 138 μg, about 139 μg, about 140 μg, about 141 μg, about 142 μg, about 143 μg, about 144 μg, about 145 μg, about 146 μg, about 147 μg, about 148 μg, about 149 μg, about 150 μg, about 151 μg, about 152 μg, about 153 μg, about 154 μg, about 155 μg, about 156 μg, about 157 μg, about 158 μg, about 159 μg, about 160 μg, about 161 μg, about 162 μg, about 163 μg, about 164 μg, about 165 μg, about 166 μg, about 167 μg, about 168 μg, about 169 μg, about 170 μg, about 171 μg, about 172 μg, about 173 μg, about 174 μg, about 175 μg, about 176 μg, about 177 μg, about 178 μg, about 179 μg, about 180 μg, about 181 μg, about 182 μg, about 183 μg, about 184 μg, about 185 μg, about 186 μg, about 187 μg, about 188 μg, about 189 μg, about 190 μg, about 191 μg, about 192 μg, about 193 μg, about 194 μg, about 195 μg, about 196 μg, about 197 μg, about 198 μg, about 199 μg, about 200 μg, about 201 μg, about 202 μg, about 203 μg, about 204 μg, about 205 μg, about 206 μg, about 207 μg, about 208 μg, about 209 μg, about 210 μg, about 211 μg, about 212 μg, about 213 μg, about 214 μg, about 215 μg, about 216 μg, about 217 μg, about 218 μg, about 219 μg, about 220 μg, about 221 μg, about 222 μg, about 223 μg, about 224 μg, about 225 μg, about 226 μg, about 227 μg, about 228 μg, about 229 μg, about 230 μg, about 231 μg, about 232 μg, about 233 μg, about 234 μg, about 235 μg, about 236 μg, about 237 μg, about 238 μg, about 239 μg, about 240 μg, about 241 μg, about 242 μg, about 243 μg, about 244 μg, about 245 μg, about 246 μg, about 247 μg, about 248 μg, about 249 μg, about 250 μg, about 251 μg, about 252 μg, about 253 μg, about 254 μg, about 255 μg, about 256 μg, about 257 μg, about 258 μg, about 259 μg, about 260 μg, about 261 μg, about 262 μg, about 263 μg, about 264 μg, about 265 μg, about 266 μg, about 267 μg, about 268 μg, about 269 μg, about 270 μg, about 271 μg, about 272 μg, about 273 μg, about 274 μg, about 275 μg, about 276 μg, about 277 μg, about 278 μg, about 279 μg, about 280 μg, about 281 μg, about 282 μg, about 283 μg, about 284 μg, about 285 μg, about 286 μg, about 287 μg, about 288 μg, about 289 μg, about 290 μg, about 291 μg, about 292 μg, about 293 μg, about 294 μg, about 295 μg, about 296 μg, about 297 μg, about 298 μg, about 299 μg, about 300 μg, about 301 μg, about 302 μg, about 303 μg, about 304 μg, about 305 μg, about 306 μg, about 307 μg, about 308 μg, about 309 μg, about 310 μg, about 311 μg, about 312 μg, about 313 μg, about 314 μg, about 315 μg, about 316 μg, about 317 μg, about 318 μg, about 319 μg, about 320 μg, about 321 μg, about 322 μg, about 323 μg, about 324 μg, about 325 μg, about 326 μg, about 327 μg, about 328 μg, about 329 μg, about 330 μg, about 331 μg, about 332 μg, about 333 μg, about 334 μg, about 335 μg, about 336 μg, about 337 μg, about 338 μg, about 339 μg, about 340 μg, about 341 μg, about 342 μg, about 343 μg, about 344 μg, about 345 μg, about 346 μg, about 347 μg, about 348 μg, about 349 μg, about 350 μg, about 351 μg, about 352 μg, about 353 μg, about 354 μg, about 355 μg, about 356 μg, about 357 μg, about 358 μg, about 359 μg, about 360 μg, about 361 μg, about 362 μg, about 363 μg, about 364 μg, about 365 μg, about 366 μg, about 367 μg, about 368 μg, about 369 μg, about 370 μg, about 371 μg, about 372 μg, about 373 μg, about 374 μg, about 375 μg, about 376 μg, about 377 μg, about 378 μg, about 379 μg, about 380 μg, about 381 μg, about 382 μg, about 383 μg, about 384 μg, about 385 μg, about 386 μg, about 387 μg, about 388 μg, about 389 μg, about 390 μg, about 391 μg, about 392 μg, about 393 μg, about 394 μg, about 395 μg, about 396 μg, about 397 μg, about 398 μg, about 399 μg, or about 400 μg of adjuvant. In embodiments, the immunogenic composition comprises about 50 μg of adjuvant. In embodiments, the immunogenic composition comprises about 75 μg of adjuvant.

In embodiments, the dose, as measured in μg, may be the total weight of the composition, or the weight of the nanoparticles, or the weight of the glycoprotein.

### C. Properties of Intranasal Immunogenic Compositions

In embodiments, intranasal immunogenic compositions are formulated as dry powder compositions, gels, creams, ointments, liquid solutions, or aerosolized preparations.

In embodiments, an intranasal immunogenic composition is a dry powder composition. In embodiments, particles of the dry powder have an average diameter from about 0.1 μm to about 250 μm. In embodiments, particles of the dry powder have an average diameter of about 0.1 μm, about 0.2 μm, about 0.3 μm, about 0.4 μm, about 0.5 μm, about 0.6 μm, about 0.7 μm, about 0.8 μm, about 0.9 μm, about 1 μm, about 2 μm, about 3 μm, about 4 μm, about 5 μm, about 6 μm, about 7 μm, about 8 μm, about 9 μm, about 10 μm, about 11 μm, about 12 μm, about 13 μm, about 14 μm, about 15 μm, about 16 μm, about 17 μm, about 18 μm, about 19 μm, about 20 μm, about 21 μm, about 22 μm, about 23 μm, about 24 μm, about 25 μm, about 26 μm, about 27 μm, about 28 μm, about 29 μm, about 30 μm, about 31 μm, about 32 μm, about 33 μm, about 34 μm, about 35 μm, about 36 μm, about 37 μm, about 38 μm, about 39 μm, about 40 μm, about 41 μm, about 42 μm, about 43 μm, about 44 μm, about 45 μm, about 46 μm, about 47 μm, about 48 μm, about 49 μm, about 50 μm, about 51 μm, about 52 μm, about 53 μm, about 54 μm, about 55 μm, about 56 μm, about 57 μm, about 58 μm, about 59 μm, about 60 μm, about 61 μm, about 62 μm, about 63 μm, about 64 μm, about 65 μm, about 66 μm, about 67 μm, about 68 μm, about 69 μm, about 70 μm, about 71 μm, about 72 μm, about 73 μm, about 74 μm, about 75 μm, about 76 μm, about 77 μm, about 78 μm, about 79 μm, about 80 μm, about 81 μm, about 82 μm, about 83 μm, about 84 μm, about 85 μm, about 86 μm, about 87 μm, about 88 μm, about 89 μm, about 90 μm, about 91 μm, about 92 μm, about 93 μm, about 94 μm, about 95 μm, about 96 μm, about 97 μm, about 98 μm, about 99 μm, about 100 μm, about 101 μm, about 102 μm, about 103 μm, about 104 μm, about 105 μm, about 106 μm, about 107 μm, about 108 μm, about 109 μm, about 110 μm, about 111 μm, about 112 μm, about 113 μm, about 114 μm, about 115 μm, about 116 μm, about 117 μm, about 118 μm, about 119 μm, about 120 μm, about 121 μm, about 122 μm, about 123 μm, about 124 μm, about 125 μm, about 126 μm, about 127 μm, about 128 μm, about 129 μm, about 130 μm, about 131 μm, about 132 μm, about 133 μm, about 134 μm, about 135 μm, about 136 μm, about 137 μm, about 138 μm, about 139 μm, about 140 μm, about 141 μm, about 142 μm, about 143 μm, about 144 μm, about 145 μm, about 146 μm, about 147 μm, about 148 μm, about 149 μm, about 150 μm, about 151 μm, about 152 μm, about 153 μm, about 154 μm, about 155 μm, about 156 μm, about 157 μm, about 158 μm, about 159 μm, about 160 μm, about 161 μm, about 162 μm, about 163 μm, about 164 μm, about 165 μm, about 166 μm, about 167 μm, about 168 μm, about 169 μm, about 170 μm, about 171 μm, about 172 μm, about 173 μm, about 174 μm, about 175 μm, about 176 μm, about 177 μm, about 178 μm, about 179 μm, about 180 μm, about 181 μm, about 182 μm, about 183 μm, about 184 μm, about 185 μm, about 186 μm, about 187 μm, about 188 μm, about 189 μm, about 190 μm, about 191 μm, about 192 μm, about 193 μm, about 194 μm, about 195 μm, about 196 μm, about 197 μm, about 198 μm, about 199 μm, about 200 μm, about 201 μm, about 202 μm, about 203 μm, about 204 μm, about 205 μm, about 206 μm, about 207 μm, about 208 μm, about 209 μm, about 210 μm, about 211 μm, about 212 μm, about 213 μm, about 214 μm, about 215 μm, about 216 μm, about 217 μm, about 218 μm, about 219 μm, about 220 μm, about 221 μm, about 222 μm, about 223 μm, about 224 μm, about 225 μm, about 226 μm, about 227 μm, about 228 μm, about 229 μm, about 230 μm, about 231 μm, about 232 μm, about 233 μm, about 234 μm, about 235 μm, about 236 μm, about 237 μm, about 238 μm, about 239 μm, about 240 μm, about 241 μm, about 242 μm, about 243 μm, about 244 μm, about 245 μm, about 246 μm, about 247 μm, about 248 μm, about 249 μm, or about 250 μm, including all values and subranges therebetween.

In embodiments, dry powder compositions comprise a dispersant.

In embodiments, the intranasal immunogenic compositions are formulated as liquids. In embodiments, a single dose of intranasal immunogenic composition comprises from about 0.05 mL to about 1 mL. In embodiments, a single dose of the intranasal immunogenic composition comprises about 0.05 mL, about 0.1 mL, about 0.2 mL, about 0.3 mL, about 0.4 mL, about 0.5 mL, about 0.6 mL, about 0.7 mL, about 0.8 mL, about 0.9 mL, or about 1 mL.

In embodiments, the intranasal immunogenic compositions comprise from about 10 μg/mL to about 4 mg/mL of total viral glycoprotein. In embodiments, the intranasal immunogenic compositions comprise from about 10 μg/mL to about 50 μg/mL of total viral glycoprotein. In embodiments, the intranasal immunogenic compositions comprise from about 120 μg/mL to about 600 μg/mL of total viral glycoprotein. In embodiments, the intranasal immunogenic compositions comprise from about 600 μg/mL to about 3 mg/mL of total viral glycoprotein. In embodiments, the intranasal immunogenic compositions comprise about 10 μg/mL, about 20 μg/mL, about 30 μg/mL, about 40 μg/mL, about 50 μg/mL, about 60 μg/mL, about 70 μg/mL, about 80 μg/mL, about 90 μg/mL, about 100 μg/mL, about 110 μg/mL, about 120 μg/mL, about 130 μg/mL, about 140 μg/mL, about 150 μg/mL, about 160 μg/mL, about 170 μg/mL, about 180 μg/mL, about 190 μg/mL, about 200 μg/mL, about 210 μg/mL, about 220 μg/mL, about 230 μg/mL, about 240 μg/mL, about 250 μg/mL, about 260 μg/mL, about 270 μg/mL, about 280 μg/mL, about 290 μg/mL, about 300 μg/mL, about 310 μg/mL, about 320 μg/mL, about 330 μg/mL, about 340 μg/mL, about 350 μg/mL, about 360 μg/mL, about 370 μg/mL, about 380 μg/mL, about 390 μg/mL, about 400 μg/mL, about 410 μg/mL, about 420 μg/mL, about 430 μg/mL, about 440 μg/mL, about 450 μg/mL, about 460 μg/mL, about 470 μg/mL, about 480 μg/mL, about 490 μg/mL, about 500 μg/mL, about 510 μg/mL, about 520 μg/mL, about 530 μg/mL, about 540 μg/mL, about 550 μg/mL, about 560 μg/mL, about 570 μg/mL, about 580 μg/mL, about 590 μg/mL, about 600 μg/mL, about 610 μg/mL, about 620 μg/mL, about 630 μg/mL, about 640 μg/mL, about 650 μg/mL, about 660 μg/mL, about 670 μg/mL, about 680 μg/mL, about 690 μg/mL, about 700 μg/mL, about 710 μg/mL, about 720 μg/mL, about 730 μg/mL, about 740 μg/mL, about 750 μg/mL, about 760 μg/mL, about 770 μg/mL, about 780 μg/mL, about 790 μg/mL, about 800 μg/mL, about 810 μg/mL, about 820 μg/mL, about 830 μg/mL, about 840 μg/mL, about 850 μg/mL, about 860 μg/mL, about 870 μg/mL, about 880 μg/mL, about 890 μg/mL, about 900 μg/mL, about 910 μg/mL, about 920 μg/mL, about 930 μg/mL, about 940 μg/mL, about 950 μg/mL, about 960 μg/mL, about 970 μg/mL, about 980 μg/mL, about 990 μg/mL, about 1000 μg/mL, about 1100 μg/mL, about 1200 μg/mL, about 1300 μg/mL, about 1400 μg/mL, about 1500 μg/mL, about 1600 μg/mL, about 1700 μg/mL, about 1800 μg/mL, about 1900 μg/mL, about 2000 μg/mL, about 2100 μg/mL, about 2200 μg/mL, about 2300 μg/mL, about 2400 μg/mL, about 2500 μg/mL, about 2600 μg/mL, about 2700 μg/mL, about 2800 μg/mL, about 2900 μg/mL, about 3000 μg/mL, about 3100 μg/mL, about 3200 μg/mL, about 3300 μg/mL, about 3400 μg/mL, about 3500 μg/mL, about 3600 μg/mL, about 3700 μg/mL, about 3800 μg/mL, about 3900 μg/mL, or about 4000 μg/mL of total viral glycoprotein, including all values and ranges therebetween.

In embodiments, the intranasal immunogenic compositions comprise from about 10 μg/mL to about 4 mg/mL of each viral glycoprotein. In embodiments, the intranasal immunogenic compositions comprise from about 10 μg/mL to about 50 μg/mL of each viral glycoprotein. In embodiments, the intranasal immunogenic compositions comprise from about 120 μg/mL to about 600 μg/mL of each viral glycoprotein. In embodiments, the intranasal immunogenic compositions comprise from about 600 μg/mL to about 3 mg/mL of each viral glycoprotein. In embodiments, the intranasal immunogenic compositions comprise about 10 μg/mL, about 20 μg/mL, about 30 μg/mL, about 40 μg/mL, about 50 μg/mL, about 60 μg/mL, about 70 μg/mL, about 80 μg/mL, about 90 μg/mL, about 100 μg/mL, about 110 μg/mL, about 120 μg/mL, about 130 μg/mL, about 140 μg/mL, about 150 μg/mL, about 160 μg/mL, about 170 μg/mL, about 180 μg/mL, about 190 μg/mL, about 200 μg/mL, about 210 μg/mL, about 220 μg/mL, about 230 μg/mL, about 240 μg/mL, about 250 μg/mL, about 260 μg/mL, about 270 μg/mL, about 280 μg/mL, about 290 μg/mL, about 300 μg/mL, about 310 μg/mL, about 320 μg/mL, about 330 μg/mL, about 340 μg/mL, about 350 μg/mL, about 360 μg/mL, about 370 μg/mL, about 380 μg/mL, about 390 μg/mL, about 400 μg/mL, about 410 μg/mL, about 420 μg/mL, about 430 μg/mL, about 440 μg/mL, about 450 μg/mL, about 460 μg/mL, about 470 μg/mL, about 480 μg/mL, about 490 μg/mL, about 500 μg/mL, about 510 μg/mL, about 520 μg/mL, about 530 μg/mL, about 540 μg/mL, about 550 μg/mL, about 560 μg/mL, about 570 μg/mL, about 580 μg/mL, about 590 μg/mL, about 600 μg/mL, about 610 μg/mL, about 620 μg/mL, about 630 μg/mL, about 640 μg/mL, about 650 μg/mL, about 660 μg/mL, about 670 μg/mL, about 680 μg/mL, about 690 μg/mL, about 700 μg/mL, about 710 μg/mL, about 720 μg/mL, about 730 μg/mL, about 740 μg/mL, about 750 μg/mL, about 760 μg/mL, about 770 μg/mL, about 780 μg/mL, about 790 μg/mL, about 800 μg/mL, about 810 μg/mL, about 820 μg/mL, about 830 μg/mL, about 840 μg/mL, about 850 μg/mL, about 860 μg/mL, about 870 μg/mL, about 880 μg/mL, about 890 μg/mL, about 900 μg/mL, about 910 μg/mL, about 920 μg/mL, about 930 μg/mL, about 940 μg/mL, about 950 μg/mL, about 960 μg/mL, about 970 μg/mL, about 980 μg/mL, about 990 μg/mL, about 1000 μg/mL, about 1100 μg/mL, about 1200 μg/mL, about 1300 μg/mL, about 1400 μg/mL, about 1500 μg/mL, about 1600 μg/mL, about 1700 μg/mL, about 1800 μg/mL, about 1900 μg/mL, about 2000 μg/mL, about 2100 μg/mL, about 2200 μg/mL, about 2300 μg/mL, about 2400 μg/mL, about 2500 μg/mL, about 2600 μg/mL, about 2700 μg/mL, about 2800 μg/mL, about 2900 μg/mL, about 3000 μg/mL, about 3100 μg/mL, about 3200 μg/mL, about 3300 μg/mL, about 3400 μg/mL, about 3500 μg/mL, about 3600 μg/mL, about 3700 μg/mL, about 3800 μg/mL, about 3900 μg/mL, or about 4000 μg/mL of each viral glycoprotein, including all values and ranges therebetween.

In embodiments, the intranasal immunogenic composition comprises atomized particles. In embodiments, the intranasal immunogenic composition comprises aerosolized particles. U.S. Publication No. 2021/0146068 describes properties of aerosols and atomized particles and is incorporated by reference herein in its entirety for all purposes.

### D. Intranasal Devices

In embodiments, provided herein are intranasal delivery devices comprising an intranasal immunogenic composition described herein. In embodiments, the delivery device is an intranasal device for the administration of liquids. Non-limiting examples of devices useful for the administration of liquid compositions include vapor devices (e.g., vapor inhalers), drop devices (e.g., catheters, single-dose droppers, multi-dose droppers, and unit-dose pipettes), mechanical spray pump devices (e.g., squeeze bottles, multi-dose metered-dose spray pumps, and single/duo-dose spray pumps), bi-directional spray pumps (e.g., breath-actuated nasal delivery devices), gas-driven spray systems/atomizers (e.g., single- or multi-dose HFA or nitrogen propellant-driven metered-dose inhalers, including traditional and circumferential velocity inhalers), and electrically powered nebulizers/atomizers (e.g., pulsation membrane nebulizers, vibrating mechanical nebulizers, and hand-held mechanical nebulizers). In embodiments, the delivery device is an intranasal device for the administration of powders or gels. Non-limiting examples of devices useful for the administration of powder compositions (e.g., lyophilized or otherwise dried intranasal immunogenic compositions) include mechanical powder sprayers (e.g., hand-actuated capsule-based powder spray devices and hand-actuated powder spray devices, hand actuated gel delivery devices), breath-actuated inhalers (e.g., single- or multi-dose nasal inhalers and capsule-based single- or multi-dose nasal inhalers), and insufflators (e.g., breath-actuated nasal delivery devices). In embodiments, the intranasal delivery device is pressurized. Additional intranasal delivery devices are described in International Publication No. 2012/105236 and U.S. Pat. No. 10,441,436, which are incorporated by reference herein in their entireties for all purposes. In embodiments, the intranasal delivery device is a prefilled syringe comprising the intranasal immunogenic composition.

In embodiments, the intranasal device is a single-use, disposable device. In another embodiment, the intranasal device is a multi- or repeat-use device. In embodiments, the single-use or multi-use device is pre-metered. In embodiments, the single-use or multi-use device is pre-filled. In certain embodiments, the multi- or repeat-use device is refillable.

Methods for configuring pressurized delivery devices to achieve a particular delivery profile are known in the field. In embodiments, a pressurized nasal delivery device is configured to produce a stream, spray, puff, etc., have a particular characteristic. For example, in one embodiment, to achieve administration to the upper third of the nasal epithelium, the device is configured to produce a strong, focused stream, spray, puff, etc. In one embodiment, the strong focused spray is created by imparting circumferential and/or axial velocity onto the stream of the therapeutic composition being administered into the nose. In another embodiment, to achieve administration to a greater portion of the nasal epithelium (e.g., the entire or the lower two thirds of the nasal epithelium), the device is configured to produce a diffuse and/or weaker stream, spray, puff, etc. In some embodiments, the tip of the delivery device is configured to physically direct the stream, spray, puff, etc., to the desired intranasal location when inserted into the subject's nose. For example, a kink or bend may be introduced into the tip of the delivery device to “point” the stream, spray, puff, etc., at a targeted epithelium. In some embodiments, the delivery pattern of the device is adjustable, such that the device can be differentially configured to target the intranasal immunogenic composition to a particular epithelium, structure, or location within the nose. In certain embodiments, the pooled human immunoglobulin compositions are administered by a breath-powered technology device. In certain embodiments, the breath-powered technology provides positive pressure during administration. In certain embodiments, the positive pressure expands narrow nasal passages. In some embodiments, exhalation into the device propels the therapeutic into the nose, while at the same time closing the soft-palette, thereby reducing deposition of the therapeutic into the throat and/or lungs. In one embodiment, the breath-powered technology device administers an intranasal immunogenic composition described herein into one nostril. In one embodiment, the breath-powered technology device administers an intranasal immunogenic composition described herein into two nostrils.

Non-limiting examples of commercial intranasal delivery devices include the EQUADEL® nasal spray pump (Aptar Pharma), the Solovent dry powder device (BD Technologies), the Unidose nasal drug delivery device (Consort Medical PLC), the NasoNeb® Nasal Nebulizer (MedInvent, LLC), the VeriDoser® nasal delivery device (Mystic Pharmaceuticals), the VRx2TM nasal delivery device (Mystic Pharmaceuticals), the DirectHaler™ Nasal device (Direct-Haler A/S), the TriViar™ single-use unit-dose dry powder inhaler (Trimel Pharmaceuticals), the SinuStar™ Aerosol Delivery System (Pari USA), the Aero Pump (Aero Pump GmbH), the Fit-Lizer™ nasal delivery device (Shin Nippon Biomedical Laboratories), the LMA MAD Nasal™ device (LMA North America, Inc.), the Compleo intranasal bioadhesive gel delivery system (Trimel Pharmaceuticals), Impel's Pressurized Olfactory Delivery (POD) device (Impel Neuropharma), the ViaNase™ electronic atomizer (Kurve Technology, Inc.), the OptiNose powder delivery device (OptiNose US Inc.), and the Optinose liquid delivery device (OptiNose US Inc.). In embodiments, the intranasal delivery device is a BD Accuspray™ Nasal Spray System. In embodiments, the intranasal delivery device is an intranasal mucosal atomization device (e.g., a MAD Nasal™ atomization device or the BD Accuspray™ Nasal Spray System). When intranasal immunogenic compositions described herein are administered using an intranasal mucosal atomization device, the composition is delivered to the nasal cavity as an atomized mist.

In embodiments, the intranasal delivery device delivers the intranasal immunogenic composition to the upper respiratory tract. In embodiments, the intranasal delivery device delivers the intranasal immunogenic composition to the lower respiratory tract. In embodiments, the intranasal delivery device delivers the intranasal immunogenic composition to the lungs.

### E. Methods of Administration of Intranasal Immunogenic Compositions

In embodiments, the disclosure provides a method for eliciting an immune response against a virus, comprising intranasally administering an immunogenic composition described herein. In embodiments, the virus is SARS-COV-2 or a variant thereof, influenza, RSV, or a combination thereof.

In embodiments, the immunogenic compositions lack an adjuvant. In embodiments, the immunogenic compositions comprise an adjuvant. Non-limiting examples of adjuvants are described herein.

In embodiments, the immunogenic compositions described herein have an efficacy against a virus that is between about 50% and about 99%, between about 80% and about 99%, between about 75% and about 99%, between about 80% and about 95%, between about 90% and about 98%, between about 75% and about 95%, at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99%.

Compositions may be administered on a single dose schedule or a multiple dose schedule. Multiple doses may be used in a primary immunization schedule or in a booster immunization schedule. In embodiments, about 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 doses are administered. In embodiments, doses are administered via the same route. For example, the first dose is intranasal, and the second dose is intranasal. In embodiments, doses are administered via different routes. For example, the first dose and the second dose are administered intramuscularly, and the third dose is administered intranasally.

In embodiments, the immunogenic compositions described herein are administered about 2 weeks, about 3 weeks, about 4 weeks, about 5 weeks, about 6 weeks, about 7 weeks, about 8 weeks, about 9 weeks, about 10 weeks, about 11 weeks, about 12 weeks, about 13 weeks, about 14 weeks, about 15 weeks, about 16 weeks, about 17 weeks, about 18 weeks, about 19 weeks, about 20 weeks, about 21 weeks, about 22 weeks, about 23 weeks, about 24 weeks, about 25 weeks, about 26 weeks, about 27 weeks, about 28 weeks, about 29 weeks, about 30 weeks, about 31 weeks, about 32 weeks, about 33 weeks, about 34 weeks, about 35 weeks, about 36, weeks, about 37 weeks, about 38 weeks, about 39 weeks, about 40 weeks, about 41 weeks, about 42 weeks, about 43 weeks, about 44 weeks, about 45 weeks, about 46 weeks, about 47 weeks, about 48 weeks, about 49 weeks, about 50 weeks, about 51 weeks, about 52 weeks, about 1 year, about 18 months, about 2 years, about 2.5 years, about 3 years, about 3.5 years, about 4 years, about 4.5 years, about 5 years, about 5.5 years, about 6 years, about 6.5 years, about 7 years, about 7.5 years, about 8 years, about 8.5 years, about 9 years, about 9.5 years, or about 10 years after a first, second, or subsequent dose of an immunogenic composition.

In embodiments, a boost dose is administered about 2 weeks, about 3 weeks, about 4 weeks, about 5 weeks, about 6 weeks, about 2 months, about 3 months, about 4 months, about 5 months, about 6 months, about 7 months, about 8 months, about 9 months, about 10 months, about 11 months, about 12 months (1 year), about 2 years, about 3 years, about 4 years, about 5 years, about 6 years, about 7 years, about 8 years, about 9 years, or about 10 years after the first dose. In embodiments, a boost dose is administered every year after administration of the initial dose. In embodiments, the follow-on boost dose is administered 3 weeks or 4 weeks after administration of the prior dose. In embodiments, the first dose is administered at day 0, and the boost dose is administered at day 21. In embodiments, the first dose is administered at day 0, and the boost dose is administered at day 28. In embodiments, the first dose is administered at day 0, a boost dose is administered at day 21, and a second boost dose is administered about six months after administration of the first dose or second dose. In embodiments, the first dose is administered at day 0, and the boost dose is administered at day 28, and a second boost dose is administered about six months after administration of the first dose. In embodiments, the first dose is administered at day 0, a boost dose is administered at day 21, and a second boost dose is administered about six months after administration of the second dose. In embodiments, the first dose is administered at day 0, and the boost dose is administered at day 28, and a second boost dose is administered about six months after administration of the second dose.

In embodiments, the first dose is administered at day 0, a boost dose is administered at day 21, and a second boost dose is administered about 1 year after administration of the first dose or the first boost dose. In embodiments, the first dose is administered at day 0, a first boost dose is administered at day 28, and a second boost dose is administered about 1 year after administration of the first dose. In embodiments, the first dose is administered at day 0, a boost dose is administered at day 21, and a second boost dose is administered about 1 year after administration of the second dose. In embodiments, the first dose is administered at day 0, a first boost dose is administered at day 28, and a second boost dose is administered about 1 year after administration of the second dose. In embodiments, the second boost dose is administered from 6 months to 24 months or from 12 to 24 months after the first boost dose.

In embodiments, the boost dose comprises the same immunogenic composition as the initial dose. In embodiments, the boost dose comprises a different immunogenic composition than the initial dose. In embodiments, the initial or boost dose comprises an immunogenic composition comprising a SARS-COV-2 Spike glycoprotein, an mRNA encoding a SARS-Cov-2 Spike glycoprotein, a plasmid DNA encoding a SARS-Cov-2 Spike glycoprotein, an viral vector encoding a SARS-Cov-2 Spike glycoprotein, or an inactivated SARS-COV-2 virus. In embodiments, the boost dose comprises the initial immunogenic composition. In embodiments, the initial dose comprises a SARS-COV-2 S glycoprotein (e.g., a SARS COV-2 S glycoprotein having the amino acid sequence of SEQ ID NO: 87), and the boost dose comprises the same SARS-CoV-2 S glycoprotein (e.g., a SARS COV-2 S glycoprotein having the amino acid sequence of SEQ ID NO: 87). In embodiments, the initial dose comprises a SARS-COV-2 S glycoprotein (e.g., a SARS COV-2 S glycoprotein having the amino acid sequence of SEQ ID NO: 87), and the boost dose comprises a different SARS-COV-2 S glycoprotein (e.g., a SARS COV-2 S glycoprotein having the amino acid sequence of SEQ ID NO: 132).

In embodiments, the initial dose comprises a combination of SARS-COV-2 S glycoproteins (e.g., a SARS COV-2 S glycoprotein having the amino acid sequence of SEQ ID NO: 87 and a SARS COV-2 S glycoprotein having the amino acid sequence of SEQ ID NO: 132). In embodiments, the boost dose comprises a combination of SARS-COV-2 S glycoproteins (e.g., a SARS COV-2 S glycoprotein having the amino acid sequence of SEQ ID NO: 87 and a SARS CoV-2 S glycoprotein having the amino acid sequence of SEQ ID NO: 132). In embodiments, the initial dose comprises a SARS-COV-2 S glycoprotein, a plasmid DNA encoding a SARS-Cov-2 S glycoprotein, an viral vector encoding a SARS-COV-2 Spike glycoprotein, or an inactivated SARS-COV-2 virus. In embodiments, the initial dose comprises a SARS-COV-2 Spike glycoprotein, a plasmid DNA encoding a SARS-COV-2 Spike glycoprotein, an viral vector encoding a SARS-Cov-2 Spike glycoprotein, or an inactivated SARS-COV-2 virus, and the boost dose comprises one or more SARS-COV-2 S glycoproteins.

In embodiments, intranasal administration of the composition includes administration of a liquid drop of the composition directly onto the nasal epithelium. In embodiments, the methods comprise administering from 1 to 100, from 1 to 90, from 1 to 80, from 1 to 70, from 1 to 60, from 1 to 50, from 1 to 40, from 1 to 30, from 1 to 20, from 1 to 10, or from 1 to 5 drops of the immunogenic composition are intranasally. In embodiments, the liquid drop is delivered by an intranasal delivery device described herein.

In embodiments, the methods comprise administering an aerosol of an immunogenic composition described herein to the nasal epithelium. In embodiments, the aerosol is a liquid aerosol. In embodiments, the aerosol is a powder aerosol. A powder aerosol comprises a dry powder composition. In embodiments, the methods comprise administering from 1 to 100, from 1 to 90, from 1 to 80, from 1 to 70, from 1 to 60, from 1 to 50, from 1 to 40, from 1 to 30, from 1 to 20, from 1 to 10, or from 1 to 5 sprays of the aerosol intranasally. In embodiments, the aerosol is delivered by an intranasal delivery device described herein.

In embodiments, the methods comprise administering an intranasal immunogenic composition that is in solution. In embodiments, the methods comprise administering an intranasal immunogenic composition that is in suspension.

In embodiments, the methods comprise intranasally administering a mist or puff of an intranasal immunogenic composition to a patient's nostril. In embodiments, the methods comprise intranasally administering from 1 to about 100 mists or puffs of an intranasal immunogenic composition to a patient's nostril. In embodiments, the methods comprise intranasally administering about 1, about 2, about 3, about 4, about 5, about 6, about 7, about 8, about 9, about 10, about 11, about 12, about 13, about 14, about 15, about 16, about 17, about 18, about 19, about 20, about 21, about 22, about 23, about 24, about 25, about 26, about 27, about 28, about 29, about 30, about 31, about 32, about 33, about 34, about 35, about 36, about 37, about 38, about 39, about 40, about 41, about 42, about 43, about 44, about 45, about 46, about 47, about 48, about 49, about 50, about 51, about 52, about 53, about 54, about 55, about 56, about 57, about 58, about 59, about 60, about 61, about 62, about 63, about 64, about 65, about 66, about 67, about 68, about 69, about 70, about 71, about 72, about 73, about 74, about 75, about 76, about 77, about 78, about 79, about 80, about 81, about 82, about 83, about 84, about 85, about 86, about 87, about 88, about 89, about 90, about 91, about 92, about 93, about 94, about 95, about 96, about 97, about 98, about 99, or about 100 puffs or mists, including all values and ranges therebetween, of an intranasal immunogenic composition to a patient's nostril. In embodiments, a mist or puff is delivered to each of the patient's nostrils.

In embodiments, the methods described herein allow the immunogenic composition to reach the lungs of a patient. In embodiments, the methods described herein allow the immunogenic composition to reach the upper respiratory tract of a patient. In embodiments, the methods described herein allow the immunogenic composition to reach the lower respiratory tract of a patient.

In embodiments, the methods comprise intranasally administering from 0.05 mL to about 1 mL of an intranasal immunogenic composition described herein. In embodiments, the methods comprise intranasally administering from about 0.05 mL, about 0.1 mL, about 0.2 mL, about 0.3 mL, about 0.4 mL, about 0.5 mL, about 0.6 mL, about 0.7 mL, about 0.8 mL, about 0.9 mL, or about 1 mL of an intranasal immunogenic composition described herein, including all values and ranges therebetween. In embodiments the methods comprise intranasally administering 0.2 mL of an intranasal immunogenic composition described herein. In embodiments the methods comprise intranasally administering 0.5 mL of an intranasal immunogenic composition described herein. In embodiments the methods comprise intranasally administering 0.25 mL of an intranasal immunogenic composition described herein to each nostril of a patient.

In embodiments, provided herein are methods of producing IgA and/or IgG antibodies against SARS-COV-2, influenza, and/or RSV in a subject. In embodiments, the antibodies are neutralizing antibodies. In embodiments, provided herein are methods of inducing mucosal immunity against SARS-COV-2, influenza, RSV, or a combination thereof in a patient comprising intranasally administering the intranasal immunogenic composition described herein. In embodiments, the antibodies are located in the bronchoalveolar lavage of the subject.

In some embodiments, the disclosure provides co-formulation (i.e., prefilled syringes or pre-mix) strategies for intranasal immunogenic compositions comprising nanoparticles. Typical vaccine administration strategies currently being utilized are bedside mix formulations. That is, vaccine compositions and adjuvants are stored separately and are mixed prior to administration. Pre-mix, co-formulation, or prefilled syringe strategies for vaccine are less common due to the concerns of the stability of the antigens and their subsequent immunogenic capabilities. The present disclosure provides intranasal immunogenic compositions that can be pre-mixed and stored in advance. The disclosed vaccination strategies and formulations may improve the efficiency of vaccination and may reduce the risks of bedside mixing errors, while maintaining the overall safety and immunogenicity.

A variety of containers may be used to store and transport the pre-mix formulations, including syringes for single administrations and plastic ampules. In some instances, plastic ampules can be manufactured using the blow-fill-seal manufacturing technique or method. In general, the blow-fill-seal (BFS) manufacturing method includes extruding a plastic material (e.g., resin) to form a parison, which is then placed into a mold and cut to size. A filling needle or mandrel is then used to inflate the plastic, which in turn, results in a hollow ampule that substantially conforms to the shape of the mold. Once inflated, a desired volume of liquid can be injected into the ampule, the filling needle or mandrel can be removed, and the ampule can be sealed. Accordingly, BFS can be an automated process that can be performed in a sterile environment without direct human intervention.

In some instances, the ability to aseptically manufacture sterile ampules containing a desired liquid can make BFS manufactured ampules particularly well suited for the pharmaceutical industry. BFS technology, however, has not been compatible with all pharmaceutical liquids, products, etc. For example, some known BFS manufacturing methods include delivering the liquid or product into the ampule while the plastic is still relatively hot, which can result in adverse effects to temperature sensitive liquids and/or products such as vaccines, biologics, etc. Advances in cool BFS technology, however, have increased the variety of suitable products, liquids, etc. allowing some vaccines, biologics, and/or other temperature sensitive pharmaceuticals to be contained in BFS ampules.

In some instances, a BFS ampule can have a size, shape, and/or configuration that is at least partially based on a desired use and/or a desired pharmaceutical liquid or dosage that the ampule is configured to contain. For example, some known BFS ampules can include a pierce through top, a twist-off top, a top including a male or female luer, and/or the like. Some known BFS ampules can have a size and/or shape based on volume of the liquid or dosage configured to be disposed therein. In addition, some known BFS ampules can be manufactured in a strip of multiple, temporarily connected ampules, which can increase manufacturing, packaging, and/or storing efficiencies and/or the like.

In embodiments, the immunogenic compositions described herein are provided in pre-filled syringes. When the immunogenic composition is prepared in a pre-filled syringe, an antigen and adjuvant is combined in advance of administration. In embodiments, the pre-filled syringe contains hemagglutinin, a SARS-COV-2 S glycoprotein, an RSV F glycoprotein, or a combination thereof.

In embodiments, a subject is administered an intranasal immunogenic composition from a pre-filled syringe.

In embodiments, the methods comprise administering from about 1 μg to about 400 μg, from 1 μg to about 350 μg, from 1 μg to about 300 μg, from 1 μg to about 250 μg, from 1 μg to about 200 μg, from 1 μg to about 150 μg, from 1 μg to about 100 μg, from 1 μg to about 50 μg, or from 1 μg to about 25 μg of each viral glycoprotein. In embodiments, the methods comprise administering about 1 μg, about 2 μg, about 3 μg, about 4 μg, about 5 μg, about 6 μg, about 7 μg, about 8 μg, about 9 μg, about 10 μg, about 11 μg, about 12 μg, about 13 μg, about 14 μg, about 15 μg, about 16 μg, about 17 μg, about 18 μg, about 19 μg, about 20 μg, about 21 μg, about 22 μg, about 23 μg, about 24 μg, about 25 μg, about 26 μg, about 27 μg, about 28 μg, about 29 μg, about 30 μg, about 31 μg, about 32 μg, about 33 μg, about 34 μg, about 35 μg, about 36 μg, about 37 μg, about 38 μg, about 39 μg, about 40 μg, about 41 μg, about 42 μg, about 43 μg, about 44 μg, about 45 μg, about 46 μg, about 47 μg, about 48 μg, about 49 μg, about 50 μg, about 51 μg, about 52 μg, about 53 μg, about 54 μg, about 55 μg, about 56 μg, about 57 μg, about 58 μg, about 59 μg, about 60 μg, about 61 μg, about 62 μg, about 63 μg, about 64 μg, about 65 μg, about 66 μg, about 67 μg, about 68 μg, about 69 μg, about 70 μg, about 71 μg, about 72 μg, about 73 μg, about 74 μg, about 75 μg, about 76 μg, about 77 μg, about 78 μg, about 79 μg, about 80 μg, about 81 μg, about 82 μg, about 83 μg, about 84 μg, about 85 μg, about 86 μg, about 87 μg, about 88 μg, about 89 μg, about 90 μg, about 91 μg, about 92 μg, about 93 μg, about 94 μg, about 95 μg, about 96 μg, about 97 μg, about 98 μg, about 99 μg, about 100 μg, about 101 μg, about 102 μg, about 103 μg, about 104 μg, about 105 μg, about 106 μg, about 107 μg, about 108 μg, about 109 μg, about 110 μg, about 111 μg, about 112 μg, about 113 μg, about 114 μg, about 115 μg, about 116 μg, about 117 μg, about 118 μg, about 119 μg, about 120 μg, about 121 μg, about 122 μg, about 123 μg, about 124 μg, about 125 μg, about 126 μg, about 127 μg, about 128 μg, about 129 μg, about 130 μg, about 131 μg, about 132 μg, about 133 μg, about 134 μg, about 135 μg, about 136 μg, about 137 μg, about 138 μg, about 139 μg, about 140 μg, about 141 μg, about 142 μg, about 143 μg, about 144 μg, about 145 μg, about 146 μg, about 147 μg, about 148 μg, about 149 μg, about 150 μg, about 151 μg, about 152 μg, about 153 μg, about 154 μg, about 155 μg, about 156 μg, about 157 μg, about 158 μg, about 159 μg, about 160 μg, about 161 μg, about 162 μg, about 163 μg, about 164 μg, about 165 μg, about 166 μg, about 167 μg, about 168 μg, about 169 μg, about 170 μg, about 171 μg, about 172 μg, about 173 μg, about 174 μg, about 175 μg, about 176 μg, about 177 μg, about 178 μg, about 179 μg, about 180 μg, about 181 μg, about 182 μg, about 183 μg, about 184 μg, about 185 μg, about 186 μg, about 187 μg, about 188 μg, about 189 μg, about 190 μg, about 191 μg, about 192 μg, about 193 μg, about 194 μg, about 195 μg, about 196 μg, about 197 μg, about 198 μg, about 199 μg, about 200 μg, about 201 μg, about 202 μg, about 203 μg, about 204 μg, about 205 μg, about 206 μg, about 207 μg, about 208 μg, about 209 μg, about 210 μg, about 211 μg, about 212 μg, about 213 μg, about 214 μg, about 215 μg, about 216 μg, about 217 μg, about 218 μg, about 219 μg, about 220 μg, about 221 μg, about 222 μg, about 223 μg, about 224 μg, about 225 μg, about 226 μg, about 227 μg, about 228 μg, about 229 μg, about 230 μg, about 231 μg, about 232 μg, about 233 μg, about 234 μg, about 235 μg, about 236 μg, about 237 μg, about 238 μg, about 239 μg, about 240 μg, about 241 μg, about 242 μg, about 243 μg, about 244 μg, about 245 μg, about 246 μg, about 247 μg, about 248 μg, about 249 μg, about 250 μg, about 251 μg, about 252 μg, about 253 μg, about 254 μg, about 255 μg, about 256 μg, about 257 μg, about 258 μg, about 259 μg, about 260 μg, about 261 μg, about 262 μg, about 263 μg, about 264 μg, about 265 μg, about 266 μg, about 267 μg, about 268 μg, about 269 μg, about 270 μg, about 271 μg, about 272 μg, about 273 μg, about 274 μg, about 275 μg, about 276 μg, about 277 μg, about 278 μg, about 279 μg, about 280 μg, about 281 μg, about 282 μg, about 283 μg, about 284 μg, about 285 μg, about 286 μg, about 287 μg, about 288 μg, about 289 μg, about 290 μg, about 291 μg, about 292 μg, about 293 μg, about 294 μg, about 295 μg, about 296 μg, about 297 μg, about 298 μg, about 299 μg, about 300 μg, about 301 μg, about 302 μg, about 303 μg, about 304 μg, about 305 μg, about 306 μg, about 307 μg, about 308 μg, about 309 μg, about 310 μg, about 311 μg, about 312 μg, about 313 μg, about 314 μg, about 315 μg, about 316 μg, about 317 μg, about 318 μg, about 319 μg, about 320 μg, about 321 μg, about 322 μg, about 323 μg, about 324 μg, about 325 μg, about 326 μg, about 327 μg, about 328 μg, about 329 μg, about 330 μg, about 331 μg, about 332 μg, about 333 μg, about 334 μg, about 335 μg, about 336 μg, about 337 μg, about 338 μg, about 339 μg, about 340 μg, about 341 μg, about 342 μg, about 343 μg, about 344 μg, about 345 μg, about 346 μg, about 347 μg, about 348 μg, about 349 μg, about 350 μg, about 351 μg, about 352 μg, about 353 μg, about 354 μg, about 355 μg, about 356 μg, about 357 μg, about 358 μg, about 359 μg, about 360 μg, about 361 μg, about 362 μg, about 363 μg, about 364 μg, about 365 μg, about 366 μg, about 367 μg, about 368 μg, about 369 μg, about 370 μg, about 371 μg, about 372 μg, about 373 μg, about 374 μg, about 375 μg, about 376 μg, about 377 μg, about 378 μg, about 379 μg, about 380 μg, about 381 μg, about 382 μg, about 383 μg, about 384 μg, about 385 μg, about 386 μg, about 387 μg, about 388 μg, about 389 μg, about 390 μg, about 391 μg, about 392 μg, about 393 μg, about 394 μg, about 395 μg, about 396 μg, about 397 μg, about 398 μg, about 399 μg, or about 400 μg of each viral glycoprotein.

In embodiments, the methods comprise administering about 1 μg of each viral glycoprotein. In embodiments, the methods comprise administering about 5 μg of each viral glycoprotein. In embodiments, the methods comprise administering about 25 μg of each viral glycoprotein. In embodiments, the methods comprise administering about 60 μg of each viral glycoprotein. In embodiments, the methods comprise administering about 240 μg of each viral glycoprotein.

In embodiments, the methods comprise administering from about 1 μg to about 100 μg of each CoV S glycoprotein. In embodiments, the methods comprise administering from about 2.5 μg to about 35 μg of each CoV S glycoprotein. In embodiments, the methods comprise administering from about 5 μg to about 25 μg of each CoV S glycoprotein. In embodiments, the methods comprise administering from about 14 μg to about 15 μg of each CoV S glycoprotein. In embodiments, the methods comprise administering from about 15 μg to about 35 μg of each CoV S glycoprotein. In embodiments, the methods comprise administering from about 1 μg to about 100 μg of total CoV S glycoproteins. In embodiments, the methods comprise administering about 1 μg, about 2 μg, about 3 μg, about 4 μg, about 5 μg, about 6 μg, about 7 μg, about 8 μg, about 9 μg, about 10 μg, about 11 μg, about 12 μg, about 13 μg, about 14 μg, about 15 μg, about 16 μg, about 17 μg, about 18 μg, about 19 μg, about 20 μg, about 21 μg, about 22 μg, about 23 μg, about 24 μg, about 25 μg, about 26 μg, about 27 μg, about 28 μg, about 29 μg, about 30 μg, about 31 μg, about 32 μg, about 33 μg, about 34 μg, about 35 μg, about 36 μg, about 37 μg, about 38 μg, about 39 μg, about 40 μg, about 41 μg, about 42 μg, about 43 μg, about 44 μg, about 45 μg, about 46 μg, about 47 μg, about 48 μg, about 49 μg, about 50 μg, about 51 μg, about 52 μg, about 53 μg, about 54 μg, about 55 μg, about 56 μg, about 57 μg, about 58 μg, about 59 μg, about 60 μg, about 61 μg, about 62 μg, about 63 μg, about 64 μg, about 65 μg, about 66 μg, about 67 μg, about 68 μg, about 69 μg, about 70 μg, about 71 μg, about 72 μg, about 73 μg, about 74 μg, about 75 μg, about 76 μg, about 77 μg, about 78 μg, about 79 μg, about 80 μg, about 81 μg, about 82 μg, about 83 μg, about 84 μg, about 85 μg, about 86 μg, about 87 μg, about 88 μg, about 89 μg, about 90 μg, about 91 μg, about 92 μg, about 93 μg, about 94 μg, about 95 μg, about 96 μg, about 97 μg, about 98 μg, about 99 μg, about 100 μg, including all values and ranges therebetween, of each CoV S glycoprotein. In embodiments, the methods comprise administering about 1 μg, about 2 μg, about 3 μg, about 4 μg, about 5 μg, about 6 μg, about 7 μg, about 8 μg, about 9 μg, about 10 μg, about 11 μg, about 12 μg, about 13 μg, about 14 μg, about 15 μg, about 16 μg, about 17 μg, about 18 μg, about 19 μg, about 20 μg, about 21 μg, about 22 μg, about 23 μg, about 24 μg, about 25 μg, about 26 μg, about 27 μg, about 28 μg, about 29 μg, about 30 μg, about 31 μg, about 32 μg, about 33 μg, about 34 μg, about 35 μg, about 36 μg, about 37 μg, about 38 μg, about 39 μg, about 40 μg, about 41 μg, about 42 μg, about 43 μg, about 44 μg, about 45 μg, about 46 μg, about 47 μg, about 48 μg, about 49 μg, about 50 μg, about 51 μg, about 52 μg, about 53 μg, about 54 μg, about 55 μg, about 56 μg, about 57 μg, about 58 μg, about 59 μg, about 60 μg, about 61 μg, about 62 μg, about 63 μg, about 64 μg, about 65 μg, about 66 μg, about 67 μg, about 68 μg, about 69 μg, about 70 μg, about 71 μg, about 72 μg, about 73 μg, about 74 μg, about 75 μg, about 76 μg, about 77 μg, about 78 μg, about 79 μg, about 80 μg, about 81 μg, about 82 μg, about 83 μg, about 84 μg, about 85 μg, about 86 μg, about 87 μg, about 88 μg, about 89 μg, about 90 μg, about 91 μg, about 92 μg, about 93 μg, about 94 μg, about 95 μg, about 96 μg, about 97 μg, about 98 μg, about 99 μg, about 100 μg of total CoV S glycoproteins, including all values and ranges therebetween. In embodiments, the methods comprise administering about 1 μg of a CoV S glycoprotein. In embodiments, the methods comprise administering about 13 μg of a CoV S glycoprotein. In embodiments, the immunogenic compositions comprise about 5 μg of a CoV S glycoprotein. In embodiments, the methods comprise administering about 25 μg of a CoV S glycoprotein.

In embodiments, the methods comprise administering from about 1 μg to about 300 μg of each HA glycoprotein. In embodiments, the methods comprise administering from about 5 μg to about 60 μg of each HA glycoprotein. In embodiments, the methods comprise administering from about 24 μg to about 40 μg of each HA glycoprotein. In embodiments, the methods comprise administering from about 30 μg to about 40 μg of each HA glycoprotein. In embodiments, the methods comprise administering from about 30 μg to about 60 μg of each HA glycoprotein. In embodiments, the methods comprise administering from about 33 μg to about 39 μg of each HA glycoprotein. In embodiments, the methods comprise administering from about 20 μg to about 240 μg of each HA glycoprotein. In embodiments, the methods comprise administering from about 30 μg to about 60 μg of each HA glycoprotein. In embodiments, the methods comprise administering from about 24 μg to about 40 μg of each HA glycoprotein. In embodiments, the methods comprise administering about 1 μg, about 2 μg, about 3 μg, about 4 μg, about 5 μg, about 6 μg, about 7 μg, about 8 μg, about 9 μg, about 10 μg, about 11 μg, about 12 μg, about 13 μg, about 14 μg, about 15 μg, about 16 μg, about 17 μg, about 18 μg, about 19 μg, about 20 μg, about 21 μg, about 22 μg, about 23 μg, about 24 μg, about 25 μg, about 26 μg, about 27 μg, about 28 μg, about 29 μg, about 30 μg, about 31 μg, about 32 μg, about 33 μg, about 34 μg, about 35 μg, about 36 μg, about 37 μg, about 38 μg, about 39 μg, about 40 μg, about 41 μg, about 42 μg, about 43 μg, about 44 μg, about 45 μg, about 46 μg, about 47 μg, about 48 μg, about 49 μg, about 50 μg, about 51 μg, about 52 μg, about 53 μg, about 54 μg, about 55 μg, about 56 μg, about 57 μg, about 58 μg, about 59 μg, about 60 μg, about 61 μg, about 62 μg, about 63 μg, about 64 μg, about 65 μg, about 66 μg, about 67 μg, about 68 μg, about 69 μg, about 70 μg, about 71 μg, about 72 μg, about 73 μg, about 74 μg, about 75 μg, about 76 μg, about 77 μg, about 78 μg, about 79 μg, about 80 μg, about 81 μg, about 82 μg, about 83 μg, about 84 μg, about 85 μg, about 86 μg, about 87 μg, about 88 μg, about 89 μg, about 90 μg, about 91 μg, about 92 μg, about 93 μg, about 94 μg, about 95 μg, about 96 μg, about 97 μg, about 98 μg, about 99 μg, about 100 μg, about 101 μg, about 102 μg, about 103 μg, about 104 μg, about 105 μg, about 106 μg, about 107 μg, about 108 μg, about 109 μg, about 110 μg, about 111 μg, about 112 μg, about 113 μg, about 114 μg, about 115 μg, about 116 μg, about 117 μg, about 118 μg, about 119 μg, about 120 μg, about 121 μg, about 122 μg, about 123 μg, about 124 μg, about 125 μg, about 126 μg, about 127 μg, about 128 μg, about 129 μg, about 130 μg, about 131 μg, about 132 μg, about 133 μg, about 134 μg, about 135 μg, about 136 μg, about 137 μg, about 138 μg, about 139 μg, about 140 μg, about 141 μg, about 142 μg, about 143 μg, about 144 μg, about 145 μg, about 146 μg, about 147 μg, about 148 μg, about 149 μg, about 150 μg, about 151 μg, about 152 μg, about 153 μg, about 154 μg, about 155 μg, about 156 μg, about 157 μg, about 158 μg, about 159 μg, about 160 μg, about 161 μg, about 162 μg, about 163 μg, about 164 μg, about 165 μg, about 166 μg, about 167 μg, about 168 μg, about 169 μg, about 170 μg, about 171 μg, about 172 μg, about 173 μg, about 174 μg, about 175 μg, about 176 μg, about 177 μg, about 178 μg, about 179 μg, about 180 μg, about 181 μg, about 182 μg, about 183 μg, about 184 μg, about 185 μg, about 186 μg, about 187 μg, about 188 μg, about 189 μg, about 190 μg, about 191 μg, about 192 μg, about 193 μg, about 194 μg, about 195 μg, about 196 μg, about 197 μg, about 198 μg, about 199 μg, about 200 μg, about 201 μg, about 202 μg, about 203 μg, about 204 μg, about 205 μg, about 206 μg, about 207 μg, about 208 μg, about 209 μg, about 210 μg, about 211 μg, about 212 μg, about 213 μg, about 214 μg, about 215 μg, about 216 μg, about 217 μg, about 218 μg, about 219 μg, about 220 μg, about 221 μg, about 222 μg, about 223 μg, about 224 μg, about 225 μg, about 226 μg, about 227 μg, about 228 μg, about 229 μg, about 230 μg, about 231 μg, about 232 μg, about 233 μg, about 234 μg, about 235 μg, about 236 μg, about 237 μg, about 238 μg, about 239 μg, about 240 μg, about 241 μg, about 242 μg, about 243 μg, about 244 μg, about 245 μg, about 246 μg, about 247 μg, about 248 μg, about 249 μg, about 250 μg, about 251 μg, about 252 μg, about 253 μg, about 254 μg, about 255 μg, about 256 μg, about 257 μg, about 258 μg, about 259 μg, about 260 μg, about 261 μg, about 262 μg, about 263 μg, about 264 μg, about 265 μg, about 266 μg, about 267 μg, about 268 μg, about 269 μg, about 270 μg, about 271 μg, about 272 μg, about 273 μg, about 274 μg, about 275 μg, about 276 μg, about 277 μg, about 278 μg, about 279 μg, about 280 μg, about 281 μg, about 282 μg, about 283 μg, about 284 μg, about 285 μg, about 286 μg, about 287 μg, about 288 μg, about 289 μg, about 290 μg, about 291 μg, about 292 μg, about 293 μg, about 294 μg, about 295 μg, about 296 μg, about 297 μg, about 298 μg, about 299 μg, or about 300 μg of each HA glycoprotein, including all values and ranges therebetween. In embodiments, the methods comprise administering about 30 μg of each HA glycoprotein. In embodiments, the methods comprise administering about 33 μg of each HA glycoprotein. In embodiments, the methods comprise administering about 39 μg of each HA glycoprotein. In embodiments, the methods comprise administering about 54 μg of each HA glycoprotein. In embodiments, the methods comprise administering about 60 μg of each HA glycoprotein. In embodiments, the methods comprise administering about 240 μg of each HA glycoprotein.

In embodiments, the methods comprise administering from about 1 μg to about 1000 μg of total HA glycoproteins. In embodiments, the methods comprise administering about 1 μg, about 2 μg, about 3 μg, about 4 μg, about 5 μg, about 6 μg, about 7 μg, about 8 μg, about 9 μg, about 10 μg, about 11 μg, about 12 μg, about 13 μg, about 14 μg, about 15 μg, about 16 μg, about 17 μg, about 18 μg, about 19 μg, about 20 μg, about 21 μg, about 22 μg, about 23 μg, about 24 μg, about 25 μg, about 26 μg, about 27 μg, about 28 μg, about 29 μg, about 30 μg, about 31 μg, about 32 μg, about 33 μg, about 34 μg, about 35 μg, about 36 μg, about 37 μg, about 38 μg, about 39 μg, about 40 μg, about 41 μg, about 42 μg, about 43 μg, about 44 μg, about 45 μg, about 46 μg, about 47 μg, about 48 μg, about 49 μg, about 50 μg, about 51 μg, about 52 μg, about 53 μg, about 54 μg, about 55 μg, about 56 μg, about 57 μg, about 58 μg, about 59 μg, about 60 μg, about 61 μg, about 62 μg, about 63 μg, about 64 μg, about 65 μg, about 66 μg, about 67 μg, about 68 μg, about 69 μg, about 70 μg, about 71 μg, about 72 μg, about 73 μg, about 74 μg, about 75 μg, about 76 μg, about 77 μg, about 78 μg, about 79 μg, about 80 μg, about 81 μg, about 82 μg, about 83 μg, about 84 μg, about 85 μg, about 86 μg, about 87 μg, about 88 μg, about 89 μg, about 90 μg, about 91 μg, about 92 μg, about 93 μg, about 94 μg, about 95 μg, about 96 μg, about 97 μg, about 98 μg, about 99 μg, about 100 μg, about 101 μg, about 102 μg, about 103 μg, about 104 μg, about 105 μg, about 106 μg, about 107 μg, about 108 μg, about 109 μg, about 110 μg, about 111 μg, about 112 μg, about 113 μg, about 114 μg, about 115 μg, about 116 μg, about 117 μg, about 118 μg, about 119 μg, about 120 μg, about 121 μg, about 122 μg, about 123 μg, about 124 μg, about 125 μg, about 126 μg, about 127 μg, about 128 μg, about 129 μg, about 130 μg, about 131 μg, about 132 μg, about 133 μg, about 134 μg, about 135 μg, about 136 μg, about 137 μg, about 138 μg, about 139 μg, about 140 μg, about 141 μg, about 142 μg, about 143 μg, about 144 μg, about 145 μg, about 146 μg, about 147 μg, about 148 μg, about 149 μg, about 150 μg, about 151 μg, about 152 μg, about 153 μg, about 154 μg, about 155 μg, about 156 μg, about 157 μg, about 158 μg, about 159 μg, about 160 μg, about 161 μg, about 162 μg, about 163 μg, about 164 μg, about 165 μg, about 166 μg, about 167 μg, about 168 μg, about 169 μg, about 170 μg, about 171 μg, about 172 μg, about 173 μg, about 174 μg, about 175 μg, about 176 μg, about 177 μg, about 178 μg, about 179 μg, about 180 μg, about 181 μg, about 182 μg, about 183 μg, about 184 μg, about 185 μg, about 186 μg, about 187 μg, about 188 μg, about 189 μg, about 190 μg, about 191 μg, about 192 μg, about 193 μg, about 194 μg, about 195 μg, about 196 μg, about 197 μg, about 198 μg, about 199 μg, about 200 μg, about 201 μg, about 202 μg, about 203 μg, about 204 μg, about 205 μg, about 206 μg, about 207 μg, about 208 μg, about 209 μg, about 210 μg, about 211 μg, about 212 μg, about 213 μg, about 214 μg, about 215 μg, about 216 μg, about 217 μg, about 218 μg, about 219 μg, about 220 μg, about 221 μg, about 222 μg, about 223 μg, about 224 μg, about 225 μg, about 226 μg, about 227 μg, about 228 μg, about 229 μg, about 230 μg, about 231 μg, about 232 μg, about 233 μg, about 234 μg, about 235 μg, about 236 μg, about 237 μg, about 238 μg, about 239 μg, about 240 μg, about 241 μg, about 242 μg, about 243 μg, about 244 μg, about 245 μg, about 246 μg, about 247 μg, about 248 μg, about 249 μg, about 250 μg, about 251 μg, about 252 μg, about 253 μg, about 254 μg, about 255 μg, about 256 μg, about 257 μg, about 258 μg, about 259 μg, about 260 μg, about 261 μg, about 262 μg, about 263 μg, about 264 μg, about 265 μg, about 266 μg, about 267 μg, about 268 μg, about 269 μg, about 270 μg, about 271 μg, about 272 μg, about 273 μg, about 274 μg, about 275 μg, about 276 μg, about 277 μg, about 278 μg, about 279 μg, about 280 μg, about 281 μg, about 282 μg, about 283 μg, about 284 μg, about 285 μg, about 286 μg, about 287 μg, about 288 μg, about 289 μg, about 290 μg, about 291 μg, about 292 μg, about 293 μg, about 294 μg, about 295 μg, about 296 μg, about 297 μg, about 298 μg, about 299 μg, about 300 μg, about 301 μg, about 302 μg, about 303 μg, about 304 μg, about 305 μg, about 306 μg, about 307 μg, about 308 μg, about 309 μg, about 310 μg, about 311 μg, about 312 μg, about 313 μg, about 314 μg, about 315 μg, about 316 μg, about 317 μg, about 318 μg, about 319 μg, about 320 μg, about 321 μg, about 322 μg, about 323 μg, about 324 μg, about 325 μg, about 326 μg, about 327 μg, about 328 μg, about 329 μg, about 330 μg, about 331 μg, about 332 μg, about 333 μg, about 334 μg, about 335 μg, about 336 μg, about 337 μg, about 338 μg, about 339 μg, about 340 μg, about 341 μg, about 342 μg, about 343 μg, about 344 μg, about 345 μg, about 346 μg, about 347 μg, about 348 μg, about 349 μg, about 350 μg, about 351 μg, about 352 μg, about 353 μg, about 354 μg, about 355 μg, about 356 μg, about 357 μg, about 358 μg, about 359 μg, about 360 μg, about 361 μg, about 362 μg, about 363 μg, about 364 μg, about 365 μg, about 366 μg, about 367 μg, about 368 μg, about 369 μg, about 370 μg, about 371 μg, about 372 μg, about 373 μg, about 374 μg, about 375 μg, about 376 μg, about 377 μg, about 378 μg, about 379 μg, about 380 μg, about 381 μg, about 382 μg, about 383 μg, about 384 μg, about 385 μg, about 386 μg, about 387 μg, about 388 μg, about 389 μg, about 390 μg, about 391 μg, about 392 μg, about 393 μg, about 394 μg, about 395 μg, about 396 μg, about 397 μg, about 398 μg, about 399 μg, about 400 μg, about 401 μg, about 402 μg, about 403 μg, about 404 μg, about 405 μg, about 406 μg, about 407 μg, about 408 μg, about 409 μg, about 410 μg, about 411 μg, about 412 μg, about 413 μg, about 414 μg, about 415 μg, about 416 μg, about 417 μg, about 418 μg, about 419 μg, about 420 μg, about 421 μg, about 422 μg, about 423 μg, about 424 μg, about 425 μg, about 426 μg, about 427 μg, about 428 μg, about 429 μg, about 430 μg, about 431 μg, about 432 μg, about 433 μg, about 434 μg, about 435 μg, about 436 μg, about 437 μg, about 438 μg, about 439 μg, about 440 μg, about 441 μg, about 442 μg, about 443 μg, about 444 μg, about 445 μg, about 446 μg, about 447 μg, about 448 μg, about 449 μg, about 450 μg, about 451 μg, about 452 μg, about 453 μg, about 454 μg, about 455 μg, about 456 μg, about 457 μg, about 458 μg, about 459 μg, about 460 μg, about 461 μg, about 462 μg, about 463 μg, about 464 μg, about 465 μg, about 466 μg, about 467 μg, about 468 μg, about 469 μg, about 470 μg, about 471 μg, about 472 μg, about 473 μg, about 474 μg, about 475 μg, about 476 μg, about 477 μg, about 478 μg, about 479 μg, about 480 μg, about 481 μg, about 482 μg, about 483 μg, about 484 μg, about 485 μg, about 486 μg, about 487 μg, about 488 μg, about 489 μg, about 490 μg, about 491 μg, about 492 μg, about 493 μg, about 494 μg, about 495 μg, about 496 μg, about 497 μg, about 498 μg, about 499 μg, about 500 μg, about 501 μg, about 502 μg, about 503 μg, about 504 μg, about 505 μg, about 506 μg, about 507 μg, about 508 μg, about 509 μg, about 510 μg, about 511 μg, about 512 μg, about 513 μg, about 514 μg, about 515 μg, about 516 μg, about 517 μg, about 518 μg, about 519 μg, about 520 μg, about 521 μg, about 522 μg, about 523 μg, about 524 μg, about 525 μg, about 526 μg, about 527 μg, about 528 μg, about 529 μg, about 530 μg, about 531 μg, about 532 μg, about 533 μg, about 534 μg, about 535 μg, about 536 μg, about 537 μg, about 538 μg, about 539 μg, about 540 μg, about 541 μg, about 542 μg, about 543 μg, about 544 μg, about 545 μg, about 546 μg, about 547 μg, about 548 μg, about 549 μg, about 550 μg, about 551 μg, about 552 μg, about 553 μg, about 554 μg, about 555 μg, about 556 μg, about 557 μg, about 558 μg, about 559 μg, about 560 μg, about 561 μg, about 562 μg, about 563 μg, about 564 μg, about 565 μg, about 566 μg, about 567 μg, about 568 μg, about 569 μg, about 570 μg, about 571 μg, about 572 μg, about 573 μg, about 574 μg, about 575 μg, about 576 μg, about 577 μg, about 578 μg, about 579 μg, about 580 μg, about 581 μg, about 582 μg, about 583 μg, about 584 μg, about 585 μg, about 586 μg, about 587 μg, about 588 μg, about 589 μg, about 590 μg, about 591 μg, about 592 μg, about 593 μg, about 594 μg, about 595 μg, about 596 μg, about 597 μg, about 598 μg, about 599 μg, about 600 μg, about 601 μg, about 602 μg, about 603 μg, about 604 μg, about 605 μg, about 606 μg, about 607 μg, about 608 μg, about 609 μg, about 610 μg, about 611 μg, about 612 μg, about 613 μg, about 614 μg, about 615 μg, about 616 μg, about 617 μg, about 618 μg, about 619 μg, about 620 μg, about 621 μg, about 622 μg, about 623 μg, about 624 μg, about 625 μg, about 626 μg, about 627 μg, about 628 μg, about 629 μg, about 630 μg, about 631 μg, about 632 μg, about 633 μg, about 634 μg, about 635 μg, about 636 μg, about 637 μg, about 638 μg, about 639 μg, about 640 μg, about 641 μg, about 642 μg, about 643 μg, about 644 μg, about 645 μg, about 646 μg, about 647 μg, about 648 μg, about 649 μg, about 650 μg, about 651 μg, about 652 μg, about 653 μg, about 654 μg, about 655 μg, about 656 μg, about 657 μg, about 658 μg, about 659 μg, about 660 μg, about 661 μg, about 662 μg, about 663 μg, about 664 μg, about 665 μg, about 666 μg, about 667 μg, about 668 μg, about 669 μg, about 670 μg, about 671 μg, about 672 μg, about 673 μg, about 674 μg, about 675 μg, about 676 μg, about 677 μg, about 678 μg, about 679 μg, about 680 μg, about 681 μg, about 682 μg, about 683 μg, about 684 μg, about 685 μg, about 686 μg, about 687 μg, about 688 μg, about 689 μg, about 690 μg, about 691 μg, about 692 μg, about 693 μg, about 694 μg, about 695 μg, about 696 μg, about 697 μg, about 698 μg, about 699 μg, about 700 μg, about 701 μg, about 702 μg, about 703 μg, about 704 μg, about 705 μg, about 706 μg, about 707 μg, about 708 μg, about 709 μg, about 710 μg, about 711 μg, about 712 μg, about 713 μg, about 714 μg, about 715 μg, about 716 μg, about 717 μg, about 718 μg, about 719 μg, about 720 μg, about 721 μg, about 722 μg, about 723 μg, about 724 μg, about 725 μg, about 726 μg, about 727 μg, about 728 μg, about 729 μg, about 730 μg, about 731 μg, about 732 μg, about 733 μg, about 734 μg, about 735 μg, about 736 μg, about 737 μg, about 738 μg, about 739 μg, about 740 μg, about 741 μg, about 742 μg, about 743 μg, about 744 μg, about 745 μg, about 746 μg, about 747 μg, about 748 μg, about 749 μg, about 750 μg, about 751 μg, about 752 μg, about 753 μg, about 754 μg, about 755 μg, about 756 μg, about 757 μg, about 758 μg, about 759 μg, about 760 μg, about 761 μg, about 762 μg, about 763 μg, about 764 μg, about 765 μg, about 766 μg, about 767 μg, about 768 μg, about 769 μg, about 770 μg, about 771 μg, about 772 μg, about 773 μg, about 774 μg, about 775 μg, about 776 μg, about 777 μg, about 778 μg, about 779 μg, about 780 μg, about 781 μg, about 782 μg, about 783 μg, about 784 μg, about 785 μg, about 786 μg, about 787 μg, about 788 μg, about 789 μg, about 790 μg, about 791 μg, about 792 μg, about 793 μg, about 794 μg, about 795 μg, about 796 μg, about 797 μg, about 798 μg, about 799 μg, about 800 μg, about 801 μg, about 802 μg, about 803 μg, about 804 μg, about 805 μg, about 806 μg, about 807 μg, about 808 μg, about 809 μg, about 810 μg, about 811 μg, about 812 μg, about 813 μg, about 814 μg, about 815 μg, about 816 μg, about 817 μg, about 818 μg, about 819 μg, about 820 μg, about 821 μg, about 822 μg, about 823 μg, about 824 μg, about 825 μg, about 826 μg, about 827 μg, about 828 μg, about 829 μg, about 830 μg, about 831 μg, about 832 μg, about 833 μg, about 834 μg, about 835 μg, about 836 μg, about 837 μg, about 838 μg, about 839 μg, about 840 μg, about 841 μg, about 842 μg, about 843 μg, about 844 μg, about 845 μg, about 846 μg, about 847 μg, about 848 μg, about 849 μg, about 850 μg, about 851 μg, about 852 μg, about 853 μg, about 854 μg, about 855 μg, about 856 μg, about 857 μg, about 858 μg, about 859 μg, about 860 μg, about 861 μg, about 862 μg, about 863 μg, about 864 μg, about 865 μg, about 866 μg, about 867 μg, about 868 μg, about 869 μg, about 870 μg, about 871 μg, about 872 μg, about 873 μg, about 874 μg, about 875 μg, about 876 μg, about 877 μg, about 878 μg, about 879 μg, about 880 μg, about 881 μg, about 882 μg, about 883 μg, about 884 μg, about 885 μg, about 886 μg, about 887 μg, about 888 μg, about 889 μg, about 890 μg, about 891 μg, about 892 μg, about 893 μg, about 894 μg, about 895 μg, about 896 μg, about 897 μg, about 898 μg, about 899 μg, about 900 μg, about 901 μg, about 902 μg, about 903 μg, about 904 μg, about 905 μg, about 906 μg, about 907 μg, about 908 μg, about 909 μg, about 910 μg, about 911 μg, about 912 μg, about 913 μg, about 914 μg, about 915 μg, about 916 μg, about 917 μg, about 918 μg, about 919 μg, about 920 μg, about 921 μg, about 922 μg, about 923 μg, about 924 μg, about 925 μg, about 926 μg, about 927 μg, about 928 μg, about 929 μg, about 930 μg, about 931 μg, about 932 μg, about 933 μg, about 934 μg, about 935 μg, about 936 μg, about 937 μg, about 938 μg, about 939 μg, about 940 μg, about 941 μg, about 942 μg, about 943 μg, about 944 μg, about 945 μg, about 946 μg, about 947 μg, about 948 μg, about 949 μg, about 950 μg, about 951 μg, about 952 μg, about 953 μg, about 954 μg, about 955 μg, about 956 μg, about 957 μg, about 958 μg, about 959 μg, about 960 μg, about 961 μg, about 962 μg, about 963 μg, about 964 μg, about 965 μg, about 966 μg, about 967 μg, about 968 μg, about 969 μg, about 970 μg, about 971 μg, about 972 μg, about 973 μg, about 974 μg, about 975 μg, about 976 μg, about 977 μg, about 978 μg, about 979 μg, about 980 μg, about 981 μg, about 982 μg, about 983 μg, about 984 μg, about 985 μg, about 986 μg, about 987 μg, about 988 μg, about 989 μg, about 990 μg, about 991 μg, about 992 μg, about 993 μg, about 994 μg, about 995 μg, about 996 μg, about 997 μg, about 998 μg, about 999 μg, about 1000 μg, of total HA glycoproteins, including all values and ranges therebetween.

In embodiments, the methods comprise administering from about 1 μg to about 300 μg of each RSV F glycoprotein. In embodiments, the methods comprise administering from about 5 μg to about 60 μg of each RSV F glycoprotein. In embodiments, the methods comprise administering from about 24 μg to about 40 μg of each RSV F glycoprotein. In embodiments, the methods comprise administering from about 30 μg to about 40 μg of each RSV F glycoprotein. In embodiments, the methods comprise administering from about 30 μg to about 60 μg of each RSV F glycoprotein. In embodiments, the methods comprise administering from about 33 μg to about 39 μg of each RSV F glycoprotein. In embodiments, the methods comprise administering from about 20 μg to about 240 μg of each RSV F glycoprotein. In embodiments, the methods comprise administering from about 30 μg to about 60 μg of each RSV F glycoprotein. In embodiments, the methods comprise administering from about 24 μg to about 40 μg of each RSV F glycoprotein. In embodiments, the methods comprise administering about 1 μg, about 2 μg, about 3 μg, about 4 μg, about 5 μg, about 6 μg, about 7 μg, about 8 μg, about 9 μg, about 10 μg, about 11 μg, about 12 μg, about 13 μg, about 14 μg, about 15 μg, about 16 μg, about 17 μg, about 18 μg, about 19 μg, about 20 μg, about 21 μg, about 22 μg, about 23 μg, about 24 μg, about 25 μg, about 26 μg, about 27 μg, about 28 μg, about 29 μg, about 30 μg, about 31 μg, about 32 μg, about 33 μg, about 34 μg, about 35 μg, about 36 μg, about 37 μg, about 38 μg, about 39 μg, about 40 μg, about 41 μg, about 42 μg, about 43 μg, about 44 μg, about 45 μg, about 46 μg, about 47 μg, about 48 μg, about 49 μg, about 50 μg, about 51 μg, about 52 μg, about 53 μg, about 54 μg, about 55 μg, about 56 μg, about 57 μg, about 58 μg, about 59 μg, about 60 μg, about 61 μg, about 62 μg, about 63 μg, about 64 μg, about 65 μg, about 66 μg, about 67 μg, about 68 μg, about 69 μg, about 70 μg, about 71 μg, about 72 μg, about 73 μg, about 74 μg, about 75 μg, about 76 μg, about 77 μg, about 78 μg, about 79 μg, about 80 μg, about 81 μg, about 82 μg, about 83 μg, about 84 μg, about 85 μg, about 86 μg, about 87 μg, about 88 μg, about 89 μg, about 90 μg, about 91 μg, about 92 μg, about 93 μg, about 94 μg, about 95 μg, about 96 μg, about 97 μg, about 98 μg, about 99 μg, about 100 μg, about 101 μg, about 102 μg, about 103 μg, about 104 μg, about 105 μg, about 106 μg, about 107 μg, about 108 μg, about 109 μg, about 110 μg, about 111 μg, about 112 μg, about 113 μg, about 114 μg, about 115 μg, about 116 μg, about 117 μg, about 118 μg, about 119 μg, about 120 μg, about 121 μg, about 122 μg, about 123 μg, about 124 μg, about 125 μg, about 126 μg, about 127 μg, about 128 μg, about 129 μg, about 130 μg, about 131 μg, about 132 μg, about 133 μg, about 134 μg, about 135 μg, about 136 μg, about 137 μg, about 138 μg, about 139 μg, about 140 μg, about 141 μg, about 142 μg, about 143 μg, about 144 μg, about 145 μg, about 146 μg, about 147 μg, about 148 μg, about 149 μg, about 150 μg, about 151 μg, about 152 μg, about 153 μg, about 154 μg, about 155 μg, about 156 μg, about 157 μg, about 158 μg, about 159 μg, about 160 μg, about 161 μg, about 162 μg, about 163 μg, about 164 μg, about 165 μg, about 166 μg, about 167 μg, about 168 μg, about 169 μg, about 170 μg, about 171 μg, about 172 μg, about 173 μg, about 174 μg, about 175 μg, about 176 μg, about 177 μg, about 178 μg, about 179 μg, about 180 μg, about 181 μg, about 182 μg, about 183 μg, about 184 μg, about 185 μg, about 186 μg, about 187 μg, about 188 μg, about 189 μg, about 190 μg, about 191 μg, about 192 μg, about 193 μg, about 194 μg, about 195 μg, about 196 μg, about 197 μg, about 198 μg, about 199 μg, about 200 μg, about 201 μg, about 202 μg, about 203 μg, about 204 μg, about 205 μg, about 206 μg, about 207 μg, about 208 μg, about 209 μg, about 210 μg, about 211 μg, about 212 μg, about 213 μg, about 214 μg, about 215 μg, about 216 μg, about 217 μg, about 218 μg, about 219 μg, about 220 μg, about 221 μg, about 222 μg, about 223 μg, about 224 μg, about 225 μg, about 226 μg, about 227 μg, about 228 μg, about 229 μg, about 230 μg, about 231 μg, about 232 μg, about 233 μg, about 234 μg, about 235 μg, about 236 μg, about 237 μg, about 238 μg, about 239 μg, about 240 μg, about 241 μg, about 242 μg, about 243 μg, about 244 μg, about 245 μg, about 246 μg, about 247 μg, about 248 μg, about 249 μg, about 250 μg, about 251 μg, about 252 μg, about 253 μg, about 254 μg, about 255 μg, about 256 μg, about 257 μg, about 258 μg, about 259 μg, about 260 μg, about 261 μg, about 262 μg, about 263 μg, about 264 μg, about 265 μg, about 266 μg, about 267 μg, about 268 μg, about 269 μg, about 270 μg, about 271 μg, about 272 μg, about 273 μg, about 274 μg, about 275 μg, about 276 μg, about 277 μg, about 278 μg, about 279 μg, about 280 μg, about 281 μg, about 282 μg, about 283 μg, about 284 μg, about 285 μg, about 286 μg, about 287 μg, about 288 μg, about 289 μg, about 290 μg, about 291 μg, about 292 μg, about 293 μg, about 294 μg, about 295 μg, about 296 μg, about 297 μg, about 298 μg, about 299 μg, or about 300 μg of each RSV F glycoprotein, including all values and ranges therebetween. In embodiments, the methods comprise administering about 60 μg of each RSV F glycoprotein. In embodiments, the methods comprise administering about 240 μg of each RSV F glycoprotein.

In embodiments, the methods comprise administering from about 1 μg to about 1000 μg of total RSV F glycoproteins. In embodiments, the methods comprise administering about 1 μg, about 2 μg, about 3 μg, about 4 μg, about 5 μg, about 6 μg, about 7 μg, about 8 μg, about 9 μg, about 10 μg, about 11 μg, about 12 μg, about 13 μg, about 14 μg, about 15 μg, about 16 μg, about 17 μg, about 18 μg, about 19 μg, about 20 μg, about 21 μg, about 22 μg, about 23 μg, about 24 μg, about 25 μg, about 26 μg, about 27 μg, about 28 μg, about 29 μg, about 30 μg, about 31 μg, about 32 μg, about 33 μg, about 34 μg, about 35 μg, about 36 μg, about 37 μg, about 38 μg, about 39 μg, about 40 μg, about 41 μg, about 42 μg, about 43 μg, about 44 μg, about 45 μg, about 46 μg, about 47 μg, about 48 μg, about 49 μg, about 50 μg, about 51 μg, about 52 μg, about 53 μg, about 54 μg, about 55 μg, about 56 μg, about 57 μg, about 58 μg, about 59 μg, about 60 μg, about 61 μg, about 62 μg, about 63 μg, about 64 μg, about 65 μg, about 66 μg, about 67 μg, about 68 μg, about 69 μg, about 70 μg, about 71 μg, about 72 μg, about 73 μg, about 74 μg, about 75 μg, about 76 μg, about 77 μg, about 78 μg, about 79 μg, about 80 μg, about 81 μg, about 82 μg, about 83 μg, about 84 μg, about 85 μg, about 86 μg, about 87 μg, about 88 μg, about 89 μg, about 90 μg, about 91 μg, about 92 μg, about 93 μg, about 94 μg, about 95 μg, about 96 μg, about 97 μg, about 98 μg, about 99 μg, about 100 μg, about 101 μg, about 102 μg, about 103 μg, about 104 μg, about 105 μg, about 106 μg, about 107 μg, about 108 μg, about 109 μg, about 110 μg, about 111 μg, about 112 μg, about 113 μg, about 114 μg, about 115 μg, about 116 μg, about 117 μg, about 118 μg, about 119 μg, about 120 μg, about 121 μg, about 122 μg, about 123 μg, about 124 μg, about 125 μg, about 126 μg, about 127 μg, about 128 μg, about 129 μg, about 130 μg, about 131 μg, about 132 μg, about 133 μg, about 134 μg, about 135 μg, about 136 μg, about 137 μg, about 138 μg, about 139 μg, about 140 μg, about 141 μg, about 142 μg, about 143 μg, about 144 μg, about 145 μg, about 146 μg, about 147 μg, about 148 μg, about 149 μg, about 150 μg, about 151 μg, about 152 μg, about 153 μg, about 154 μg, about 155 μg, about 156 μg, about 157 μg, about 158 μg, about 159 μg, about 160 μg, about 161 μg, about 162 μg, about 163 μg, about 164 μg, about 165 μg, about 166 μg, about 167 μg, about 168 μg, about 169 μg, about 170 μg, about 171 μg, about 172 μg, about 173 μg, about 174 μg, about 175 μg, about 176 μg, about 177 μg, about 178 μg, about 179 μg, about 180 μg, about 181 μg, about 182 μg, about 183 μg, about 184 μg, about 185 μg, about 186 μg, about 187 μg, about 188 μg, about 189 μg, about 190 μg, about 191 μg, about 192 μg, about 193 μg, about 194 μg, about 195 μg, about 196 μg, about 197 μg, about 198 μg, about 199 μg, about 200 μg, about 201 μg, about 202 μg, about 203 μg, about 204 μg, about 205 μg, about 206 μg, about 207 μg, about 208 μg, about 209 μg, about 210 μg, about 211 μg, about 212 μg, about 213 μg, about 214 μg, about 215 μg, about 216 μg, about 217 μg, about 218 μg, about 219 μg, about 220 μg, about 221 μg, about 222 μg, about 223 μg, about 224 μg, about 225 μg, about 226 μg, about 227 μg, about 228 μg, about 229 μg, about 230 μg, about 231 μg, about 232 μg, about 233 μg, about 234 μg, about 235 μg, about 236 μg, about 237 μg, about 238 μg, about 239 μg, about 240 μg, about 241 μg, about 242 μg, about 243 μg, about 244 μg, about 245 μg, about 246 μg, about 247 μg, about 248 μg, about 249 μg, about 250 μg, about 251 μg, about 252 μg, about 253 μg, about 254 μg, about 255 μg, about 256 μg, about 257 μg, about 258 μg, about 259 μg, about 260 μg, about 261 μg, about 262 μg, about 263 μg, about 264 μg, about 265 μg, about 266 μg, about 267 μg, about 268 μg, about 269 μg, about 270 μg, about 271 μg, about 272 μg, about 273 μg, about 274 μg, about 275 μg, about 276 μg, about 277 μg, about 278 μg, about 279 μg, about 280 μg, about 281 μg, about 282 μg, about 283 μg, about 284 μg, about 285 μg, about 286 μg, about 287 μg, about 288 μg, about 289 μg, about 290 μg, about 291 μg, about 292 μg, about 293 μg, about 294 μg, about 295 μg, about 296 μg, about 297 μg, about 298 μg, about 299 μg, about 300 μg, about 301 μg, about 302 μg, about 303 μg, about 304 μg, about 305 μg, about 306 μg, about 307 μg, about 308 μg, about 309 μg, about 310 μg, about 311 μg, about 312 μg, about 313 μg, about 314 μg, about 315 μg, about 316 μg, about 317 μg, about 318 μg, about 319 μg, about 320 μg, about 321 μg, about 322 μg, about 323 μg, about 324 μg, about 325 μg, about 326 μg, about 327 μg, about 328 μg, about 329 μg, about 330 μg, about 331 μg, about 332 μg, about 333 μg, about 334 μg, about 335 μg, about 336 μg, about 337 μg, about 338 μg, about 339 μg, about 340 μg, about 341 μg, about 342 μg, about 343 μg, about 344 μg, about 345 μg, about 346 μg, about 347 μg, about 348 μg, about 349 μg, about 350 μg, about 351 μg, about 352 μg, about 353 μg, about 354 μg, about 355 μg, about 356 μg, about 357 μg, about 358 μg, about 359 μg, about 360 μg, about 361 μg, about 362 μg, about 363 μg, about 364 μg, about 365 μg, about 366 μg, about 367 μg, about 368 μg, about 369 μg, about 370 μg, about 371 μg, about 372 μg, about 373 μg, about 374 μg, about 375 μg, about 376 μg, about 377 μg, about 378 μg, about 379 μg, about 380 μg, about 381 μg, about 382 μg, about 383 μg, about 384 μg, about 385 μg, about 386 μg, about 387 μg, about 388 μg, about 389 μg, about 390 μg, about 391 μg, about 392 μg, about 393 μg, about 394 μg, about 395 μg, about 396 μg, about 397 μg, about 398 μg, about 399 μg, about 400 μg, about 401 μg, about 402 μg, about 403 μg, about 404 μg, about 405 μg, about 406 μg, about 407 μg, about 408 μg, about 409 μg, about 410 μg, about 411 μg, about 412 μg, about 413 μg, about 414 μg, about 415 μg, about 416 μg, about 417 μg, about 418 μg, about 419 μg, about 420 μg, about 421 μg, about 422 μg, about 423 μg, about 424 μg, about 425 μg, about 426 μg, about 427 μg, about 428 μg, about 429 μg, about 430 μg, about 431 μg, about 432 μg, about 433 μg, about 434 μg, about 435 μg, about 436 μg, about 437 μg, about 438 μg, about 439 μg, about 440 μg, about 441 μg, about 442 μg, about 443 μg, about 444 μg, about 445 μg, about 446 μg, about 447 μg, about 448 μg, about 449 μg, about 450 μg, about 451 μg, about 452 μg, about 453 μg, about 454 μg, about 455 μg, about 456 μg, about 457 μg, about 458 μg, about 459 μg, about 460 μg, about 461 μg, about 462 μg, about 463 μg, about 464 μg, about 465 μg, about 466 μg, about 467 μg, about 468 μg, about 469 μg, about 470 μg, about 471 μg, about 472 μg, about 473 μg, about 474 μg, about 475 μg, about 476 μg, about 477 μg, about 478 μg, about 479 μg, about 480 μg, about 481 μg, about 482 μg, about 483 μg, about 484 μg, about 485 μg, about 486 μg, about 487 μg, about 488 μg, about 489 μg, about 490 μg, about 491 μg, about 492 μg, about 493 μg, about 494 μg, about 495 μg, about 496 μg, about 497 μg, about 498 μg, about 499 μg, about 500 μg, about 501 μg, about 502 μg, about 503 μg, about 504 μg, about 505 μg, about 506 μg, about 507 μg, about 508 μg, about 509 μg, about 510 μg, about 511 μg, about 512 μg, about 513 μg, about 514 μg, about 515 μg, about 516 μg, about 517 μg, about 518 μg, about 519 μg, about 520 μg, about 521 μg, about 522 μg, about 523 μg, about 524 μg, about 525 μg, about 526 μg, about 527 μg, about 528 μg, about 529 μg, about 530 μg, about 531 μg, about 532 μg, about 533 μg, about 534 μg, about 535 μg, about 536 μg, about 537 μg, about 538 μg, about 539 μg, about 540 μg, about 541 μg, about 542 μg, about 543 μg, about 544 μg, about 545 μg, about 546 μg, about 547 μg, about 548 μg, about 549 μg, about 550 μg, about 551 μg, about 552 μg, about 553 μg, about 554 μg, about 555 μg, about 556 μg, about 557 μg, about 558 μg, about 559 μg, about 560 μg, about 561 μg, about 562 μg, about 563 μg, about 564 μg, about 565 μg, about 566 μg, about 567 μg, about 568 μg, about 569 μg, about 570 μg, about 571 μg, about 572 μg, about 573 μg, about 574 μg, about 575 μg, about 576 μg, about 577 μg, about 578 μg, about 579 μg, about 580 μg, about 581 μg, about 582 μg, about 583 μg, about 584 μg, about 585 μg, about 586 μg, about 587 μg, about 588 μg, about 589 μg, about 590 μg, about 591 μg, about 592 μg, about 593 μg, about 594 μg, about 595 μg, about 596 μg, about 597 μg, about 598 μg, about 599 μg, about 600 μg, about 601 μg, about 602 μg, about 603 μg, about 604 μg, about 605 μg, about 606 μg, about 607 μg, about 608 μg, about 609 μg, about 610 μg, about 611 μg, about 612 μg, about 613 μg, about 614 μg, about 615 μg, about 616 μg, about 617 μg, about 618 μg, about 619 μg, about 620 μg, about 621 μg, about 622 μg, about 623 μg, about 624 μg, about 625 μg, about 626 μg, about 627 μg, about 628 μg, about 629 μg, about 630 μg, about 631 μg, about 632 μg, about 633 μg, about 634 μg, about 635 μg, about 636 μg, about 637 μg, about 638 μg, about 639 μg, about 640 μg, about 641 μg, about 642 μg, about 643 μg, about 644 μg, about 645 μg, about 646 μg, about 647 μg, about 648 μg, about 649 μg, about 650 μg, about 651 μg, about 652 μg, about 653 μg, about 654 μg, about 655 μg, about 656 μg, about 657 μg, about 658 μg, about 659 μg, about 660 μg, about 661 μg, about 662 μg, about 663 μg, about 664 μg, about 665 μg, about 666 μg, about 667 μg, about 668 μg, about 669 μg, about 670 μg, about 671 μg, about 672 μg, about 673 μg, about 674 μg, about 675 μg, about 676 μg, about 677 μg, about 678 μg, about 679 μg, about 680 μg, about 681 μg, about 682 μg, about 683 μg, about 684 μg, about 685 μg, about 686 μg, about 687 μg, about 688 μg, about 689 μg, about 690 μg, about 691 μg, about 692 μg, about 693 μg, about 694 μg, about 695 μg, about 696 μg, about 697 μg, about 698 μg, about 699 μg, about 700 μg, about 701 μg, about 702 μg, about 703 μg, about 704 μg, about 705 μg, about 706 μg, about 707 μg, about 708 μg, about 709 μg, about 710 μg, about 711 μg, about 712 μg, about 713 μg, about 714 μg, about 715 μg, about 716 μg, about 717 μg, about 718 μg, about 719 μg, about 720 μg, about 721 μg, about 722 μg, about 723 μg, about 724 μg, about 725 μg, about 726 μg, about 727 μg, about 728 μg, about 729 μg, about 730 μg, about 731 μg, about 732 μg, about 733 μg, about 734 μg, about 735 μg, about 736 μg, about 737 μg, about 738 μg, about 739 μg, about 740 μg, about 741 μg, about 742 μg, about 743 μg, about 744 μg, about 745 μg, about 746 μg, about 747 μg, about 748 μg, about 749 μg, about 750 μg, about 751 μg, about 752 μg, about 753 μg, about 754 μg, about 755 μg, about 756 μg, about 757 μg, about 758 μg, about 759 μg, about 760 μg, about 761 μg, about 762 μg, about 763 μg, about 764 μg, about 765 μg, about 766 μg, about 767 μg, about 768 μg, about 769 μg, about 770 μg, about 771 μg, about 772 μg, about 773 μg, about 774 μg, about 775 μg, about 776 μg, about 777 μg, about 778 μg, about 779 μg, about 780 μg, about 781 μg, about 782 μg, about 783 μg, about 784 μg, about 785 μg, about 786 μg, about 787 μg, about 788 μg, about 789 μg, about 790 μg, about 791 μg, about 792 μg, about 793 μg, about 794 μg, about 795 μg, about 796 μg, about 797 μg, about 798 μg, about 799 μg, about 800 μg, about 801 μg, about 802 μg, about 803 μg, about 804 μg, about 805 μg, about 806 μg, about 807 μg, about 808 μg, about 809 μg, about 810 μg, about 811 μg, about 812 μg, about 813 μg, about 814 μg, about 815 μg, about 816 μg, about 817 μg, about 818 μg, about 819 μg, about 820 μg, about 821 μg, about 822 μg, about 823 μg, about 824 μg, about 825 μg, about 826 μg, about 827 μg, about 828 μg, about 829 μg, about 830 μg, about 831 μg, about 832 μg, about 833 μg, about 834 μg, about 835 μg, about 836 μg, about 837 μg, about 838 μg, about 839 μg, about 840 μg, about 841 μg, about 842 μg, about 843 μg, about 844 μg, about 845 μg, about 846 μg, about 847 μg, about 848 μg, about 849 μg, about 850 μg, about 851 μg, about 852 μg, about 853 μg, about 854 μg, about 855 μg, about 856 μg, about 857 μg, about 858 μg, about 859 μg, about 860 μg, about 861 μg, about 862 μg, about 863 μg, about 864 μg, about 865 μg, about 866 μg, about 867 μg, about 868 μg, about 869 μg, about 870 μg, about 871 μg, about 872 μg, about 873 μg, about 874 μg, about 875 μg, about 876 μg, about 877 μg, about 878 μg, about 879 μg, about 880 μg, about 881 μg, about 882 μg, about 883 μg, about 884 μg, about 885 μg, about 886 μg, about 887 μg, about 888 μg, about 889 μg, about 890 μg, about 891 μg, about 892 μg, about 893 μg, about 894 μg, about 895 μg, about 896 μg, about 897 μg, about 898 μg, about 899 μg, about 900 μg, about 901 μg, about 902 μg, about 903 μg, about 904 μg, about 905 μg, about 906 μg, about 907 μg, about 908 μg, about 909 μg, about 910 μg, about 911 μg, about 912 μg, about 913 μg, about 914 μg, about 915 μg, about 916 μg, about 917 μg, about 918 μg, about 919 μg, about 920 μg, about 921 μg, about 922 μg, about 923 μg, about 924 μg, about 925 μg, about 926 μg, about 927 μg, about 928 μg, about 929 μg, about 930 μg, about 931 μg, about 932 μg, about 933 μg, about 934 μg, about 935 μg, about 936 μg, about 937 μg, about 938 μg, about 939 μg, about 940 μg, about 941 μg, about 942 μg, about 943 μg, about 944 μg, about 945 μg, about 946 μg, about 947 μg, about 948 μg, about 949 μg, about 950 μg, about 951 μg, about 952 μg, about 953 μg, about 954 μg, about 955 μg, about 956 μg, about 957 μg, about 958 μg, about 959 μg, about 960 μg, about 961 μg, about 962 μg, about 963 μg, about 964 μg, about 965 μg, about 966 μg, about 967 μg, about 968 μg, about 969 μg, about 970 μg, about 971 μg, about 972 μg, about 973 μg, about 974 μg, about 975 μg, about 976 μg, about 977 μg, about 978 μg, about 979 μg, about 980 μg, about 981 μg, about 982 μg, about 983 μg, about 984 μg, about 985 μg, about 986 μg, about 987 μg, about 988 μg, about 989 μg, about 990 μg, about 991 μg, about 992 μg, about 993 μg, about 994 μg, about 995 μg, about 996 μg, about 997 μg, about 998 μg, about 999 μg, about 1000 μg, of total RSV F glycoproteins, including all values and ranges therebetween.

In embodiments, the methods comprise administering from about 1 μg to about 400 μg, from 1 μg to about 350 μg, from 1 μg to about 300 μg, from 1 μg to about 250 μg, from 1 μg to about 200 μg, from 1 μg to about 150 μg, from 1 μg to about 100 μg, from 1 μg to about 50 μg, or from 1 μg to about 25 μg of adjuvant. In embodiments, the immunogenic compositions comprise about 1 μg, about 2 μg, about 3 μg, about 4 μg, about 5 μg, about 6 μg, about 7 μg, about 8 μg, about 9 μg, about 10 μg, about 11 μg, about 12 μg, about 13 μg, about 14 μg, about 15 μg, about 16 μg, about 17 μg, about 18 μg, about 19 μg, about 20 μg, about 21 μg, about 22 μg, about 23 μg, about 24 μg, about 25 μg, about 26 μg, about 27 μg, about 28 μg, about 29 μg, about 30 μg, about 31 μg, about 32 μg, about 33 μg, about 34 μg, about 35 μg, about 36 μg, about 37 μg, about 38 μg, about 39 μg, about 40 μg, about 41 μg, about 42 μg, about 43 μg, about 44 μg, about 45 μg, about 46 μg, about 47 μg, about 48 μg, about 49 μg, about 50 μg, about 51 μg, about 52 μg, about 53 μg, about 54 μg, about 55 μg, about 56 μg, about 57 μg, about 58 μg, about 59 μg, about 60 μg, about 61 μg, about 62 μg, about 63 μg, about 64 μg, about 65 μg, about 66 μg, about 67 μg, about 68 μg, about 69 μg, about 70 μg, about 71 μg, about 72 μg, about 73 μg, about 74 μg, about 75 μg, about 76 μg, about 77 μg, about 78 μg, about 79 μg, about 80 μg, about 81 μg, about 82 μg, about 83 μg, about 84 μg, about 85 μg, about 86 μg, about 87 μg, about 88 μg, about 89 μg, about 90 μg, about 91 μg, about 92 μg, about 93 μg, about 94 μg, about 95 μg, about 96 μg, about 97 μg, about 98 μg, about 99 μg, about 100 μg, about 101 μg, about 102 μg, about 103 μg, about 104 μg, about 105 μg, about 106 μg, about 107 μg, about 108 μg, about 109 μg, about 110 μg, about 111 μg, about 112 μg, about 113 μg, about 114 μg, about 115 μg, about 116 μg, about 117 μg, about 118 μg, about 119 μg, about 120 μg, about 121 μg, about 122 μg, about 123 μg, about 124 μg, about 125 μg, about 126 μg, about 127 μg, about 128 μg, about 129 μg, about 130 μg, about 131 μg, about 132 μg, about 133 μg, about 134 μg, about 135 μg, about 136 μg, about 137 μg, about 138 μg, about 139 μg, about 140 μg, about 141 μg, about 142 μg, about 143 μg, about 144 μg, about 145 μg, about 146 μg, about 147 μg, about 148 μg, about 149 μg, about 150 μg, about 151 μg, about 152 μg, about 153 μg, about 154 μg, about 155 μg, about 156 μg, about 157 μg, about 158 μg, about 159 μg, about 160 μg, about 161 μg, about 162 μg, about 163 μg, about 164 μg, about 165 μg, about 166 μg, about 167 μg, about 168 μg, about 169 μg, about 170 μg, about 171 μg, about 172 μg, about 173 μg, about 174 μg, about 175 μg, about 176 μg, about 177 μg, about 178 μg, about 179 μg, about 180 μg, about 181 μg, about 182 μg, about 183 μg, about 184 μg, about 185 μg, about 186 μg, about 187 μg, about 188 μg, about 189 μg, about 190 μg, about 191 μg, about 192 μg, about 193 μg, about 194 μg, about 195 μg, about 196 μg, about 197 μg, about 198 μg, about 199 μg, about 200 μg, about 201 μg, about 202 μg, about 203 μg, about 204 μg, about 205 μg, about 206 μg, about 207 μg, about 208 μg, about 209 μg, about 210 μg, about 211 μg, about 212 μg, about 213 μg, about 214 μg, about 215 μg, about 216 μg, about 217 μg, about 218 μg, about 219 μg, about 220 μg, about 221 μg, about 222 μg, about 223 μg, about 224 μg, about 225 μg, about 226 μg, about 227 μg, about 228 μg, about 229 μg, about 230 μg, about 231 μg, about 232 μg, about 233 μg, about 234 μg, about 235 μg, about 236 μg, about 237 μg, about 238 μg, about 239 μg, about 240 μg, about 241 μg, about 242 μg, about 243 μg, about 244 μg, about 245 μg, about 246 μg, about 247 μg, about 248 μg, about 249 μg, about 250 μg, about 251 μg, about 252 μg, about 253 μg, about 254 μg, about 255 μg, about 256 μg, about 257 μg, about 258 μg, about 259 μg, about 260 μg, about 261 μg, about 262 μg, about 263 μg, about 264 μg, about 265 μg, about 266 μg, about 267 μg, about 268 μg, about 269 μg, about 270 μg, about 271 μg, about 272 μg, about 273 μg, about 274 μg, about 275 μg, about 276 μg, about 277 μg, about 278 μg, about 279 μg, about 280 μg, about 281 μg, about 282 μg, about 283 μg, about 284 μg, about 285 μg, about 286 μg, about 287 μg, about 288 μg, about 289 μg, about 290 μg, about 291 μg, about 292 μg, about 293 μg, about 294 μg, about 295 μg, about 296 μg, about 297 μg, about 298 μg, about 299 μg, about 300 μg, about 301 μg, about 302 μg, about 303 μg, about 304 μg, about 305 μg, about 306 μg, about 307 μg, about 308 μg, about 309 μg, about 310 μg, about 311 μg, about 312 μg, about 313 μg, about 314 μg, about 315 μg, about 316 μg, about 317 μg, about 318 μg, about 319 μg, about 320 μg, about 321 μg, about 322 μg, about 323 μg, about 324 μg, about 325 μg, about 326 μg, about 327 μg, about 328 μg, about 329 μg, about 330 μg, about 331 μg, about 332 μg, about 333 μg, about 334 μg, about 335 μg, about 336 μg, about 337 μg, about 338 μg, about 339 μg, about 340 μg, about 341 μg, about 342 μg, about 343 μg, about 344 μg, about 345 μg, about 346 μg, about 347 μg, about 348 μg, about 349 μg, about 350 μg, about 351 μg, about 352 μg, about 353 μg, about 354 μg, about 355 μg, about 356 μg, about 357 μg, about 358 μg, about 359 μg, about 360 μg, about 361 μg, about 362 μg, about 363 μg, about 364 μg, about 365 μg, about 366 μg, about 367 μg, about 368 μg, about 369 μg, about 370 μg, about 371 μg, about 372 μg, about 373 μg, about 374 μg, about 375 μg, about 376 μg, about 377 μg, about 378 μg, about 379 μg, about 380 μg, about 381 μg, about 382 μg, about 383 μg, about 384 μg, about 385 μg, about 386 μg, about 387 μg, about 388 μg, about 389 μg, about 390 μg, about 391 μg, about 392 μg, about 393 μg, about 394 μg, about 395 μg, about 396 μg, about 397 μg, about 398 μg, about 399 μg, or about 400 μg of adjuvant. In embodiments, the methods comprise administering about 50 μg of adjuvant. In embodiments, the methods comprise administering about 75 μg of adjuvant.

### Example 1: Preparation of SARS-COV-2 S Glycoprotein Nanoparticles

SARS-COV-2 (“CoV”) Spike(S) glycoproteins (e.g., CoV S glycoproteins with amino acid sequences with at least 90%, at least 91%, at least 92%, at least 93%, at least 94%, at least 95%, at least 96%, at least 97%, at least 98%, at least 99%, or 100% identity to any one of SEQ ID NOS: 1-5, 38, 40-42, 44-46, 48-49, 51-52, 54, 56, 58-59, 61-67, 72, 74, 76, 77, 80-81, 84-89, 105-110, 112-115, 118, 121, 130, 132, 133, 134, 136, 137, 138, 140-141, 143, 144, 146, 147, 149, 151-153, 155-160, 167, 170, 173-192, 194-195, 200, 203, 207, 209, 211, 213, 215, 217-236, 242-328) were expressed in a baculovirus expression system. Recombinant plaques expressing the CoV S glycoproteins were picked and confirmed. The recombinant baculovirus was amplified by infection of Sf9 or rhabdovirus-free Sf22a insect cells. A culture of insect cells was infected at ˜0.6 MOI (Multiplicity of infection=virus ffu or pfu/cell) with baculovirus. The culture and supernatant were harvested 48-72 hrs post-infection. The crude cell harvest, approximately 30 mL, was clarified by centrifugation for 15 minutes at approximately 800× g. The resulting crude cell harvests containing the coronavirus Spike(S) protein were purified as nanoparticles as described below.

To produce nanoparticles, the non-ionic surfactant TERGITOL® nonylphenol ethoxylate NP-9 was used in the membrane protein extraction protocol. The crude extraction was further purified by passing through anion exchange chromatography, lentil lectin affinity/HIC and cation exchange chromatography. The washed cells were lysed by detergent treatment and then subjected to low pH treatment which leads to precipitation of BV and Sf9 host cell DNA and protein. The neutralized low pH treatment lysate was clarified and further purified on anion exchange and affinity chromatography before a second low pH treatment was performed.

Affinity chromatography was used to remove Sf9/BV proteins, DNA and NP-9, as well as to concentrate the CoV S glycoprotein. Lentil lectin is a metalloprotein containing calcium and manganese, which reversibly binds polysaccharides and glycosylated proteins containing glucose or mannose. The coronavirus Spike(S) protein-containing anion exchange flow through fraction was loaded onto the lentil lectin affinity chromatography resin (Capto Lentil Lectin, GE Healthcare). The glycosylated coronavirus Spike(S) protein was selectively bound to the resin while non-glycosylated proteins and DNA were removed in the column flow through. Weakly bound glycoproteins were removed with buffers containing high salt and low molar concentration of methyl alpha-D-mannopyranoside (MMP).

The column washes were used to detergent exchange the NP-9 detergent with the surfactant polysorbate 80 (PS80). The CoV S glycoproteins were eluted as nanoparticles from the lentil lectin column with a high concentration of MMP. After elution, the coronavirus Spike(S) protein trimers were assembled into nanoparticles composed of coronavirus Spike(S) protein trimers and PS80 contained in a detergent core.

### Example 2: Preparation of Immunogenic Compositions Comprising CoV S Glycoprotein Nanoparticles

Immunogenic compositions comprising one or more CoV S glycoproteins of Example 1 were prepared by suspending the CoV S glycoprotein in a pharmaceutically acceptable buffer (e.g., 25 mM sodium phosphate, 0.3 mM NaCl, about 0.03% PS80 at a pH of 7.2). A saponin adjuvant was optionally added to the composition. The saponin adjuvant contained two iscom particles: (i) a first iscom particle containing Fraction A of Quillaja Saponaria Molina and not Fraction C of Quillaja Saponaria Molina, and (ii) a second iscom particle containing Fraction C of Quillaja Saponaria Molina and not Fraction A of Quillaja Saponaria Molina. Fraction A and Fraction C accounted for 85% and 15% by weight, respectively, of the sum of the weights of fraction A of Quillaja Saponaria Molina and fraction C of Quillaja Saponaria Molina in the adjuvant.

### Example 3: Intranasal Administration of Immunogenic Compositions of Example 2 Results in a Superior Immune Response Against SARS-COV-2 Compared to Intramuscular Administration of the Same Compositions

The immune response resulting from intranasal and intramuscular boost with the compositions of Example 2 was compared.

Methods: BALB/c mice were administered an immunogenic composition containing a saponin adjuvant (5 μg), 1 μg of a first CoV S glycoprotein having the amino acid sequence of SEQ ID NO: 87, and 1 μg of a second CoV S glycoprotein of SEQ ID NO: 222 on days 0 and 14 (“primary series”). On day 48, mice were administered a boost dose of an immunogenic composition containing 1 μg of a CoV S glycoprotein of SEQ ID NO: 274 and optionally a saponin adjuvant intranasally or intramuscularly. FIG. 1 shows an image of the study design.

The saponin adjuvant contained two iscom particles: (i) a first iscom particle containing Fraction A of Quillaja Saponaria Molina and not Fraction C of Quillaja Saponaria Molina, and (ii) a second iscom particle containing Fraction C of Quillaja Saponaria Molina and not Fraction A of Quillaja Saponaria Molina. Fraction A and Fraction C accounted for 85% and 15% by weight, respectively, of the sum of the weights of fraction A of Quillaja Saponaria Molina and fraction C of Quillaja Saponaria Molina in the adjuvant.

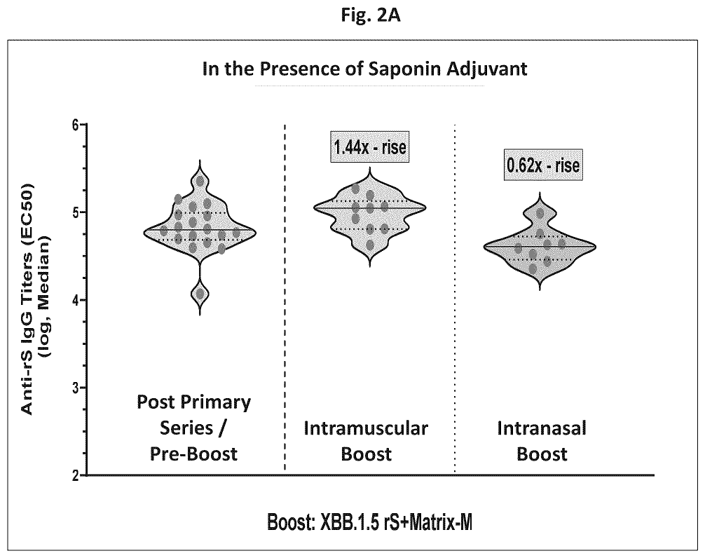

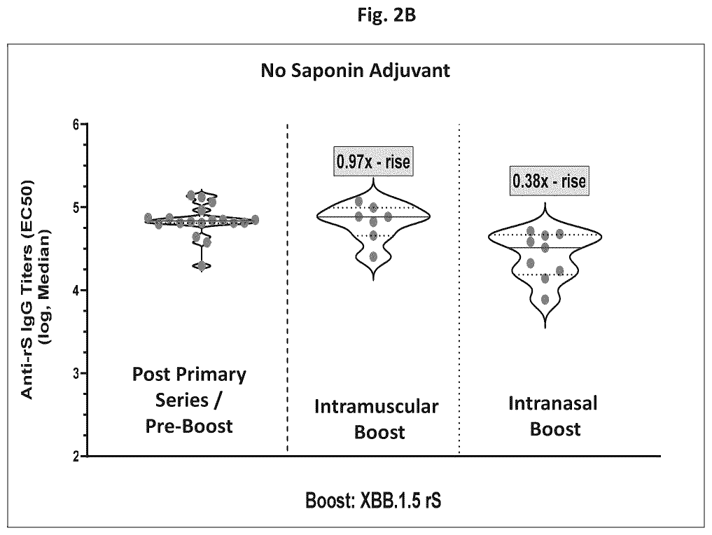

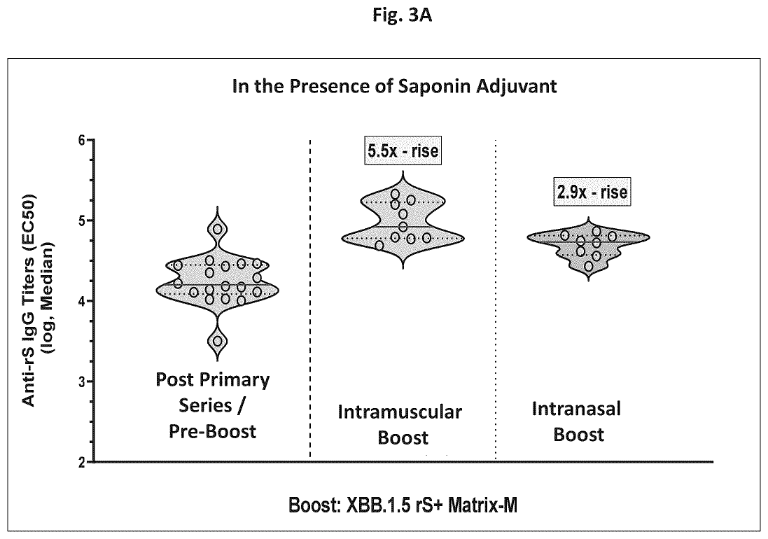

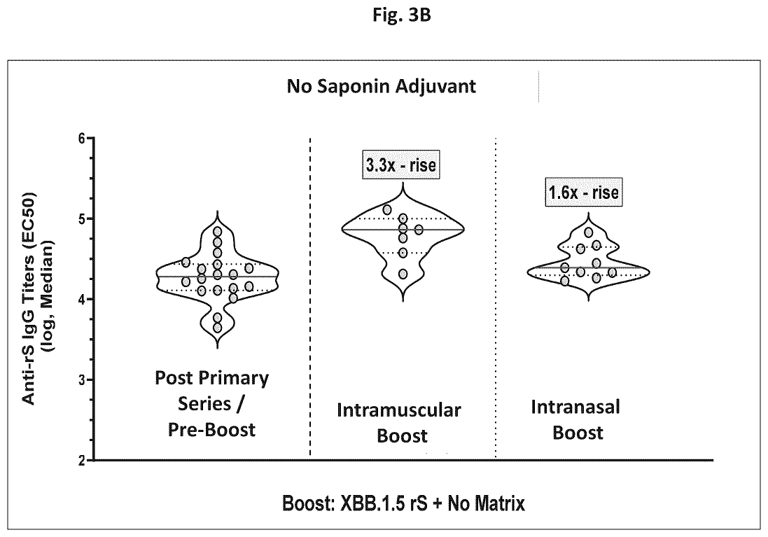

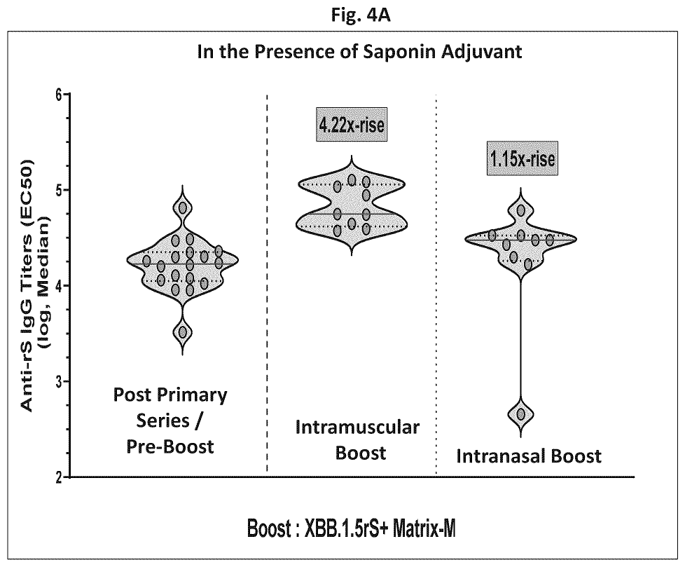

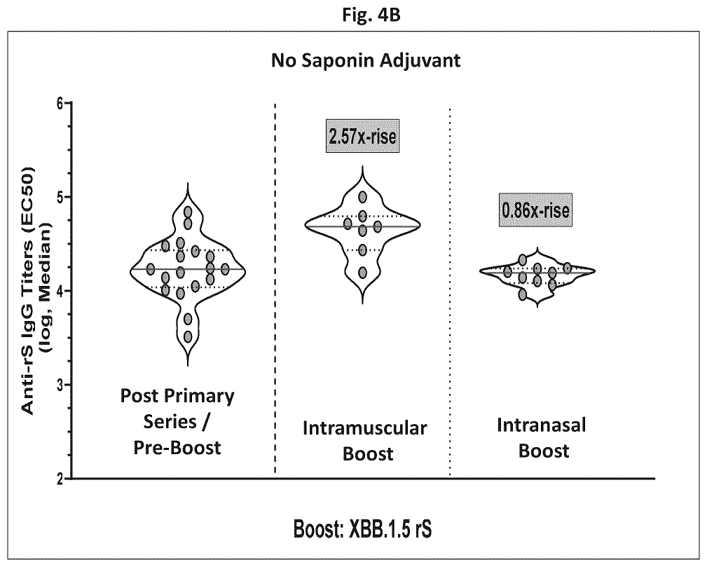

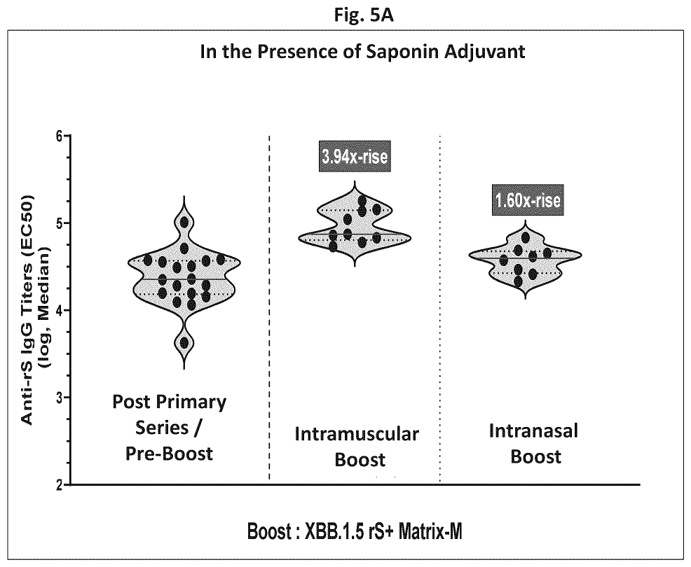

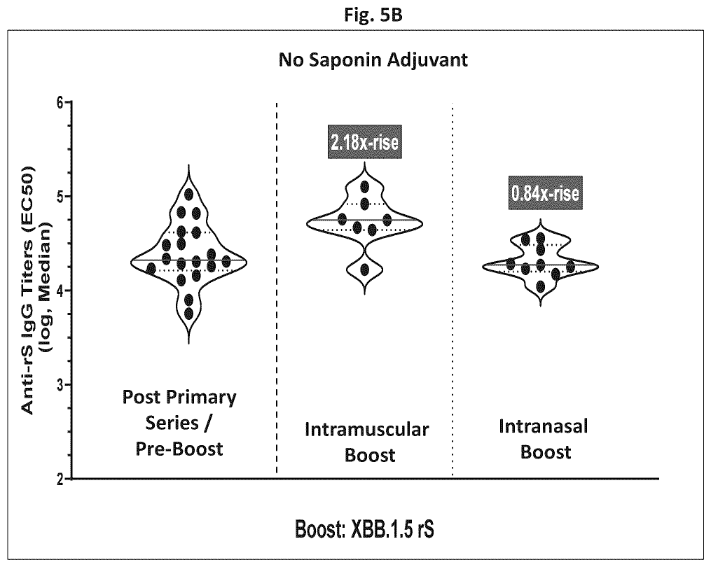

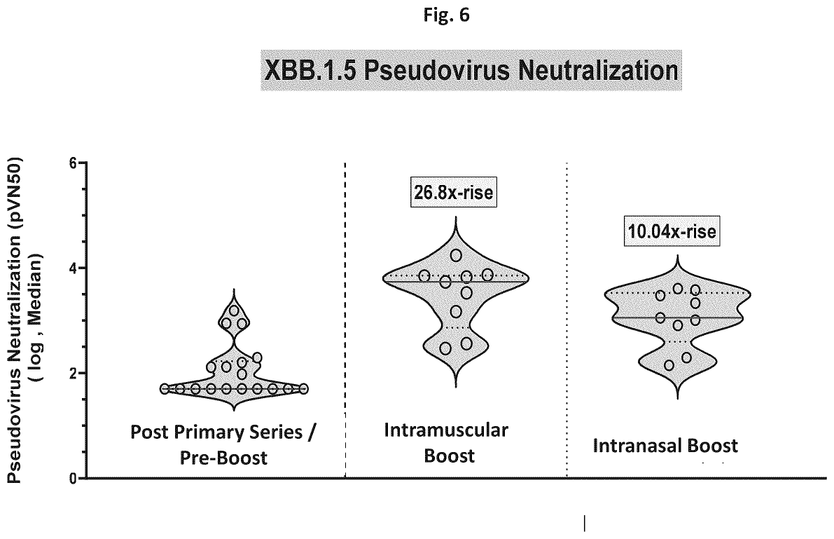

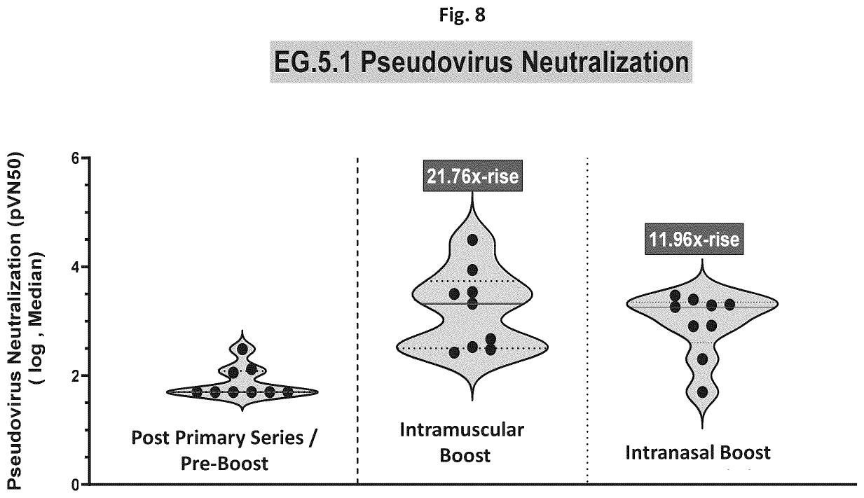

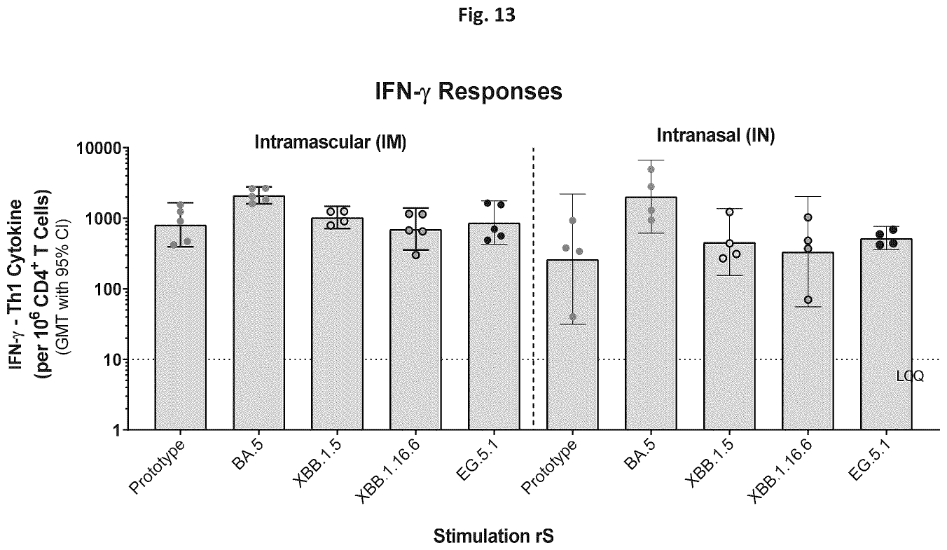

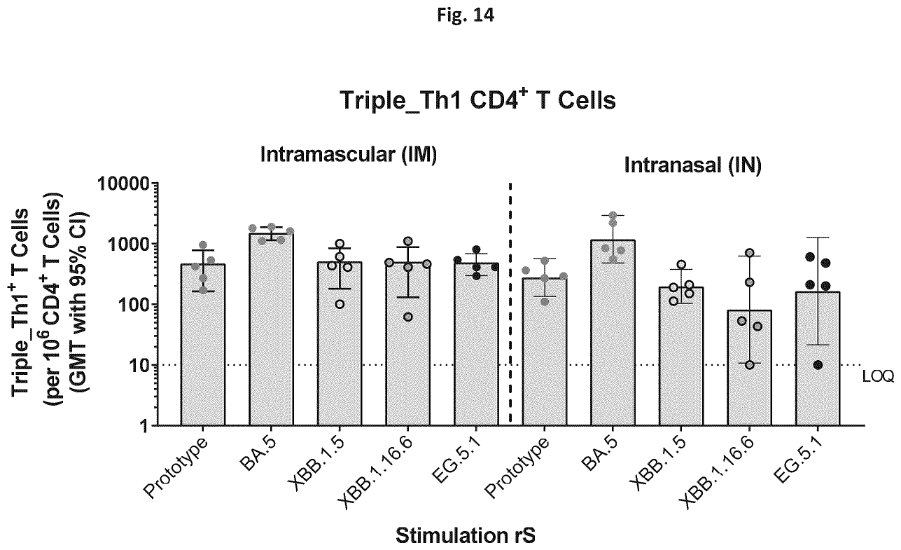

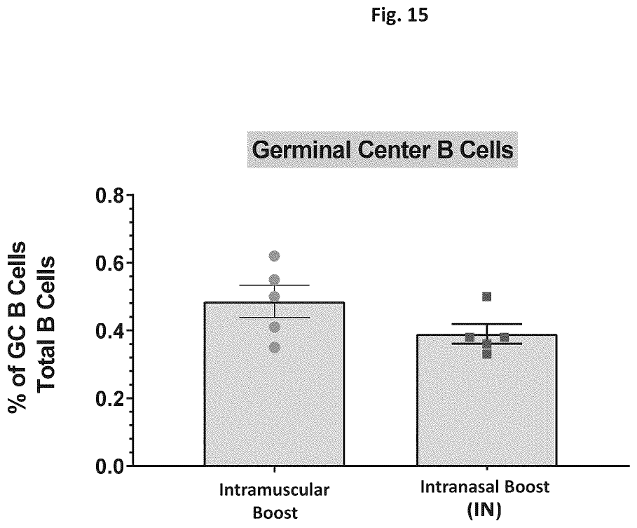

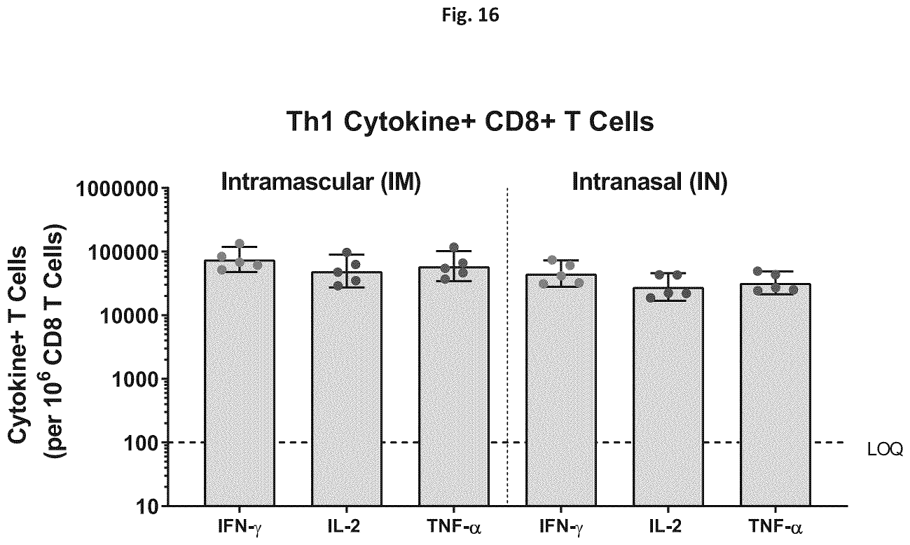

Results: Intramuscular and intranasal boosting induced increased total anti-SARS-COV-2 S glycoprotein antibodies against SARS-COV-2 S glycoproteins from the SARS-COV-2 prototype strain (FIG. 2A, FIG. 2B), the SARS-COV-2 XBB.1.5 strain (FIG. 3A, FIG. 3B), the SARS-COV-2 XBB.1.16.6 strain (FIG. 4A, FIG. 4B), the SARS-COV-2 EG.5.1 IgG strain (FIG. 5A, FIG. 5B) compared to preboost in both the presence and absence of saponin adjuvant. Intramuscular and intranasal boosting also increased antibodies that neutralized pseudoviruses expressing SARS-CoV-2 S glycoprotein from the SARS-COV-2 XBB.1.5 strain (FIG. 6), from the SARS-COV-2 XBB.1.16.6 strain (FIG. 7), and from the SARS-COV-2 EG.5.1 strain (FIG. 8). Intramuscular and intranasal administration also induced IFN-γ secretion (FIG. 13), the production of triple Th1 CD4+ T cells (FIG. 14), the production of germinal center B cells (FIG. 15), and the production of Th1 Cytokine+CD8+ T cells (FIG. 16).

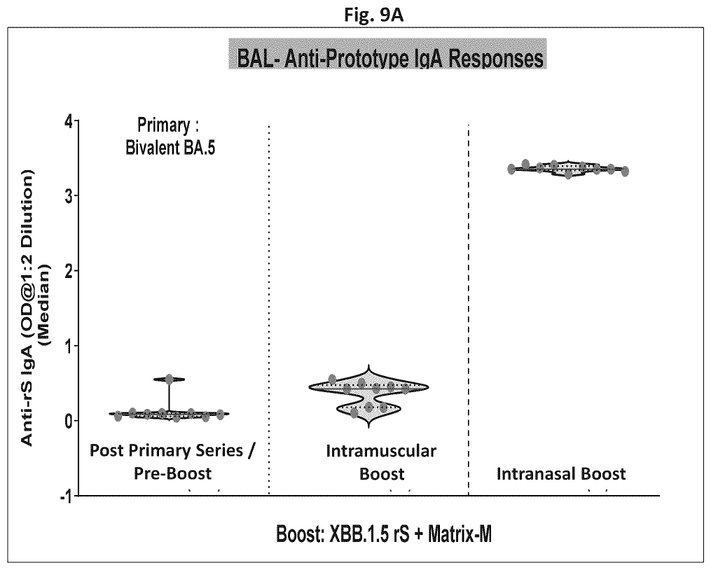

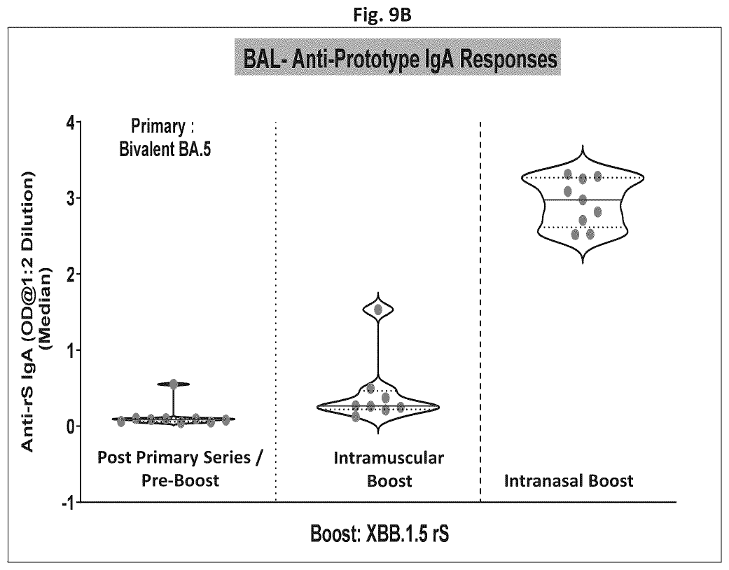

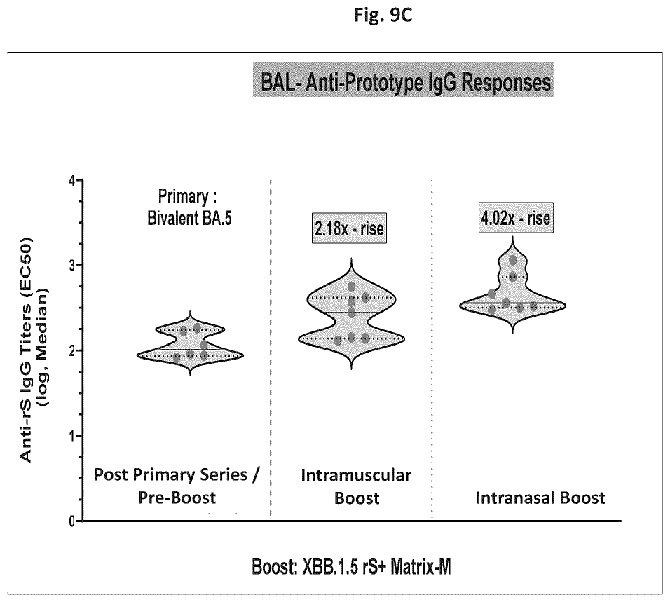

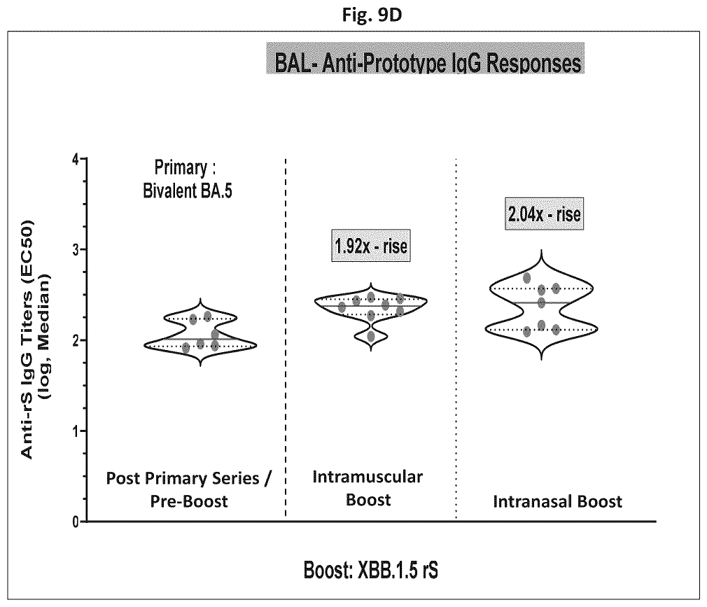

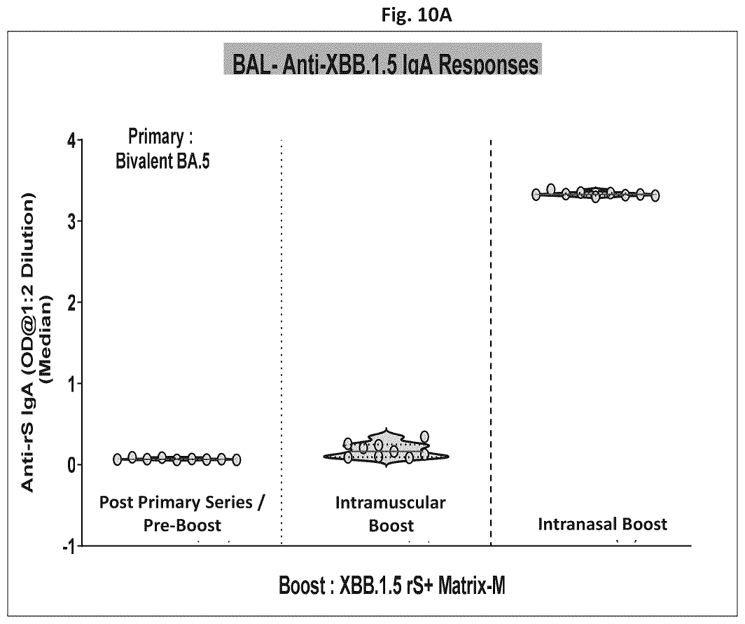

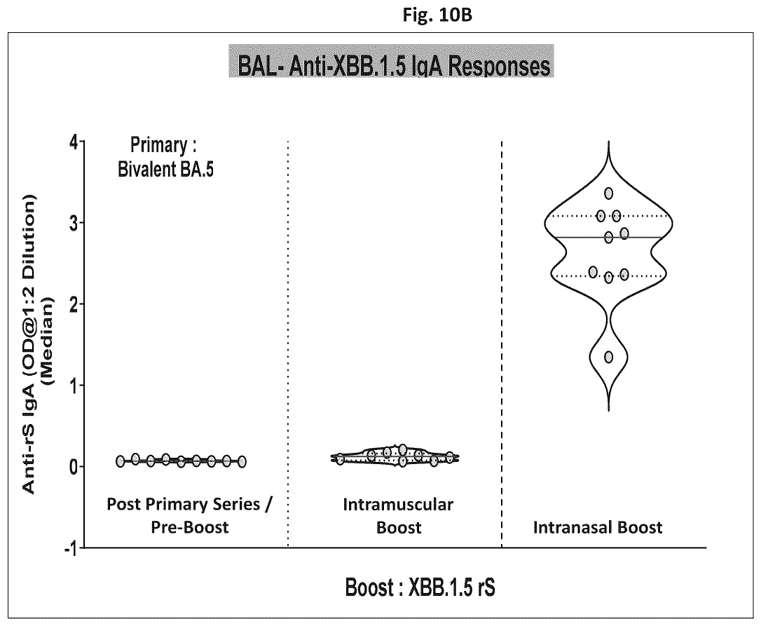

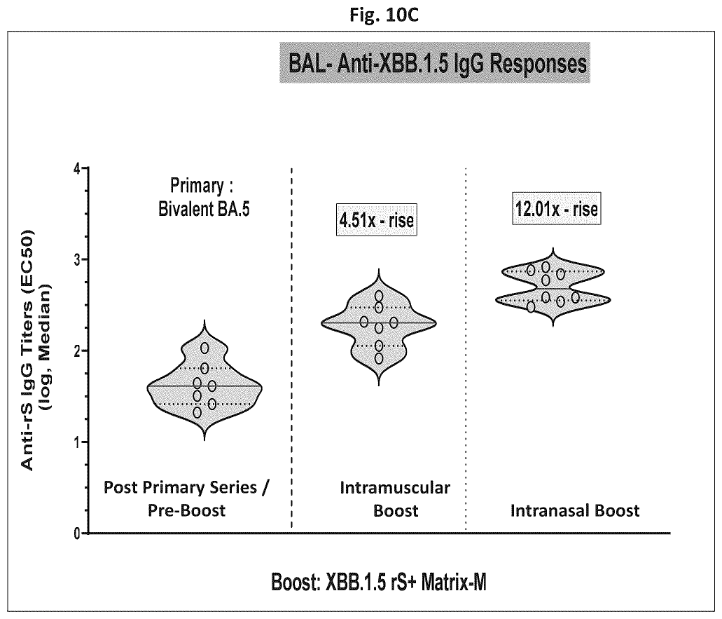

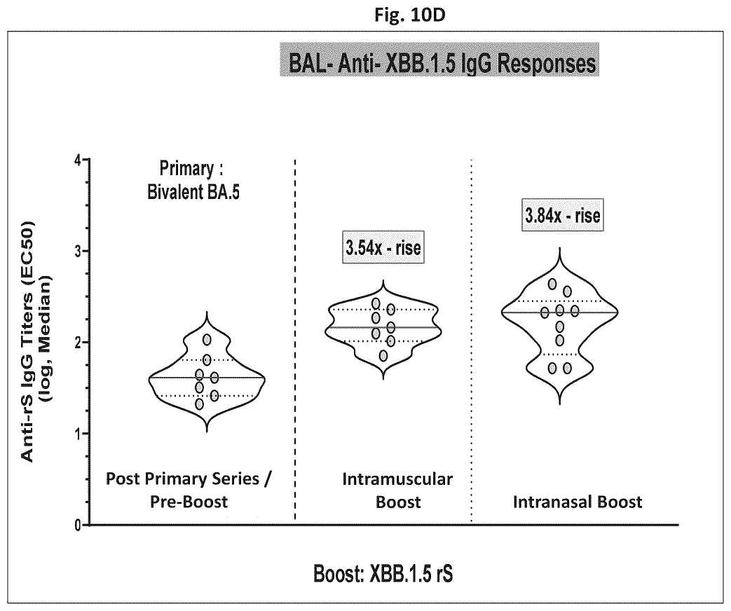

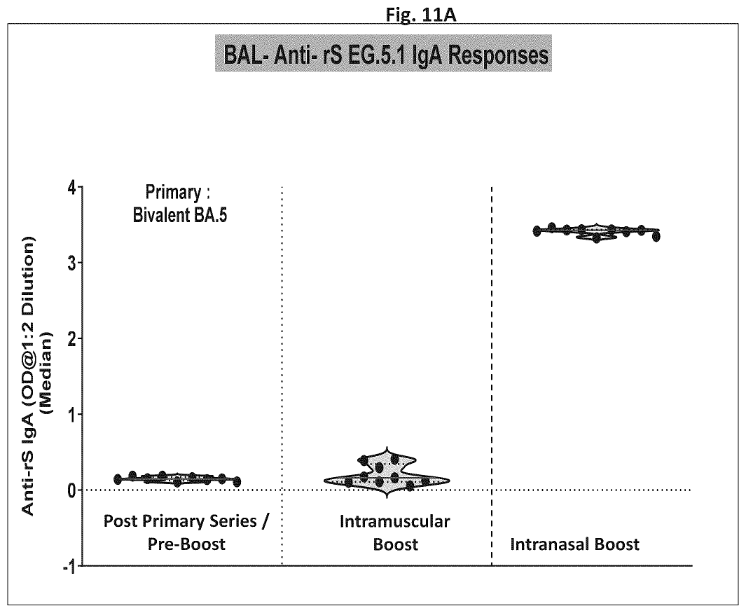

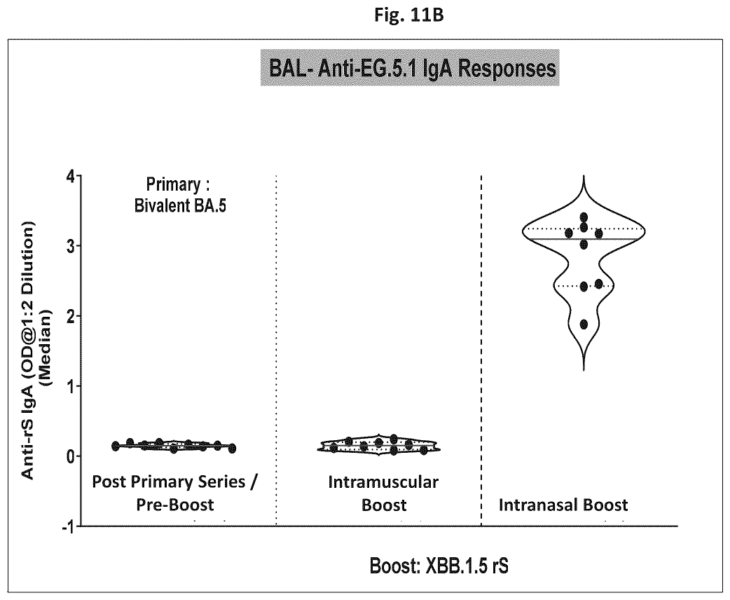

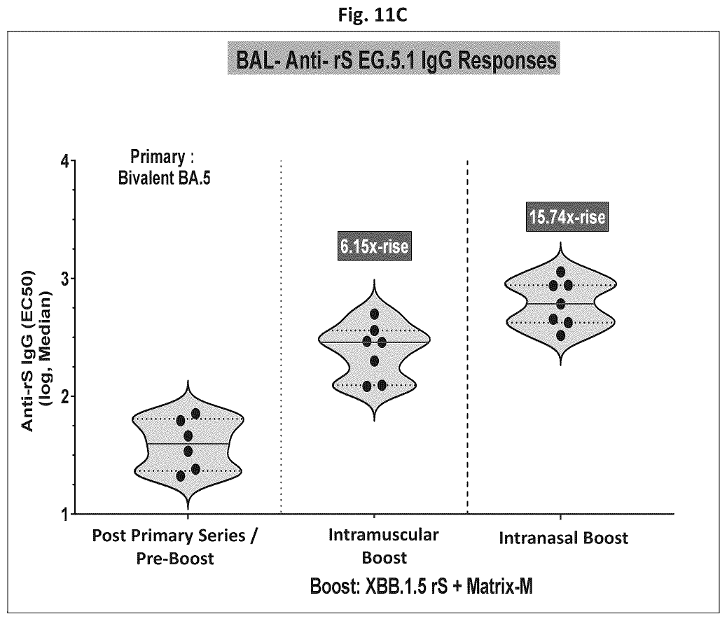

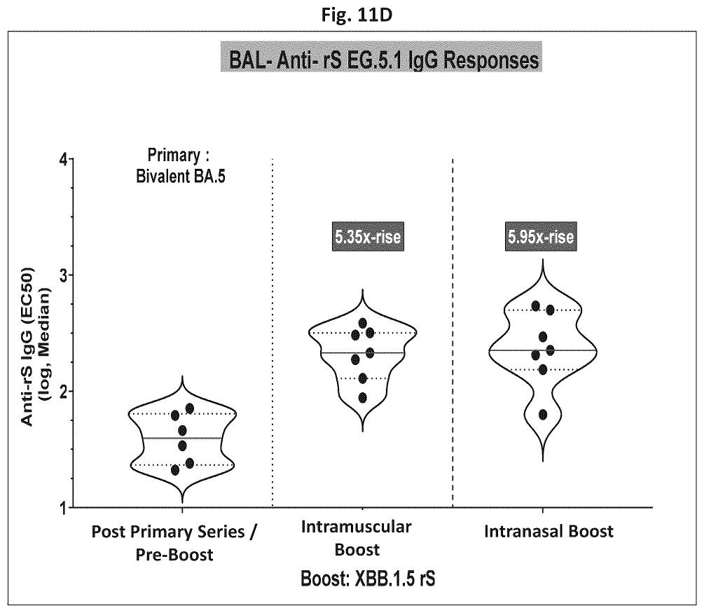

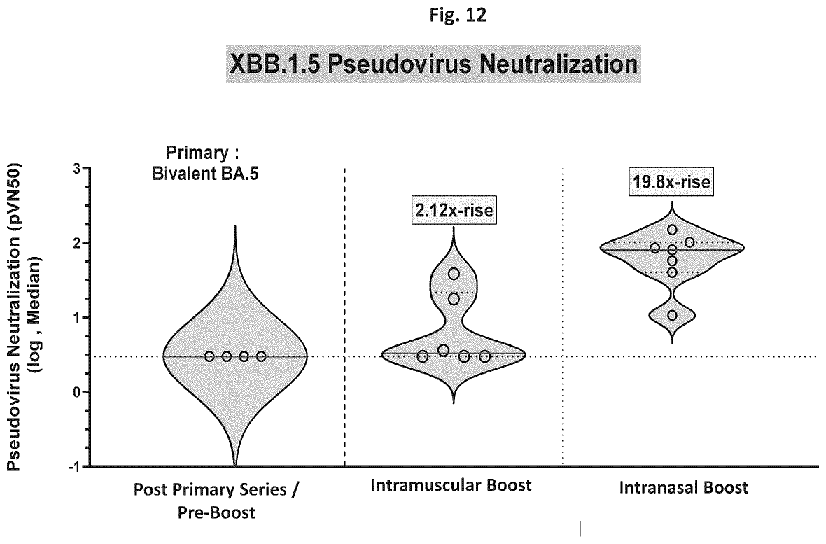

Surprisingly and unexpectedly, intranasal administration, but not intramuscular administration, induced secretion of anti SARS-COV-2 IgA and IgG antibodies in the bronchiole alveolar lavage (BAL), specific for SARS-COV-2 S glycoprotein from the SARS-COV-2 prototype strain (FIGS. 9A-9D), the SARS-COV-2 XBB.1.5 strain (FIGS. 10A-10D), or the SARS-COV-2 EG.5.1 strain (FIGS. 11A-11D). Secretion of anti-SARS-COV-2 IgA and IgG antibodies in the BAL was enhanced when compositions containing saponin adjuvant were administered. FIG. 12 shows that intranasal administration induces a 19.8 fold increase in neutralizing antibodies against a pseudovirus expressing the SARS-COV-2 S glycoprotein of XBB.1.5, whereas intramuscular administration only induces a 2.12 fold increase in neutralizing antibodies against the same pseudovirus.

Collectively, this data shows that intranasal administration of the immunogenic compositions described herein induces both mucosal and systemic immunity, whereas intramuscular administration primarily induces systemic immunity. The induction of mucosal immunity results in the stimulation of adaptive immune cells, such as CD4+ and CD8+ T cells and B cells that secrete IgA and IgG. The extent of induction of mucosal immunity is particularly remarkable here because the mucosal layer in the nasal environment contains proteolytic enzymes that would be expected to break down the SARS-COV-2 S glycoprotein in the composition. This is especially true given the structure of the nanoparticles, which present the glycoproteins from a detergent core, exposing them to the proteases. Flumist, which was withdrawn after becoming the only approved vaccine for nasal administration would, as a viral vector, have expressed proteins de novo intranasally and would thus have been predicted to be less vulnerable to nasal proteases. The fact that Flumist was withdrawn due to low efficacy further underscores the surprising nature of the strong immune response obtained with the nanoparticle approach disclosed herein.

### Example 4: Primary Intranasal Administration of Immunogenic Compositions

The immune response resulting from intranasal (IN) and intramuscular (IM) administration of the compositions of Example 2 is compared. Mice are intranasally or intramuscularly administered an immunogenic composition of Example 2 on day 0 and day 14. Mice are boosted intramuscularly or intranasally with an immunogenic composition 1 month from administration of the first dose of the immunogenic composition.

A saponin adjuvant is optionally added to each composition. The saponin adjuvant contained two iscom particles: (i) a first iscom particle containing Fraction A of Quillaja Saponaria Molina and not Fraction C of Quillaja Saponaria Molina, and (ii) a second iscom particle containing Fraction C of Quillaja Saponaria Molina and not Fraction A of Quillaja Saponaria Molina. Fraction A and Fraction C accounted for 85% and 15% by weight, respectively, of the sum of the weights of fraction A of Quillaja Saponaria Molina and fraction C of Quillaja Saponaria Molina in the adjuvant.

Exemplary compositions in the table below are evaluated.

| | | Primary Series (Days 0 and 14) | | | | Boost (1 month after administration of first dose) | | | |
|---|---|---|---|---|---|---|---|---|---|
| | | SARS-CoV-2 S | Amount of | Amount | | SARS-CoV-2 S | Amount of | Amount | |
| | | glycoproteins in | each SARS- | of | | glycoproteins in | SARS-CoV-2 | of | |
| | | immunogenic | CoV-2 S | saponin | | immunogenic | S | saponin | |
| Group | N | composition | glycoprotein | adjuvant | Route | composition | glycoprotein | adjuvant | Route |
| 1 | 8 | SARS-COV-2 S | 1 μg | 5 μg | IM | SARS-COV-2 S | 1 μg | 5 μg | IM |
| | | glycoproteins of | | | | glycoprotein of | | | |
| | | SEQ ID NO: 87 | | | | SEQ ID NO: | | | |
| | | (referred to as | | | | 274 (referred to | | | |
| | | “prototype”)and | | | | as “XBB.1.5”) | | | |
| | | SEQ ID NO: 222 | | | | | | | |
| | | (referred to as | | | | | | | |
| | | ″BA.5″) | | | | | | | |
| 2 | 8 | Prototype and | 1 μg | 5 μg | IN | XBB.1.5 | 1 μg | 5 μg | IN |
| | | BA.5 | | | | | | | |
| 3 | 8 | Prototype and | 5 μg | 5 μg | IN | XBB.1.5 | 5 μg | 5 μg | IN |
| | | BA.5 | | | | | | | |
| 4 | 8 | XBB.1.5 | 1 μg | 5 μg | IM | XBB.1.5 | 1 μg | 5 μg | IM |
| 5 | 8 | XBB.1.5 | 1 μg | 5μg | IN | XBB.1.5 | 1 μg | 5 μg | IN |
| 6 | 8 | XBB.1.5 | 5 μg | 5 μg | IN | XBB.1.5 | 5 μg | 5 μg | IN |
| 7 | 5 | Prototype and | 5 μg | 5 μg | IN | No booster | | | |
| | | BA.5 | | | | | | | |
| 8 | 5 | XBB.1.5 | 5 μg | 5 μg | IN | No booster | | | |
| 9 | 5 | Prototype and | 5 μg | 5 μg | IN | No booster | | | |
| | | BA.5 | | | | | | | |
| 10 | 5 | XBB.1.5 | 5 μg | 5 μg | IN | No booster | | | |

Serum and the bronchoalveolar lavage (BAL) of mice is collected. The amount of total IgG, IgA, and neutralizing antibodies in the serum and BAL are evaluated. The ratios of CD4+ to CD8+ T cells is also evaluated. Intracellular cytokine staining assays are performed to determine if CD4+ or CD8+ T cells secrete Type 1 cytokines (e.g., IFN-γ, TNF-α, IL-2) and/or Type 2 cytokines (CD4+ T cells). B cells are analyzed for germinal center formation (GL7+, CD95+).

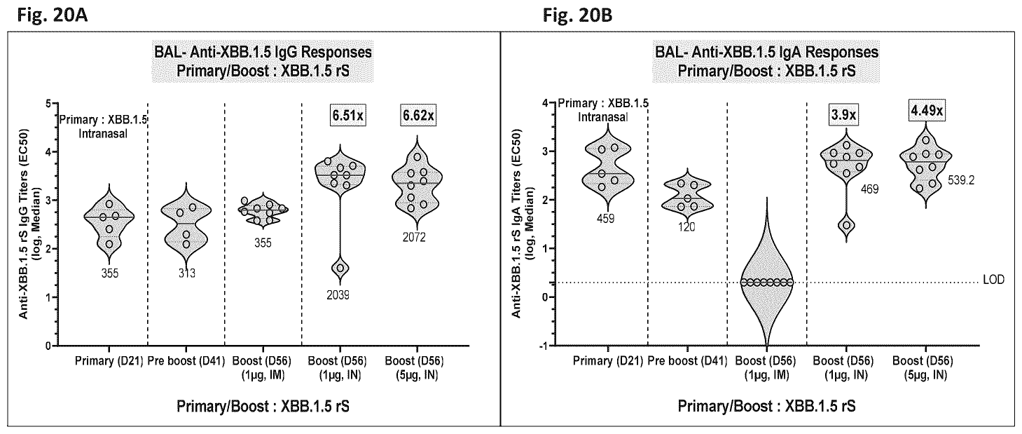

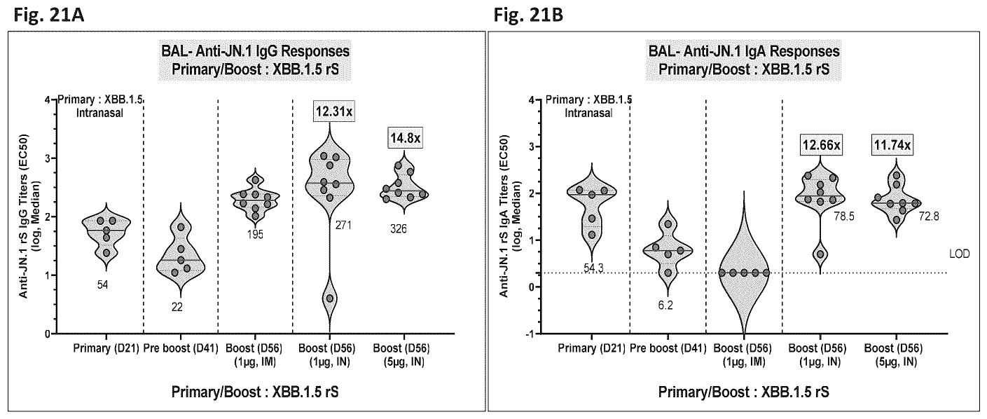

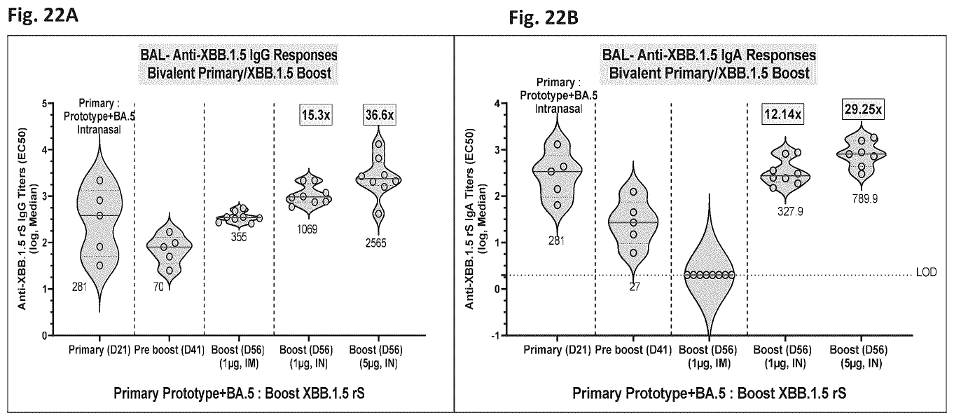

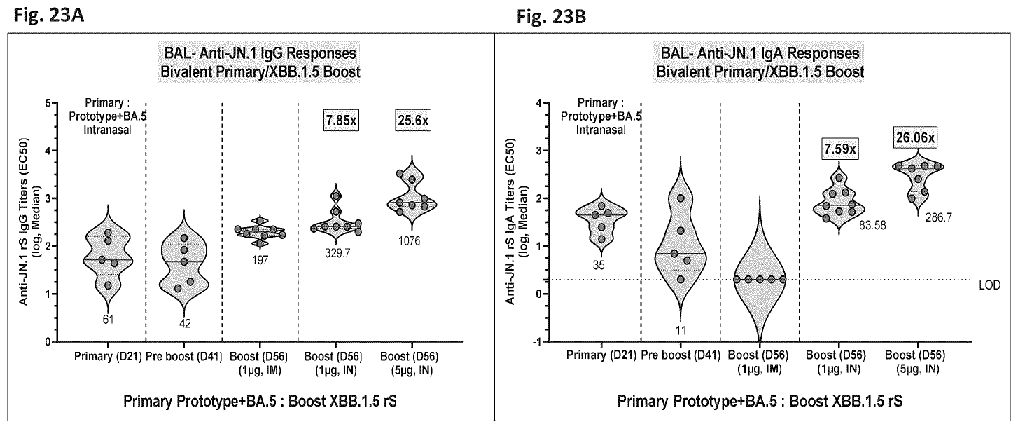

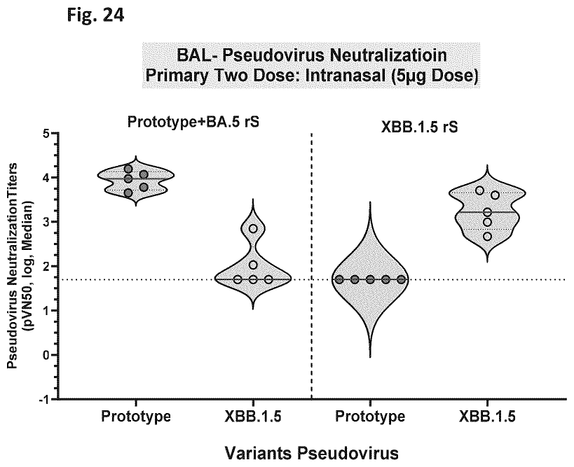

Results: Bronchiole Alveolar Lavage: XBB.1.5 Primary and XBB.1.5 Boost-Intranasal Vaccination, Anti-XBB.1.5 IgG and IgA Responses, BAL XBB.1.5 IgG and IgA responses increased post intranasal boost (FIG. 20A, FIG. 20B) and Anti-JN.1 IgG and IgA Responses-BAL JN.1 IgG and IgA responses increased post intranasal boost (FIG. 21A, FIG. 21B). Bivalent BA.5 Primary and XBB.1.5 Boost-Intranasal: Anti-XBB.1.5 IgG and IgA Responses, BAL XBB.1.5 IgG and IgA responses increased post intranasal boost (FIG. 22A, FIG. 22B), and Anti-JN.1 IgG and IgA Responses, BAL JN.1 IgG and IgA responses increased post intranasal boost (FIG. 23A, FIG. 23B). Primary Two Dose of Bivalent BA.5 and XBB.1.5-Intranasal, Pseudovirus Neutralization Responses, Neutralization responses observed in BAL post Intranasal Primary Two dose (FIG. 24). Nasal Wash: XBB.1.5 Primary and XBB.1.5 Boost-Intranasal Vaccination, Anti-XBB.1.5 IgA Responses (FIG. 25), and Bivalent BA.5 Primary and XBB.1.5 Boost-Intranasal Vaccination, Anti-XBB.1.5 IgA Responses (FIG. 26).

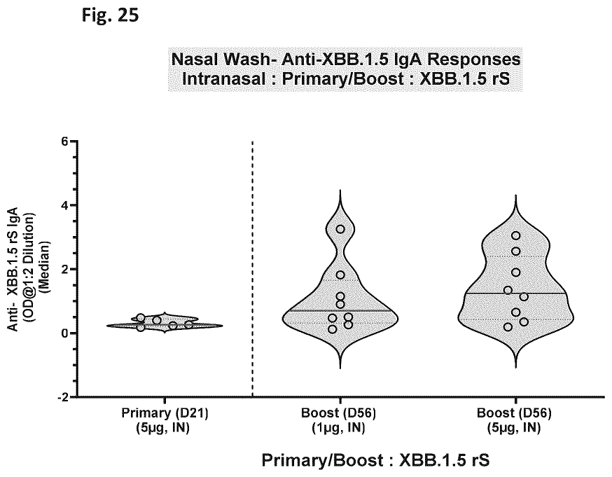

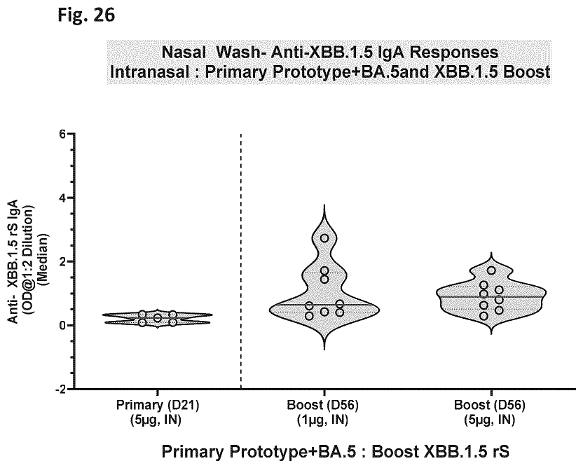

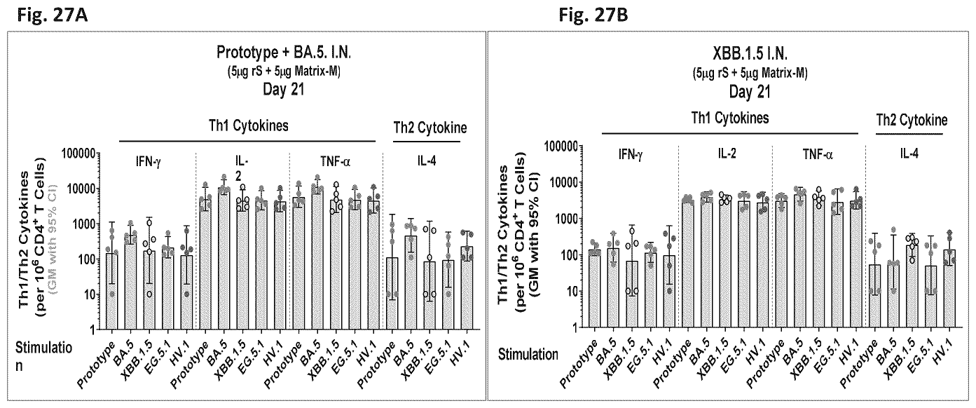

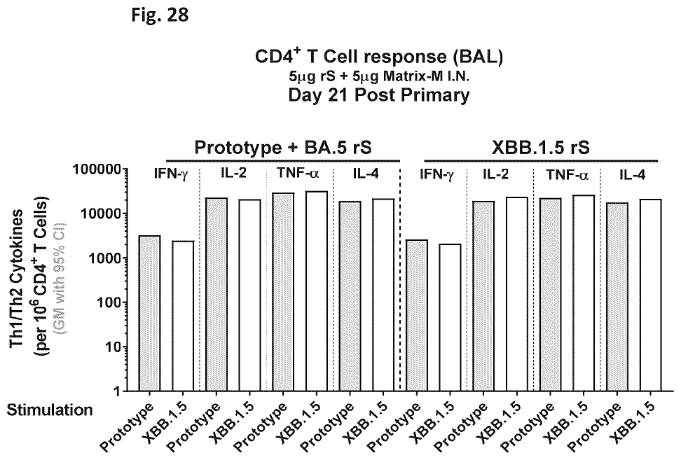

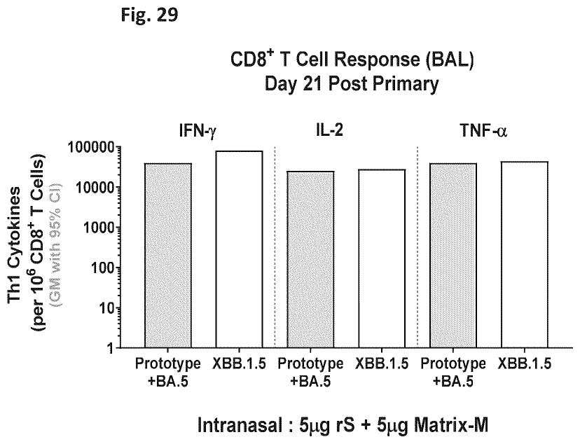

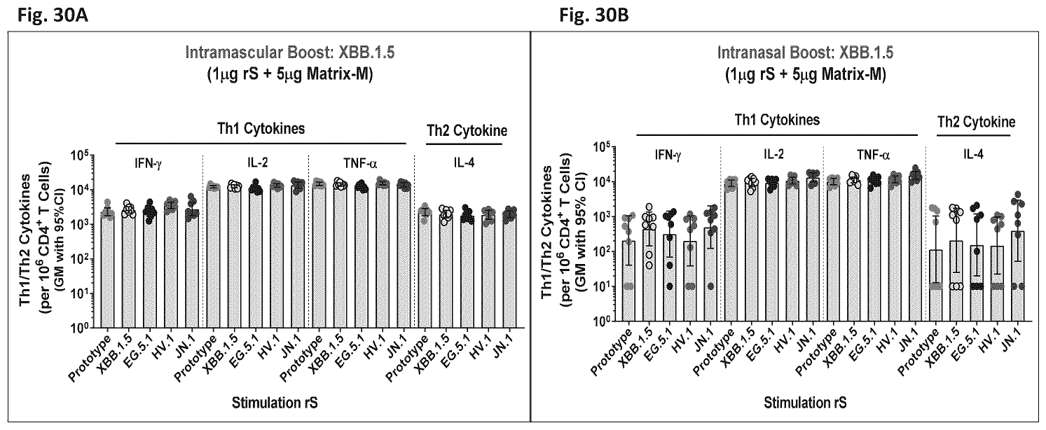

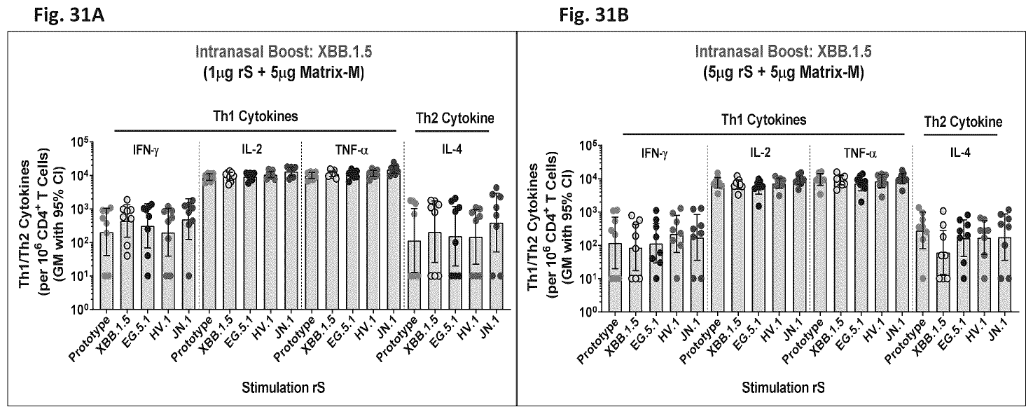

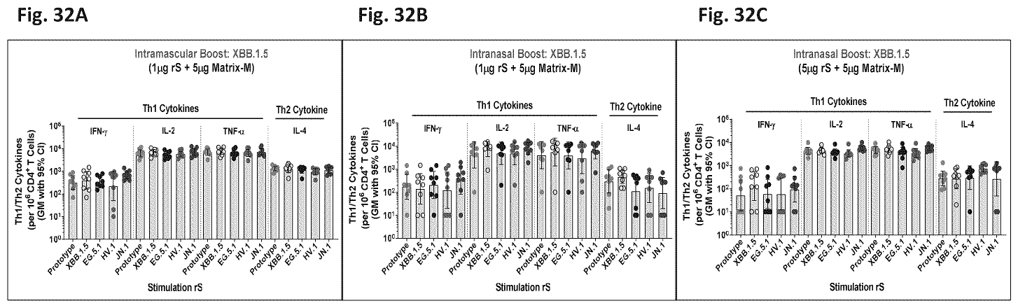

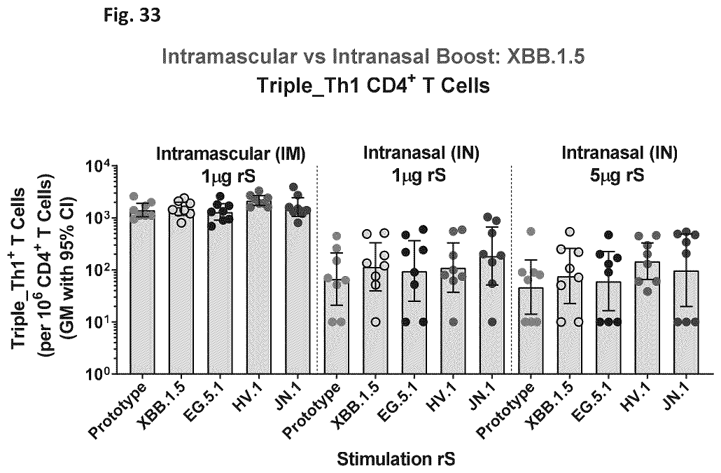

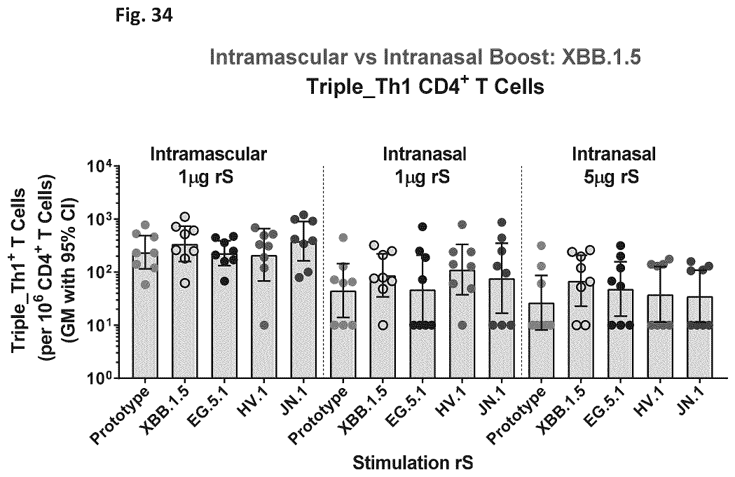

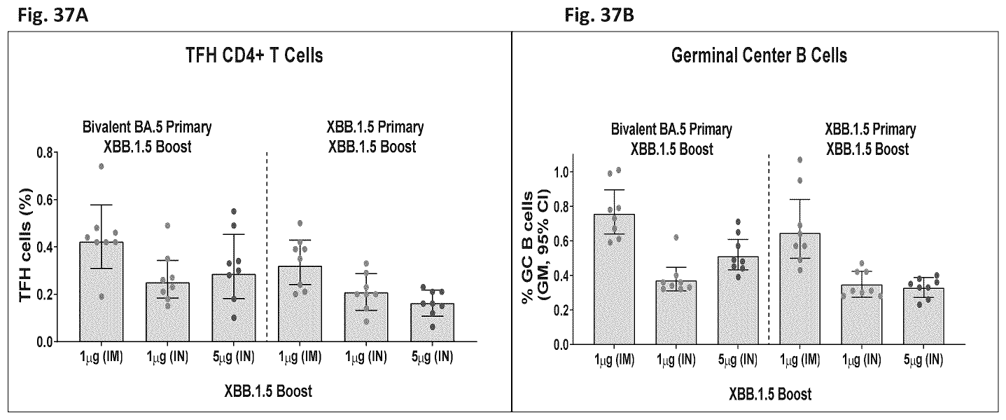

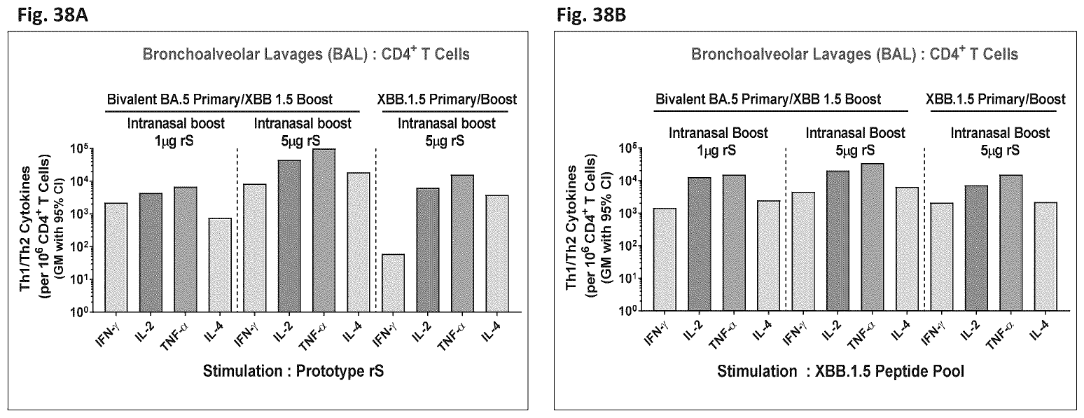

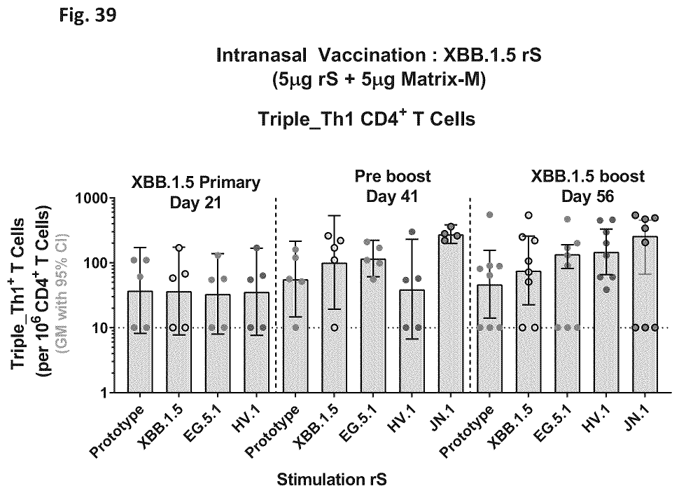

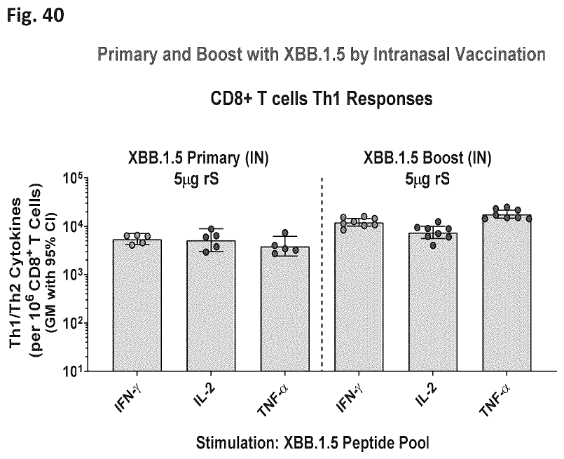

Intracellular Cytokine Staining (ICCS) Assay results: Effector CD4+ T cells Th1/Th2 responses 21 days post primary two doses of Prototype+BA.5 (Intranasal) (5 μg rS+5 μg Matrix-M) versus XBB.1.5 (Intranasal) (5 μg rS+5 μg Matrix-M) (FIG. 27A, FIG. 27B), CD4+ T cells Th1/Th2 responses in BAL 21 days post primary dose Prototype+BA.5 (Intranasal) (5 μg rS+5 μg Matrix-M) versus XBB.1.5 (Intranasal) (5 μg rS+5 μg Matrix-M) (FIG. 28), CD8+ T cells Th1 responses in BAL 21 days post primary dose Prototype+BA.5 (Intranasal) (5 μg rS+5 μg Matrix-M) versus XBB.1.5 (Intranasal) (5 μg rS+5 μg Matrix-M) (FIG. 29). D4+ T cells Th1/Th2 responses to two doses of Bivalent BA.5 Primary followed by XBB.1.5 Intramuscular Boost (1 μg rS+5 μg Matrix-M) versus Bivalent BA.5 Primary followed by XBB.1.5 Intranasal Boost (1 μg rS+5 μg Matrix-M) (FIG. 30A, FIG. 30B), CD4+ T cells Th1/Th2 responses to two doses of Bivalent BA.5 Primary followed by XBB.1.5 Intranasal Boost (1 μg rS+5 μg Matrix-M) versus two doses of Bivalent BA.5 Primary followed by XBB.1.5 Intranasal Boost (5 μg rS+5 μg Matrix-M) (FIG. 31A, FIG. 31B). CD4+ T cells Th1/Th2 responses to two doses of XBB.1.5 Primary followed by XBB.1.5 Intramuscular Boost (1 μg rS+5 μg Matrix-M) versus two doses of XBB.1.5 Primary followed by XBB.1.5 Intranasal Boost (1 μg rS+5 μg Matrix-M) versus two doses of XBB.1.5 Primary followed by XBB.1.5 Intranasal Boost (5 μg rS+5 μg Matrix-M) (FIG. 32A, FIG. 32B, FIG. 32C). Triple Th1 CD4+ T cells response two doses of Bivalent BA.5 Primary followed by XBB.1.5 Intramuscular Boost (1 μg rS) versus two doses of Bivalent BA.5 Primary followed by XBB.1.5 Intranasal Boost (1 μg rS) versus two doses of Bivalent BA.5 Primary followed by XBB.1.5 Intranasal Boost (5 μg rS) (FIG. 33), and Triple Th1 CD4+ T cells response two doses of XBB.1.5 Primary followed by XBB.1.5 Intramuscular Boost (1 μg rS) versus two doses of XBB.1.5 Primary followed by XBB.1.5 Intranasal Boost (1 μg rS) versus two doses of XBB.1.5 Primary followed by XBB.1.5 Intranasal Boost (5 μg rS) (FIG. 34). T follicular helper cells (Tfh) and germinal center B cells in response to XBB.1.5 Boost following primary Bivalent BA.5 or XBB.1.5 (FIG. 37A, FIG. 37B). Bronchoalveolar lavage CD4+ T cells Th1/Th2 responses to post XBB.1.5 intranasal boost following primary Bivalent BA.5 or XBB.1.5 (FIG. 38A, FIG. 38B). Triple Th1 CD4+ T cells in spleen cells in response to intranasal vaccination of XBB.1.5 rS (5 μg rS+5 μg Matrix-M) at day 21, day 41 and day 56 using Intracellular Cytokine Staining (ICCS) assay (FIG. 39). CD8+ T cells Th1 responses to Primary and Boost with XBB.1.5 (5 μg rS) by intranasal vaccination (FIG. 40).

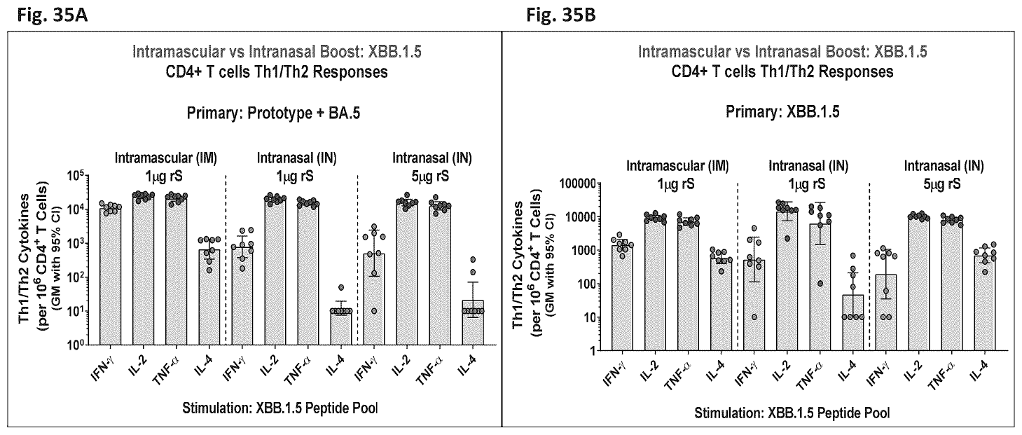

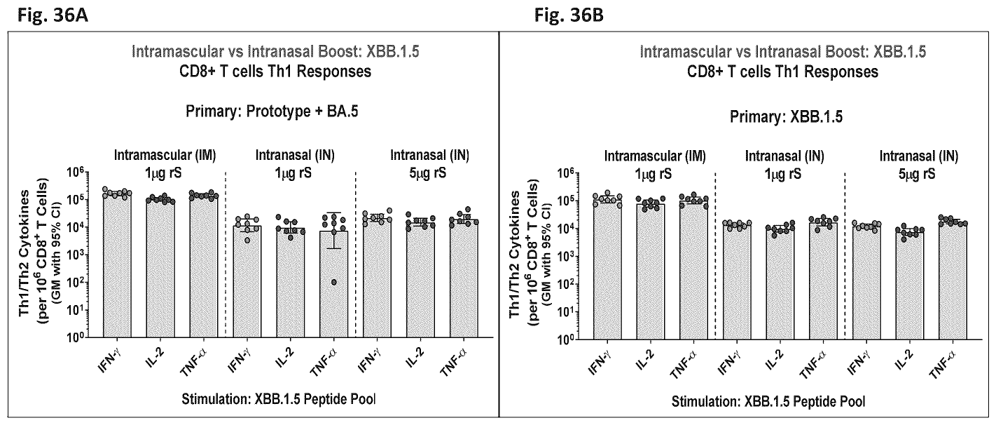

Peptide Pool Simulation: CD4+ T cells Th1/Th2 responses to intramuscular versus intranasal XBB.1.5 Boost following primary Prototype+BA.5 or XBB.1.5 (FIG. 35A, FIG. 35B), and CD8+ T cells Th1 responses to intramuscular versus intranasal XBB.1.5 Boost following primary Prototype+BA.5 or XBB.1.5 (FIG. 36A, FIG. 36B).

Collectively, this data shows that intranasal administration of the immunogenic compositions described herein induces both mucosal and systemic immunity, with increased immunogenic responses post Intranasal Boost.

### Example 5: Evaluation of Immunogenic Compositions in Rhesus Macaques

The ability of immunogenic compositions containing viral glycoprotein nanoparticles and optionally saponin adjuvant to induce an immune response when the compositions are delivered intranasally or intramuscularly is evaluated in Rhesus Macaques. The saponin adjuvant contained two iscom particles: (i) a first iscom particle containing Fraction A of Quillaja Saponaria Molina and not Fraction C of Quillaja Saponaria Molina, and (ii) a second iscom particle containing Fraction C of Quillaja Saponaria Molina and not Fraction A of Quillaja Saponaria Molina. Fraction A and Fraction C accounted for 85% and 15% by weight, respectively, of the sum of the weights of fraction A of Quillaja Saponaria Molina and fraction C of Quillaja Saponaria Molina in the adjuvant.

Exemplary compositions in the table below are evaluated.

| | | Primary Series | | | | | Boost 1 | | |
|---|---|---|---|---|---|---|---|---|---|
| | | Viral | | | | | Viral | | |
| | | glycoproteins | Amount | Amount | | | glycoproteins | Amount | Amount |
| | | in immunogenic | of Viral | of saponin | | Days | in immunogenic | of Viral | of saponin |
| Group | N | composition | glycoprotein | adjuvant | Route | Administered | composition | glycoprotein | adjuvant |
| 1 | 5 | SARS- | 5 μg | 50 μg | IM | Days 0 | SARS- | 1 μg | 5 μg |
| | | CoV-2 S | | | | and 21 | CoV-2 S | | |
| | | glycoprotein | | | | | glycoprotein | | |
| | | of SEQ ID NO: | | | | | of SEQ ID NO: | | |
| | | 87 (referred | | | | | 227 (referred | | |
| | | to as | | | | | to as “BQ.1.1”) | | |
| | | “prototype”) | | | | | | | |
| 2 | 5 | Prototype | 5 μg | 50 μg | IM | Days 0 | SARS- | 5 μg | 50 μg |
| | | | | | | and 21 | CoV-2 S | | |
| | | | | | | | glycoprotein | | |
| | | | | | | | of SEQ ID NO: | | |
| | | | | | | | 274 (referred | | |
| | | | | | | | to as “XBB.1.5”) | | |
| 3 | 5 | SARS- | 5 μg | 50 μg | IM | Days 0 | XBB.1.5 | 5 μg | 50 μg |
| | | CoV-2 S | | | | and 21 | | | |
| | | glycoprotein | | | | | | | |
| | | of SEQ ID NO: | | | | | | | |
| | | 222 (referred | | | | | | | |
| | | to as BA.5) | | | | | | | |
| 4 | 5 | Prototype | 5 μg | 50 μg | IM | Days 0 | XBB.1.5 | 5 μg | 50 μg |
| | | and BA.5 | | | | and 21 | | | |
| 5 | 8 | XBB.1.5 | 5 μg | 50 μg | IM | Days 0 | XBB.1.5 | 5 μg | 50 μg |
| | | | | | | and 21 | | | |
| 6 | 8 | XBB.1.5 | 5 μg | 50 μg | IM | Days 0 | XBB.1.5 | 25 μg | 50 μg |
| | | | | | | and 21 | | | |

| | | | Boost 2 | | | | |
|---|---|---|---|---|---|---|---|
| | | | Viral | | | | |
| | Boost 1 | | glycoproteins | Amount | Amount | | |
| | | Days | in immunogenic | of Viral | of saponin | | Days |
| Group | Route | Administered | composition | glycoprotein | adjuvant | Route | Administered |
| 1 | IM | Day 246 | RSV F | 240 μg | 50 μg | IN | 447 |
| | | | glycoprotein | | | | |
| | | | of SEQ ID NO: | | | | |
| | | | 337 | | | | |
| 2 | IM | Day 246 | RSV F | 60 μg | 50 μg | IM | 447 |
| | | | glycoprotein | | | | |
| | | | of SEQ ID NO: | | | | |
| | | | 337 | | | | |
| 3 | IM | Day 246 | Hemagglutinin | 25 μg | 75 μg | IN | 447 |
| | | | (HA) from four | XBB.1.5 | | | |
| | | | different | and 240 μg | | | |
| | | | influenza | of HA per | | | |
| | | | strains | strain | | | |
| | | | and XBB.1.5 | | | | |
| 4 | IM | Day 246 | Hemagglutinin | 5 μg | 75 μg | IM | 447 |
| | | | (HA) from four | XBB.1.5 | | | |
| | | | different | and 60 μg | | | |
| | | | influenza | of HA per | | | |
| | | | strains | strain | | | |
| | | | and XBB.1.5 | | | | |

| | | | |
|---|---|---|---|
| 5 | IM | Day 202 | To be determined |
| 6 | IN | Day 202 | To be determined |

The immunogenic compositions of Boost 2 for Groups 1 and 2 are formulated in a buffer containing 22 mM sodium phosphate, 150 mM NaCl, 1% w/w histidine, and 0.03% w/w PS80 at a pH of 6.8. The immunogenic compositions of Boost 2 for Groups 3 and 4 are formulated in a buffer containing 25 mM sodium phosphate, 225 mM NaCl, 500 mM arginine-HCl, 2.5% w/w trehalose, and 0.03% w/w PS80 at a pH of 7.3. The immunogenic compositions of Boost 1 for Groups 5 and 6 are formulated in a buffer containing 25 mM sodium phosphate, 300 mM NaCl, 0.01% w/w PS80 at a pH of 7.2.

Serum, nasal wash, PBMCs, the bronchoalveolar lavage (BAL) of the Rhesus macaques are collected. The amount of total IgG, IgA, and neutralizing antibodies in the serum and BAL are evaluated. The ratios of CD4+ to CD8+ T cells is also evaluated. Intracellular cytokine staining assays are performed to determine if CD4+ or CD8+ T cells secrete Type 1 cytokines (e.g., IFN-Y, TNF-α, IL-2) and/or Type 2 cytokines (CD4+ T cells). B cells are analyzed for germinal center formation (GL7+, CD95+).

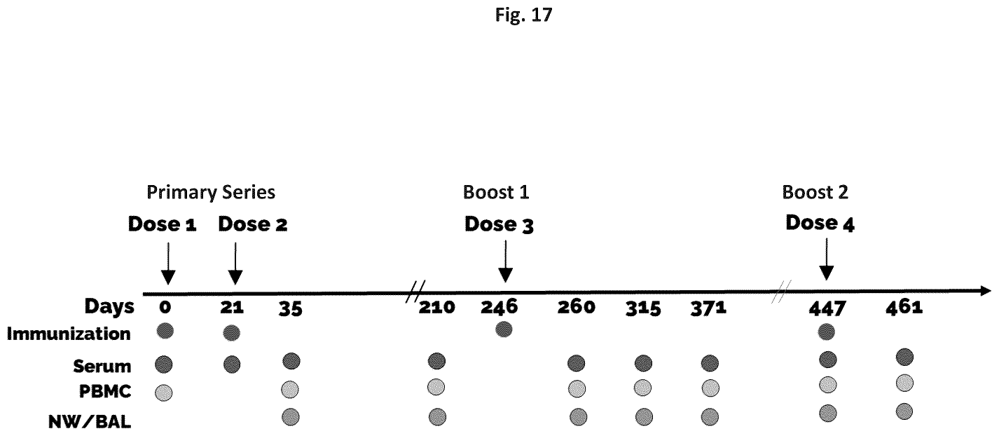

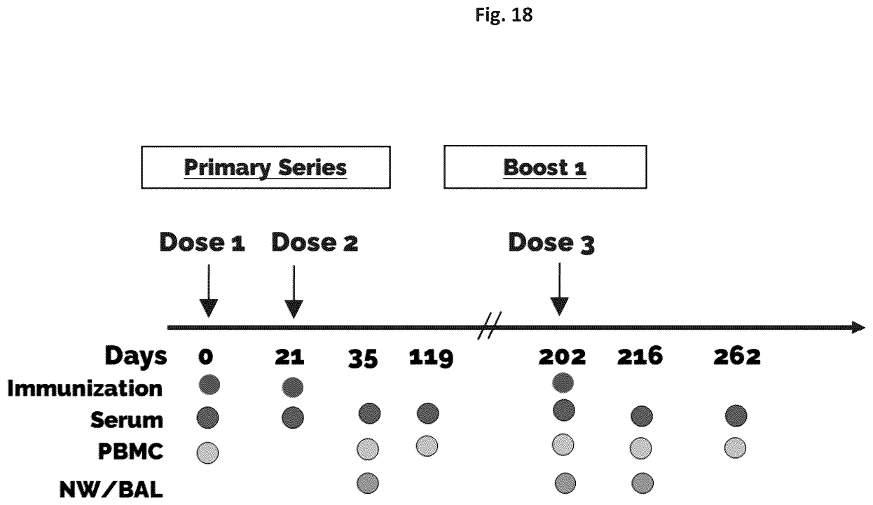

FIG. 17 is a timeline of the experiments for Groups 1˜4 and FIG. 18 is a timeline of the experiments for Groups 5-6.

Anti-SARS-COV-2 S glycoprotein antibodies, anti-RSV F glycoprotein antibodies, influenza hemagglutination inhibition (HAI) titer, and pseudovirus neutralization titers are measured before and after immunization. The level of IgA versus IgG antibodies is also quantitated. The cellular response to immunization is also evaluated. For example, the number of CD4+ Th1 and Th2 cells is quantitated.

### Enumerated Embodiments

1. An intranasal immunogenic composition, comprising:
   (i) a glycoprotein nanoparticle comprising a non-ionic detergent and a viral glycoprotein comprising a transmembrane domain, wherein the viral glycoprotein is anchored to the detergent core via the transmembrane domain of the viral glycoprotein; and
   (ii) a pharmaceutically acceptable buffer.
2. The intranasal immunogenic composition of enumerated embodiment 1, wherein the viral glycoprotein is selected from one or more of a hemagglutinin (HA) glycoprotein, a SARS-COV-2 Spike(S) glycoprotein, and a respiratory syncytial virus (RSV) fusion (F) glycoprotein.
3. The intranasal immunogenic composition of enumerated embodiment 1 or 2, comprising from 1 to about 20, from 1 to about 19, from 1 to about 18, from 1 to about 17, from 1 to about 16, from 1 to about 15, from 1 to about 14, from 1 to about 13, from 1 to about 12, from 1 to about 11, from 1 to about 10, from 1 to about 9, from 1 to about 8, from 1 to about 7, from 1 to about 6, from 1 to about 5, from 1 to about 4, from 1 to about 3, from 1 to about 2, from about 2 to about 20, from about 2 to about 19, from about 2 to about 18, from about 2 to about 17, from about 2 to about 16, from about 2 to about 15, from about 2 to about 14, from about 2 to about 13, from about 2 to about 12, from about 2 to about 11, from about 2 to about 10, from about 2 to about 9, from about 2 to about 8, from about 2 to about 7, from about 2 to about 6, from about 2 to about 5, from about 2 to about 4, from about 2 to about 3, from about 3 to about 20, from about 3 to about 19, from about 3 to about 18, from about 3 to about 17, from about 3 to about 16, from about 3 to about 15, from about 3 to about 14, from about 3 to about 13, from about 3 to about 12, from about 3 to about 11, from about 3 to about 10, from about 3 to about 9, from about 3 to about 8, from about 3 to about 7, from about 3 to about 6, from about 3 to about 5, from about 3 to about 4, from about 4 to about 20, from about 4 to about 19, from about 4 to about 18, from about 4 to about 17, from about 4 to about 16, from about 4 to about 15, from about 4 to about 14, from about 4 to about 13, from about 4 to about 12, from about 4 to about 11, from about 4 to about 10, from about 4 to about 9, from about 4 to about 8, from about 4 to about 7, from about 4 to about 6, from about 4 to about 5, from about 5 to about 20, from about 5 to about 19, from about 5 to about 18, from about 5 to about 17, from about 5 to about 16, from about 5 to about 15, from about 5 to about 14, from about 5 to about 13, from about 5 to about 12, from about 5 to about 11, from about 5 to about 10, from about 5 to about 9, from about 5 to about 8, from about 5 to about 7, from about 5 to about 6, from about 6 to about 20, from about 6 to about 19, from about 6 to about 18, from about 6 to about 17, from about 6 to about 16, from about 6 to about 15, from about 6 to about 14, from about 6 to about 13, from about 6 to about 12, from about 6 to about 11, from about 6 to about 10, from about 6 to about 9, from about 6 to about 8, from about 6 to about 7, from about 7 to about 20, from about 7 to about 19, from about 7 to about 18, from about 7 to about 17, from about 7 to about 16, from about 7 to about 15, from about 7 to about 14, from about 7 to about 13, from about 7 to about 12, from about 7 to about 11, from about 7 to about 10, from about 7 to about 9, from about 7 to about 8, from about 8 to about 20, from about 8 to about 19, from about 8 to about 18, from about 8 to about 17, from about 8 to about 16, from about 8 to about 15, from about 8 to about 14, from about 8 to about 13, from about 8 to about 12, from about 8 to about 11, from about 8 to about 10, from about 8 to about 9, from about 9 to about 20, from about 9 to about 19, from about 9 to about 18, from about 9 to about 17, from about 9 to about 16, from about 9 to about 15, from about 9 to about 14, from about 9 to about 13, from about 9 to about 12, from about 9 to about 11, from about 9 to about 10, from about 10 to about 20, from about 10 to about 19, from about 10 to about 18, from about 10 to about 17, from about 10 to about 16, from about 10 to about 15, from about 10 to about 14, from about 10 to about 13, from about 10 to about 12, from about 10 to about 11, from about 11 to about 20, from about 11 to about 19, from about 11 to about 18, from about 11 to about 17, from about 11 to about 16, from about 11 to about 15, from about 11 to about 14, from about 11 to about 13, from about 11 to about 12, from about 12 to about 20, from about 12 to about 19, from about 12 to about 18, from about 12 to about 17, from about 12 to about 16, from about 12 to about 15, from about 12 to about 14, from about 12 to about 13, from about 13 to about 20, from about 13 to about 19, from about 13 to about 18, from about 13 to about 17, from about 13 to about 16, from about 13 to about 15, from about 13 to about 14, from about 14 to about 20, from about 14 to about 19, from about 14 to about 18, from about 14 to about 17, from about 14 to about 16, from about 14 to about 15, from about 15 to about 20, from about 15 to about 19, from about 15 to about 18, from about 15 to about 17, from about 15 to about 16, from about 16 to about 20, from about 16 to about 19, from about 16 to about 18, from about 16 to about 17, from about 17 to about 20, from about 17 to about 19, from about 17 to about 18, from about 18 to about 20, from about 18 to about 19, or from about 19 to about 20 glycoprotein nanoparticles.
4. The intranasal immunogenic composition of enumerated embodiment 3, wherein each nanoparticle comprises a different viral glycoprotein.
5. The intranasal immunogenic composition of enumerated embodiment 3, wherein each nanoparticle comprises from 1 to about 5 different viral glycoproteins.
6. The intranasal immunogenic composition of any one of enumerated embodiments 1-3, comprising a nanoparticle comprising two different viral glycoproteins.
7. The intranasal immunogenic composition of any one of enumerated embodiments 1-3, comprising a nanoparticle comprising three different viral glycoproteins.
8. The intranasal immunogenic composition of any one of enumerated embodiments 1-3, comprising a nanoparticle comprising four different viral glycoproteins.
9. The intranasal immunogenic composition of any one of enumerated embodiments 1-8, wherein the viral glycoprotein is an RSV F glycoprotein.
10. The intranasal immunogenic composition of enumerated embodiment 9, wherein the RSV F glycoprotein comprises at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or at least 100% identity to a polypeptide of any one of SEQ ID NOS: 331-340.
11. The intranasal immunogenic composition of enumerated embodiment 9, wherein the RSV F glycoprotein comprises at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or at least 100% identity to a polypeptide of SEQ ID NO: 331.
12. The intranasal immunogenic composition of enumerated embodiment 9, wherein the RSV F glycoprotein comprises at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or at least 100% identity to a polypeptide of 337.
13. The intranasal immunogenic composition of any one of enumerated embodiments 9-12, comprising from 30 μg to about 300 μg of the RSV F glycoprotein.
14. The intranasal immunogenic composition of any one of enumerated embodiments 9-12, comprising about 60 μg of the RSV F glycoprotein.
15. The intranasal immunogenic composition of any one of enumerated embodiments 9-12, comprising about 240 μg of the RSV F glycoprotein.
16. The intranasal immunogenic composition of any one of enumerated embodiments 1-8, wherein the viral glycoprotein is a SARS-COV-2 S glycoprotein.
17. The intranasal immunogenic composition of enumerated embodiment 16, wherein the SARS-COV-2 S glycoprotein contains (i) an inactive furin cleavage site, and (ii) proline at amino acid positions 973 and 974, wherein the SARS-COV-2 glycoprotein is numbered according to the polypeptide of SEQ ID NO: 2.
18. The intranasal immunogenic composition of enumerated embodiments 17, wherein the inactive furin cleavage site has an amino acid sequence selected from any one of: 7-34, 97, and 111.
19. The intranasal immunogenic composition of enumerated embodiment 17, wherein the inactive furin cleavage site has the amino acid sequence of QQAQ (SEQ ID NO: 7).
20. The intranasal immunogenic composition of enumerated embodiment 16, wherein the SARS-COV-2 S glycoprotein has at least 90% identity to the SARS-COV-2 S glycoprotein of SEQ ID NO: 87.
21. The intranasal immunogenic composition of enumerated embodiment 16, wherein the SARS-COV-2 S glycoprotein has at least 90% identity to the SARS-COV-2 S glycoprotein of SEQ ID NO: 274.
22. The intranasal immunogenic composition of enumerated embodiment 14, wherein the SARS-COV-2 S glycoprotein has at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or at least 100% identity to a polypeptide of any one of SEQ ID NOS: 87, 89, 106, 112, 113, 114, 115, 132, 144, 151, 153, 156, 158, 174, 175, 176, 181, 182, 183, 184, 186, 188, 190, 192, 195, 217, 218, 219, 220, 221, 222, 223, 224, 225, 226, 227, 228, 233, 234, 235, 236, 260, 261, 262, 263, 264, 274, 276, 278, 280, 284, 288, 292, 296, 300, 304, 308, 312, 316, 320, 324, 363, and 328.
23. The intranasal immunogenic composition of any one of enumerated embodiments 16-22, comprising from about 1 μg to about 175 μg of the SARS-COV-2 S glycoprotein.
24. The intranasal immunogenic composition of any one of enumerated embodiments 16-22, comprising about 5 μg of the SARS-COV-2 S glycoprotein.
25. The intranasal immunogenic composition of any one of enumerated embodiments 16-22, comprising about 25 μg of the SARS-COV-2 S glycoprotein.
26. The intranasal immunogenic composition of any one of enumerated embodiments 1-8, wherein the viral glycoprotein is a HA glycoprotein.
27. The intranasal immunogenic composition of enumerated embodiment 26, wherein the amino acid sequence of the HA glycoprotein has 100% identity to the amino acid sequence of the native HA glycoprotein.
28. The intranasal immunogenic composition of enumerated embodiment 26 or 27, wherein the HA glycoprotein has a subtype selected from the group consisting of H1, H3, H4, H5, and H7.
29. The intranasal immunogenic composition of any one of enumerated embodiments 26-28, comprising a first, second, and third hemagglutinin (HA) glycoprotein, wherein each HA glycoprotein is from a different influenza strain.
30. The intranasal immunogenic composition of enumerated embodiment 29, comprising a fourth HA glycoprotein, wherein the fourth HA glycoprotein is from a different influenza strain than the first, second, and third HA glycoprotein.
31. The intranasal immunogenic composition of enumerated embodiment 29 or 30, comprising a detergent core nanoparticle comprising the first HA glycoprotein and a non-ionic detergent.
32. The intranasal immunogenic composition of enumerated embodiment 29 or 30, comprising a HaSMaN comprising the first HA glycoprotein.
33. The intranasal immunogenic composition of any one of enumerated embodiments 26-30, comprising:
   (i) a first detergent-core nanoparticle comprising the first HA glycoprotein and a non-ionic detergent;
   (i) a second detergent-core nanoparticle comprising the second HA glycoprotein and the non-ionic detergent; and
   (iii) a third detergent-core nanoparticle comprising the second HA glycoprotein and the non-ionic detergent.
34. The intranasal immunogenic composition of enumerated embodiment 33, comprising: (iv) a fourth detergent-core nanoparticle comprising a fourth HA glycoprotein and a non-ionic detergent.
35. The intranasal immunogenic composition of any one of enumerated embodiments 26-30, comprising:
   (i) a detergent-core nanoparticle comprising the first HA glycoprotein and a non-ionic detergent; and
   (ii) a HaSMaN comprising the second HA glycoprotein.
36. The intranasal immunogenic composition of any one of enumerated embodiments 26-30, comprising:
   (i) a detergent-core nanoparticle comprising the first HA glycoprotein and a non-ionic detergent;
   (ii) a HaSMaN comprising the second HA glycoprotein; and
   (iii) a second HaSMaN comprising the third HA glycoprotein.
37. The intranasal immunogenic composition of enumerated embodiment 30, comprising:
   (i) a detergent-core nanoparticle comprising the first HA glycoprotein and a non-ionic detergent;
   (ii) a HaSMaN comprising the second HA glycoprotein;
   (iii) a second HaSMaN comprising the third HA glycoprotein; and
   (iii) a third HaSMaN comprising the fourth HA glycoprotein.
38. The intranasal immunogenic composition of any one of enumerated embodiments 33-37, wherein the HA glycoproteins of the detergent-core nanoparticles is from a Type A influenza strain.
39. The intranasal immunogenic composition of any one of enumerated embodiments 33-37, wherein the HA glycoproteins of the detergent-core nanoparticles is from a Type B influenza strain.
40. The intranasal immunogenic composition of any one of enumerated embodiments 35-37, wherein the HA glycoproteins of the HaSMaNs are from a Type A influenza strain.
41. The intranasal immunogenic composition of any one of enumerated embodiments 26-40, comprising from about 30 μg to about 300 μg of each HA glycoprotein.
42. The intranasal immunogenic composition of any one of enumerated embodiments 26-40, comprising about 60 μg of each HA glycoprotein.
43. The intranasal immunogenic composition of any one of enumerated embodiments 26-40, comprising about 240 μg of each HA glycoprotein.
44. The intranasal immunogenic composition of any one of enumerated embodiments 1-43, comprising from 1 to about 10 SARS-COV-2 S glycoproteins and from 1 to about 5 HA glycoproteins.
45. The intranasal immunogenic composition of any one of enumerated embodiments 1-43, comprising from 1 to about 10 RSV F glycoproteins and from 1 to about 5 HA glycoproteins.
46. The intranasal immunogenic composition of any one of enumerated embodiments 1-43, comprising from 1 to about 10 SARS-COV-2 S glycoproteins and from 1 to about 5 RSV F glycoproteins.
47. The intranasal immunogenic composition of any one of enumerated embodiments 1-43, comprising from 1 to about 10 SARS-COV-2 S glycoproteins, from 1 to about 5 HA glycoproteins, and from 1 to about 5 RSV F glycoproteins.
48. The intranasal immunogenic composition of enumerated embodiment 44 or enumerated embodiment 47, comprising from about 5 μg to about 60 μg of each HA glycoprotein and from about 2.5 μg to about 35 μg of SARS-COV-2 S glycoprotein.
49. The intranasal immunogenic composition of enumerated embodiment 48, comprising from about 2.5 μg to about 22.5 μg of SARS-COV-2 S glycoprotein.
50. The intranasal immunogenic composition of enumerated embodiment 48, comprising from about 15 μg to about 35 μg of SARS-COV-2 S glycoprotein.
51. The intranasal immunogenic composition of enumerated embodiment 48, comprising from about 30 μg to about 60 μg of each HA glycoprotein.
52. The intranasal immunogenic composition of enumerated embodiment 48, comprising from about 24 μg to about 40 μg of each HA glycoprotein and from about 5 μg to about 50 μg of SARS-CoV-2 S glycoprotein.
53. The intranasal immunogenic composition of enumerated embodiment 48, comprising about 33 μg of each HA glycoprotein and about 25 μg of SARS-COV-2 S glycoprotein.
54. The intranasal immunogenic composition of enumerated embodiment 48, comprising about 54 μg of each HA glycoprotein and about 25 μg of SARS-COV-2 S glycoprotein.
55. The intranasal immunogenic composition of enumerated embodiment 48, comprising about 39 μg of each HA glycoprotein and about 13 μg of SARS-COV-2 S glycoprotein.
56. The intranasal immunogenic composition of enumerated embodiment 48, comprising from about 30 μg to about 40 μg of each HA glycoprotein and about 20 μg of SARS-COV-2 S glycoprotein.
57. The intranasal immunogenic composition of enumerated embodiment 48, comprising from about 33 μg to about 39 μg of each HA glycoprotein and from about 14 μg to about 15 μg of SARS-COV-2 S glycoprotein.
58. The intranasal immunogenic composition of enumerated embodiment 48, comprising about 30 μg of each HA glycoprotein and about 25 μg of SARS-COV-2 S glycoprotein.
59. The intranasal immunogenic composition of enumerated embodiment 48, comprising about 60 μg of each HA glycoprotein and about 35 μg of SARS-COV-2 S glycoprotein.
60. The intranasal immunogenic composition of enumerated embodiment 48, comprising about 60 μg of each HA glycoprotein and about 5 μg of SARS-COV-2 S glycoprotein.
61. The intranasal immunogenic composition of enumerated embodiment 48, comprising about 240 μg of each HA glycoprotein and about 25 μg of SARS-COV-2 S glycoprotein.
62. The intranasal immunogenic composition of any one of enumerated embodiments 45-61, comprising from about 30 μg to about 300 μg of RSV F glycoprotein.
63. The intranasal immunogenic composition of enumerated embodiment 62, comprising about 60 μg of RSV F glycoprotein.
64. The intranasal immunogenic composition of enumerated embodiment 62, comprising about 240 μg of RSV F glycoprotein.
65. The intranasal immunogenic composition of any one of enumerated embodiments 1-64, wherein the pharmaceutically acceptable buffer comprises one or more of sodium phosphate and sodium chloride.
66. The intranasal immunogenic composition of enumerated embodiment 65, wherein the pharmaceutically acceptable buffer comprises from about 15 mM to about 50 mM sodium phosphate.
67. The intranasal immunogenic composition of enumerated embodiment 65 or 66, wherein the pharmaceutically acceptable buffer comprises from about 150 to about 250 mM sodium chloride.
68. The intranasal immunogenic composition of any one of enumerated embodiments 1-66, wherein the pharmaceutically acceptable buffer comprises about 25 mM sodium phosphate and about 0.3 M NaCl.
69. The intranasal immunogenic composition of any one of enumerated embodiments 1-68, wherein the non-ionic detergent is selected from the group consisting of polysorbate-20 (PS20), polysorbate-40 (PS40), polysorbate-60 (PS60), polysorbate-65 (PS65), and polysorbate-80 (PS80).
70. The intranasal immunogenic composition of enumerated embodiment 69, wherein the non-ionic detergent is PS80.
71. The intranasal immunogenic composition of enumerated embodiment 70, comprising about 0.03% PS80 by weight.
72. The intranasal immunogenic composition of enumerated embodiment 71, comprising from about 0.01% to about 0.5% PS80 by weight.
73. The intranasal immunogenic composition of any one of enumerated embodiments 1-72, comprising an adjuvant.
74. The intranasal immunogenic composition of enumerated embodiment 73, wherein the adjuvant is a saponin adjuvant.
75. The intranasal immunogenic composition of enumerated embodiment 74, wherein the saponin adjuvant comprises:
   (i) a first iscom particle comprising fraction A of Quillaja Saponaria Molina and not fraction C of Quillaja Saponaria Molina; and
   (ii) a second iscom particle comprising fraction C of Quillaja Saponaria Molina and not fraction A of Quillaja Saponaria Molina.
76. The intranasal immunogenic composition of enumerated embodiment 75, wherein fraction A of Quillaja Saponaria Molina and fraction C of Quillaja Saponaria Molina account for about 85% by weight and about 15% by weight, respectively, of the sum of the weights of fraction A of Quillaja Saponaria Molina and fraction C of Quillaja Saponaria Molina in the saponin adjuvant.
77. The intranasal immunogenic composition of enumerated embodiment 75, wherein fraction A of Quillaja Saponaria Molina accounts for at least about 70% by weight of the sum of the weights of Fraction A of Quillaja Saponaria Molina and fraction C of Quillaja Saponaria Molina in the saponin adjuvant, and wherein fraction C accounts for the remainder.
78. The intranasal immunogenic composition of any one of enumerated embodiments 1-77, comprising a volume of about 0.05 mL to about 1 mL.
79. The intranasal immunogenic composition of any one of enumerated embodiments 1-78, comprising a volume of about 0.2 mL.
80. The intranasal immunogenic composition of any one of enumerated embodiments 1-78, comprising a volume of about 0.5 mL.
81. The intranasal immunogenic composition of any one of enumerated embodiments 1-78, comprising a volume of about 0.25 mL 82. The intranasal immunogenic composition of any one of enumerated embodiments 73-81, comprising from about 50 μg to about 200 μg, from about 50 μg to about 150 μg, from about 50 μg to about 100 μg, or from about 50 μg to about 75 μg adjuvant.
83. The intranasal immunogenic composition of any one of enumerated embodiments 73-81, comprising about 50 μg adjuvant.
84. The intranasal immunogenic composition of any one of enumerated embodiments 73-81, comprising about 75 μg adjuvant.
85. The intranasal immunogenic composition of any one of enumerated embodiments 73-81, comprising about 100 μg adjuvant.
86. The intranasal immunogenic composition of any one of enumerated embodiments 1-85, wherein the total concentration of viral glycoproteins in the composition is from about 10 μg/mL to about 4 mg/mL.
87. The intranasal immunogenic composition of any one of enumerated embodiments 1-85, wherein the total concentration of viral glycoproteins in the composition is from about 10 μg/mL to about 50 μg/mL.
88. The intranasal immunogenic composition of any one of enumerated embodiments 1-85, wherein the total concentration of viral glycoproteins in the composition is from about 120 μg/mL to about 600 μg/mL.
89. The intranasal immunogenic composition of any one of enumerated embodiments 1-85, wherein the total concentration of viral glycoproteins in the composition is from about 600 μg/mL to about 3 mg/mL.
90. The intranasal immunogenic composition of any one of enumerated embodiments 1-89, wherein the composition is atomized.
91. The intranasal immunogenic composition of any one of enumerated embodiments 1-89, wherein the composition is aerosolized.
92. A prefilled syringe comprising the intranasal immunogenic composition of any one of enumerated embodiments 1-91.
93. A dry powder composition comprising a dried intranasal immunogenic composition of any one of enumerated embodiments 1-91.
94. An aerosol comprising an intranasal immunogenic composition of any one of enumerated embodiments 1-91.
95. An intranasal delivery device comprising the dry powder composition of enumerated embodiment 91.
96. An intranasal delivery device comprising the aerosol of enumerated embodiment 94.
97. The intranasal delivery device of enumerated embodiment 96, wherein the aerosol is a dry powder.
98. The intranasal delivery device of enumerated embodiment 96, wherein the aerosol is a liquid.
99. An intranasal delivery device comprising the intranasal immunogenic composition of enumerated embodiment 90.
100. An intranasal delivery device comprising a suspension comprising an intranasal immunogenic composition of any one of enumerated embodiments 1-91.
101. A method of stimulating an immune response against SARS-COV-2, influenza, RSV, or a combination thereof in a patient comprising intranasally administering the intranasal immunogenic composition of any one of enumerated embodiments 1-91 to a patient.
102. A method of inducing mucosal immunity against SARS-COV-2, influenza, RSV, or a combination thereof in a patient comprising intranasally administering the intranasal immunogenic composition of any one of enumerated embodiments 1-91.
103. A method of inducing production of neutralizing antibodies against SARS-COV-2, influenza, RSV, or a combination thereof in a patient comprising administering to the patient the intranasal immunogenic composition of any one of enumerated embodiments 1-91.
104. A method of stimulating an immune response against SARS-COV-2, influenza, RSV, or a combination thereof in the nasal cavity of a patient, comprising administering the intranasal immunogenic composition of any one of enumerated embodiments 1-91.
105. The method of any one of enumerated embodiments 101-104, wherein the method induces the production of neutralizing antibodies against SARS-COV-2, influenza, RSV, or a combination thereof in the bronchoalveolar lavage of the subject.
106. The method of enumerated embodiment 103 or 105, wherein the neutralizing antibodies are IgA antibodies.
107. The method of enumerated embodiment 103 or 105, wherein the neutralizing antibodies are IgG antibodies.
108. The method of any one of enumerated embodiments 101-107, wherein the patient has previously been administered an immunogenic composition intramuscularly.
109. The method of any one of enumerated embodiments 101-108, comprising administering 0.25 mL of the intranasal immunogenic composition to each nostril of the patient.
110. The method of any one of enumerated embodiments 101-109, wherein the composition is administered as a mist.

What is claimed:

1. An intranasal immunogenic composition, comprising:
(i) a glycoprotein nanoparticle comprising a non-ionic detergent core and a viral glycoprotein comprising a transmembrane domain, wherein the viral glycoprotein is anchored to the detergent core via the transmembrane domain of the viral glycoprotein; and
(ii) a pharmaceutically acceptable buffer.

2. The intranasal immunogenic composition of claim 1, wherein the viral glycoprotein is selected from one or more of a hemagglutinin (HA) glycoprotein, a SARS-COV-2 Spike(S) glycoprotein, and a respiratory syncytial virus (RSV) fusion (F) glycoprotein.

3. The intranasal immunogenic composition of claim 1, wherein the glycoprotein nanoparticle comprises two different viral glycoproteins.

4. The intranasal immunogenic composition of claim 1, wherein the viral glycoprotein comprises an RSV F glycoprotein having at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or at least 100% identity to a polypeptide of any one of SEQ ID NOS: 331-340.

5. The intranasal immunogenic composition of claim 4, comprising from 30 μg to about 300 μg of the RSV F glycoprotein.

6. The intranasal immunogenic composition of claim 1, wherein the viral glycoprotein comprises a SARS-COV-2 S glycoprotein containing (i) an inactive furin cleavage site, and (ii) proline at amino acid positions 973 and 974, wherein the SARS-COV-2 glycoprotein is numbered according to the polypeptide of SEQ ID NO: 2.

7. The intranasal immunogenic composition of claim 6, wherein the SARS-COV-2 S glycoprotein has at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or at least 100% identity to a polypeptide of any one of SEQ ID NOS: 87, 89, 106, 112, 113, 114, 115, 132, 144, 151, 153, 156, 158, 174, 175, 176, 181, 182, 183, 184, 186, 188, 190, 192, 195, 217, 218, 219, 220, 221, 222, 223, 224, 225, 226, 227, 228, 233, 234, 235, 236, 260, 261, 262, 263, 264, 274, 276, 278, 280, 284, 288, 292, 296, 300, 304, 308, 312, 316, 320, 324, 363 and 328.

8. The intranasal immunogenic composition of claim 6, comprising from about 1 μg to about 175 μg of the SARS-COV-2 S glycoprotein.

9. The intranasal immunogenic composition of claim 1, wherein the viral glycoprotein is a HA glycoprotein having a subtype selected from the group consisting of H1, H3, H4, H5, and H7.

10. The intranasal immunogenic composition of claim 9, wherein the HA glycoproteins of the detergent-core nanoparticles are from a Type A influenza strain or a Type B influenza strain.

11. The intranasal immunogenic composition of claim 9, wherein each HA glycoprotein present in the composition is present in an amount from about 30 μg to about 300 μg.

12. The intranasal immunogenic composition of claim 1, comprising between 1 to 10 SARS-CoV-2 S glycoproteins and from between 1 and 5 HA glycoproteins.

13. The intranasal immunogenic composition of claim 1, comprising between 1 to and 10 RSV F glycoproteins and between 1 and 5 HA glycoproteins.

14. The intranasal immunogenic composition of claim 1, comprising between 1 to 10 SARS-CoV-2 S glycoproteins and from 1 to 5 RSV F glycoproteins.

15. The intranasal immunogenic composition of claim 1, wherein the pharmaceutically acceptable buffer comprises one or more of sodium phosphate and sodium chloride.

16. The intranasal immunogenic composition of any claim 1, wherein the non-ionic detergent is selected from the group consisting of polysorbate-20 (PS20), polysorbate-40 (PS40), polysorbate-60 (PS60), polysorbate-65 (PS65), and polysorbate-80 (PS80).

17. The intranasal immunogenic composition of claim 1, comprising a saponin-based adjuvant comprising:
(i) a first iscom particle comprising fraction A of Quillaja Saponaria Molina and not fraction C of Quillaja Saponaria Molina; and
(ii) a second iscom particle comprising fraction C of Quillaja Saponaria Molina and not fraction A of Quillaja Saponaria Molina.

18. A dry powder composition comprising a dried intranasal immunogenic composition of claim 1.

19. An aerosol comprising the intranasal immunogenic composition of claim 1.

20. A method of stimulating an immune response against SARS-COV-2, influenza, RSV, or a combination thereof in a patient, comprising intranasally administering the intranasal immunogenic composition of claim 1 to the patient.
